(19) United States
(12) Patent Application Publication
(10) Pub. No.: US 2026/0130046 A1
(43) Pub. Date: May 7, 2026

(54) ORGANIC COMPOUND, ORGANIC SEMICONDUCTOR DEVICE, AND LIGHT-EMITTING DEVICE

(71) Applicant: SEMICONDUCTOR ENERGY LABORATORY CO., LTD., Atsugi-shi (JP)

(72) Inventors: Kazuki KAJIYAMA, Hadano (JP); Naoaki HASHIMOTO, Sagamihara (JP); Sachiko KAWAKAMI, Atsugi (JP); Tsunenori SUZUKI, Yokohama (JP); Tomohiro KUBOTA, Atsugi (JP)

(21) Appl. No.: 19/364,614

(22) Filed: Oct. 21, 2025

(30) Foreign Application Priority Data
Nov. 1, 2024 (JP) 2024-193065

(51) Int. Cl. H10K 50/11; C07D 307/87; C09K 11/06; H10K 50/19; H10K 85/30; H10K 85/60; H10K 101/00; H10K 101/25

(52) U.S. Cl. CPC H10K 50/11; C07D 307/87; C09K 11/06; H10K 50/19; H10K 85/346; H10K 85/626; H10K 85/633; H10K 85/636; H10K 85/654; H10K 85/656; H10K 85/6572; H10K 85/6574; H10K 85/6576; C09K 2211/1088; H10K 2101/25; H10K 2101/90

(57) ABSTRACT

An organic compound having high heat resistance is provided. The organic compound is represented by General Formula (G1). In General Formula (G1), α¹ represents a substituted or unsubstituted phenylene group; n is 1 or 2; α² represents a substituted or unsubstituted phenylene group or a substituted or unsubstituted naphthalene-diyl group; m is 0, 1, or 2; Ar² represents a group represented by General Formula (Ar²-a) or General Formula (Ar²-b); any one of R⁸ to R¹⁷ represents a bond; and X represents oxygen or sulfur.

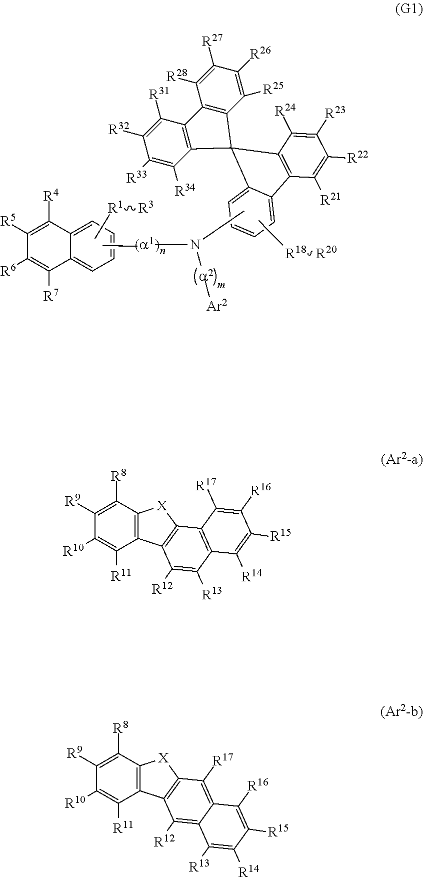

### BACKGROUND OF THE INVENTION

### 1. Field of the Invention

One embodiment of the present invention relates to an organic compound, an organic semiconductor device, a light-emitting device, a photoelectric conversion device, a light-emitting apparatus, a light-receiving apparatus, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor apparatus, a display device, a liquid crystal display apparatus, a light-emitting apparatus, a lighting device, a power storage device, a memory device, an imaging device, a driving method thereof, and a manufacturing method thereof.

### 2. Description of the Related Art

In recent years, organic semiconductor devices have been expected to be applied to a variety of uses. Specific examples of the organic semiconductor devices include a light-emitting device such as an organic light-emitting diode (OLED), a photoelectric conversion device such as an organic optical sensor or an organic thin film solar cell, and an organic field-effect transistor. Among them, light-emitting devices utilizing electroluminescence (hereinafter referred to as EL) have features such as ease of reduction in thickness and weight, high-speed response to input signals, and driving with a constant DC voltage power source, and thus have been used in display devices.

In improving device characteristics of the organic semiconductor device, there are many problems which depend on a substance contained in the organic semiconductor device, such as an organic compound, a metal, and a metal compound. In order to solve the problems, improvement of a device structure, development of a substance, and the like have been carried out. For example, Patent Document 1 discloses a hole-transport material, a kind of organic compound that can increase emission efficiency of a light-emitting device, a kind of organic semiconductor device, when used for the light-emitting device.

### REFERENCE

[Patent Document 1]Japanese Published Patent Application No. 2009-298767

### SUMMARY OF THE INVENTION

An object of one embodiment of the present invention is to provide a novel organic compound. Another object of one embodiment of the present invention is to provide a novel carrier-transport material. Another object of one embodiment of the present invention is to provide a novel hole-transport material. Another object of one embodiment of the present invention is to provide a highly heat-resistant carrier-transport material or hole-transport material.

Another object of one embodiment of the present invention is to provide an organic semiconductor device with small change in driving voltage over driving time. Another object of one embodiment of the present invention is to provide an organic semiconductor device having a long driving lifetime. Another object of one embodiment of the present invention is to provide an organic semiconductor device, a light-emitting apparatus, an electronic appliance, a display device, and an electronic device each having low power consumption.

Note that the description of these objects does not preclude the existence of other objects. One embodiment of the present invention does not need to achieve all of these objects. Other objects will be apparent from and can be derived from the description of the specification, the drawings, the claims, and the like.

One embodiment of the present invention is an organic compound represented by General Formula (G1).

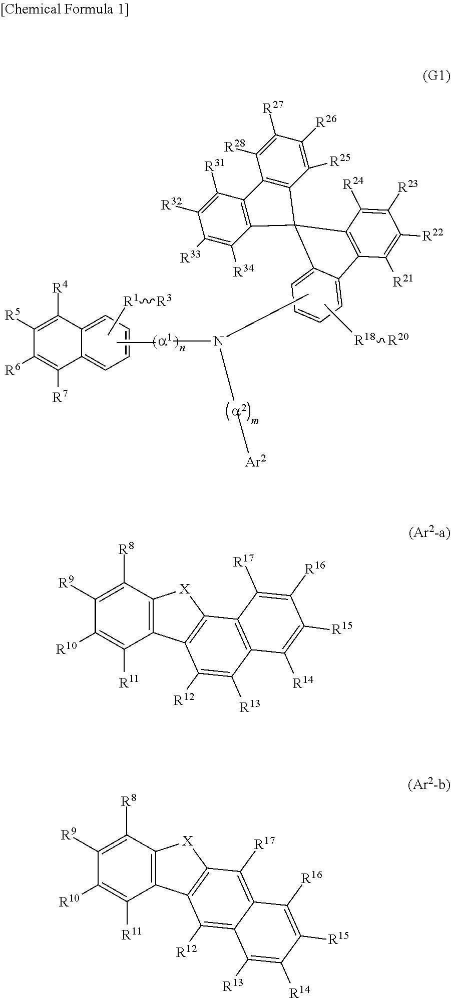

In General Formula (G1), α¹ represents a substituted or unsubstituted phenylene group; n is 1 or 2; α² represents a substituted or unsubstituted phenylene group or a substituted or unsubstituted naphthalene-diyl group; m is 0, 1, or 2; each of R¹ to R⁷ independently represents hydrogen (including deuterium), a straight-chain or branched-chain alkyl group having 1 to 6 carbon atoms, a substituted or unsubstituted cycloalkyl group having 3 to 10 carbon atoms, or a substituted or unsubstituted phenyl group; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When n is 2, a plurality of α¹'s may be the same or different from each other. When m is 2, a plurality of α²'s may be the same or different from each other.

Another embodiment of the present invention is an organic compound represented by General Formula (G2).

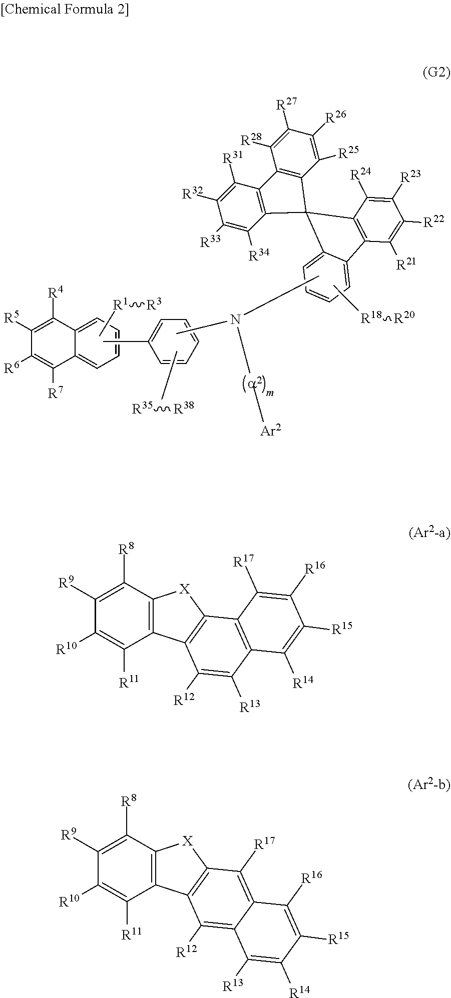

In General Formula (G2), α² represents a substituted or unsubstituted phenylene group or a substituted or unsubstituted naphthalene-diyl group; m is 0, 1, or 2; each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When m is 2, a plurality of α²'s may be the same or different from each other.

Another embodiment of the present invention is the organic compound represented by General Formula (G2) where m is 0.

Another embodiment of the present invention is an organic compound represented by General Formula (G4).

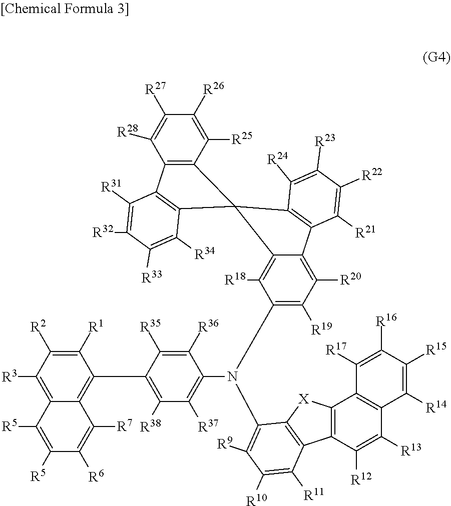

In General Formula (G4), each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; each of R⁹ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur.

Another embodiment of the present invention is an organic compound represented by General Formula (G5).

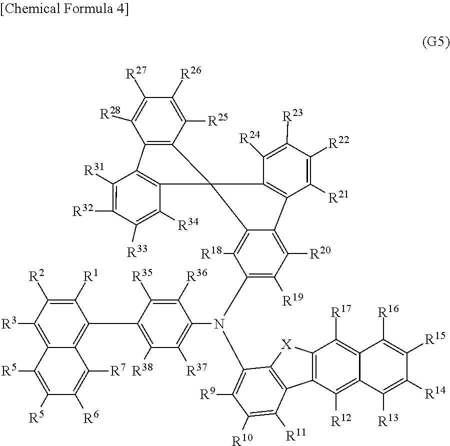

In General Formula (G5), each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; each of R⁹ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur.

Another embodiment of the present invention is an organic compound represented by General Formula (G7).

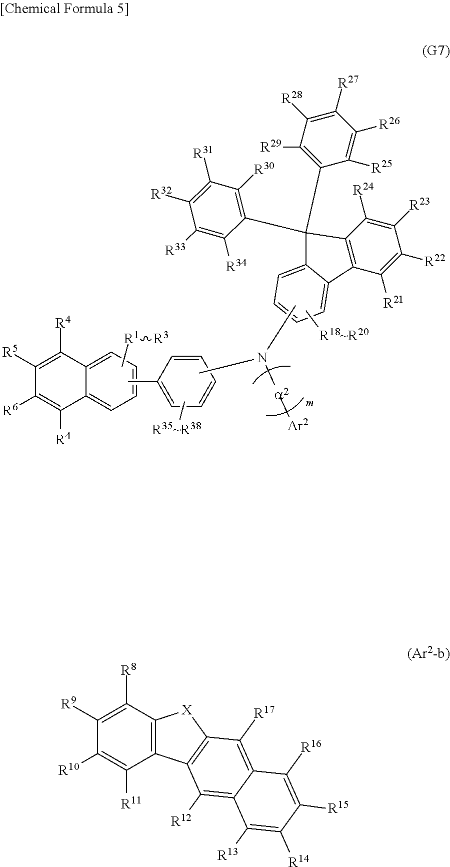

In General Formula (G7), α² represents a substituted or unsubstituted phenylene group or a substituted or unsubstituted naphthalene-diyl group; m is 0, 1, or 2; each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; Ar² represents a group represented by General Formula (Ar²-b); any one of R⁸ to R¹⁷ represents a bond; each of R⁸ to R¹⁷ other than the bond and each of R¹⁸ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When m is 2, a plurality of α²'s may be the same or different from each other.

Another embodiment of the present invention is the organic compound represented by General Formula (G7) where m is 0.

Another embodiment of the present invention is an organic compound represented by Structural Formula (100) or Structural Formula (101).

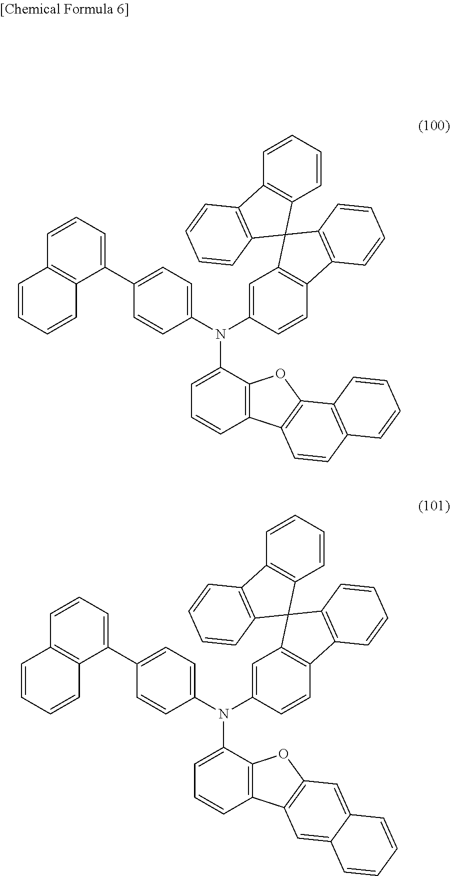 [SMILES: c1ccc2c(c1)-c1ccccc1C21c2ccccc2-c2ccc(N(c3ccc(-c4cccc5ccccc45)cc3)c3cccc4c3oc3c5ccccc5ccc43)cc21 ; c1ccc2c(c1)-c1ccccc1C21c2ccccc2-c2ccc(N(c3ccc(-c4cccc5ccccc45)cc3)c3cccc4c3oc3cc5ccccc5cc34)cc21]

Another embodiment of the present invention is an organic semiconductor device including the organic compound having any of the above structures.

Another embodiment of the present invention is a light-emitting device the organic compound having any of the above structures.

Another embodiment of the present invention is a light-emitting device including a first electrode, a second electrode, a light-emitting layer, and a first layer in which the light-emitting layer is positioned between the first electrode and the second electrode, the first layer is positioned between the first electrode and the light-emitting layer, and the first layer contains an organic compound represented by General Formula (G8).

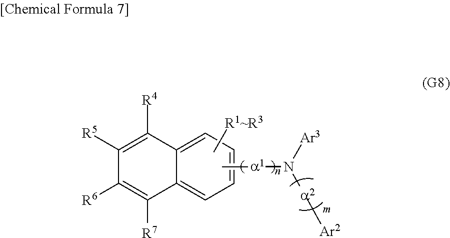

In General Formula (G8), each of α¹ and α² independently represents a substituted or unsubstituted phenylene group or a substituted or unsubstituted naphthalene-diyl group; n is 1 or 2; m is 0, 1, or 2; each of R¹ to R⁷ independently represents hydrogen (including deuterium), a straight-chain or branched-chain alkyl group having 1 to 6 carbon atoms, a substituted or unsubstituted cycloalkyl group having 3 to 10 carbon atoms, or a substituted or unsubstituted phenyl group; Ar² represents a substituted or unsubstituted benzo[b]naphtho[2,1-d]furanyl group, a substituted or unsubstituted benzo[b]naphtho[2,3-d]furanyl group, a substituted or unsubstituted benzo[b]naphtho[2,1-d]thiophenyl group, or a substituted or unsubstituted benzo[b]naphtho[2,3-d]thiophenyl group; and Ar³ represents a substituted or unsubstituted fluorenyl group or a substituted or unsubstituted spirobifluorenyl group. When n is 2, a plurality of α¹'s may be the same or different from each other. When m is 2, a plurality of α²'s may be the same or different from each other.

Another embodiment of the present invention is a light-emitting device including a first electrode, a second electrode, a light-emitting layer, and a first layer in which the light-emitting layer is positioned between the first electrode and the second electrode, the first layer is positioned between the first electrode and the light-emitting layer, and the first layer contains an organic compound represented by General Formula (G9).

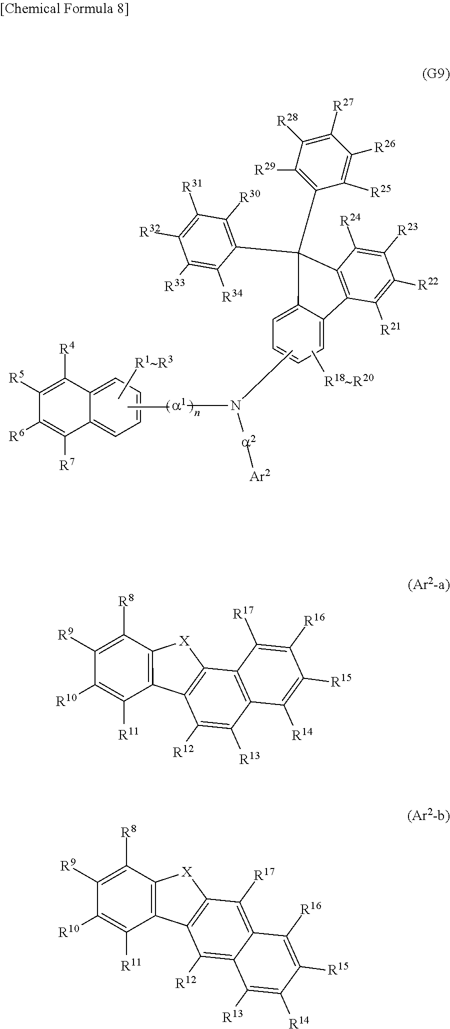

In General Formula (G9), each of α¹ and α² independently represents a substituted or unsubstituted phenylene group or a substituted or unsubstituted naphthalene-diyl group; n is 1 or 2; m is 0, 1, or 2; each of R¹ to R⁷ independently represents hydrogen (including deuterium), a straight-chain or branched-chain alkyl group having 1 to 6 carbon atoms, a substituted or unsubstituted cycloalkyl group having 3 to 10 carbon atoms, or a substituted or unsubstituted phenyl group; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; when each of R²⁹ and R³⁰ represents a bond, R²⁹ and R³⁰ are bonded to each other to form a ring; when neither R²⁹ nor R³⁰ represents a bond, each of R²⁹ and R³⁰ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When n is 2, a plurality of α¹'s may be the same or different from each other. When m is 2, a plurality of α²'s may be the same or different from each other.

In the light-emitting having any of the above structures, the first layer is preferably in contact with the light-emitting layer.

In the light-emitting having any of the above structures, it is preferable that the first layer be in contact with the light-emitting layer, the light-emitting layer include a first host material, a second host material, and a light-emitting substance, the first host material and the second host material form an exciplex in combination, and the difference between the peak wavelength of the emission spectrum of the exciplex and the peak wavelength of the emission spectrum of the light-emitting substance be less than or equal to 30 nm.

In the light-emitting having any of the above structures, it is further preferable that the first layer be in contact with the light-emitting layer and the light-emitting layer include a host material and a fluorescent substance.

According to one embodiment of the present invention, a novel organic compound can be provided. According to one embodiment of the present invention, a novel carrier-transport material can be provided. According to one embodiment of the present invention, a novel hole-transport material can be provided. According to one embodiment of the present invention, a highly heat-resistant carrier-transport material or hole-transport material can be provided.

According to another embodiment of the present invention, an organic semiconductor device with small change in driving voltage over driving time can be provided. According to another embodiment of the present invention, an organic semiconductor device having a long driving lifetime can be provided. According to another embodiment of the present invention, an organic semiconductor device, a light-emitting apparatus, an electronic appliance, a display device, and an electronic device each having low power consumption.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1C each illustrate a structure of an organic semiconductor device of an embodiment.

FIGS. 2A and 2B each illustrate a structure of a device of an embodiment.

FIGS. 3A to 3F each illustrate a structure of a light-emitting device of an embodiment.

FIGS. 4A and 4B are a top view and a cross-sectional view, respectively, of a display device.

FIGS. 5A and 5B are perspective views illustrating a structure example of a display module.

FIGS. 6A and 6B are cross-sectional views illustrating a structure example of a display device.

FIG. 7 is a perspective view illustrating a structure example of a display device.

FIG. 8A is a cross-sectional view illustrating a structure example of a display device.

FIGS. 8B and 8C are cross-sectional views illustrating a structure example of a transistor.

FIG. 9 is a cross-sectional view illustrating a structure example of a display device.

FIGS. 10A to 10C are a cross-sectional view and top views illustrating a structure example of a display device.

FIGS. 11A to 11D are cross-sectional views illustrating structure examples of a display device.

FIGS. 12A to 12C are a cross-sectional view and top views illustrating a structure example of a display device.

FIGS. 13A to 13D illustrate examples of electronic appliances.

FIGS. 14A to 14F illustrate examples of electronic appliances.

FIGS. 15A to 15G illustrate examples of electronic appliances.

FIG. 16 illustrates a structure of a light-emitting device of an example.

FIG. 17 shows a ¹H NMR spectrum of SFNBaBnf(10).

FIG. 18 shows an absorption spectrum and an emission spectrum of a toluene solution of SFNBaBnf(10).

FIG. 19 shows an absorption spectrum and an emission spectrum of a thin film of SFNBaBnf(10).

FIGS. 20A and 20B show a phosphorescence spectrum of SFNBaBnf(10).

FIG. 21 shows a ¹H NMR spectrum of SFNBBnf(II)(4).

FIG. 22 shows an absorption spectrum and an emission spectrum of a toluene solution of SFNBBnf(II)(4).

FIG. 23 shows an absorption spectrum and an emission spectrum of a thin film of SFNBBnf(II)(4).

FIGS. 24A and 24B show a phosphorescence spectrum of SFNBBnf(II)(4).

FIG. 25 shows emission spectra of a film of 8mpTP-4mDBtPBfpm, a film of βNCCP, and a mixed film of 8mpTP-4mDBtPBfpm and βNCCP.

FIG. 26 shows an absorption spectrum and an emission spectrum of Ir(5mppy-d₃)₂(mbfpypy-d₃).

FIG. 27 shows luminance-current density characteristics of a light-emitting device 1, a light-emitting device 2, a comparative light-emitting device 3, and a comparative light-emitting device 4.

FIG. 28 shows luminance-voltage characteristics of the light-emitting devices 1 and 2 and the comparative light-emitting devices 3 and 4.

FIG. 29 shows current efficiency-luminance characteristics of the light-emitting devices 1 and 2 and the comparative light-emitting devices 3 and 4.

FIG. 30 shows current density-voltage characteristics of the light-emitting devices 1 and 2 and the comparative light-emitting devices 3 and 4.

FIG. 31 shows external quantum efficiency-luminance characteristics of the light-emitting devices 1 and 2 and the comparative light-emitting devices 3 and 4.

FIG. 32 shows electroluminescence spectra of the light-emitting devices 1 and 2 and the comparative light-emitting devices 3 and 4.

FIG. 33 shows luminance-current density characteristics of a light-emitting device 5, a light-emitting device 6, a comparative light-emitting device 7, and a comparative light-emitting device 8.

FIG. 34 shows luminance-voltage characteristics of the light-emitting devices 5 and 6 and the comparative light-emitting devices 7 and 8.

FIG. 35 shows showing current efficiency-luminance characteristics of the light-emitting devices 5 and 6 and the comparative light-emitting devices 7 and 8.

FIG. 36 shows showing current density-voltage characteristics of the light-emitting devices 5 and 6 and the comparative light-emitting devices 7 and 8.

FIG. 37 shows external quantum efficiency-luminance characteristics of the light-emitting devices 5 and 6 and the comparative light-emitting devices 7 and 8.

FIG. 38 shows electroluminescence spectra of the light-emitting devices 5 and 6 and the comparative light-emitting devices 7 and 8.

FIG. 39 shows the time dependence of normalized luminance of the light-emitting devices 1 and 2 and the comparative light-emitting devices 3 and 4.

FIG. 40 shows the time dependence of normalized luminance of the light-emitting devices 5 and 6 and the comparative light-emitting devices 7 and 8.

FIG. 41 shows luminance-current density characteristics of a light-emitting device 9, a light-emitting device 10, a comparative light-emitting device 11, and a comparative light-emitting device 12.

FIG. 42 shows luminance-voltage characteristics of the light-emitting devices 9 and 10 and the comparative light-emitting devices 11 and 12.

FIG. 43 shows showing current efficiency-luminance characteristics of the light-emitting devices 9 and 10 and the comparative light-emitting devices 11 and 12.

FIG. 44 shows showing current density-voltage characteristics of the light-emitting devices 9 and 10 and the comparative light-emitting devices 11 and 12.

FIG. 45 shows external quantum efficiency-luminance characteristics of the light-emitting devices 9 and 10 and the comparative light-emitting devices 11 and 12.

FIG. 46 shows electroluminescence spectra of the light-emitting devices 9 and 10 and the comparative light-emitting devices 11 and 12.

FIG. 47 shows the time dependence of normalized luminance of the light-emitting devices 9 and 10 and the comparative light-emitting devices 11 and 12.

FIG. 48 shows a ¹H NMR spectrum of FLP(2)NBBnf(II)(4).

FIG. 49 shows an absorption spectrum and an emission spectrum of a toluene solution of FLP(2)NBBnf(II)(4).

FIG. 50 shows an absorption spectrum and an emission spectrum of a thin film of FLP(2)NBBnf(II)(4).

FIGS. 51A and 51B show a phosphorescence spectrum of FLP(2)NBBnf(II)(4).

FIG. 52 shows luminance-current density characteristics of a light-emitting device 13 and a comparative light-emitting device 14.

FIG. 53 shows luminance-voltage characteristics of the light-emitting device 13 and the comparative light-emitting device 14.

FIG. 54 shows showing current efficiency-luminance characteristics of the light-emitting device 13 and the comparative light-emitting device 14.

FIG. 55 shows showing current density-voltage characteristics of the light-emitting device 13 and the comparative light-emitting device 14.

FIG. 56 shows external quantum efficiency-luminance characteristics of the light-emitting device 13 and the comparative light-emitting device 14.

FIG. 57 shows electroluminescence spectra of the light-emitting device 13 and the comparative light-emitting device 14.

FIG. 58 shows the time dependence of normalized luminance of the light-emitting device 13.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Thus, the present invention should not be construed as being limited to the description in the following embodiments.

Note that the position, size, range, or the like of each component illustrated in drawings and the like is not accurately represented in some cases for easy understanding. Thus, the disclosed invention is not necessarily limited to the position, size, range, or the like disclosed in the drawings and the like.

Ordinal numbers such as “first” and “second” in this specification and the like are used for convenience and do not denote the order of steps or the stacking order of layers in some cases. Thus, for example, description can be made even when “first” is replaced with “second” or “third”, as appropriate. In addition, the ordinal numbers in this specification and the like are not necessarily the same as those used to specify one embodiment of the present invention.

In the description of structures of the present invention in this specification and the like with reference to the drawings, the same components in different drawings are denoted by the same reference numeral in some cases.

In this specification and the like, the terms “film” and “layer” can be interchanged with each other. For example, the term “conductive layer” can be changed to the term “conductive film” in some cases. For another example, the term “insulating film” can be changed into the term “insulating layer” in some cases.

### Embodiment 1

In this embodiment, an organic compound of one embodiment of the present invention and an organic semiconductor device of one embodiment of the present invention will be described.

The organic compound of one embodiment of the present invention is a triarylamine that has a structure in which a first aryl group, a second aryl group, and a third aryl group are bonded to one nitrogen. Triarylamines have a high lowest unoccupied molecular orbital (LUMO) level and a large highest occupied molecular orbital (HOMO)-LUMO gap, which provides excellent hole-transport properties and enables them to be used in a variety of organic semiconductor devices.

In the organic compound of one embodiment of the present invention, the first aryl group includes a naphthyl group that is bonded to the nitrogen through a phenylene group or a biphenyldiyl group. Note that the naphthyl group and the phenylene group or the biphenyldiyl group may each have a substituent. A naphthalene ring has a stable structure where two benzene rings are fused. Since a structure where a naphthyl group is bonded to a phenylene group or a biphenyldiyl group has a π-electron conjugated system extending over the naphthyl group and the phenylene group or biphenyldiyl group, the organic compound including the first aryl group with such a structure can have a high hole-transport property. The organic compound including the first aryl group with such a structure can also have high stability. Moreover, the inclusion of the first aryl with such a structure group increases the glass transition point of the organic compound, resulting in improved heat resistance. This makes the organic compound of one embodiment of the present invention highly resistant to high-temperature treatment after film formation. For example, even when the organic compound of one embodiment of the present invention is formed into a film by evaporation and a material that requires extremely high-temperature evaporation is deposited over this film, the film does not easily change in quality. Furthermore, the organic compound of one embodiment of the present invention can be formed into a film with high purity and quality because decomposition or degradation of the organic compound due to heat is unlikely to occur. Consequently, a film with a stable quality can be formed. With such an organic compound, an organic semiconductor device having high reliability and high quality can be manufactured.

The second aryl group includes a benzo[b]naphtho[2,1-d]furanyl group, a benzo[b]naphtho[2,3-d]furanyl group, a benzo[b]naphtho[2,1-d]thiophenyl group, or a benzo[b]naphtho[2,3-d]thiophenyl group that is bonded to the nitrogen directly or through an arylene group. Note that these groups and the arylene group may each have a substituent. A benzo[b]naphtho[2,1-d]furan ring, a benzo[b]naphtho[2,3-d]furan ring, a benzo[b]naphtho[2,1-d]thiophene ring, and a benzo[b]naphtho[2,3-d]thiophene ring each include a fused naphthalene ring. This extends the π-electron conjugated system, making them capable of easily accepting electrons. Therefore, owing to the second aryl group including any of these rings that is included in the organic compound, the LUMO of the organic compound tends to be distributed over the ring. Consequently, the LUMO is less likely to be distributed over the other aryl groups (first and third aryl groups) in the organic compound of one embodiment of the present invention. This makes the molecule of the organic compound as a whole more resistant to reduction. Hence, the use of the organic compound of one embodiment of the present invention can inhibit a significant change in the driving voltage of an organic semiconductor device over driving time. The organic semiconductor device can also have an extended driving lifetime.

In this specification and the like, a benzo[b]-naphtho[2,1-d]furanyl group refers to a monovalent group obtained by eliminating one hydrogen from a benzo[b]naphtho[2,1-d]furan ring, a benzo[b]naphtho[2,3-d]furanyl group refers to a monovalent group obtained by eliminating one hydrogen from a benzo[b]naphtho[2,3-d]furan ring, a benzo[b]naphtho[2,1-d]thiophenyl group refers to a monovalent group obtained by eliminating one hydrogen from a benzo[b]naphtho[2,1-d]thiophene ring, and a benzo[b]naphtho[2,3-d]thiophenyl group refers to a monovalent group obtained by eliminating one hydrogen from a benzo[b]naphtho[2,3-d]thiophene ring.

The third aryl group includes a spirobifluorenyl group or a diphenylfluorenyl group that is directly bonded to the nitrogen. Note that the spirobifluorenyl group or the diphenylfluorenyl group may each have a substituent. Since a spirobifluorenyl group and a diphenylfluorenyl group are bulky groups, introduction of a spirobifluorenyl group or a diphenylfluorenyl group can reduce intermolecular stacking or the like. The introduction into an organic compound including many aromatic rings, in particular, can lower the sublimation temperature of the organic compound to inhibit thermal decomposition during sublimation.

Next, the organic compound of one embodiment of the present invention is described using general formulae.

One embodiment of the present invention is an organic compound represented by General Formula (G1).

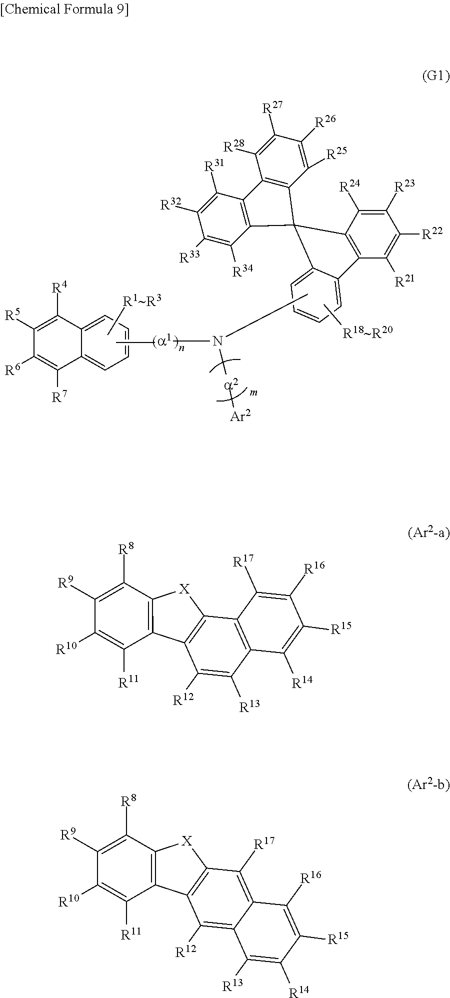

In General Formula (G1), α¹ represents a substituted or unsubstituted phenylene group; n is 1 or 2; α² represents a substituted or unsubstituted phenylene group or a substituted or unsubstituted naphthalene-diyl group; m is 0, 1, or 2; each of R¹ to R⁷ independently represents hydrogen (including deuterium), a straight-chain or branched-chain alkyl group having 1 to 6 carbon atoms, a substituted or unsubstituted cycloalkyl group having 3 to 10 carbon atoms, or a substituted or unsubstituted phenyl group; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When n is 2, a plurality of α¹'s may be the same or different from each other. When m is 2, a plurality of α²'s may be the same or different from each other.

As described above, the organic compound represented by General Formula (G1) has high stability and high heat resistance by including the naphthyl group bonded to the nitrogen through the phenylene group or the biphenyldiyl group. Accordingly, with the use of such an organic compound, an organic semiconductor device having high reliability and high quality can be manufactured.

As described above, since the organic compound represented by General Formula (G1) includes a benzo[b]naphtho[2,1-d]furanyl group, a benzo[b]naphtho[2,3-d]furanyl group, a benzo[b]naphtho[2,1-d]thiophenyl group, or a benzo[b]naphtho[2,3-d]thiophenyl group that is bonded to the nitrogen directly or through an arylene group, the use of the organic compound can inhibit a significant change in the driving voltage of an organic semiconductor device over driving time. The organic semiconductor device can also have an extended driving lifetime.

Since the organic compound represented by General Formula (G1) includes a spirobifluorene ring, the organic compound have a low sublimation temperature and can be less likely to be thermally decomposed during sublimation.

Another embodiment of the present invention is an organic compound represented by General Formula (G2).

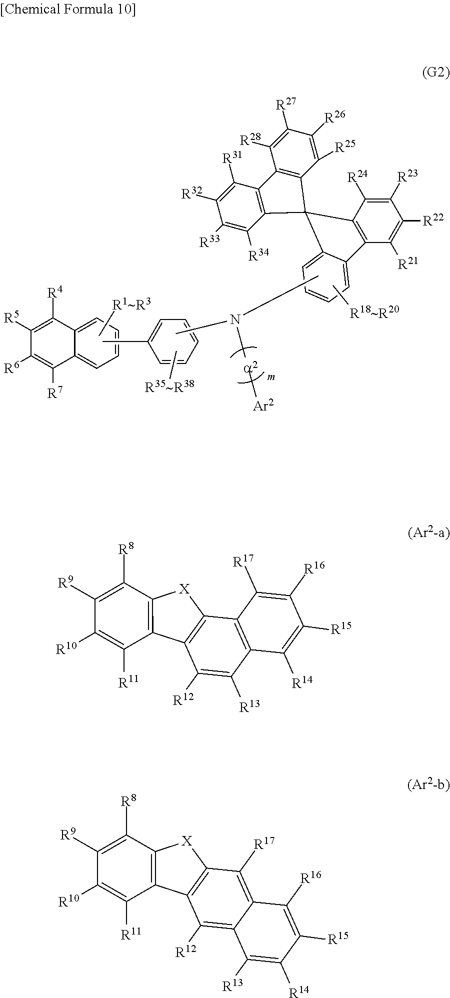

In General Formula (G2), α² represents a substituted or unsubstituted phenylene group or a substituted or unsubstituted naphthalene-diyl group; m is 0, 1, or 2; each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When m is 2, a plurality of α²'s may be the same or different from each other.

General Formula (G2) is different from General Formula (G1) in that n in General Formula (G1) is limited to 1. When n is limited to 1, the molecular weight is lower than that when n is 2, which inhibits an excessive increase in the sublimation temperature of the organic compound. This contributes to improvements of the quality and purity of a film formed by evaporation of the organic compound, resulting in higher device reliability. Furthermore, when n is limited to 1, the solubility in a solvent is less likely to decrease than that when n is 2. This facilitates purification by a common solution process, reduces the load on the purification process, and enables easier achievement of high purity of the organic compound, which is preferable. Further preferably, m in General Formula (G2) is 0 because this results in enhancement of these effects.

Another embodiment of the present invention is an organic compound represented by General Formula (G3).

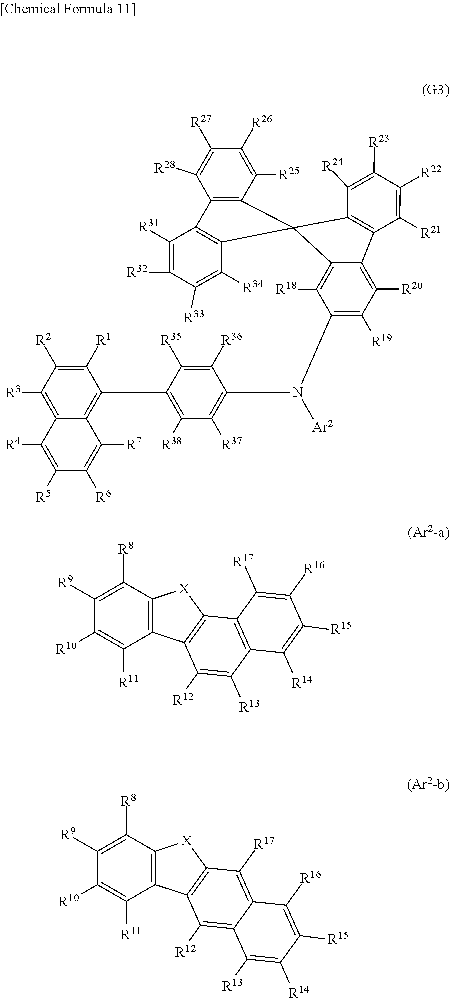

In General Formula (G3), each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When n is 2, a plurality of α¹'s may be the same or different from each other. When m is 2, a plurality of α²'s may be the same or different from each other.

General Formula (G3) is different from General Formula (G2) in that the phenylene group, the naphthyl group, and m in General Formula (G2) are limited to a p-phenylene group, a 1-naphthyl group, and 0, respectively. When the phenylene group is ap-phenylene group, the π-electron conjugated system formed by the phenylene group and the naphthyl group more easily extends than that when the phenylene group is an o-phenylene group or a m-phenylene group. This enhances the stability of the organic compound. Moreover, when the phenylene group is a p-phenylene group, distortion of the molecular structure can be small and the glass transition point can be higher than that when the phenylene group is a o-phenylene group or a m-phenylene group, which is preferable. When the naphthyl group is a 1-naphthyl group, the hole-transport property, reliability, and heat resistance of the organic compound can be more improved than those when the naphthyl group is a 2-naphthyl group. Furthermore, when m is 0, an excessive increase in the sublimation temperature of the organic compound can be inhibited, and the reliability of the organic compound can be improved. In this case, the solubility is less likely to decrease, reducing the load on the purification process and enabling easier achievement of high purity of the organic compound, which is preferable.

Another embodiment of the present invention is the organic compound represented by General Formula (G4).

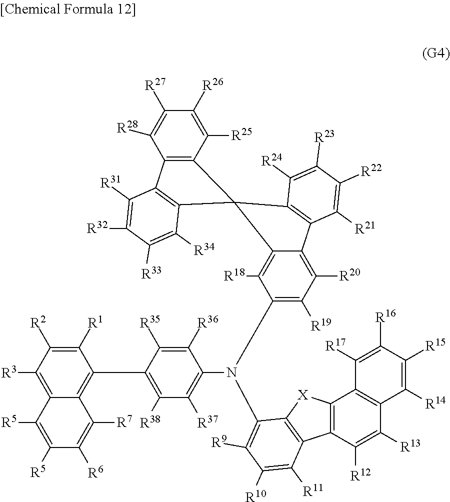

In General Formula (G4), each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; each of R⁹ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur.

General Formula (G4) is different from General Formula (G3) in that Ar² in General Formula (G3) is limited to the group represented by General Formula (Ar²-a). When Ar² is the group represented by General Formula (Ar²-a), the lowest triplet excited level (T₁ level) is higher than that when Ar² is the group represented by General Formula (Ar²-b). The use of this compound for a layer in contact with a light-emitting layer of a light-emitting device can prevent exciton diffusion from the light-emitting layer to an adjacent layer or the like, enhancing the emission efficiency of the light-emitting device.

Another embodiment of the present invention is an organic compound represented by General Formula (G5).

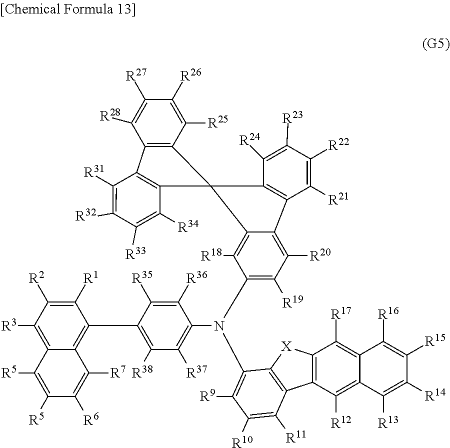

In General Formula (G5), each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; each of R⁹ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur.

General Formula (G5) is different from General Formula (G3) in that Ar² in General Formula (G3) is limited to the group represented by General Formula (Ar²-b). When Ar² is the group represented by General Formula (Ar²-b), the organic compound can have higher reliability and a higher hole-transport property. In addition, the HOMO level of the organic compound is higher than that when Ar² is the group represented by General Formula (Ar²-a). The organic compound is also highly resistant to repeated oxidation and reduction, which is preferable.

When Ar² is limited to the group represented by General Formula (Ar²-b), it is also preferable that the third aryl group in the organic compound of one embodiment of the present invention include a diphenylfluorenyl group. This is because the organic compound where Ar² is the group represented by General Formula (Ar²-b) can be highly reliable also when the third aryl group includes not a spirobifluorenyl group but a diphenylfluorenyl group.

Another embodiment of the present invention is an organic compound represented by General Formula (G6).

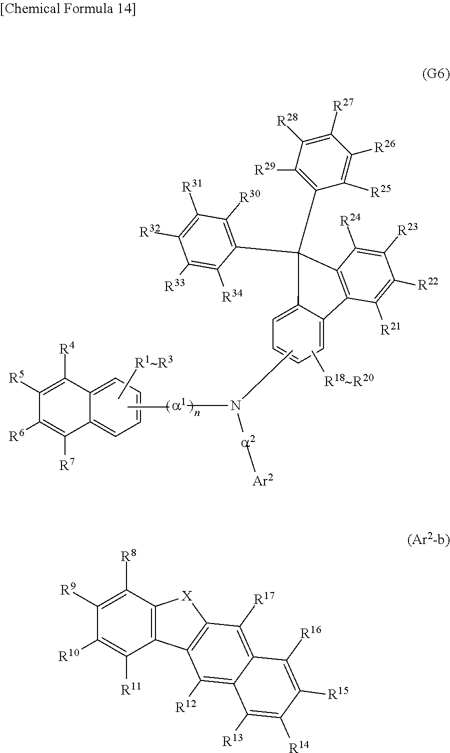

In General Formula (G7), α¹ represents a substituted or unsubstituted phenylene group; n is 1 or 2; α² represents a substituted or unsubstituted phenylene group or a substituted or unsubstituted naphthalene-diyl group; m is 0, 1, or 2; each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; Ar² represents a group represented by General Formula (Ar²-b); any one of R⁸ to R¹⁷ represents a bond; each of R⁸ to R¹⁷ other than the bond and each of R¹⁸ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When n is 2, a plurality of α¹'s may be the same or different from each other. When m is 2, a plurality of α²'s may be the same or different from each other.

General Formula (G6) is different from General Formula (G2) in that Ar² in General Formula (G2) is limited to the group represented by General Formula (Ar²-b) and the third aryl group includes a diphenylfluorenyl group. As described above, the organic compound where Ar² is limited to the group represented by General Formula (Ar²-b) can be highly reliable also when the third aryl group includes not a spirobifluorenyl group but a diphenylfluorenyl group.

Another embodiment of the present invention is an organic compound represented by General Formula (G7).

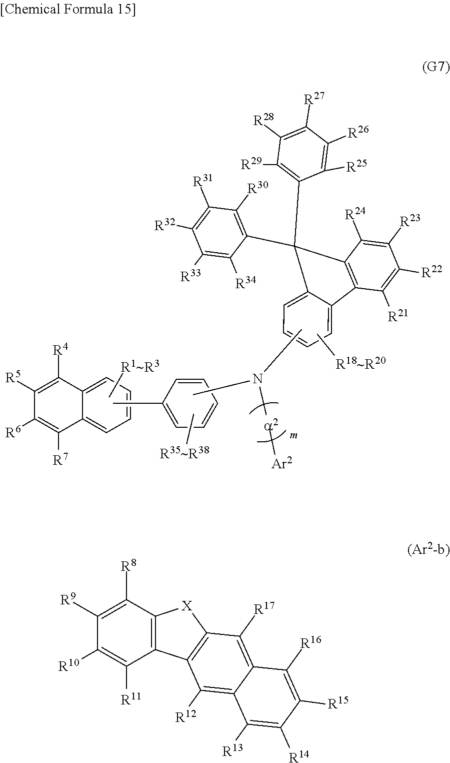

In General Formula (G7), α² represents a substituted or unsubstituted phenylene group or a substituted or unsubstituted naphthalene-diyl group; m is 0, 1, or 2; each of R¹ to R⁷ and R³⁵ to R³⁸ independently represents hydrogen (including deuterium), a straight-chain or branched-chain alkyl group having 1 to 6 carbon atoms, or a substituted or unsubstituted cycloalkyl group having 3 to 10 carbon atoms; Ar² represents a group represented by General Formula (Ar²-b); any one of R⁸ to R¹⁷ represents a bond; each of R⁸ to R¹⁷ other than the bond and each of R¹⁸ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When m is 2, a plurality of α²'s may be the same or different from each other.

General Formula (G7) is different from General Formula (G6) in that n in General Formula (G6) is limited to 1. When n is limited to 1, an excessive increase in the sublimation temperature of the organic compound can be inhibited, and the reliability of a film formed by evaporation of the organic compound can be improved. Moreover, since the solubility is less likely to decrease, high purity of the organic compound can be easily achieved in the purification process, which is preferable. Further preferably, m in General Formula (G7) is 0 because this results in enhancement of these effects.

When X in each of the above general formulae is oxygen, the refractive index of the organic compound can be lower than that when X is sulfur. The organic compound with a lower refractive index improves light extraction efficiency when used in a light-emitting device, for example, which is preferable. It is preferable that X be oxygen because this generally facilitates synthesis of the organic compound and makes industrial use easier.

Meanwhile, when X in each of the above general formulae is sulfur, the heat resistance (e.g., decomposition temperature, melting point, or sublimation point) of the organic compound can be higher than that when X is oxygen. The organic compound with higher heat resistance is preferably used in a light-emitting device, for example, in which case an organic semiconductor device capable of stable driving in a high-temperature environment can be provided.

Next, specific examples of substituents that can be used for the organic compounds represented by the above general formulae will be described. Note that groups that can be used in the above general formulae are not limited to the following specific examples. In addition, in the specific examples described below, some or all of hydrogen atoms may be deuterium.

### <<Halogen>>

Specific examples of a halogen include fluorine, chlorine, bromine, and iodine. In particular, fluorine, which is chemically stable, is preferable.

### <<Straight-Chain or Branched-Chain Alkyl Group Having 1 to 6 Carbon Atoms>>

A straight-chain or branched-chain alkyl group having 1 to 6 carbon atoms refers to a monovalent group obtained by eliminating one hydrogen (H) atom from a straight-chain or branched-chain alkane having 1 to 6 carbon atoms. Specific examples include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neo-pentyl group, a hexyl group, an isohexyl group, a sec-hexyl group, a tert-hexyl group, a neo-hexyl group, a 3-methylpentyl group, a 2-methylpentyl group, a 2-ethylbutyl group, a 1,2-dimethylbutyl group, a 2,3-dimethylbutyl group, and the like.

### <<Alkenyl Group Having 2 to 6 Carbon Atoms>>

An alkenyl group having 2 to 6 carbon atoms is a monovalent group obtained by removing one H from an alkene having 2 to 6 carbon atoms. Specific examples of an alkenyl group having 2 to 6 carbon atoms include a vinyl group, an aryl group, and a 2,2-dimethylvinyl group.

### <<Alkynyl Group Having 2 to 6 Carbon Atoms>>

An alkynyl group having 2 to 6 carbon atoms is a monovalent group obtained by removing one H from an alkyne having 2 to 6 carbon atoms. Specific examples of an alkynyl group having 2 to 6 carbon atoms include an ethinyl group and a prop-2-yn-1-yl group (also referred to as a propargyl group).

### <<Alkoxy Group Having 1 to 6 Carbon Atoms>>

An alkoxy group having 1 to 6 carbon atoms has a structure in which a straight-chain or branched-chain alkyl group having 1 to 6 carbon atoms is bonded to oxygen (O). Specific examples of an alkoxy group having 1 to 6 carbon atoms include a methoxy group, an ethoxy group, an n-propoxy group, an isopropoxy group, an n-butoxy group, a sec-butoxy group, an isobutoxy group, a tert-butoxy group, an n-pentyloxy group, an isopentyloxy group, a sec-pentyloxy group, a tert-pentyloxy group, a neo-pentyloxy group, an n-hexyloxy group, an isohexyloxy group, a sec-hexyloxy group, a tert-hexyloxy group, a neo-hexyloxy group, a cyclohexyloxy group, and the like.

### <<Silyl Group Having 3 to 18 Carbon Atoms>>

A silyl group having 3 to 18 carbon atoms has a structure in which three alkyl groups having 3 to 18 carbon atoms in total or three aryl groups having 3 to 18 carbon atoms in total are bonded to silicon (Si). Specific examples of a silyl group having 3 to 18 carbon atoms include a trimethylsilyl group, a triethylsilyl group, a tert-butyldimethylsilyl group, a triphenylsilyl group, and the like.

### <<Cycloalkyl Group Having 3 to 10 Carbon Atoms>>

A cycloalkyl group having 3 to 10 carbon atoms is a monovalent group obtained by removing one hydrogen atom from a monocyclic or polycyclic cycloalkane having 3 to 10 carbon atoms. Specific examples of a cycloalkyl group having 3 to 10 carbon atoms include a cyclopropyl group, a cyclobutyl group, a cyclopentyl group, a cyclohexyl group, a cycloheptyl group, a cyclooctyl group, a cyclononyl group, a cyclodecyl group, a norbornyl group, a bicyclo[2,2,2]octyl group, a decahydronaphthyl group, an adamantyl group, and the like. In the case where the cycloalkyl group having 3 to 10 carbon atoms includes a substituent, specific examples of the substituent include a halogen, a cyano group, a straight-chain or branched-chain alkyl group having 1 to 6 carbon atoms, an alkenyl group having 2 to 6 carbon atoms, an alkynyl group having 2 to 6 carbon atoms, an alkoxy group having 1 to 6 carbon atoms, a trialkylsilyl group having 3 to 10 carbon atoms, a phenyl group, and the like.

### <<Aryl Group Having 6 to 30 Carbon Atoms>>

An aryl group having 6 to 30 carbon atoms is a monovalent group obtained by removing one hydrogen atom from one of carbon atoms forming a ring of a monocyclic or polycyclic aromatic compound having 6 to 30 carbon atoms. Specific examples of an aryl group having 6 to 30 carbon atoms include a phenyl group, an o-tolyl group, a m-tolyl group, a p-tolyl group, a mesityl group, a biphenyl-2-yl group (o-biphenyl group), a biphenyl-3-yl group (m-biphenyl group), a biphenyl-4-yl group (p-biphenyl group), a 1-naphthyl group, a 2-naphthyl group, a phenylnaphthyl group, a naphthylphenyl group, a terphenyl group, a fluorenyl group, a 9,9-dimethylfluorenyl group, a quaterphenyl group, a spirobifluorenyl group, a phenanthryl group, an anthryl group, a binaphthylphenyl group, a fluoranthenyl group, and the like. In the case where the aryl group having 6 to 30 carbon atoms includes a substituent, specific examples of the substituent include a halogen, a cyano group, a straight-chain or branched-chain alkyl group having 1 to 6 carbon atoms, an alkenyl group having 2 to 6 carbon atoms, an alkynyl group having 2 to 6 carbon atoms, an alkoxy group having 1 to 6 carbon atoms, a trialkylsilyl group having 3 to 10 carbon atoms, a cycloalkyl group having 3 to 10 carbon atoms, a phenyl group, and the like.

### <<Heteroaryl Group Having 2 to 30 Carbon Atoms>>

A heteroaryl group having 2 to 30 carbon atoms is a monovalent group obtained by removing one hydrogen atom from one of carbon atoms forming a ring of a monocyclic or polycyclic heterocyclic aromatic compound having 2 to 30 carbon atoms. Specific examples of a heteroaryl group having 2 to 30 carbon atoms include a carbazolyl group, a dibenzothiophenyl group, a dibenzofuranyl group, a benzocarbazolyl group, a naphthobenzothiophenyl group, a naphthobenzofuranyl group, a dibenzocarbazolyl group, a dinaphthothiophenyl group, a dinaphthofuranyl group, a triazinyl group, a pyrimidinyl group, a pyrazinyl group, a triazolyl group, a pyridinyl group, a benzofuropyrimidinyl group, a benzothiopyrimidinyl group, a benzofuropyrazinyl group, a benzothiopyrazinyl group, a benzofuropyridinyl group, a benzothiopyridinyl group, a bicarbazolyl group, and the like. In the case where the heteroaryl group having 2 to 30 carbon atoms includes a substituent, specific examples of the substituent include a halogen, a cyano group, a straight-chain or branched-chain alkyl group having 1 to 6 carbon atoms, an alkenyl group having 2 to 6 carbon atoms, an alkynyl group having 2 to 6 carbon atoms, an alkoxy group having 1 to 6 carbon atoms, a trialkylsilyl group having 3 to 10 carbon atoms, a cycloalkyl group having 3 to 10 carbon atoms, a phenyl group, and the like.

The above substituents are specific examples of the substituent that can be used for the organic compounds represented by the general formulae.

Specific examples of the organic compounds of embodiments of the present invention represented by the above general formulae include organic compounds represented by Structural Formulae (100) to (237) below. Note that the organic compound of one embodiment of the present invention is not limited to the organic compounds represented by the following structural formulae.

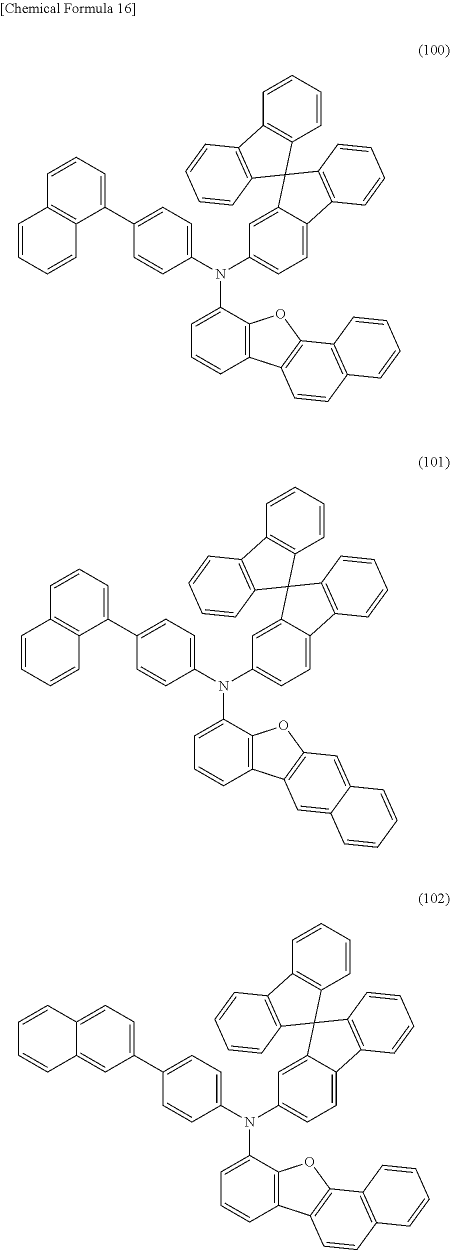

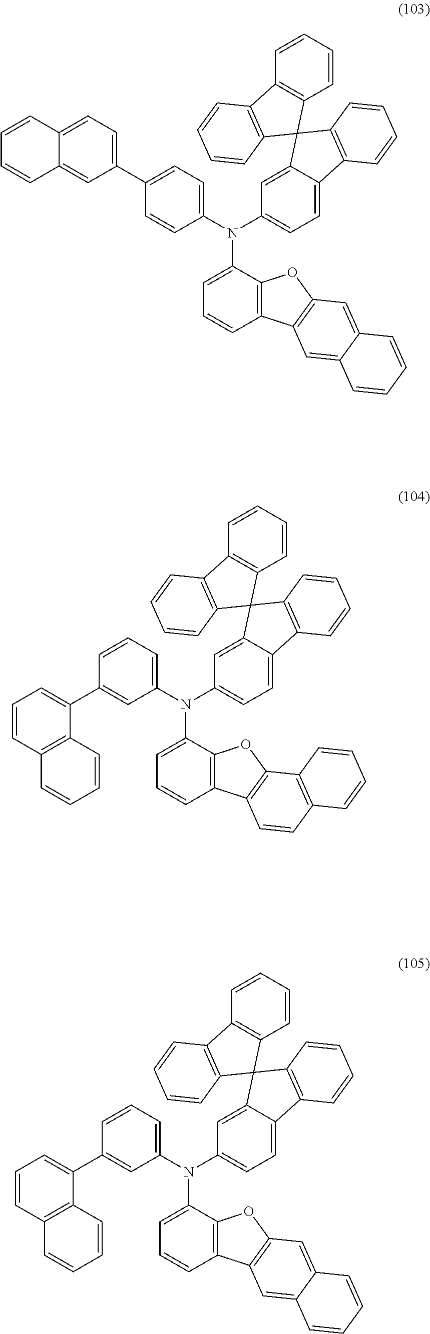

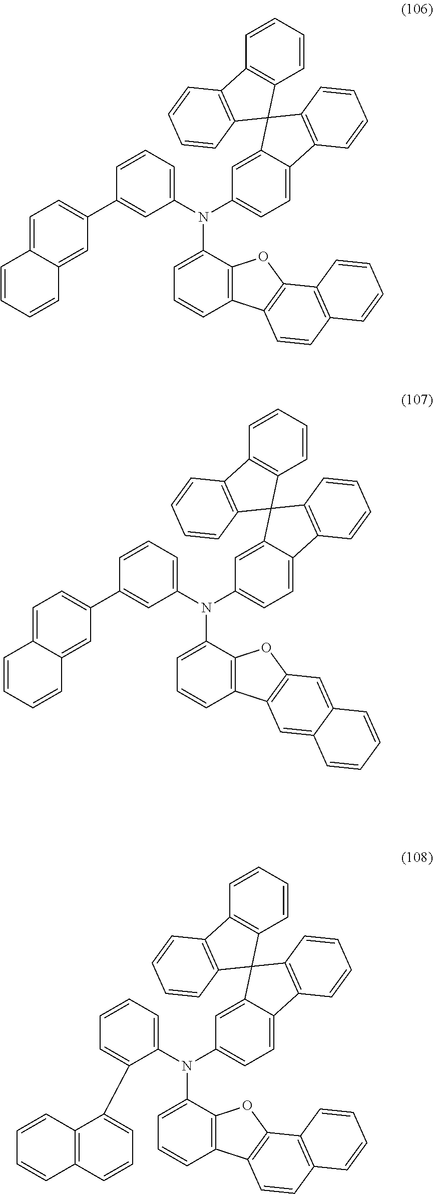

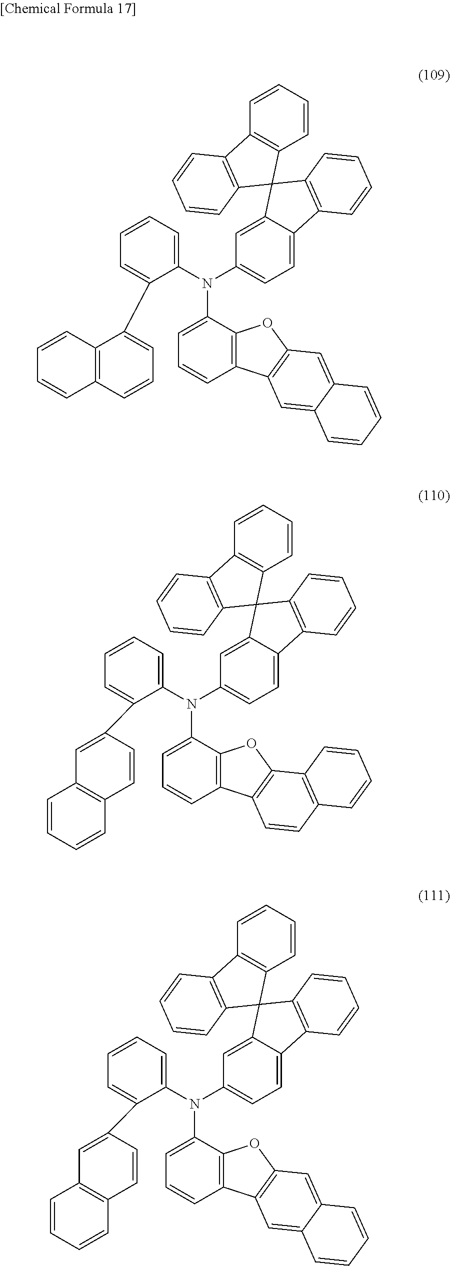

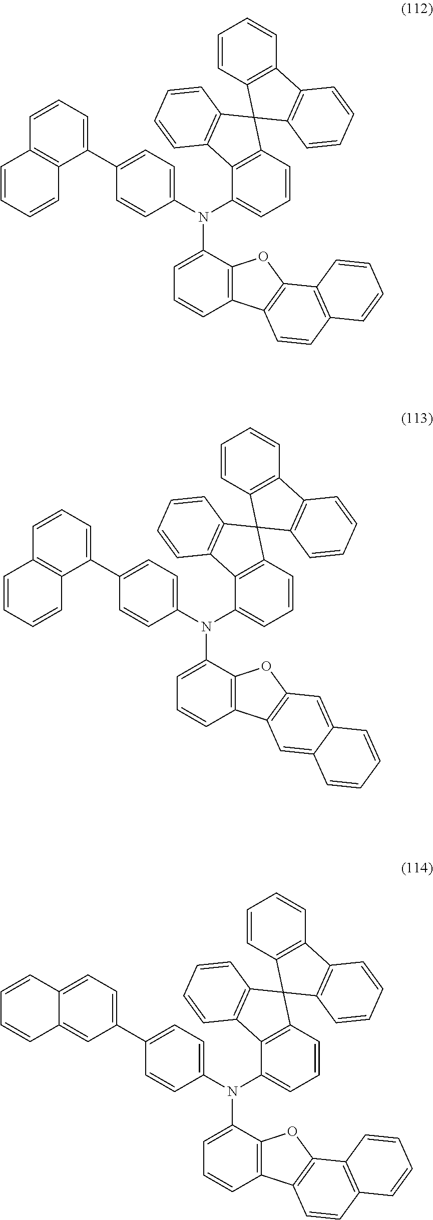

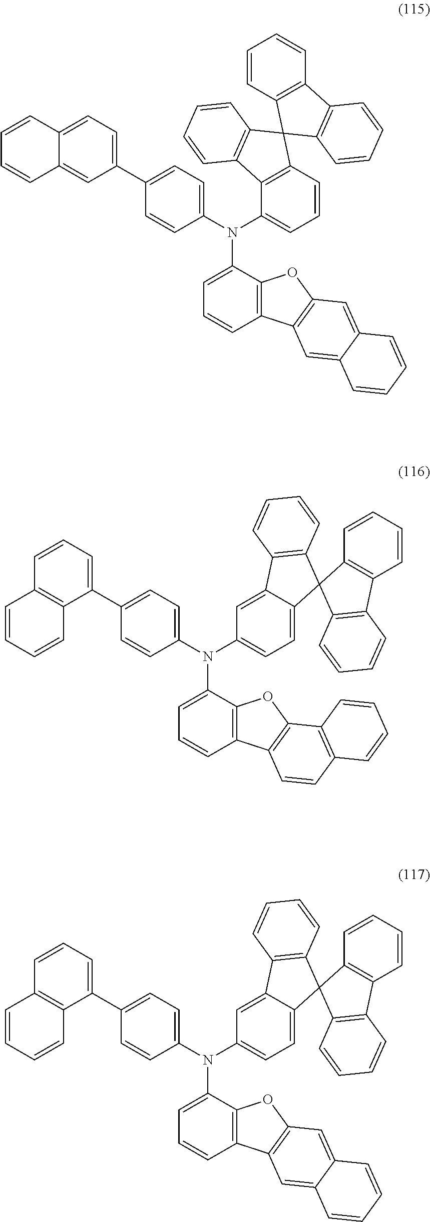

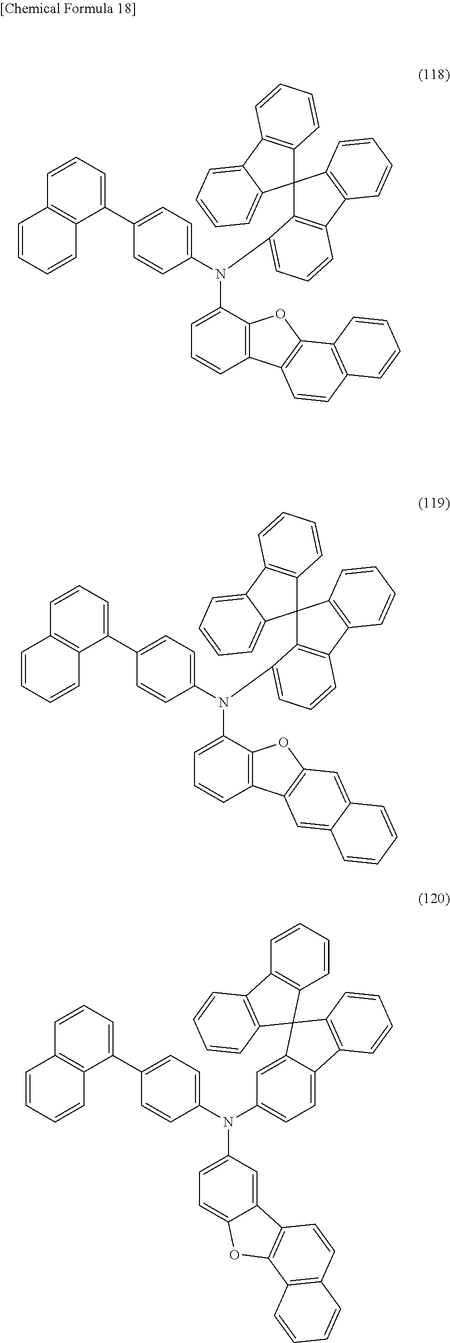

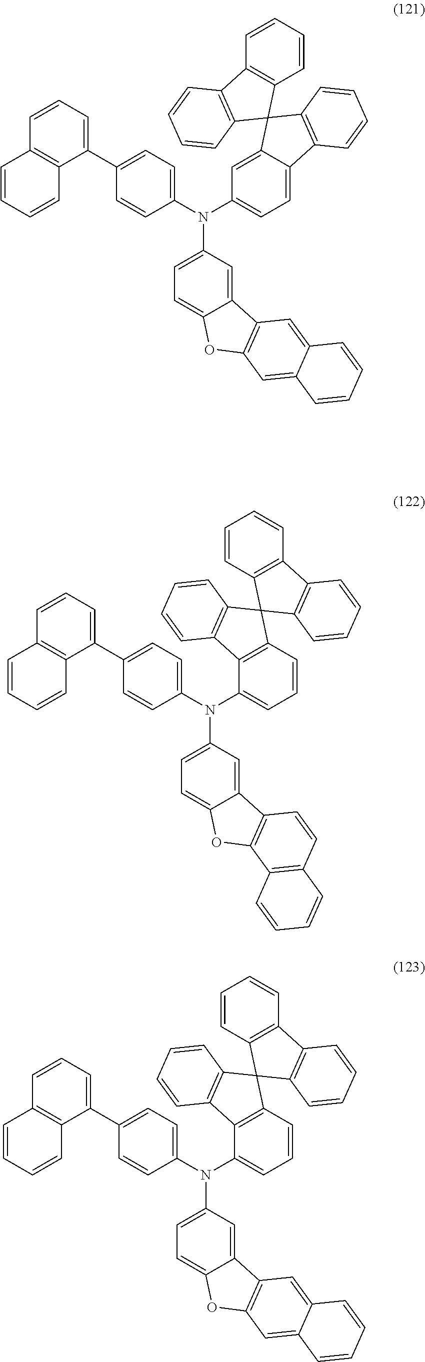

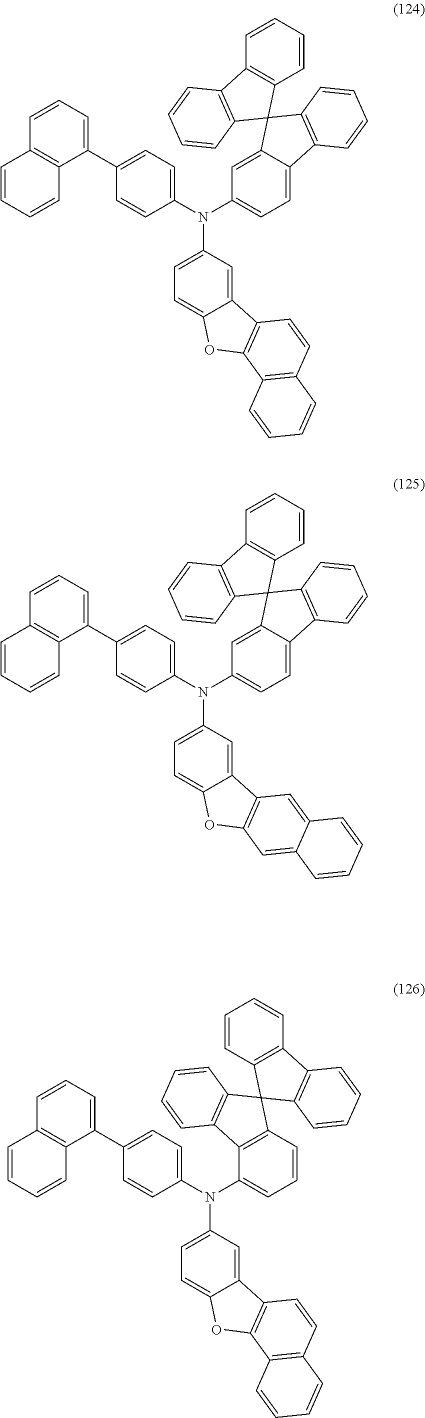

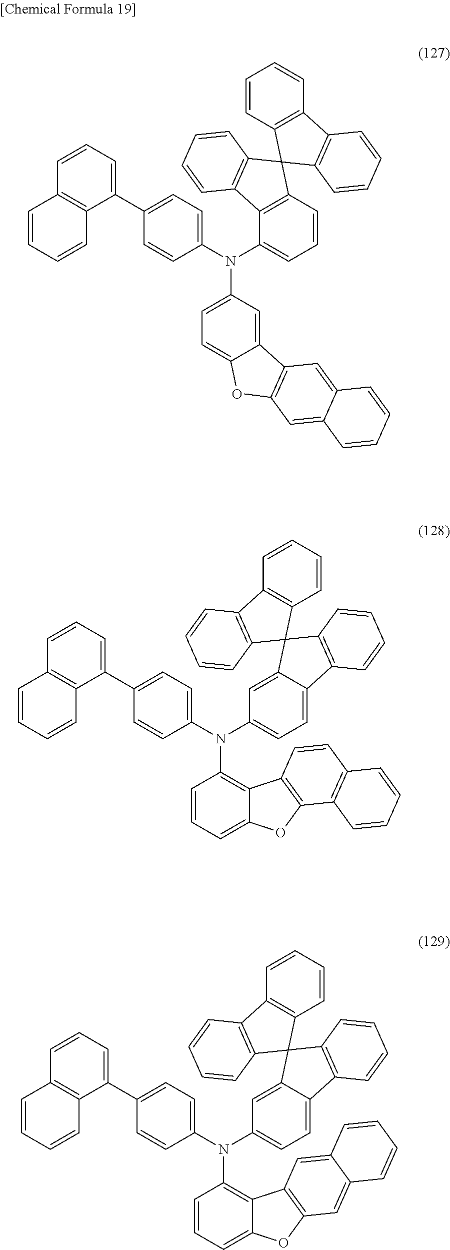

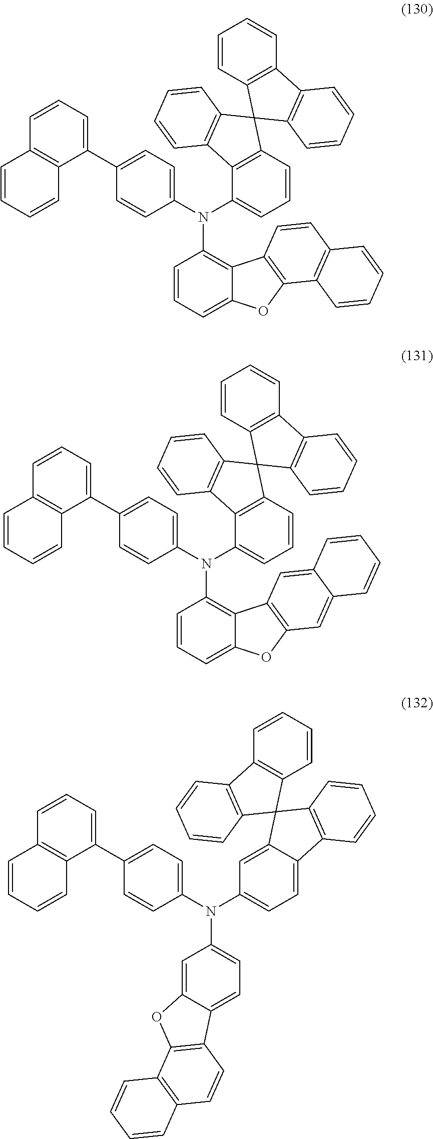

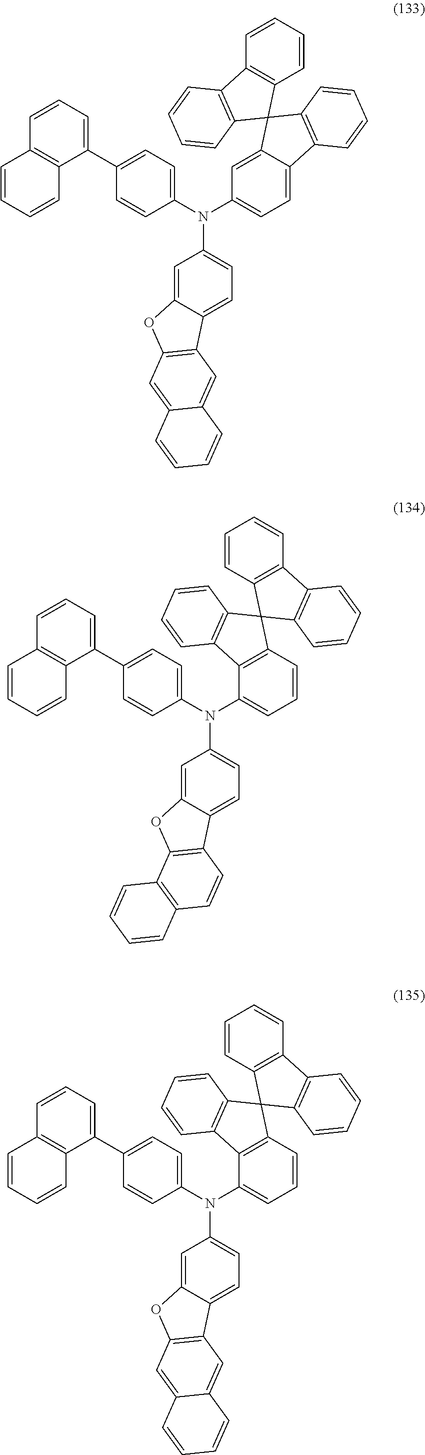

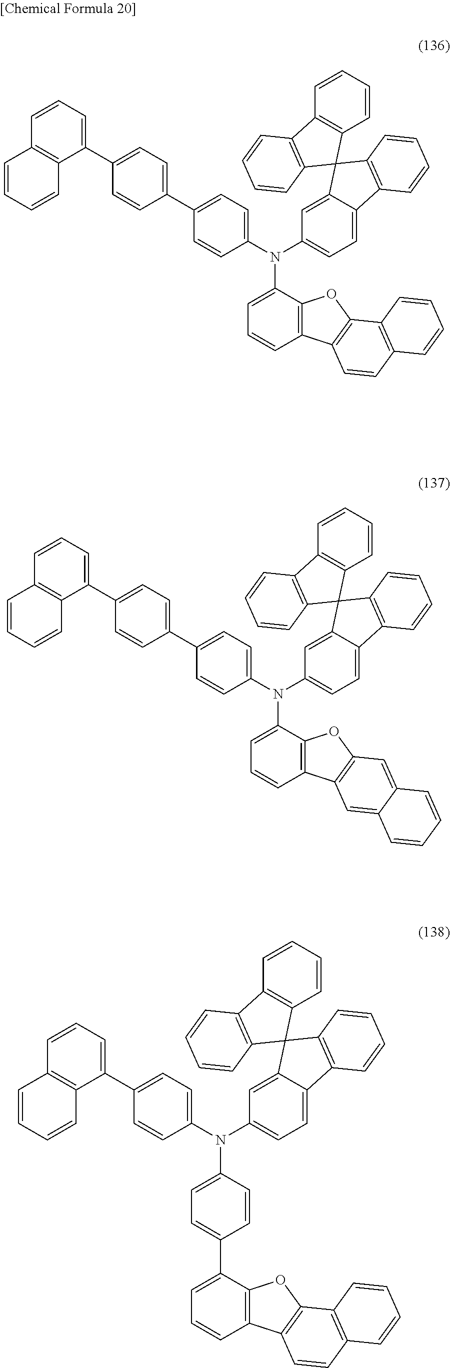

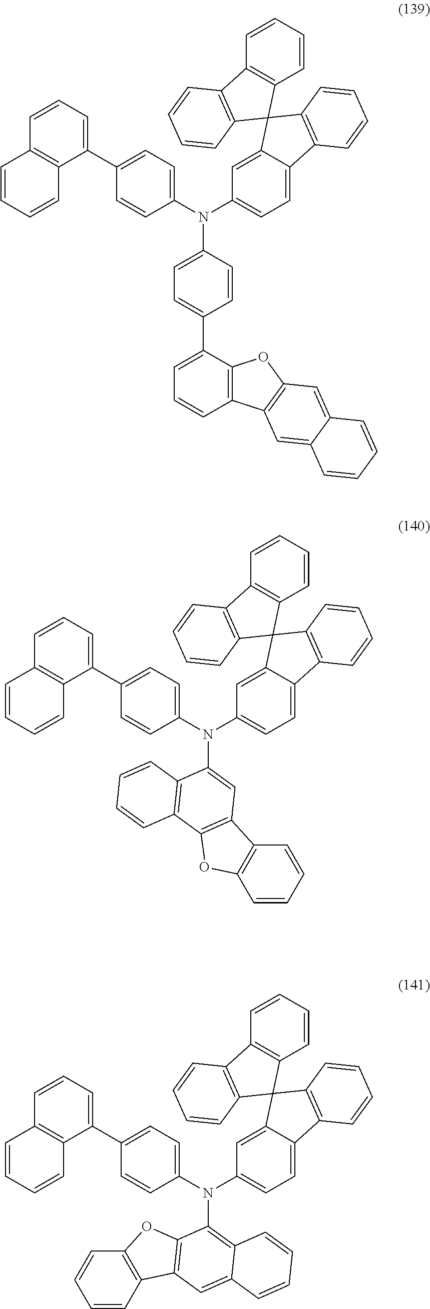

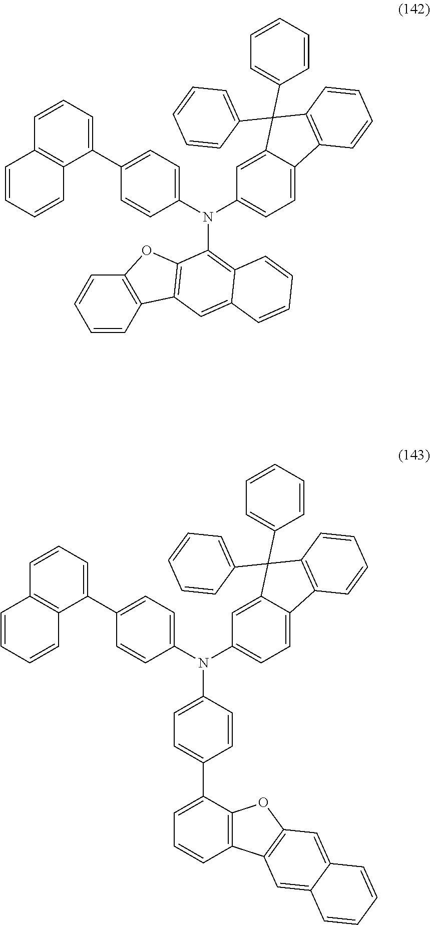

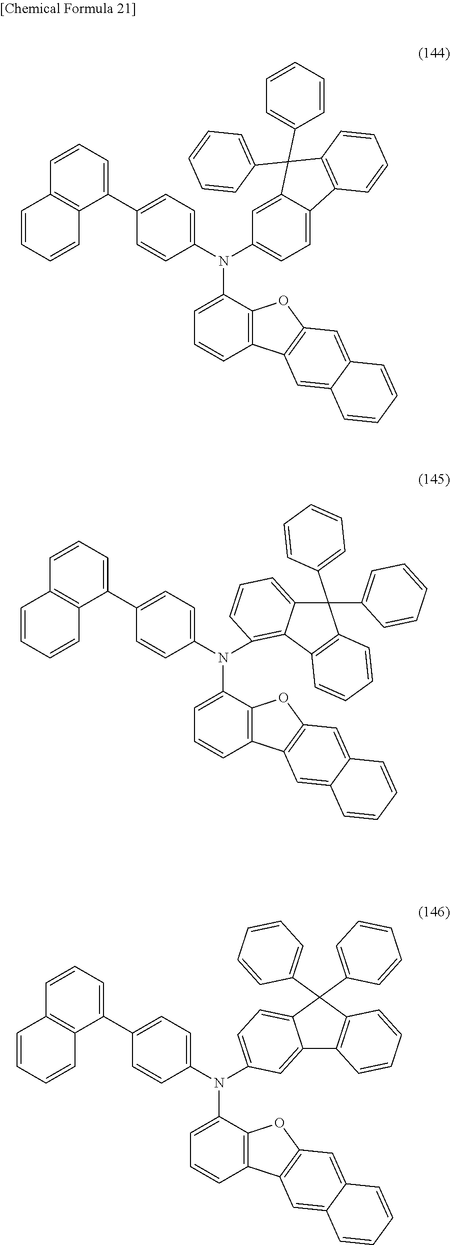

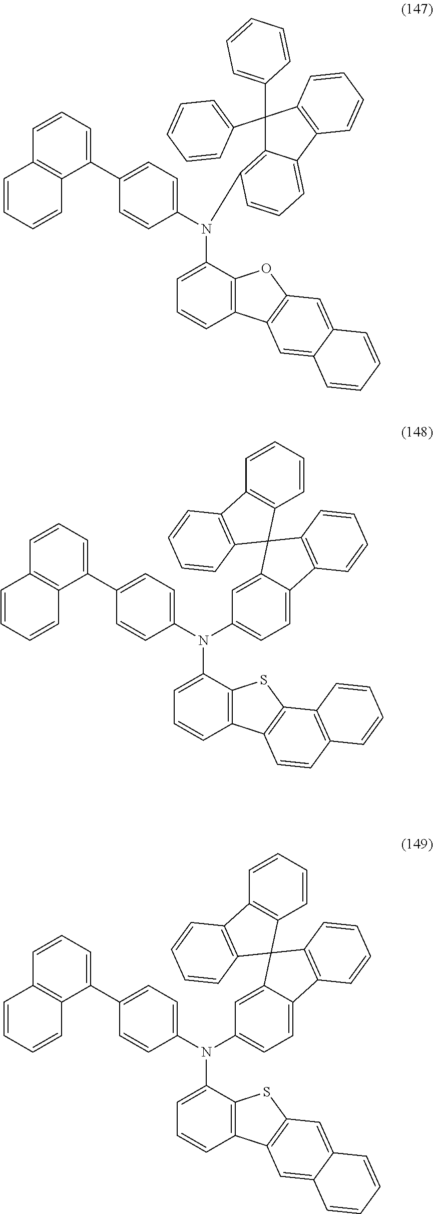

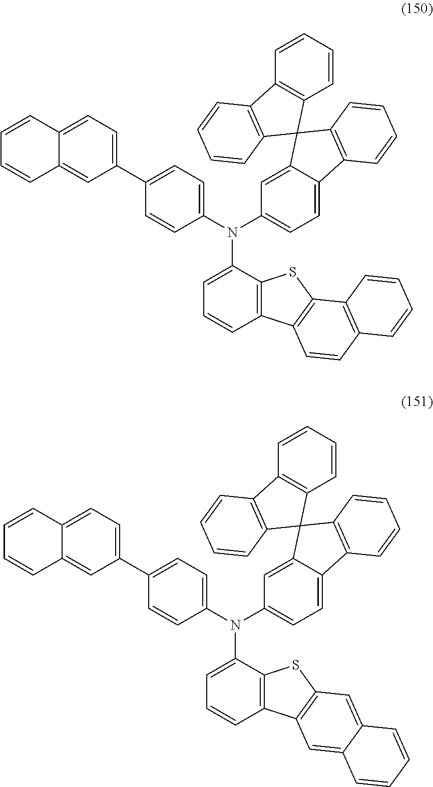

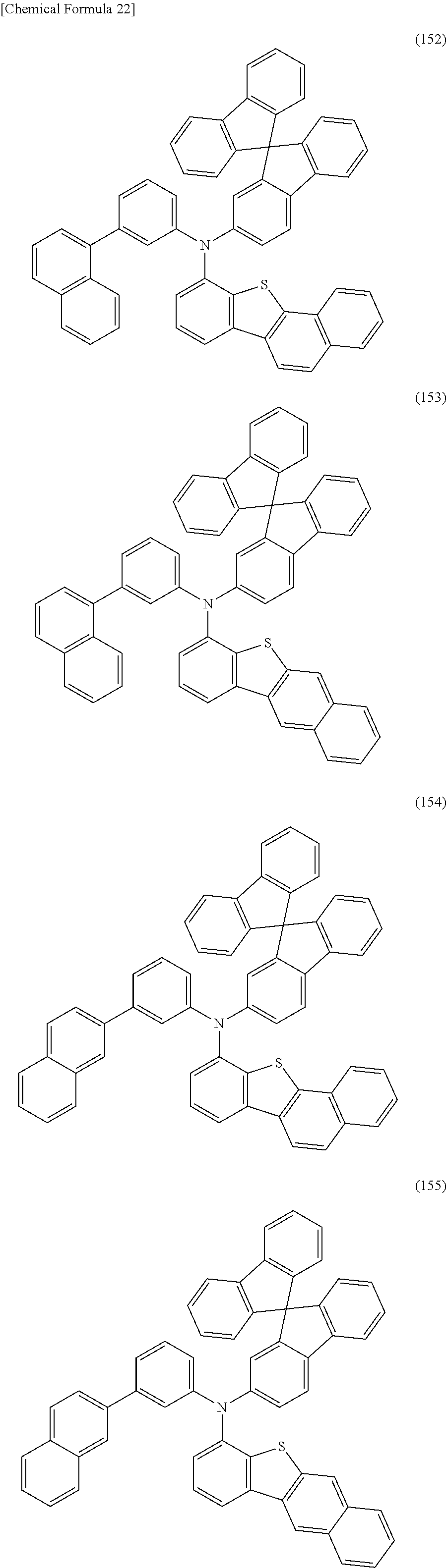

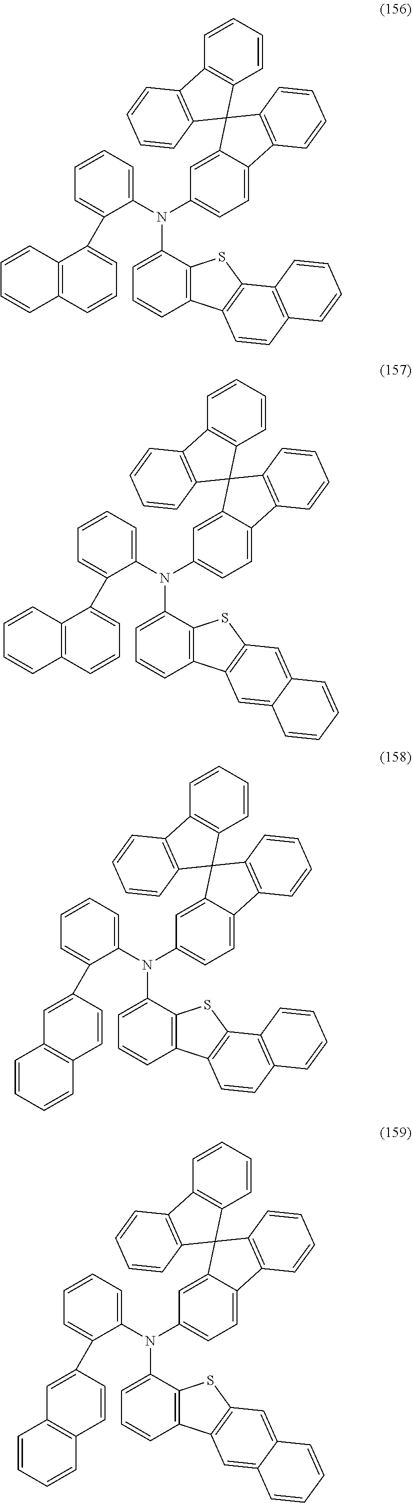

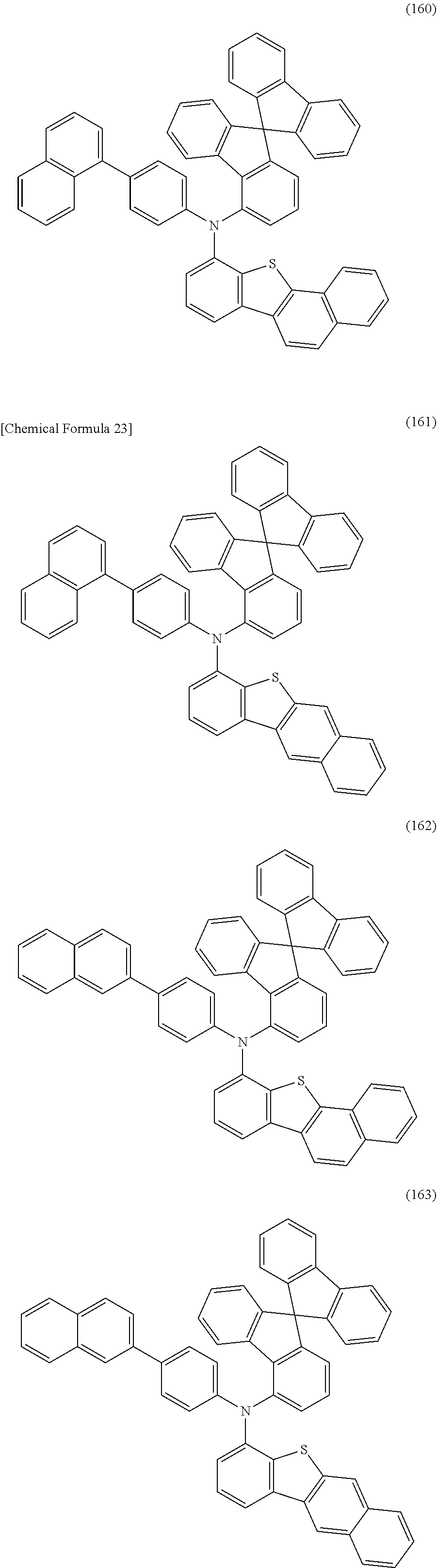

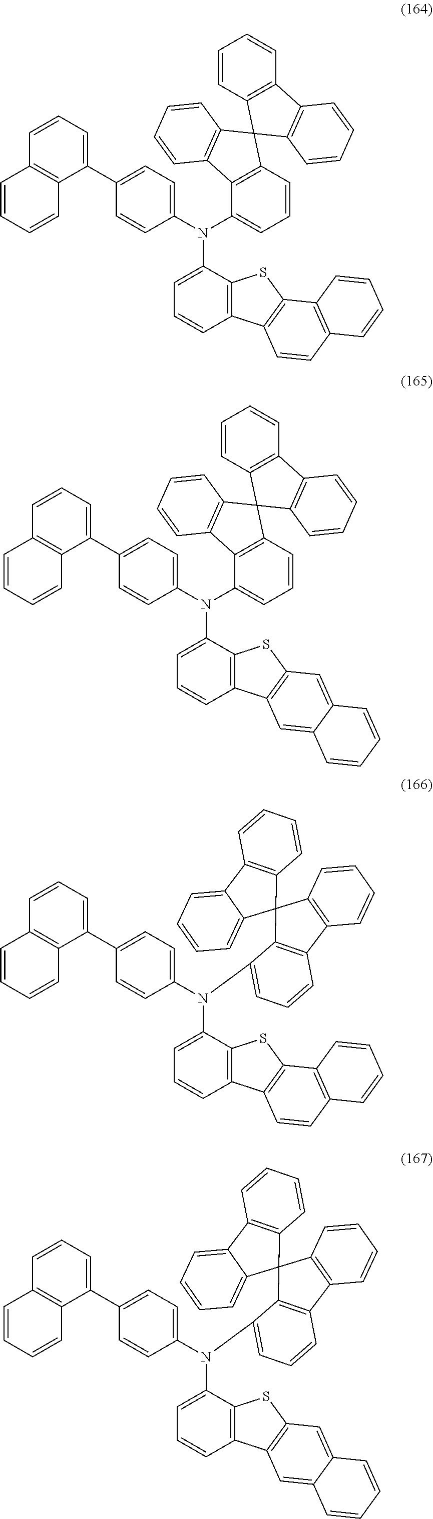

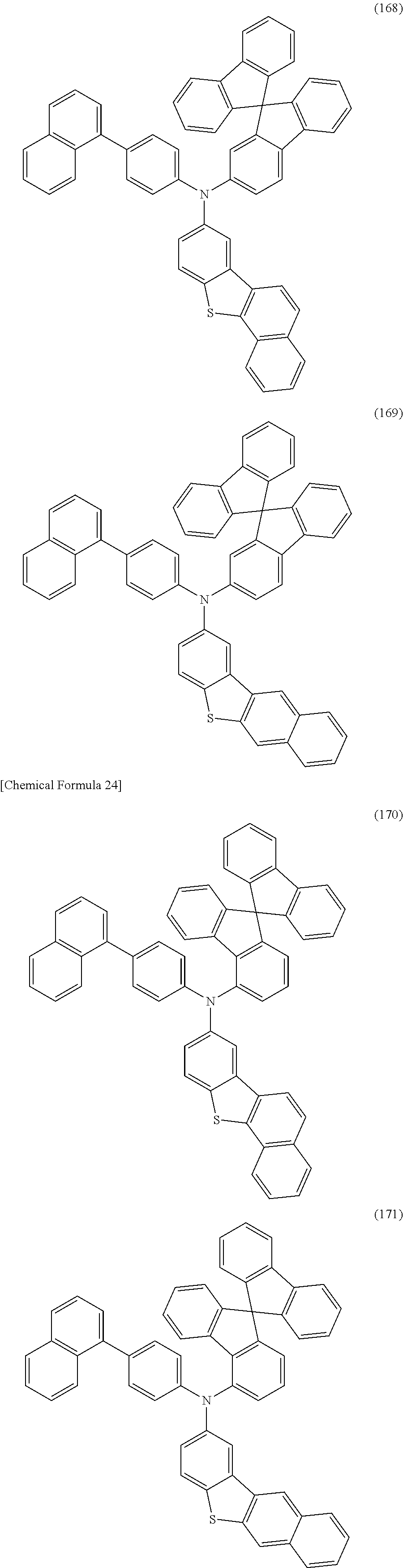

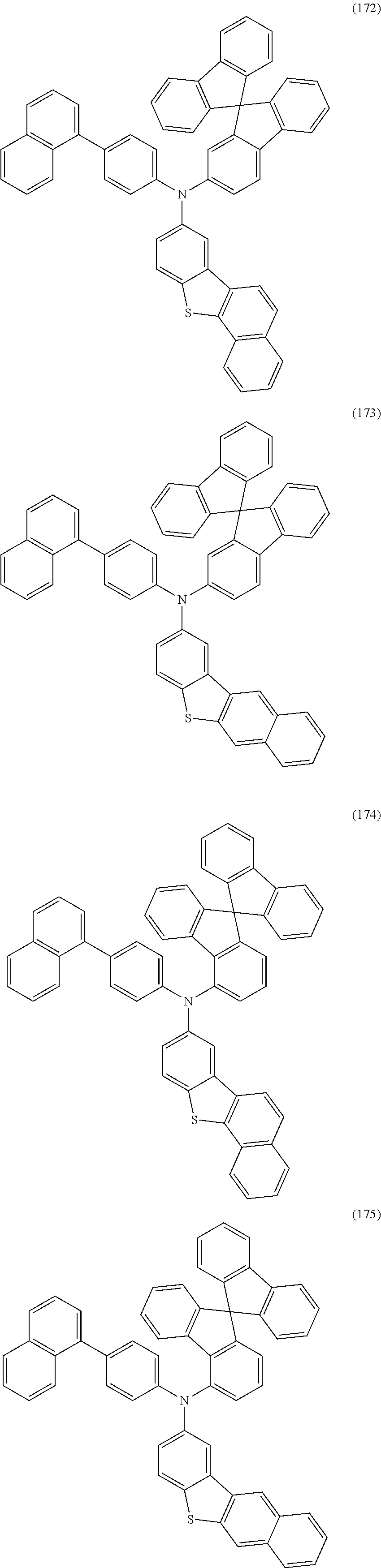

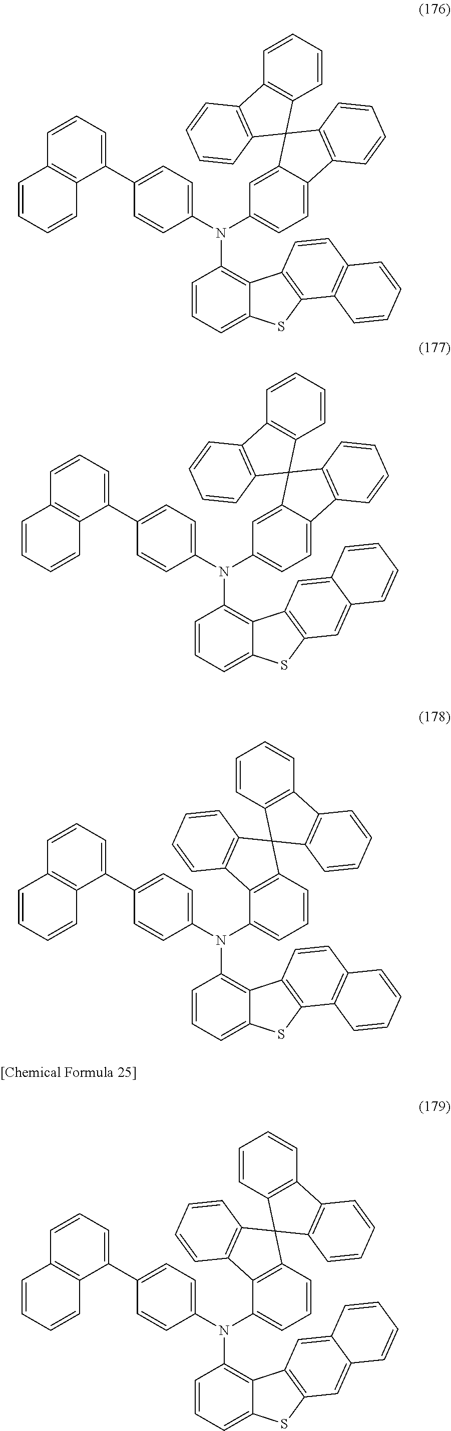

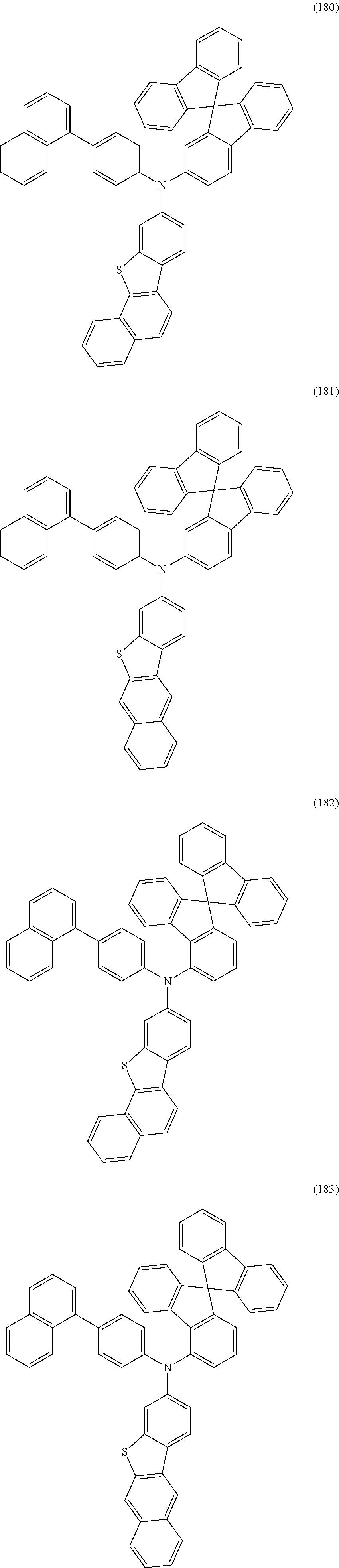

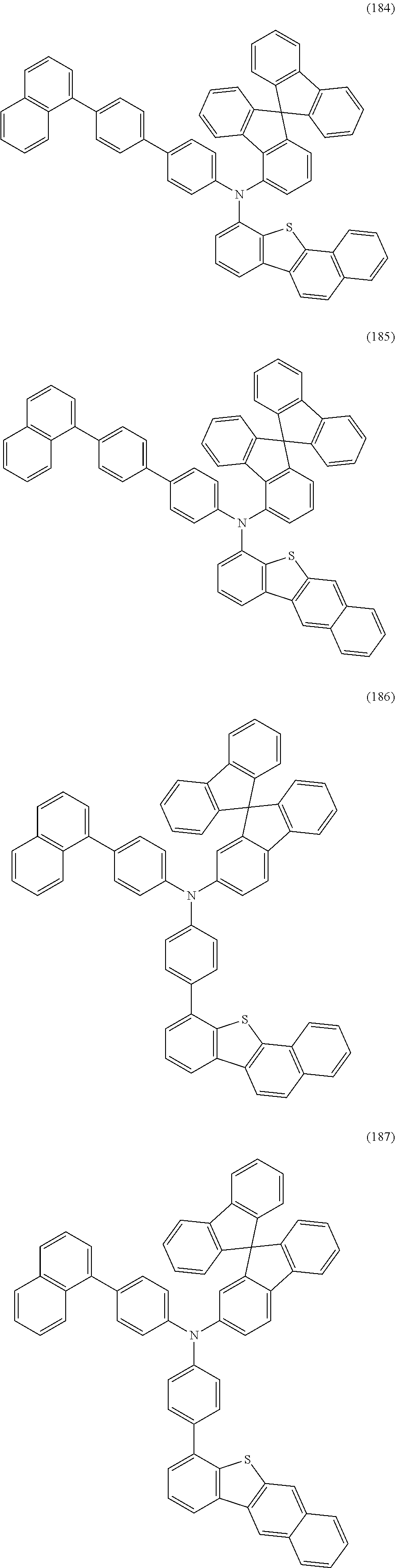

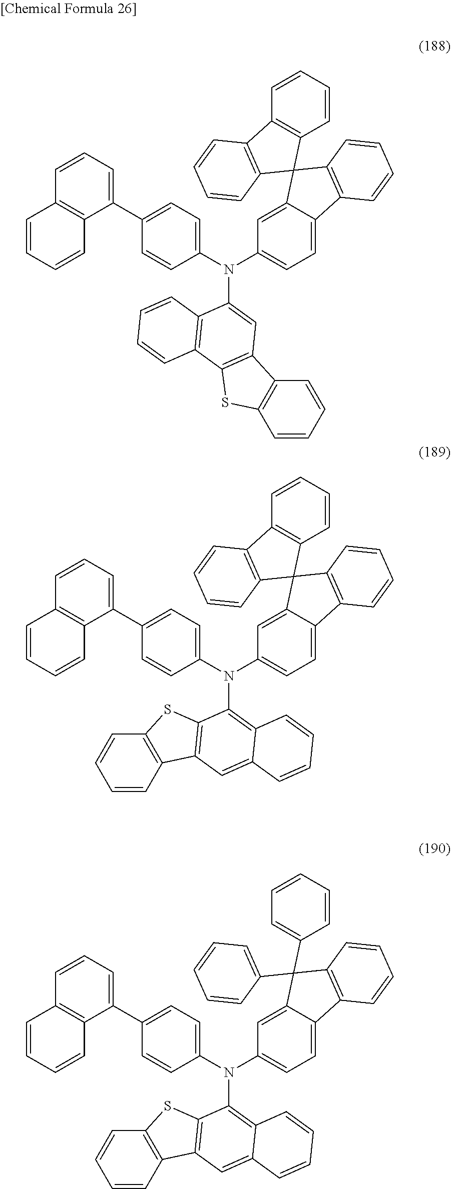

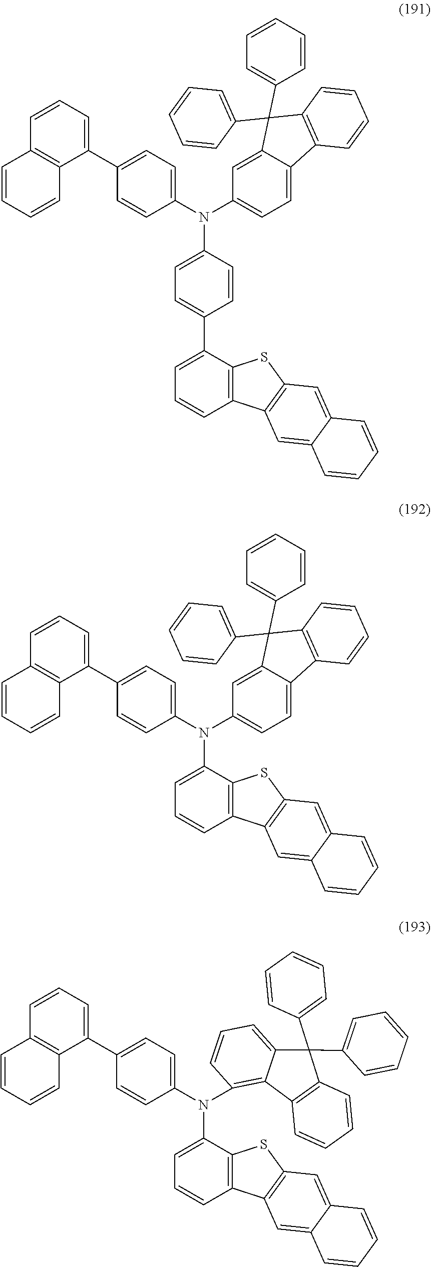

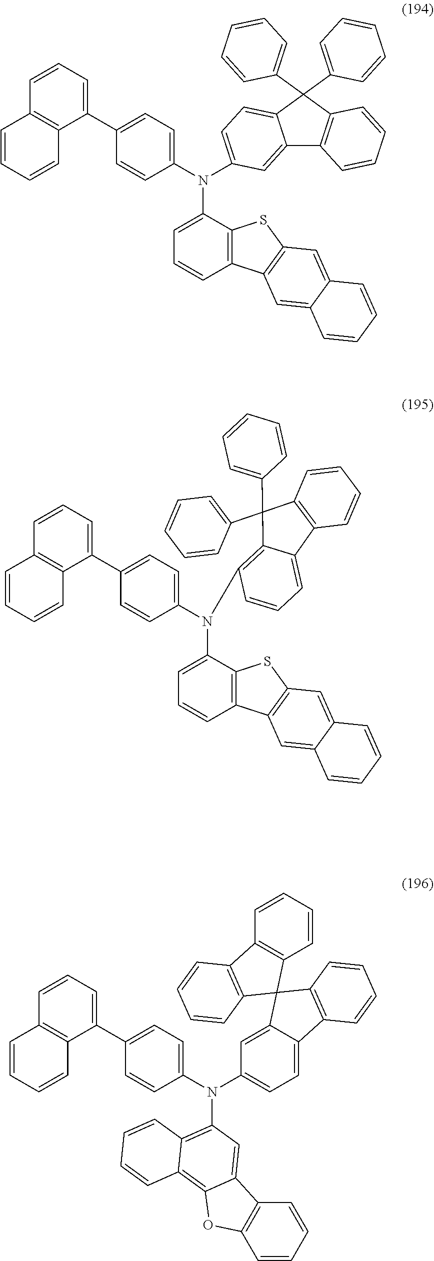

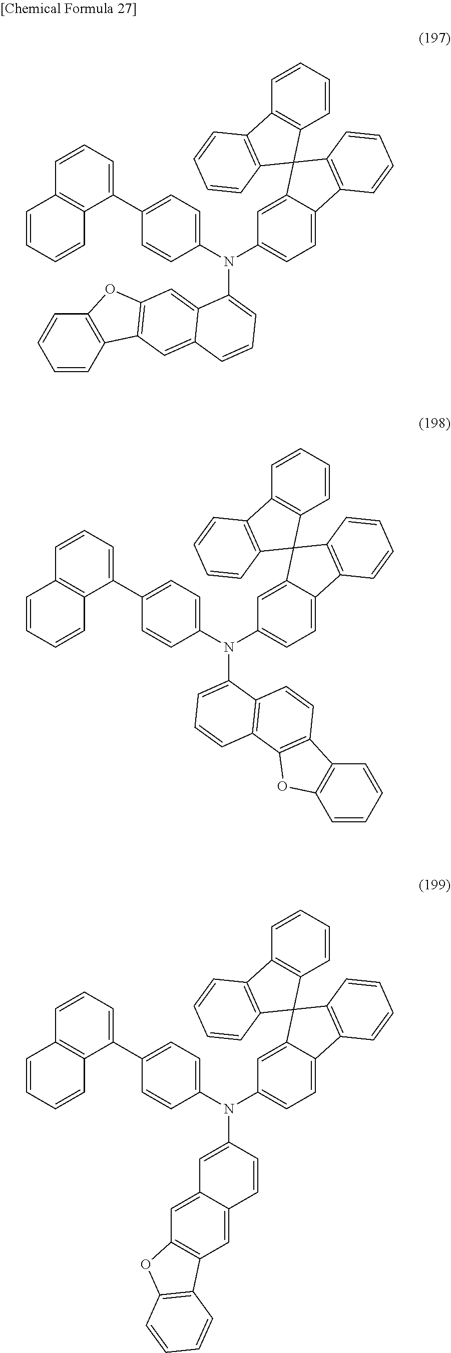

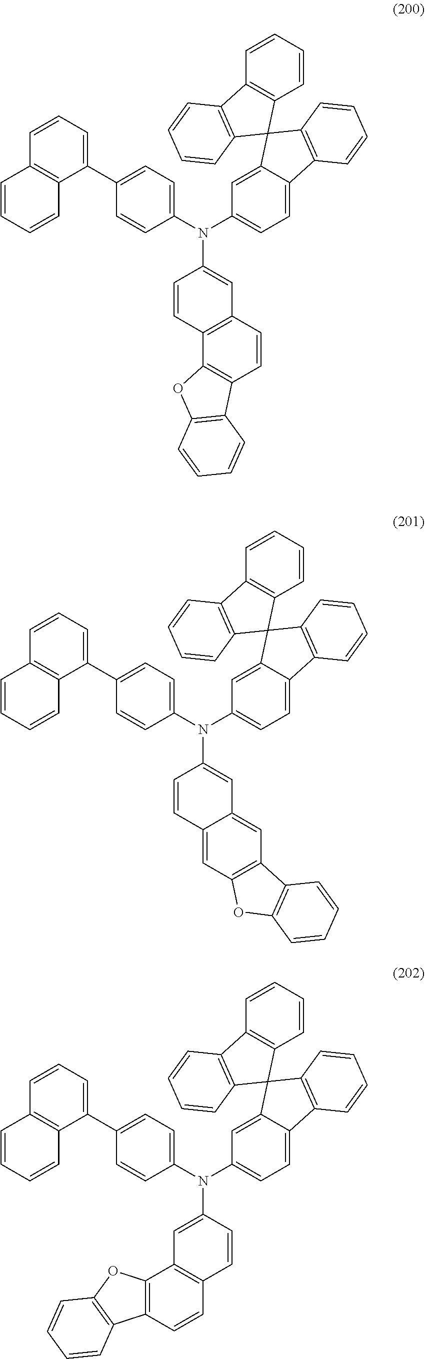

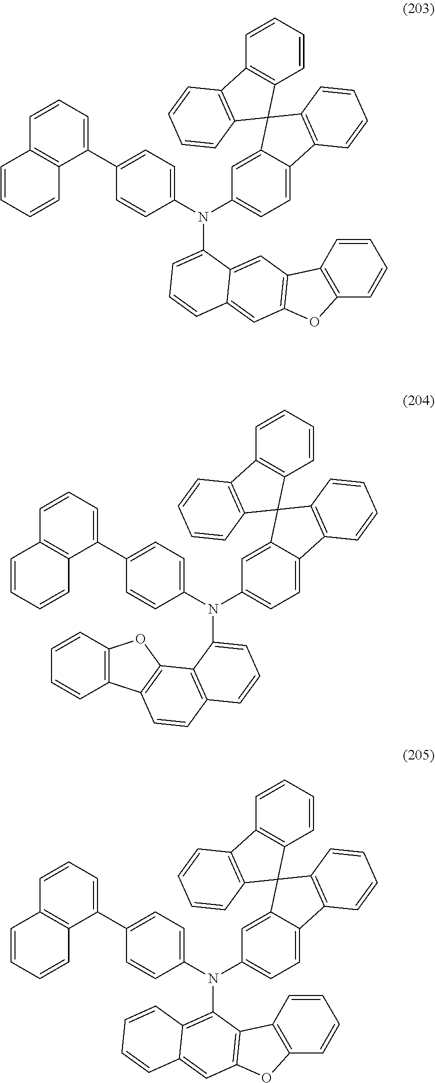

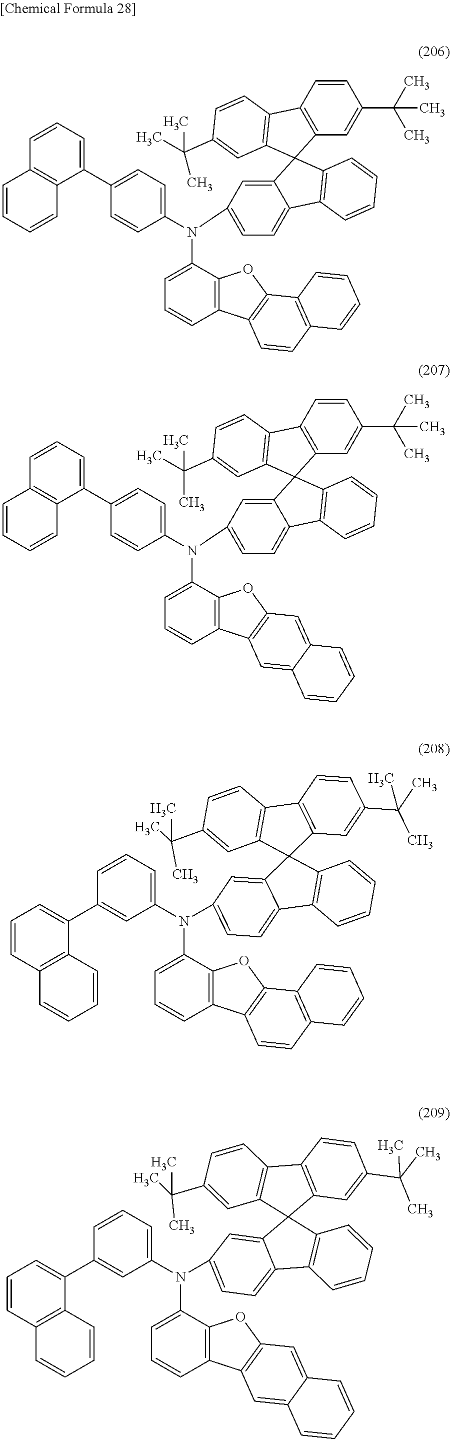

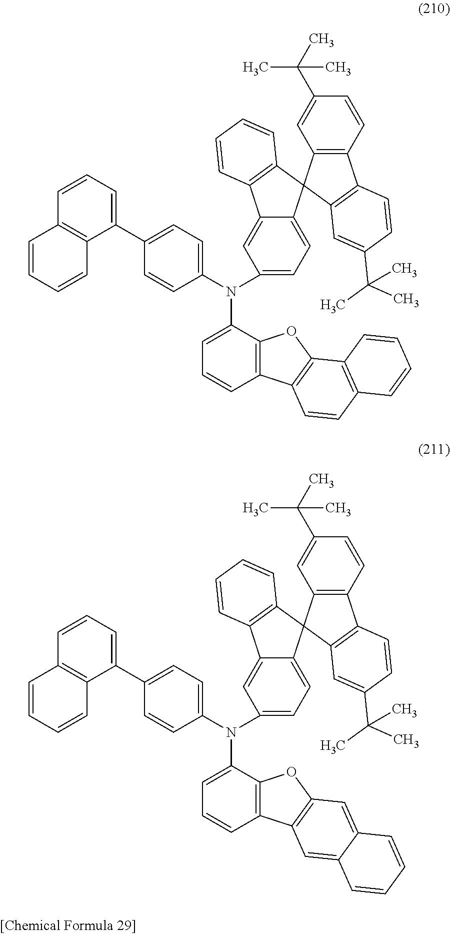

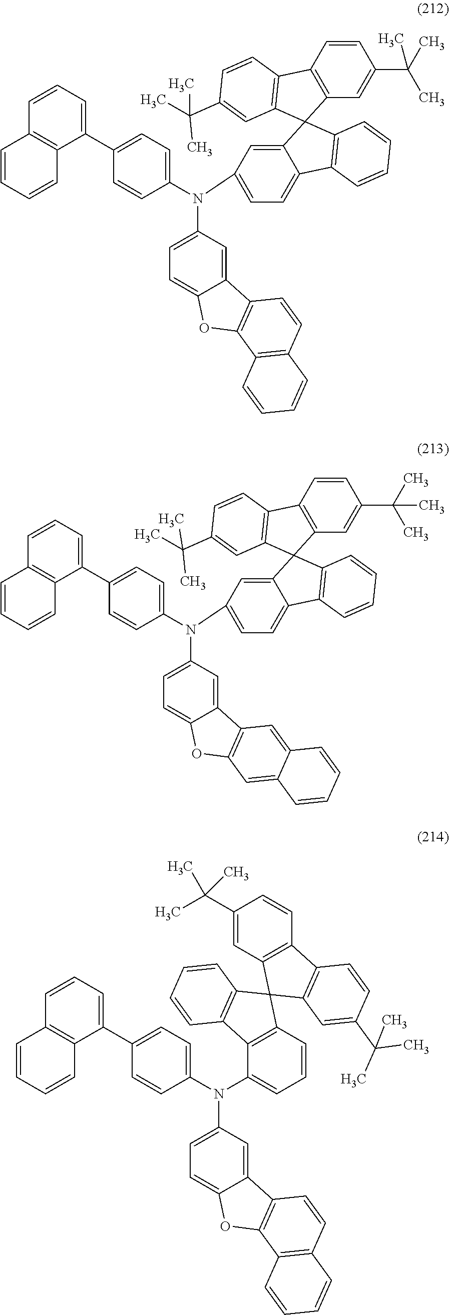

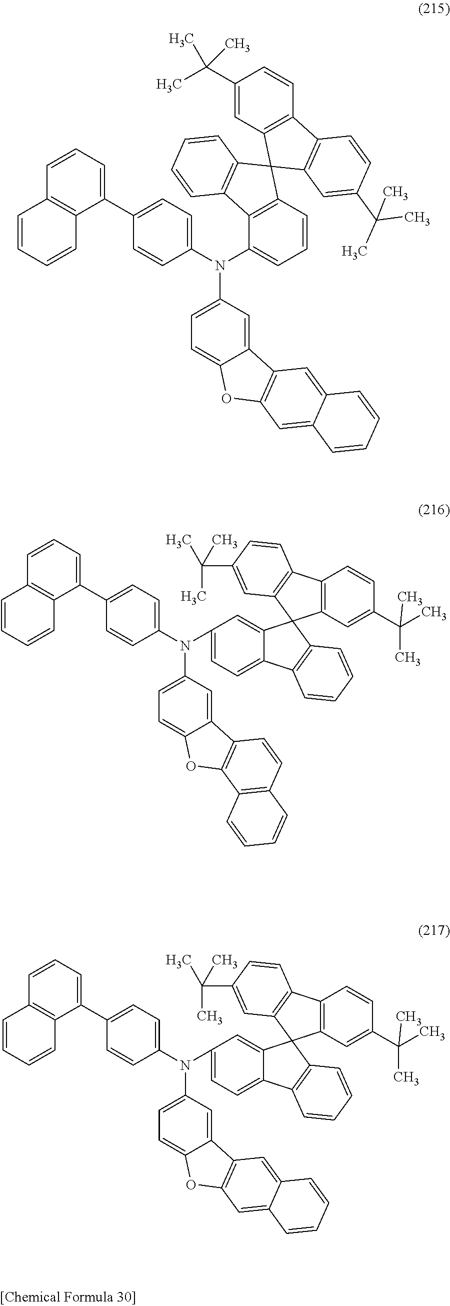

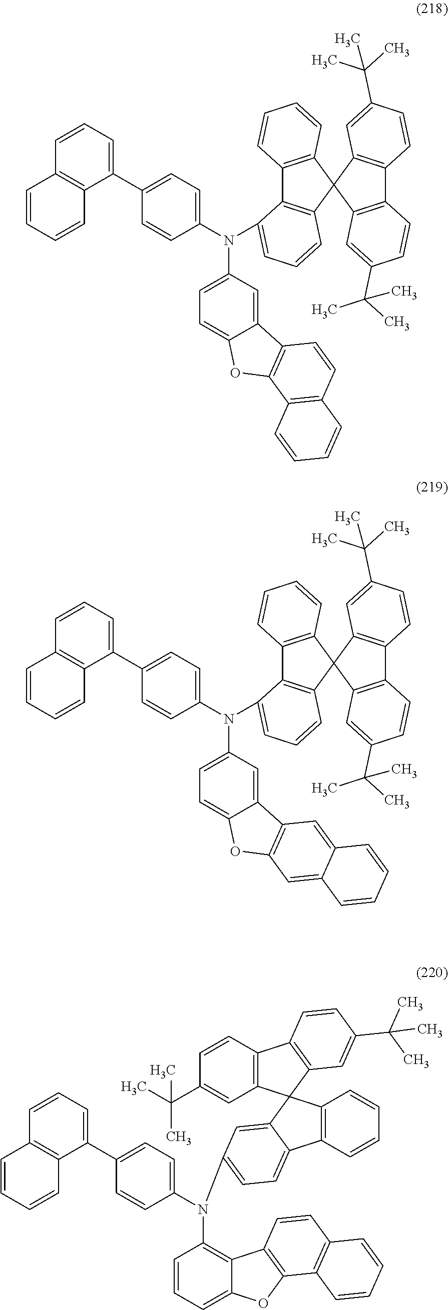

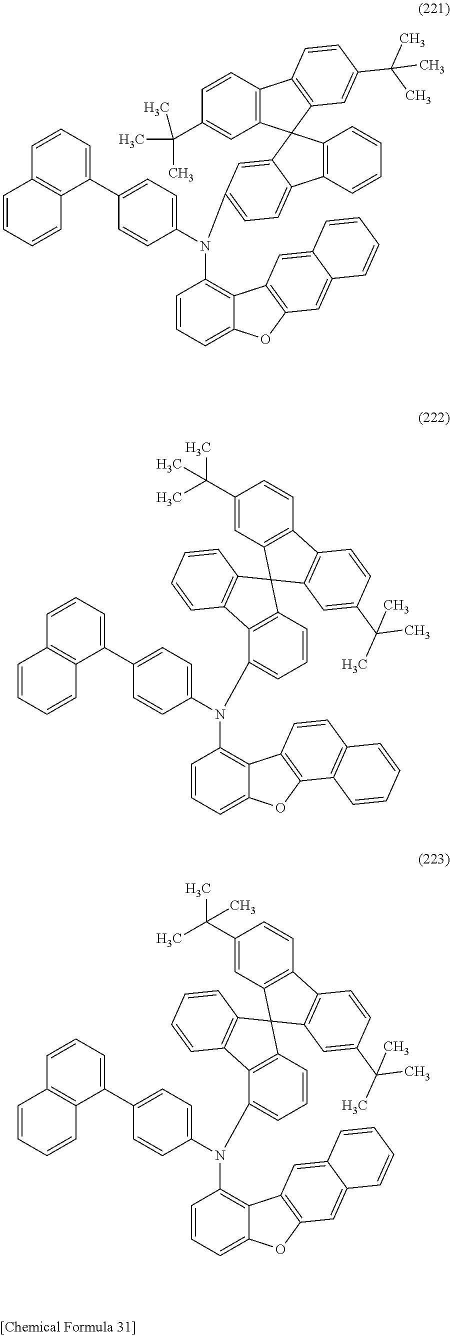

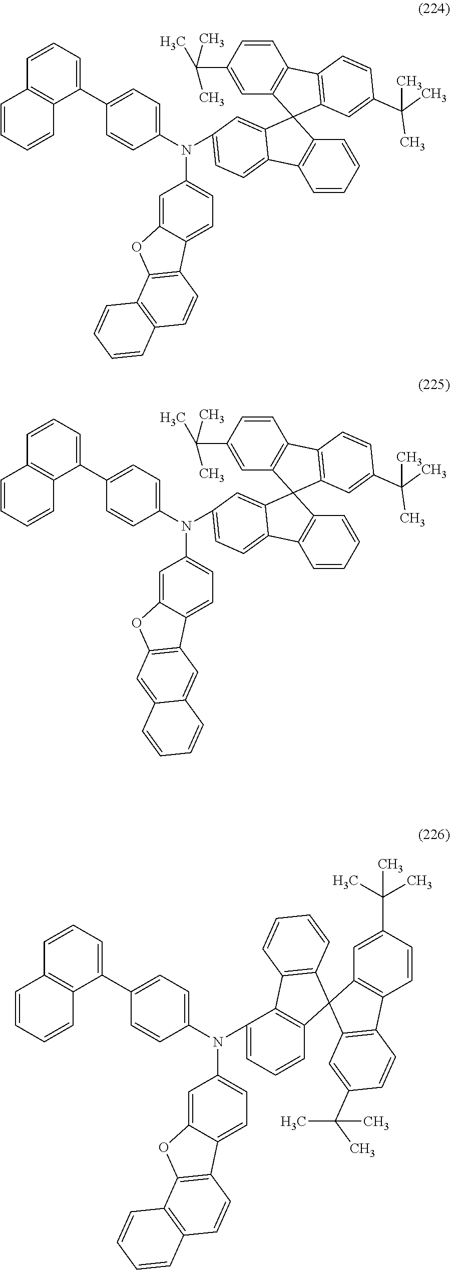

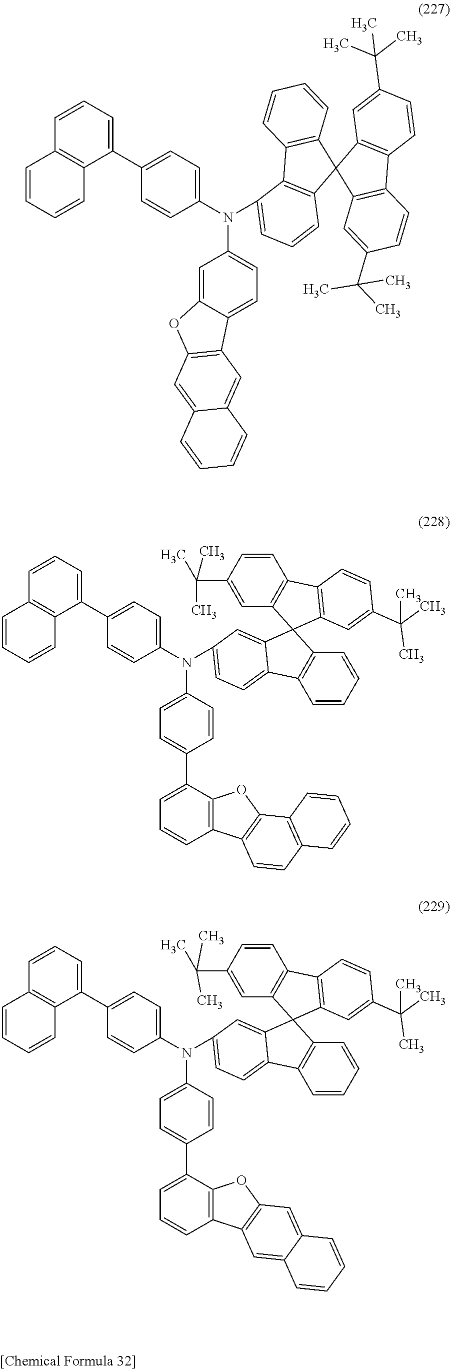

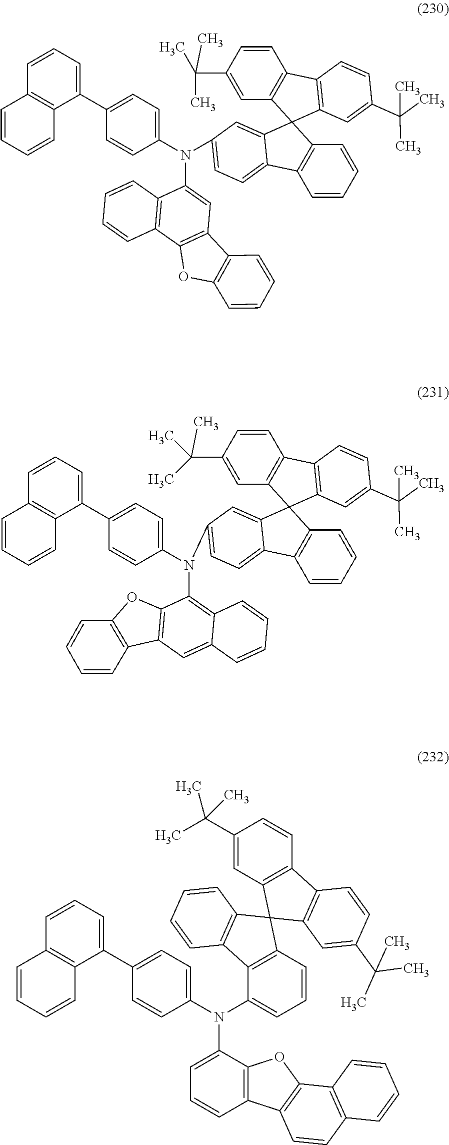

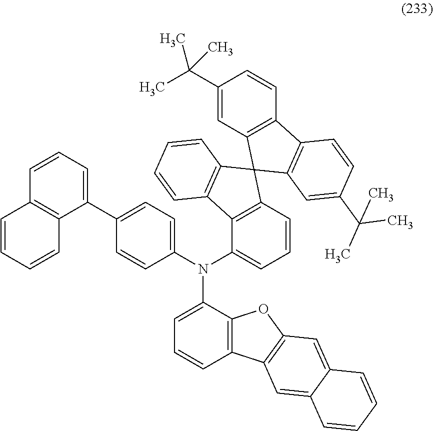

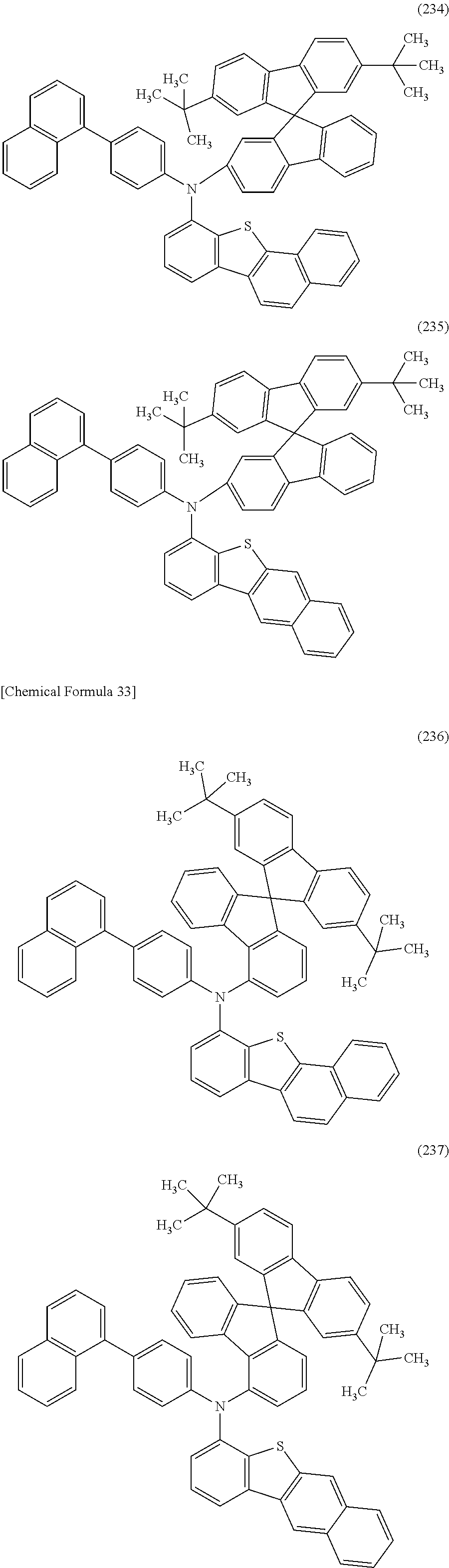

Next, as an example of a method of synthesizing the organic compound of one embodiment of the present invention, methods of synthesizing the organic compounds represented by General Formulae (G1) and (G7) will be described.

### <<Synthesis Method of Organic Compound Represented by General Formula (G1)>>

The organic compound represented by General Formula (G1) can be synthesized according to Synthesis Scheme (S-1) and Synthesis Scheme (S-9).

First, Synthesis Scheme (S-1) is described. Specifically, an amine compound including a naphthalene skeleton (Compound 1) and a spirobifluorene compound (Compound 2) are coupled to give a spirobifluorenamine compound including a naphthalene skeleton (Compound 3). Synthesis Scheme (S-1) is shown below.

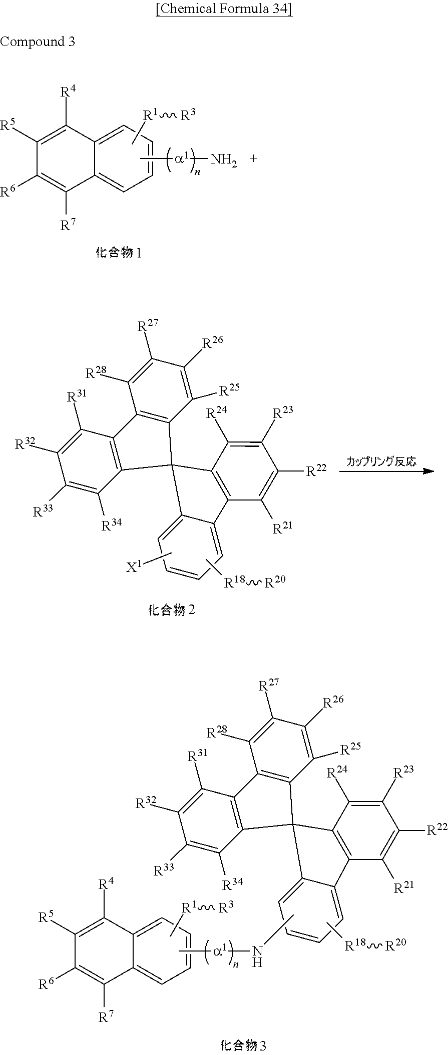

Next, Synthesis Scheme (S-2) is described. Specifically, the spirobifluorenamine compound including a naphthalene skeleton (Compound 3) and a compound including a benzonaphthofuran skeleton or a benzonaphthothiophene skeleton (Compound 4) are coupled to give the organic compound represented by General Formula (G1). Synthesis Scheme (S-2) is shown below.

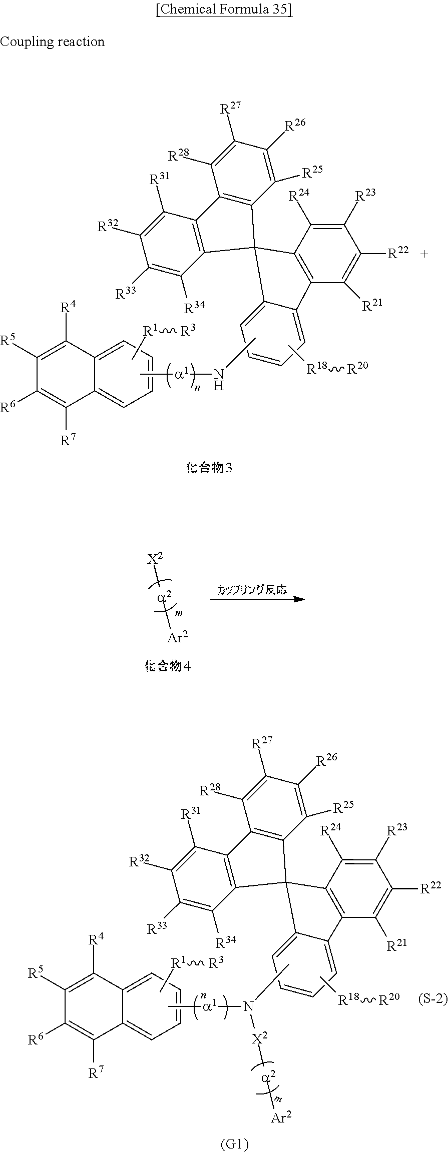

The spirobifluorenamine compound including a naphthalene skeleton (Compound 3) in Synthesis Schemes (S-1) and (S-2) can also be synthesized according to Synthesis Scheme (S-3). Specifically, a halogenated aryl compound including a naphthalene skeleton (Compound 5) and a spirobifluorenamine compound (Compound 6) are coupled to give the spirobifluorenamine compound including a naphthalene skeleton (Compound 3). Synthesis Scheme (S-3) is shown below.

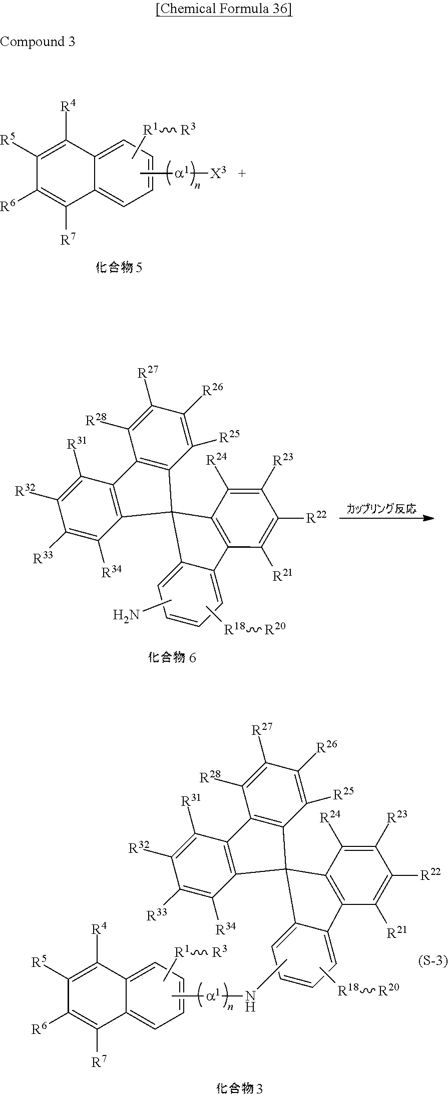

Next, Synthesis Scheme (S-4) is described. Specifically, the spirobifluorenamine compound (Compound 6) and the compound including a benzonaphthofuran skeleton or a benzonaphthothiophene skeleton (Compound 4) are coupled to give a spirobifluorenamine compound including a benzonaphthofuran skeleton or a benzonaphthothiophene skeleton (Compound 7). Synthesis Scheme (S-4) is shown below.

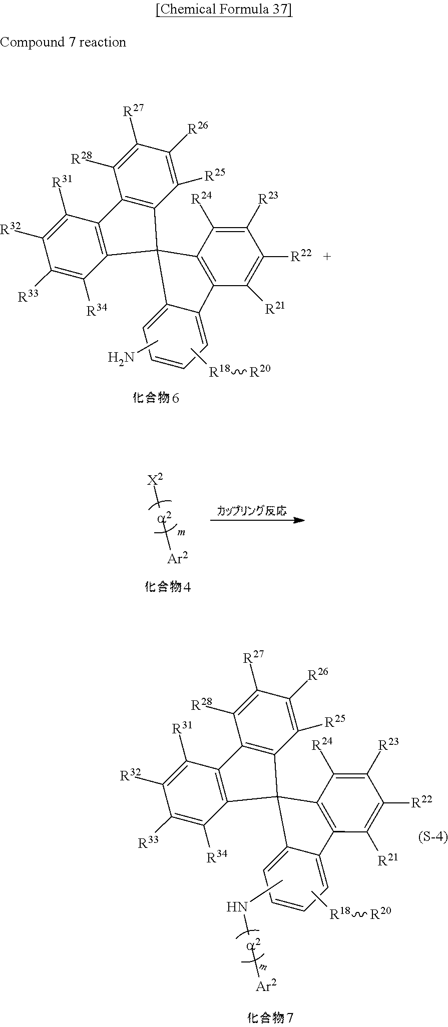

Next, Synthesis Scheme (S-5) is described. Specifically, the halogenated aryl compound including a naphthalene skeleton (Compound 5) and a spirobifluorenamine compound including a benzonaphthofuran skeleton or a benzonaphthothiophene skeleton (Compound 7) are coupled to give the organic compound represented by General Formula (G1). Synthesis Scheme (S-5) is shown below.

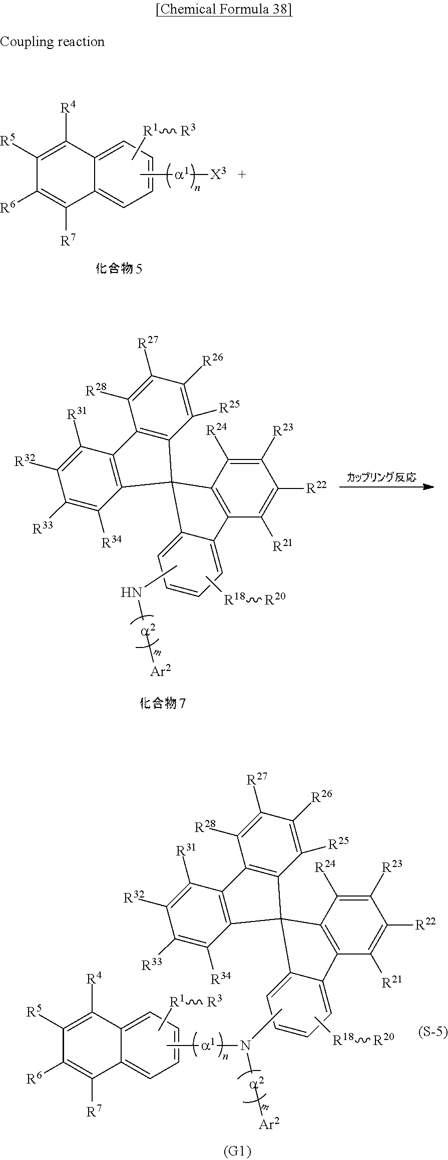

The spirobifluorenamine compound including a benzonaphthofuran skeleton or a benzonaphthothiophene skeleton (Compound 7) in Synthesis Schemes (S-4) and (S-5) can also be synthesized according to Synthesis Scheme (S-6). Specifically, the spirobifluorene compound (Compound 2) and an amine compound including a benzonaphthofuran skeleton or a benzonaphthothiophene skeleton (Compound 8) are coupled to give the spirobifluorenamine compound including a benzonaphthofuran skeleton or a benzonaphthothiophene skeleton compound (Compound 7). Synthesis Scheme (S-6) is shown below.

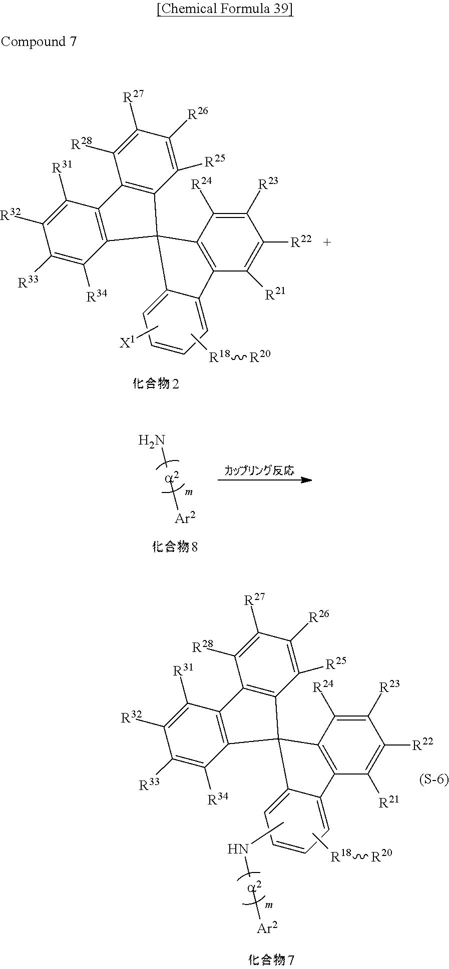

Next, Synthesis Scheme (S-7) is described. Specifically, the halogenated aryl compound including a naphthalene skeleton (Compound 5) and the amine compound including a benzonaphthofuran skeleton or a benzonaphthothiophene skeleton (Compound 8) are coupled to give an amine compound including a naphthalene skeleton and a benzonaphthofuran skeleton or a benzonaphthothiophene skeleton (Compound 9). Synthesis Scheme (S-7) is shown below.

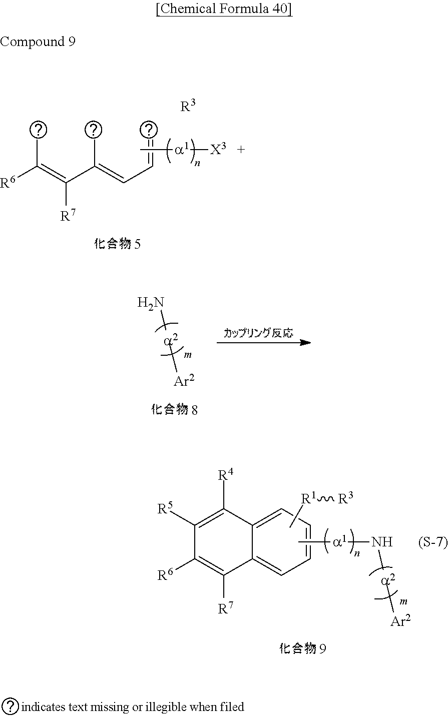

Next, Synthesis Scheme (S-8) is described. Specifically, the amine compound (Compound 9) and the spirobifluorene compound (Compound 2) are coupled to give the organic compound represented by General Formula (G1). Synthesis Scheme (S-8) is shown below.

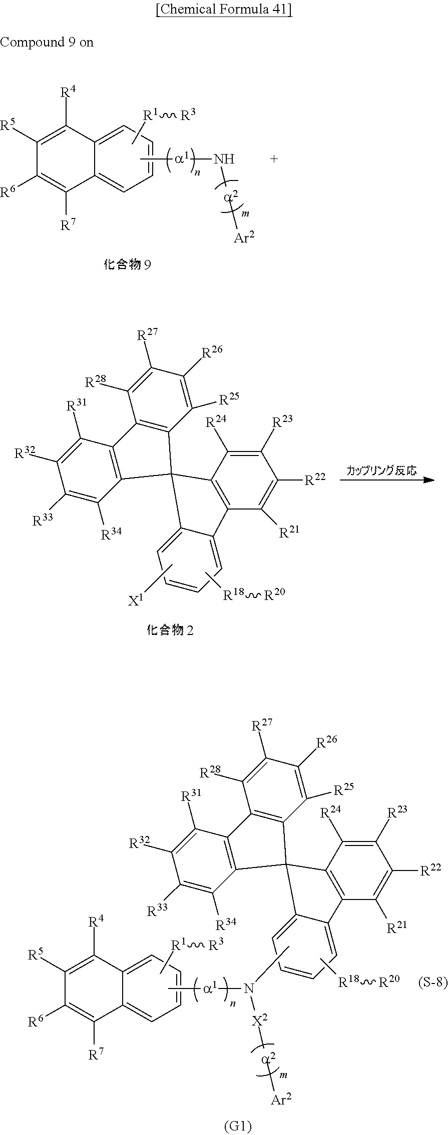

The amine compound (Compound 9) in Synthesis Schemes (S-7) and (S-8) can also be synthesized according to Synthesis Scheme (S-9). Specifically, the amine compound including a naphthalene skeleton (Compound 1) and the compound including a benzonaphthofuran skeleton or a benzonaphthothiophene skeleton (Compound 4) are coupled to give the amine compound (Compound 9). Synthesis Scheme (S-9) is shown below.

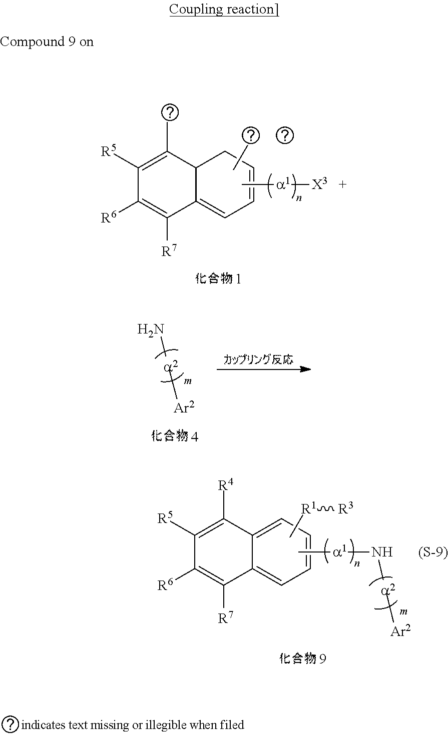

In Synthesis Schemes (S-1) to (S-9), each of X¹ to X³ independently represents chlorine, bromine, iodine, or a triflate group.

In the case where the Buchwald-Hartwig reaction using a palladium catalyst is employed in Synthesis Schemes (S-1) and (S-9), a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) can be used as the palladium catalyst; tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, di(tert-butyl)(1-methyl-2,2-diphenylcyclopropyl)phosphine (abbreviation: cBRIDP) or the like can be used as a ligand of the palladium catalyst. In the reaction, an organic base such as sodium tert-butoxide, an inorganic base such as potassium carbonate, cesium carbonate, or sodium carbonate, or the like can be used as a base. In the reaction, a functional host compound such as 18-crown 6-ether can also be used. In the reaction, toluene, xylene, benzene, tetrahydrofuran, dioxane, or the like can be used as a solvent.

A coupling reaction using copper or a copper compound can be used for each of Synthesis Schemes (S-1) to (S-9). Examples of the base to be used include an inorganic base such as potassium carbonate. As the solvent that can be used in the reaction, 1,3-dimethyl-3,4,5,6-tetrahydro-2(1H)-pyrimidinone (DMPU), toluene, xylene, benzene, and the like can be given. In the coupling reaction using copper or a copper compound, the target substance can be obtained in a shorter time and in a higher yield when the reaction temperature is higher than or equal to 100° C.; therefore, it is preferable to use DMPU or xylene, which has a high boiling point. A reaction temperature higher than or equal to 150° C. is further preferable, and accordingly, DMPU is further preferably used.

Reagents that can be used in Synthesis Schemes (S-1) to (S-9) are not limited to the above-described reagents. The method for synthesizing the organic compound of the present invention represented by General Formula (G1) is not limited to Synthesis Schemes (S-1) to (S-9).

### <<Synthesis Method of Organic Compound Represented by General Formula (G7)>>

The organic compound of the present invention represented by General Formula (G7) can be synthesized by a method similar to Synthesis Schemes (S-1) to (S-9), which are the aforementioned methods for synthesizing the organic compound represented by General Formula (G1). Specifically, Synthesis Scheme (S-10) to Synthesis Scheme (S-16) shown below can be employed for the synthesis.

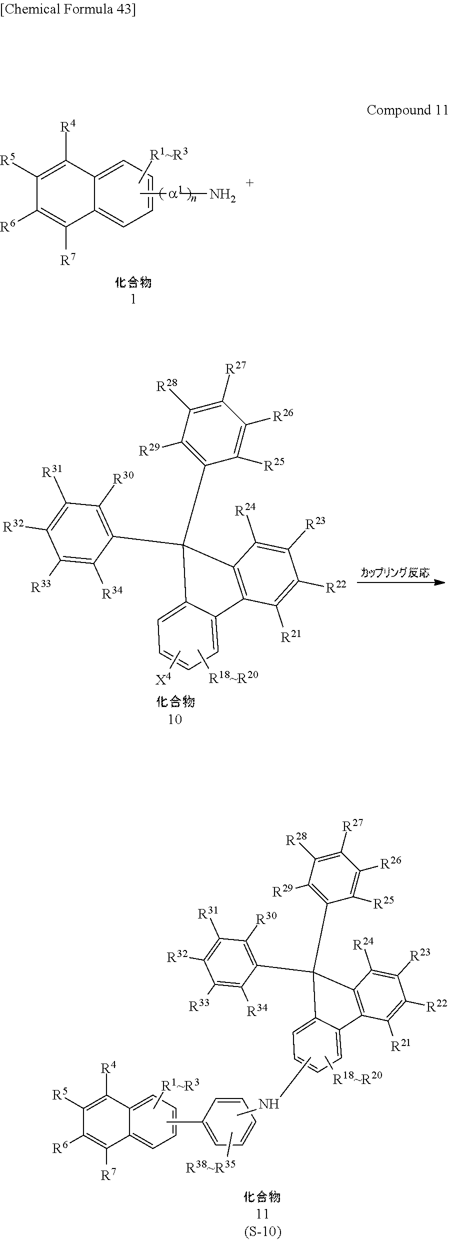

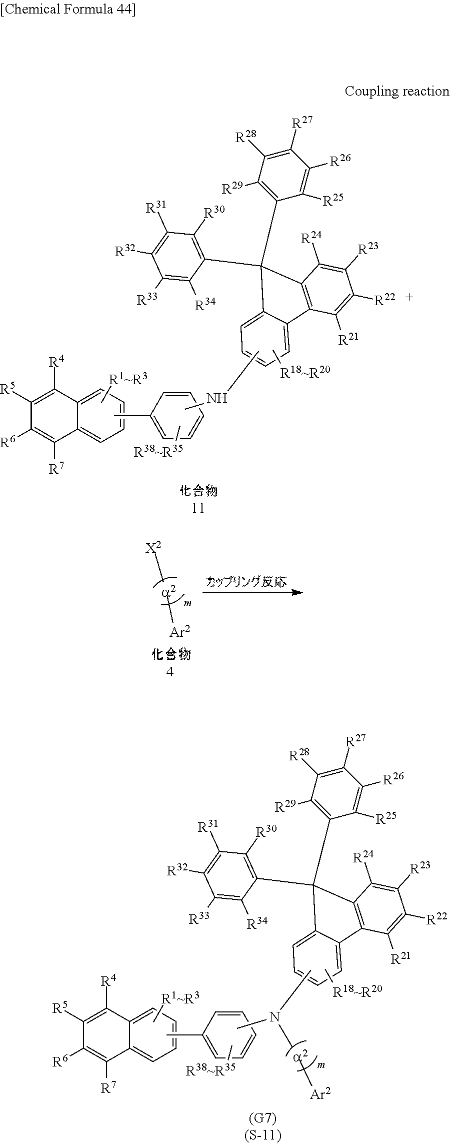

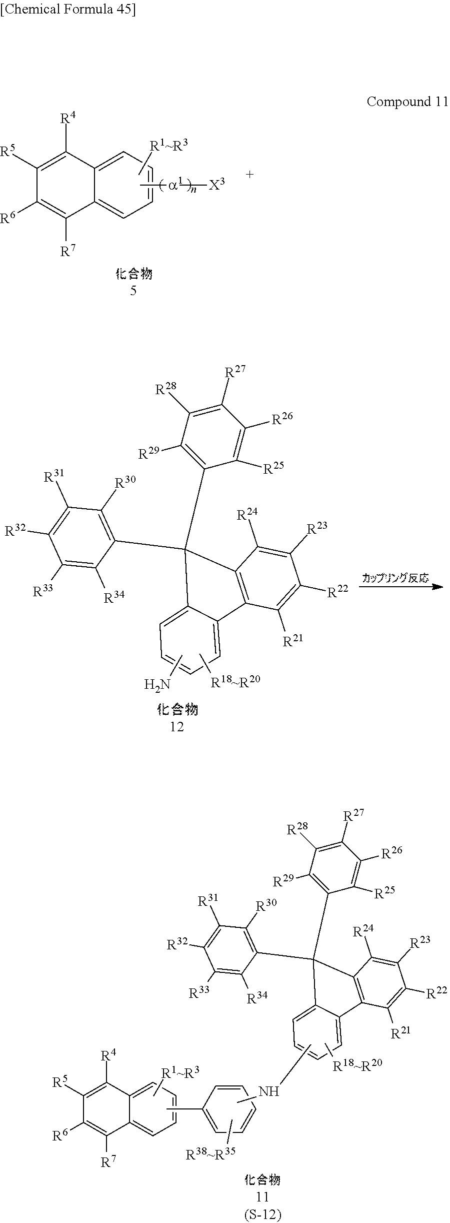

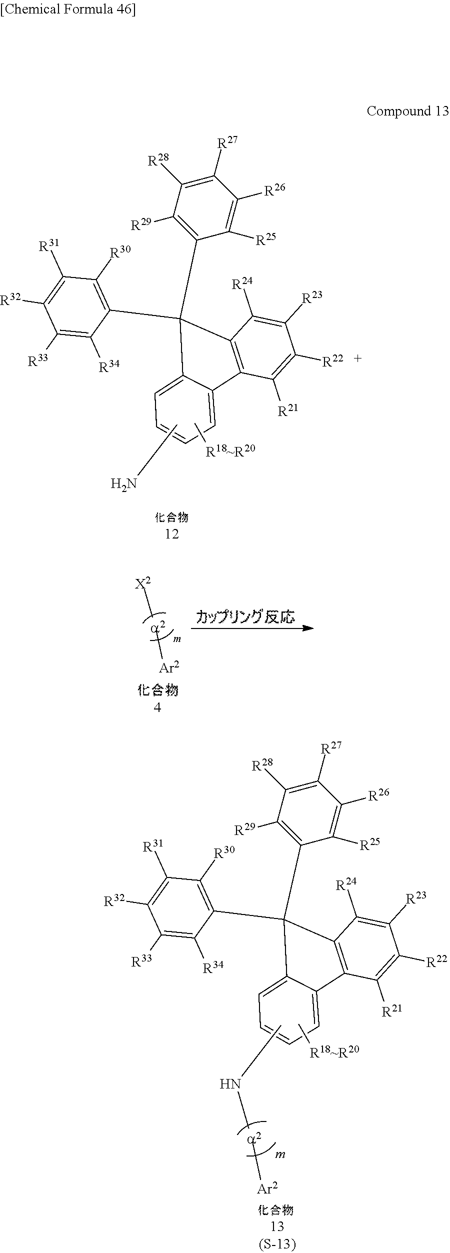

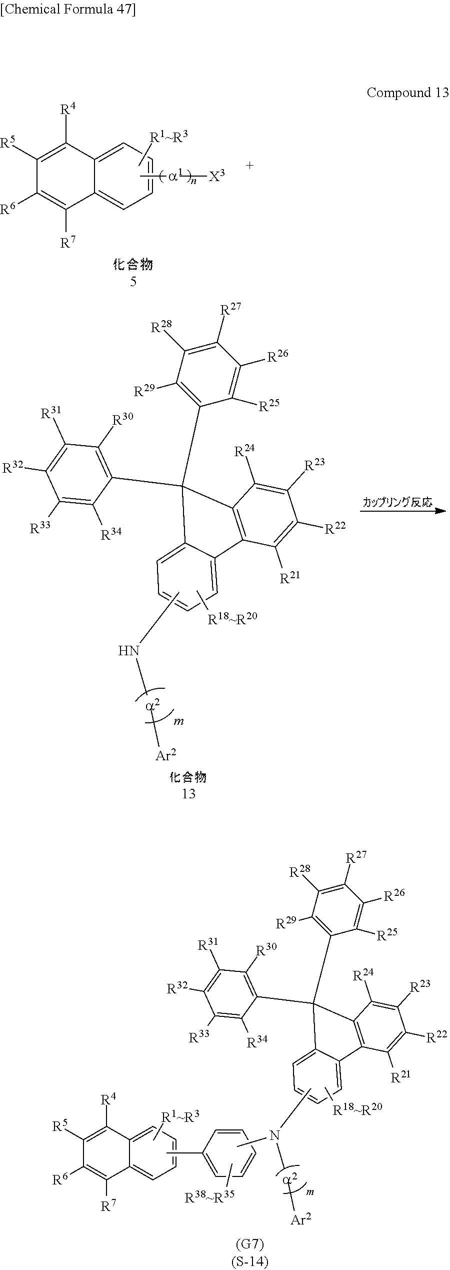

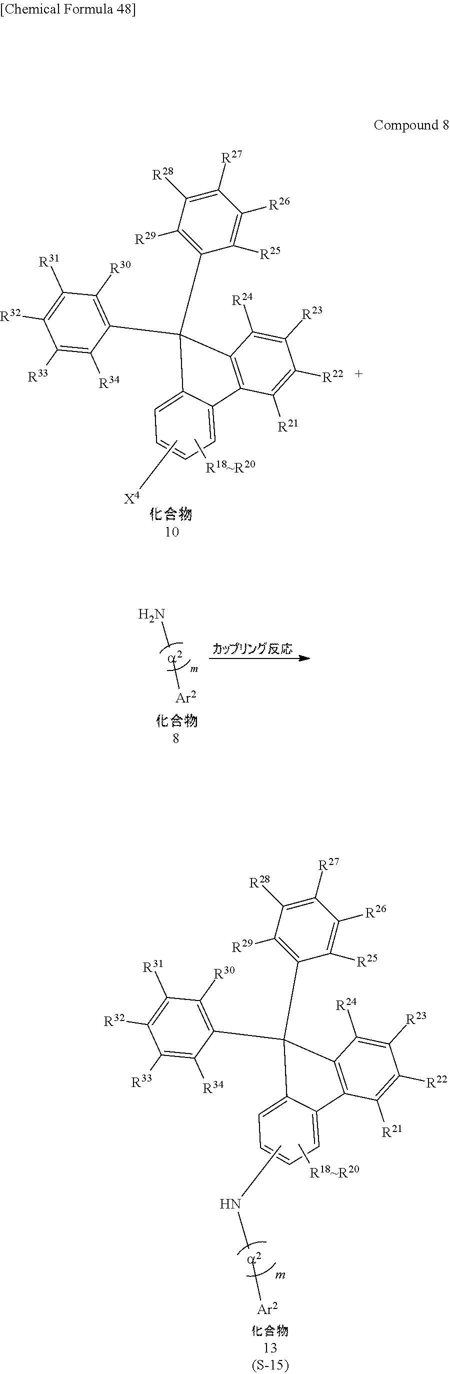

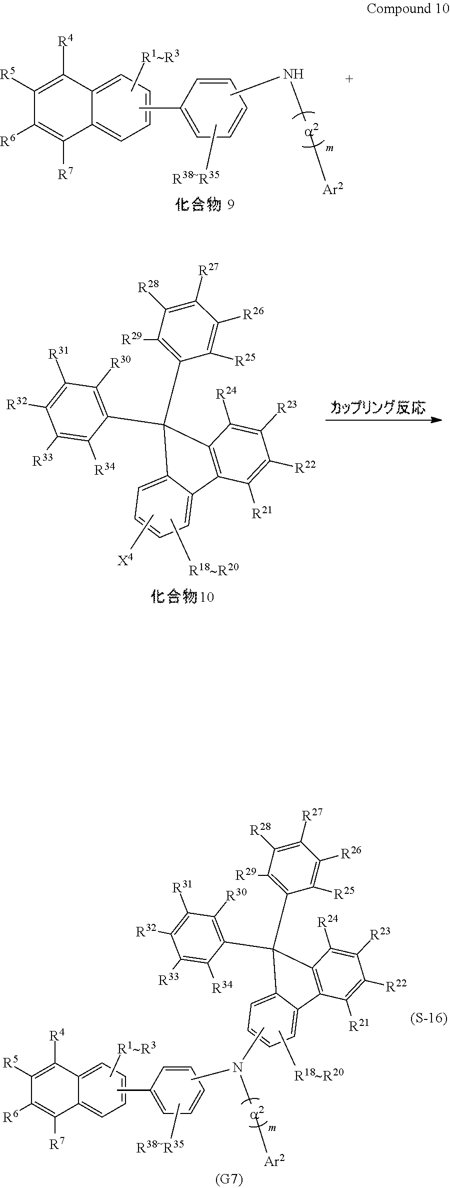

In Synthesis Schemes (S-10) to (S-16), X² and X³ are the same as those described (shown) above and thus are not described here.

In Synthesis Schemes (S-10), (S-15), and (S-16), X⁴ represents chlorine, bromine, iodine, or a triflate group.

In Synthesis Schemes (S-10) to (S-16), the same reaction conditions as those in Synthesis Schemes (S-1) to (S-9) can be used.

The method for synthesizing the organic compound of the present invention represented by General Formula (G7) is not limited to Synthesis Schemes (S-10) to (S-16).

The organic compounds of embodiments of the present invention can be synthesized by the above methods, but the present invention is not limited thereto and other synthesis methods may be employed.

The structures described in this embodiment can be used in appropriate combination with any of the structures described in the other embodiments.

### Embodiment 2

In this embodiment, an organic semiconductor device of one embodiment of the present invention is described.

For the organic semiconductor device of one embodiment of the present invention, an organic compound represented by General Formula (G8) can be used.

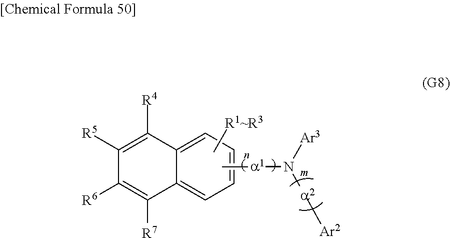

In General Formula (G8), each of α¹ and α² independently represents a substituted or unsubstituted phenylene group or a substituted or unsubstituted naphthalene-diyl group; n is 1 or 2; m is 0, 1, or 2; each of R¹ to R⁷ independently represents hydrogen (including deuterium), a straight-chain or branched-chain alkyl group having 1 to 6 carbon atoms, a substituted or unsubstituted cycloalkyl group having 3 to 10 carbon atoms, or a substituted or unsubstituted phenyl group; Ar² represents a substituted or unsubstituted benzo[b]naphtho[2,1-d]furanyl group, a substituted or unsubstituted benzo[b]naphtho[2,3-d]furanyl group, a substituted or unsubstituted benzo[b]naphtho[2,1-d]thiophenyl group, or a substituted or unsubstituted benzo[b]naphtho[2,3-d]thiophenyl group; and Ar³ represents a substituted or unsubstituted fluorenyl group or a substituted or unsubstituted spirobifluorenyl group. When n is 2, a plurality of α¹'s may be the same or different from each other. When m is 2, a plurality of α²'s may be the same or different from each other.

For the organic semiconductor device of one embodiment of the present invention, an organic compound represented by General Formula (G9) can be used.

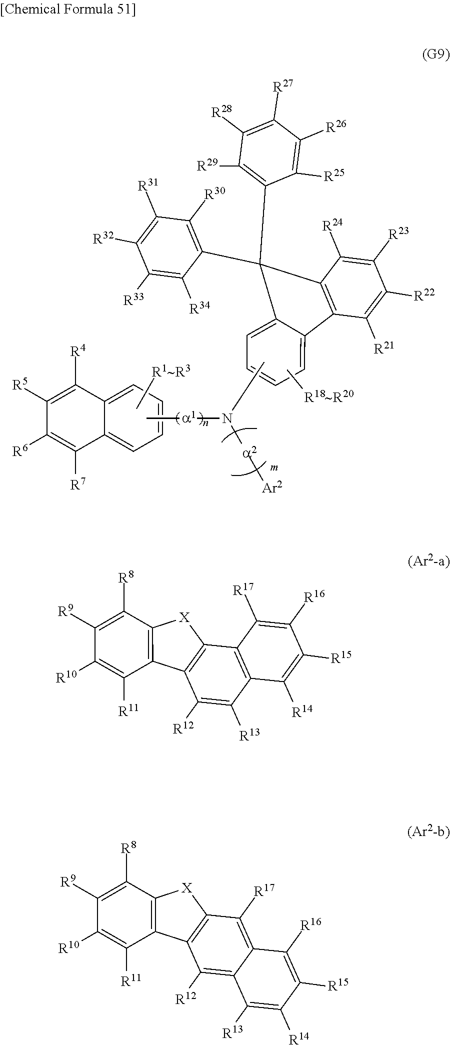

In General Formula (G9), each of α¹ and α² independently represents a substituted or unsubstituted phenylene group or a substituted or unsubstituted naphthalene-diyl group; n is 1 or 2; m is 0, 1, or 2; each of R¹ to R⁷ independently represents hydrogen (including deuterium), a straight-chain or branched-chain alkyl group having 1 to 6 carbon atoms, a substituted or unsubstituted cycloalkyl group having 3 to 10 carbon atoms, or a substituted or unsubstituted phenyl group; Ar² represents a group represented by General Formula (Ar²-a) or General Formula (Ar²-b); any one of R⁸ to R¹⁷ represents a bond; each of R⁸ to R¹⁷ other than the bond and each of R¹⁸ to R²⁸ and R³¹ to R³⁴ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; when each of R²⁹ and R³⁰ represents a bond, R²⁹ and R³⁰ are bonded to each other to form a ring; when neither R²⁹ nor R³⁰ represents a bond, each of R²⁹ and R³⁰ independently represents hydrogen (including deuterium), a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms; and X represents oxygen or sulfur. When n is 2, a plurality of α¹'s may be the same or different from each other. When m is 2, a plurality of α²'s may be the same or different from each other.

As described above, the organic compounds represented by General Formulae (G8) and (G9) each have high stability and high heat resistance by including the naphthyl group bonded to nitrogen through the phenylene group or the biphenyldiyl group. Accordingly, with the use of such an organic compound, an organic semiconductor device having high reliability and high quality can be manufactured.

Since the organic compound represented by each of General Formulae (G8) and (G9) includes a benzo[b]naphtho[2,1-d]furanyl group, a benzo[b]naphtho[2,3-d]furanyl group, a benzo[b]naphtho[2,1-d]thiophenyl group, or a benzo[b]naphtho[2,3-d]thiophenyl group, which is bonded to nitrogen directly or through an arylene group, the driving voltage of the organic semiconductor device including the organic compound can be inhibited from significantly changing over driving time. In addition, the organic semiconductor device can have a long driving lifetime.

The organic compounds represented by General Formulae (G8) and (G9) can have low sublimation temperature, inhibiting thermal decomposition during sublimation.

The specific examples of the substituents that can be used in the organic compounds represented by the general formulae described in Embodiment 1 can also be applied to the organic compounds represented by General Formulae (G8) and (G9).

Specific examples of the organic compounds represented by General Formulae (G8) and (G9) include organic compounds represented by Structural Formula (300) to Structural Formula (344) shown below. Note that the organic compounds represented by General Formulae (G8) and (G9) are not limited to the organic compounds represented by the following structural formulae.

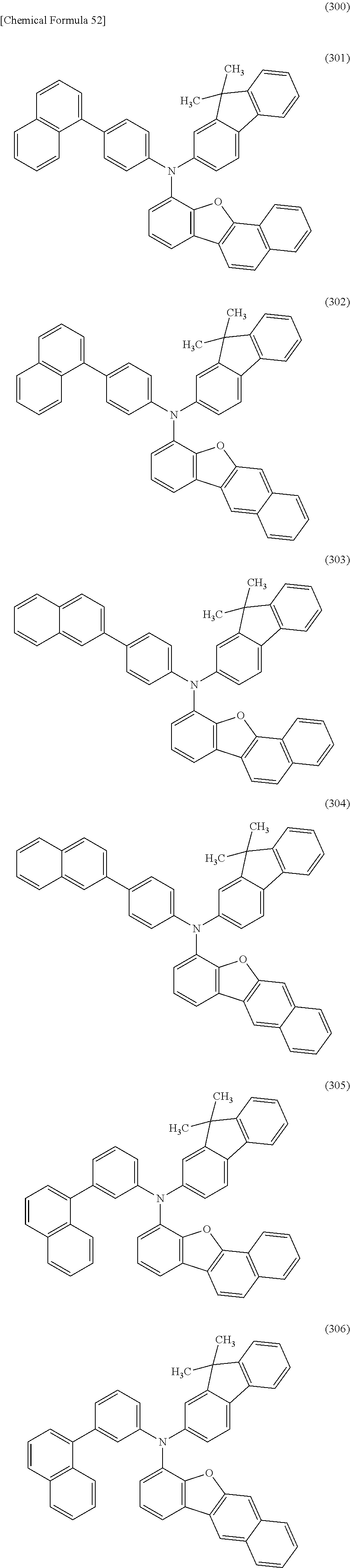

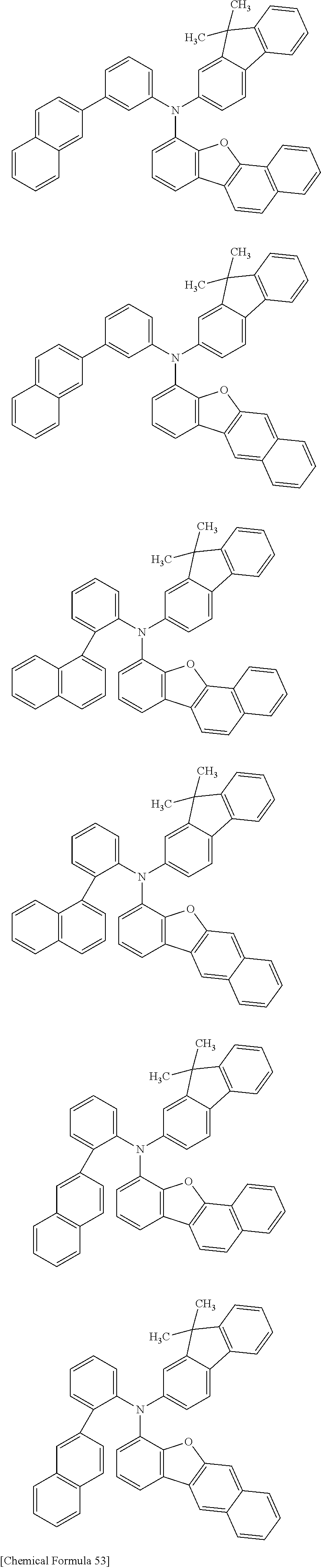

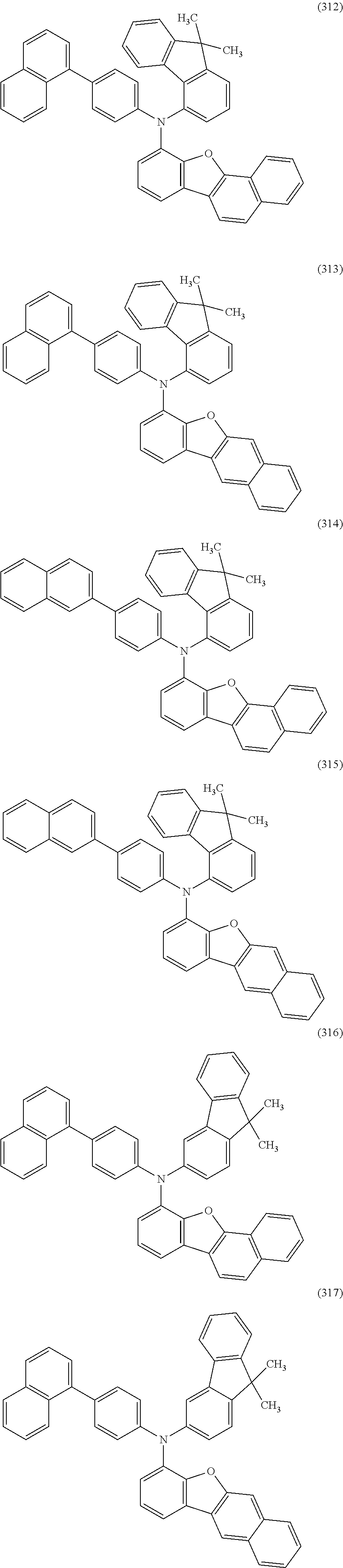

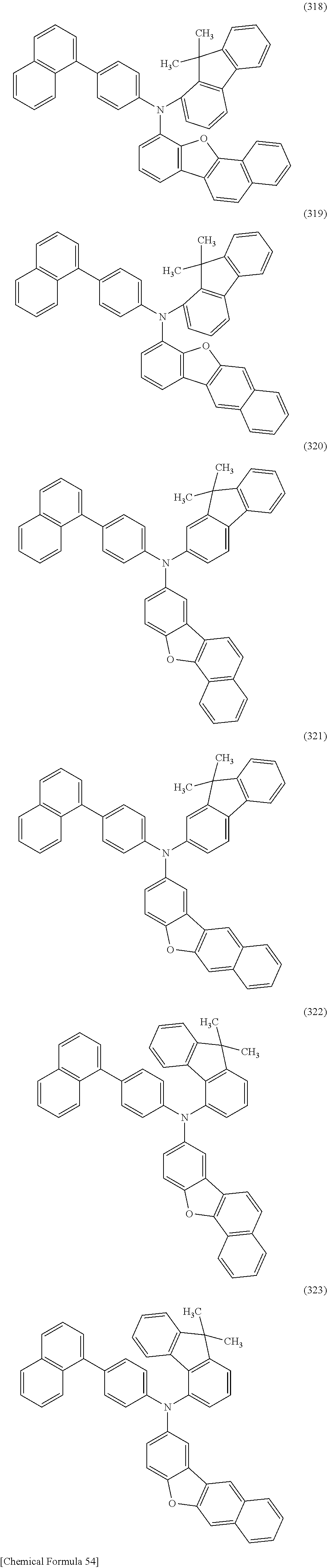

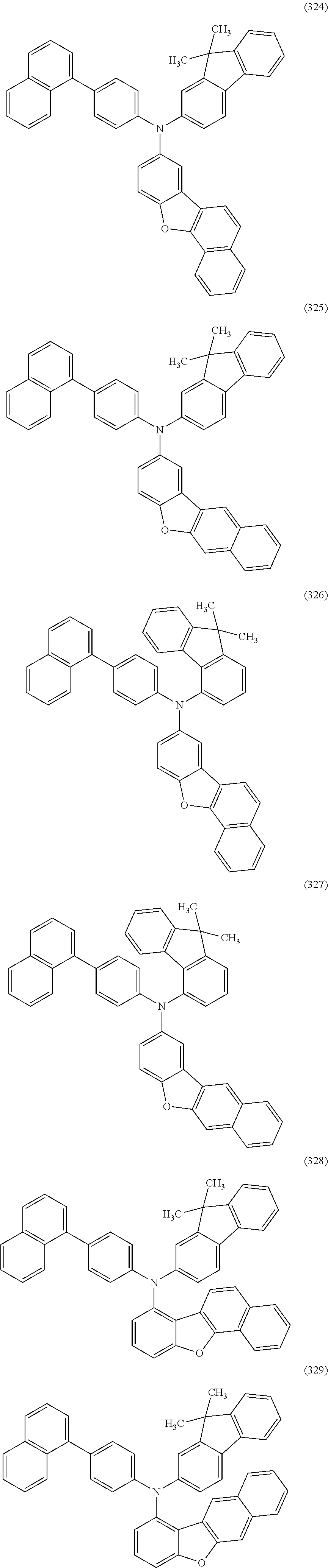

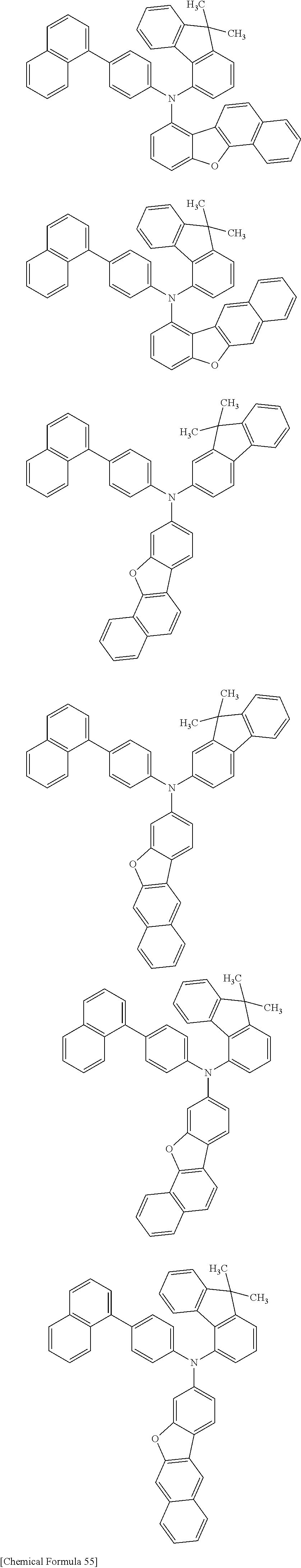

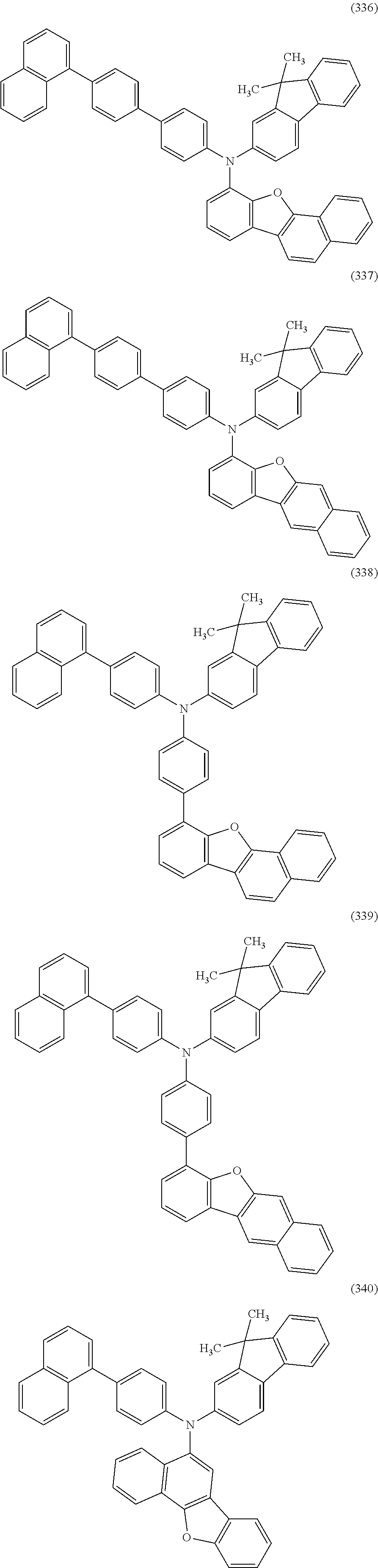

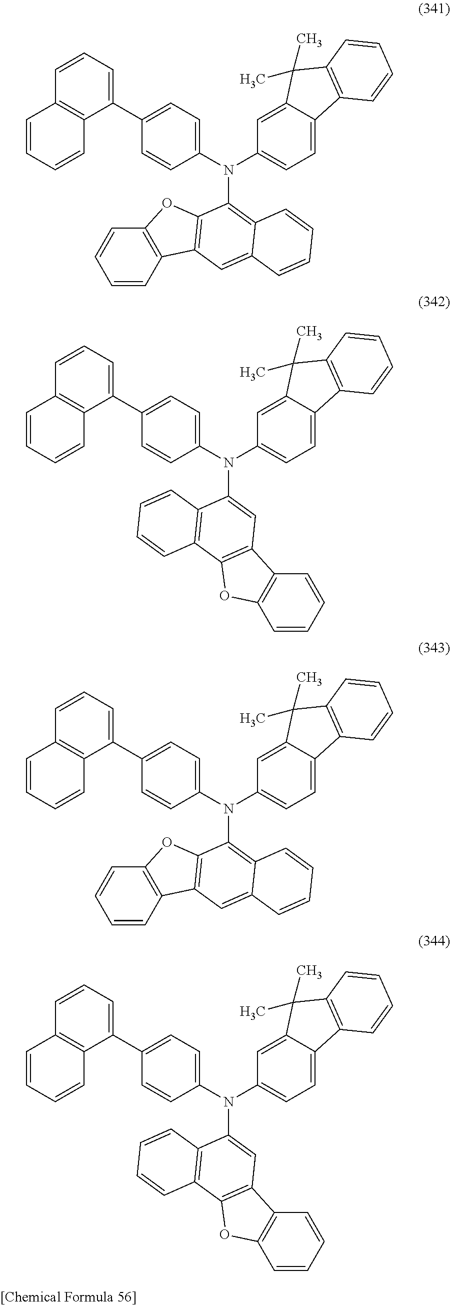

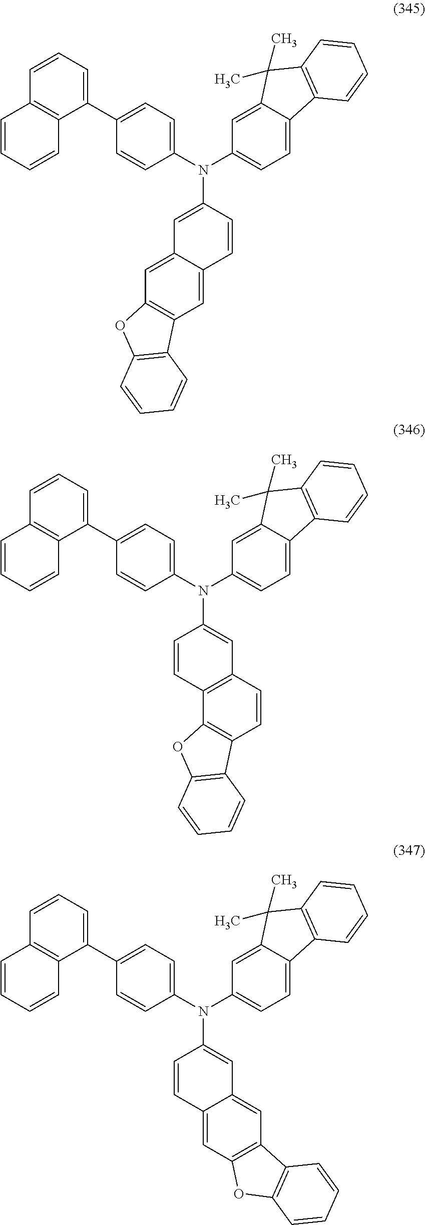

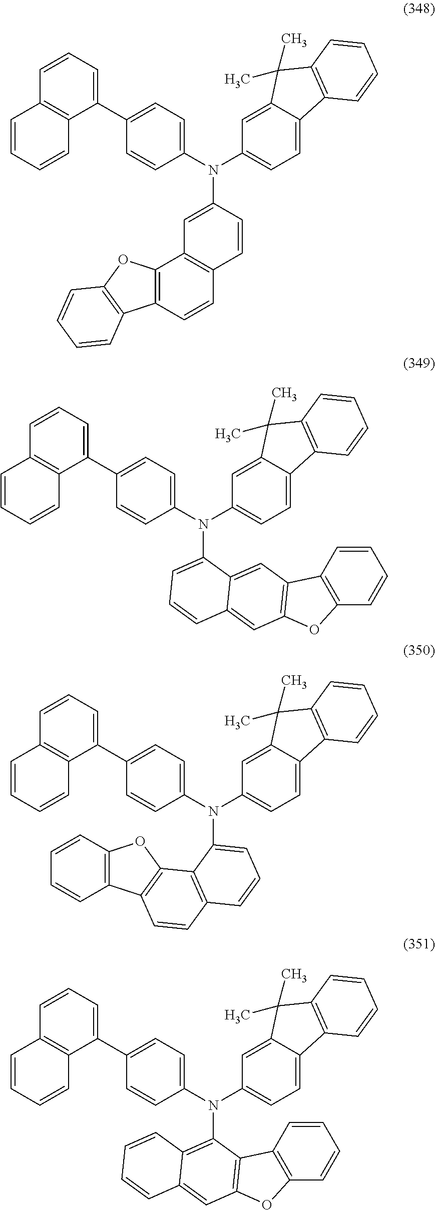

For the organic semiconductor device of one embodiment of the present invention, any of the organic compounds represented by General Formulae (G1) to (G7) described in Embodiment 1 can be used.

The organic compound represented by any of General Formulae (G1) to (G9) is suitable for a light-emitting device of organic semiconductor devices, such as an organic light-emitting diode (OLED) and can also be used for other organic semiconductor devices. Examples of other applications include photoelectric conversion devices such as an organic optical sensor and an organic thin film solar cell, an organic field-effect transistor, a semiconductor gas sensor, a diode, an inverter, and a storage device.

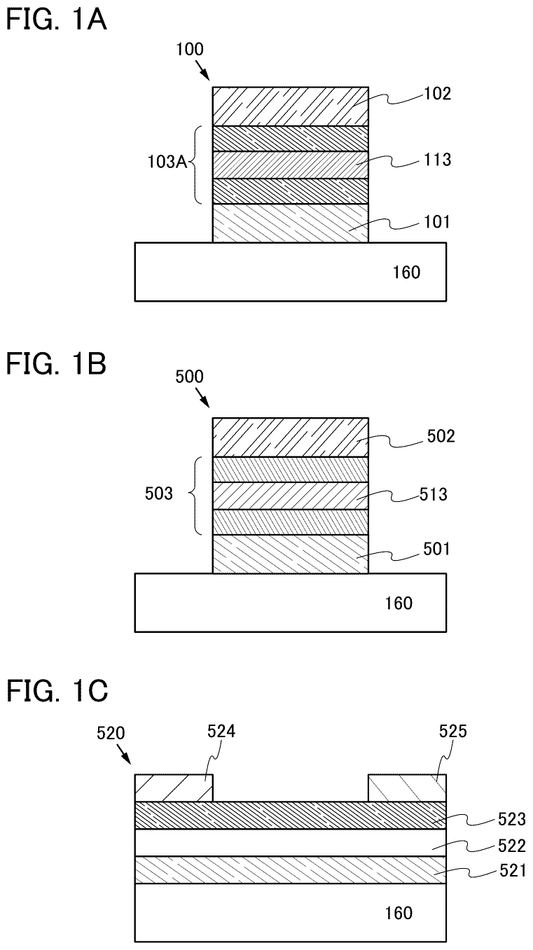

FIGS. 1A to 1C are cross-sectional views illustrating a light-emitting device, a photoelectric conversion device, and an organic field-effect transistor, each of which is an example of the organic semiconductor device of one embodiment of the present invention.

FIG. 1A is a cross-sectional view of a light-emitting device 100. The light-emitting device 100 includes a first electrode 101 and a second electrode 102 which are provided over a substrate 160, and an organic compound layer 103A held between the first electrode 101 and the second electrode 102. One of the first electrode 101 and the second electrode 102 serves as an anode and the other serves as a cathode. The organic compound layer 103A includes a light-emitting layer 113, and the light-emitting layer 113 contains a light-emitting material. When voltage is applied between the first electrode 101 and the second electrode 102, light is emitted from the organic compound layer 103A; accordingly, the light-emitting device 100 can be used as an organic light-emitting diode. Although not illustrated, the organic compound layer 103A may include, in addition to the light-emitting layer 113, a variety of layers such as a hole-injection layer, a hole-transport layer, an electron-transport layer, an electron-injection layer, a hole-blocking layer, an electron-blocking layer, a charge-generation layer, and a cap layer. The organic compound layer 103A may include a plurality of the light-emitting layers 113. In this specification, the organic compound layer included in the light-emitting device 100 is referred to as an EL layer in some cases. The organic compound represented by any of General Formulae (G1) to (G9) can be used in, among the layers included in the organic compound layer 103A, the light-emitting layer 113, the hole-injection layer, the hole-transport layer, the electron-blocking layer, the cap layer, and the like.

In the organic compound layer 103A of the light-emitting device 100, the organic compound represented by any of General Formulae (G1) to (G9) is preferably included in a layer positioned between the light-emitting layer 113 and the anode (the first electrode 101 or the second electrode 102). Here, the layer between the light-emitting layer 113 and the anode is referred to as a first layer. Specific examples of the first layer include a hole-injection layer, a hole-transport layer, and an electron-blocking layer. The organic compound represented by any of General Formulae (G1) to (G9) has a high hole-transport property and can thus be suitably used in the first layer.

The organic compound represented by any of General Formulae (G1) to (G9) has a high LUMO level and high resistance to electrons. Therefore, when the first layer including the organic compound is in contact with the anode-side surface of the light-emitting layer 113, it is possible to inhibit deterioration of the other layers due to electrons passing from the light-emitting layer 113 to the anode side, which improves the reliability of the light-emitting device 100. Furthermore, since the first layer including the organic compound represented by any of General Formulae (G1) to (G9) has a stable film quality, the film quality of the light-emitting layer 113 in contact with the first layer can be prevented from being unstable, which improves the reliability of the light-emitting device 100.

It is further preferable that the first layer include the organic compound represented by any of General Formulae (G1) to (G9) in the case where the light-emitting layer 113 has a structure utilizing exciplex-triplet energy transfer (ExTET), i.e., energy transfer from an exciplex to a light-emitting substance. It is further preferable that, specifically, the first layer include the organic compound represented by any of General Formulae (G1) to (G9) in the case where the light-emitting layer 113 includes a first host material, a second host material, and a light-emitting substance, the first and second host materials form an exciplex in combination, and the difference between the peak wavelengths of emission spectra of the exciplex and the light-emitting substance is less than or equal to 30 nm. Since the organic compound represented by any of General Formulae (G1) to (G9) has a high T₁ level, using the organic compound in the first layer can increase the efficiency of the energy transfer from the exciplex to the light-emitting substance. Since the organic compound represented by any of General Formulae (G1) to (G9) has high resistance to electrons, it is particularly preferable that the first layer including the organic compound be provided in contact with the light-emitting layer 113 to improve the reliability of the light-emitting device 100.

In the case where the light-emitting layer 113 has the structure utilizing ExTET, which is energy transfer from the exciplex to the light-emitting substance, and the light-emitting substance emits red light or green light, the first layer including the organic compound represented by any of General Formulae (G1) to (G9) is expected to achieve more efficient energy transfer from the exciplex to the light-emitting substance.

For example, in comparison of emission spectra of the first host material, the second host material, and a mixed film of these materials, whether the first and second host materials form an exciplex in combination can be confirmed by a phenomenon where the emission spectrum of the mixed film is shifted to the longer wavelength side than the emission spectrum of each material (or has an additional peak on the longer wavelength side). Alternatively, in comparison of comparing transient PL of the first host material, the second host material, and the mixed film of these materials, the confirmation is provided by a difference in transient response, such as a phenomenon where the transient PL lifetime of the mixed film has longer lifetime components or has a larger proportion of delayed components than that of each material. The transient PL can be rephrased as transient electroluminescence (EL). That is, the formation of an exciplex can also be confirmed by a difference in transient response observed by comparison of the transient EL of the first host material, the second host material, and the mixed film of these materials.

It is further preferable that, in the case where the light-emitting layer 113 includes a fluorescent substance, the first layer include the organic compound represented by any of General Formulae (G1) to (G9). It is further preferable that, specifically, in the case where the light-emitting layer 113 includes a host material and a fluorescent substance, the first layer include the organic compound represented by any of General Formulae (G1) to (G9). It is still further preferable that, in the case where the light-emitting layer 113 includes one kind of host material and a fluorescent substance, the first layer include the organic compound represented by any of General Formulae (G1) to (G9). In the case where the light-emitting layer 113 includes one kind of host material and a fluorescent substance, electrons tend to pass from the light-emitting layer 113 to the anode side in some cases. When the organic compound that is represented by any of General Formulae (G1) to (G9) and has a high LUMO level and high resistance to electrons is used in the first layer in contact with the light-emitting layer 113, it is possible to inhibit deterioration of the other layers due to the electrons passing from light-emitting layer 113 to the anode side, improving the reliability of the light-emitting device 100.

FIG. 1B is a cross-sectional view of a photoelectric conversion device 500. The photoelectric conversion device 500 includes a first electrode 501 and a second electrode 502 which are provided over the substrate 160, and an organic compound layer 503 held between the first electrode 501 and the second electrode 502. The organic compound layer 503 contains a photoelectric conversion layer 513, and the photoelectric conversion layer 513 contains a photoelectric conversion material. Examples of the photoelectric conversion material include inorganic semiconductors such as silicon and organic semiconductors such as organic compounds. The photoelectric conversion device 500 can generate electric charge from light entering the organic compound layer 503 and extract the electric charge as current; thus, the photoelectric conversion device 500 can be utilized for an organic optical sensor, an organic solar cell, or the like. Note that voltage may be applied between the first electrode 501 and the second electrode 502. Although not illustrated, the organic compound layer 503 may include, in addition to the photoelectric conversion layer 513, a variety of layers such as a hole-injection layer, a hole-transport layer, an electron-transport layer, an electron-injection layer, a hole-blocking layer, an electron-blocking layer, and a charge-generation layer. The organic compound layer 503 may include a plurality of the photoelectric conversion layers 513. The organic compound represented by any of General Formulae (G1) to (G9) can be used for, among the layers included in the organic compound layer 503, the photoelectric conversion layer 513, the hole-injection layer, the hole-transport layer, the electron-blocking layer, and the like.

FIG. 1C is a cross-sectional view of an organic field-effect transistor 520. The organic field-effect transistor 520 includes a gate electrode 521 provided over the substrate 160, a gate insulating layer 522 over the gate electrode, an organic compound layer 523 over the gate insulating layer 522, and an electrode 524 and an electrode 525 over the organic compound layer 523. The electrode 524 functions as one of a source electrode and a drain electrode, and the electrode 525 functions as the other of the source electrode and the drain electrode. For the organic compound layer 523, the organic compound represented by any of General Formulae (G1) to (G9) can be used. Although FIG. 1C illustrates the organic field-effect transistor 520 which is of a top-contact bottom-gate type, the present invention is not limited thereto. The organic field-effect transistor 520 may have a bottom-contact bottom-gate type structure, a bottom-contact top-gate type structure, a top-contact top-gate type structure, a top-bottom contact structure, a vertical metal-base structure, or a floating metal vertical structure, for example.

When the organic compound represented by any of General Formulae (G1) to (G9) is used for the organic compound layer 103A of the light-emitting device 100, the organic compound layer 503 of the photoelectric conversion device 500, and the organic compound layer 523 of the organic field-effect transistor 520, holes can be transferred smoothly in the organic compound layers. In addition, the highly reliable organic semiconductor devices having high quality can be manufactured. Moreover, the driving lifetime of the organic semiconductor devices can be extended, that is, the reliability thereof can be increased. Furthermore, power consumption of the organic semiconductor devices can be reduced.

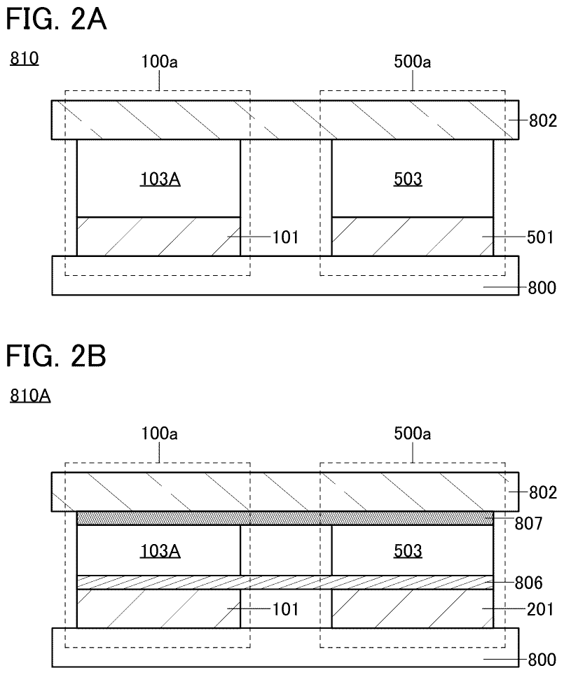

Next, a device 810 in which the light-emitting device 100 and the photoelectric conversion device 500 are provided over the same plane will be described. FIG. 2A illustrates the device 810 including a light-emitting device 100a and a photoelectric conversion device 500a over a substrate 800. Although not illustrated, in the device 810, a partition may be provided in a region surrounded by the substrate 800, the first electrode 101, the organic compound layer 103A, a second electrode 802, the organic compound layer 503, and the first electrode 501. Providing the partition can prevent a short circuit between the devices. It can also inhibit unevenness in the second electrode 802 from being generated and causing problems such as disconnection.

Note that in the device 810, the light-emitting device 100a is used as an organic light-emitting diode, and the photoelectric conversion device 500a is used as an organic optical sensor. The light-emitting device 100a and the photoelectric conversion device 500a are formed over the same substrate. Thus, the device 810 can have a structure in which an organic optical sensor is incorporated in a display device including an organic light-emitting diode, whereby the device 810 can have a function of displaying an image using the light-emitting device 100a and performing image capturing and sensing using the photoelectric conversion device 500a. The organic semiconductor device, which is easily made thin, lightweight, and large in area and has a high degree of freedom for shape and design, can be used in a variety of display devices.

Specific examples of light detected by the photoelectric conversion device 500a include visible light and infrared light. In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible wavelength range is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

In the device 810, the first electrode 101 and the first electrode 501 are provided over the same plane. In FIG. 2A, the first electrodes 101 and 501 are provided over the substrate 800. The first electrodes 101 and 501 can be formed by processing a conductive film formed over the substrate 800 into island shapes, for example. In other words, the first electrodes 101 and 501 can be formed through the same process.

As the substrate 800, a substrate having heat resistance high enough to withstand the formation of the light-emitting device 100a and the photoelectric conversion device 500a can be used. When an insulating substrate is used, a glass substrate, a quartz substrate, a sapphire substrate, a ceramic substrate, an organic resin substrate, or the like can be used as the substrate 800. Alternatively, a semiconductor substrate such as a single crystal semiconductor substrate or a polycrystalline semiconductor substrate of silicon, silicon carbide, or the like; a compound semiconductor substrate of silicon germanium or the like; an SOI substrate; or the like can be used.

In particular, it is preferable to use, as the substrate 800, the insulating substrate or the semiconductor substrate where a semiconductor circuit including a semiconductor element such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

A conductive film that transmits visible light and infrared light is used as the electrode through which light is emitted or enters, among the electrodes included in the light-emitting device 100a and the photoelectric conversion device 500a. As the electrode through which light is not emitted and does not enter, a conductive film that reflects visible light and infrared light is preferably used.

In the device 810, the second electrode 802 functions as the second electrode of each of the light-emitting device 100a and the photoelectric conversion device 500a.

The relation between the potentials of the electrodes in the case where the first electrode 101 of the light-emitting device 100a has a potential higher than that of the second electrode 802 is described. In this case, the first electrode 101 functions as an anode of the light-emitting device 100a, and the second electrode 802 functions as a cathode of the light-emitting device 100a. The first electrode 501 of the photoelectric conversion device 500a has a potential lower than that of the second electrode 802. That is, when a first potential, a second potential, and a third potential are supplied to the first electrode 101, the second electrode 802, and the first electrode 501, respectively, the first potential is higher than the second potential, and the second potential is higher than the third potential.

Next, the case where the first electrode 101 of the light-emitting device 100a has a potential lower than that of the second electrode 802 is described. In this case, the first electrode 101 functions as a cathode of the light-emitting device 100a, and the second electrode 802 functions as an anode of the light-emitting device 100a. The first electrode 501 of the photoelectric conversion device 500a has a potential lower than that of the second electrode 802 and a potential higher than that of the first electrode 101. That is, when the first potential, the second potential, and the third potential are supplied to the first electrode 101, the second electrode 802, and the first electrode 501, respectively, the second potential is higher than the third potential, and the third potential is higher than the first potential.

In the device 810, the organic compound represented by any of General Formulae (G1) to (G9) is preferably used for one or both of the organic compound layer 103A and the organic compound layer 503. In that case, holes can be transferred smoothly in the organic compound layer 103A and the organic compound layer 503. In addition, the light-emitting device 100a and the photoelectric conversion device 500a can be highly reliable organic semiconductor devices having high quality. Moreover, the driving lifetime of the light-emitting device 100a and the photoelectric conversion device 500a can be extended, that is, the reliability thereof can be improved. The process in which a hole-injection layer of the organic compound layer 103A and a hole-injection layer of the organic compound layer 503 are formed collectively and the process in which a hole-transport layer of the organic compound layer 103A and a hole-transport layer of the organic compound layer 503 are formed collectively can simplify a process and reduce production cost, which is preferable in mass production.

FIG. 2B illustrates a device 810A that is a variation example of the device 810. The device 810A is different from the device 810 in that the organic compound layer 103A and the organic compound layer 503 include a common layer 806 and a common layer 807. In the light-emitting device 100a, the common layers 806 and 807 function as part of the organic compound layer 103A. In the photoelectric conversion device 500a, the common layers 806 and 807 function as part of the organic compound layer 503. The common layer 806 includes a hole-injection layer and a hole-transport layer, for example. The common layer 807 includes an electron-transport layer and an electron-injection layer, for example.

With the common layers 806 and 807, a photoelectric conversion device can be incorporated in the device 810 without a significant increase in the number of times of separate formation of devices, whereby the device 810A can be manufactured with a high throughput.

In the device 810A, the organic compound represented by any of General Formulae (G1) to (G9) is preferably used for the common layer 806. Accordingly, holes can be transferred smoothly in the organic compound layer 103A and the organic compound layer 503. In addition, the light-emitting device 100a and the photoelectric conversion device 500a can be highly reliable organic semiconductor devices having high quality. Furthermore, the driving lifetime of the light-emitting device 100a and the photoelectric conversion device 500a can be extended, that is, the reliability thereof can be improved.

The resolution of the photoelectric conversion devices 500a described in this embodiment can be arranged at a resolution higher than or equal to 100 ppi, preferably higher than or equal to 200 ppi, further preferably higher than or equal to 300 ppi, still further preferably higher than or equal to 400 ppi, and still further preferably higher than or equal to 500 ppi, and lower than or equal to 2000 ppi, lower than or equal to 1000 ppi, or lower than or equal to 600 ppi, for example. In particular, when the photoelectric conversion devices 500a are arranged at a resolution higher than or equal to 200 ppi and lower than or equal to 600 ppi, preferably higher than or equal to 300 ppi and lower than or equal to 600 ppi, the device can be suitably used for image capturing of a fingerprint. In fingerprint authentication with a light-emitting and light-receiving apparatus of one embodiment of the present invention, the increased resolution of the photoelectric conversion device 500a enables, for example, highly accurate extraction of the minutiae of fingerprints; thus, the accuracy of the fingerprint authentication can be increased. The resolution is preferably higher than or equal to 500 ppi, in which case the authentication conforms to the standard by the National Institute of Standards and Technology (NIST) or the like. On the assumption that the resolution at which the photoelectric conversion devices are arranged is 500 ppi, the size of each pixel is 50.8 μm, which is adequate for image capturing of a fingerprint ridge distance (typically, greater than or equal to 300 μm and less than or equal to 500 μm).

The structures described in this embodiment can be used in appropriate combination with any of the structures described in the other embodiments.

### Embodiment 3

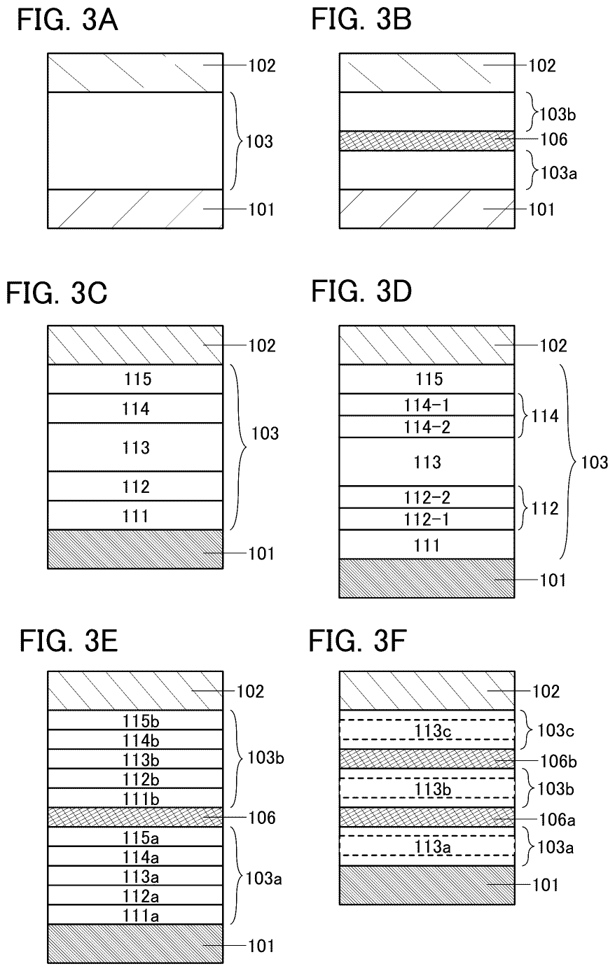

In this embodiment, structures of the organic semiconductor devices of one embodiment of the present invention are described with reference to FIGS. 3A to 3F.

### <<Basic Structure of Light-Emitting Device>>

A basic structure of a light-emitting device is described. FIG. 3A illustrates a light-emitting device including, between a pair of electrodes, an EL layer including a light-emitting layer. Specifically, the organic compound layer 103 is positioned between the first electrode 101 and the second electrode 102. Note that the organic compound layer 103 can also be referred to as an EL layer.

FIG. 3B illustrates a light-emitting device that has a stacked-layer structure (tandem structure) in which a plurality of EL layers (two EL layers 103a and 103b in FIG. 3B) are provided between a pair of electrodes and a charge-generation layer 106 is provided between the EL layers. A light-emitting device having a tandem structure enables manufacturing of a display device that has high efficiency without changing the amount of current.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when voltage is applied in FIG. 3B such that the potential of the first electrode 101 is higher than that of the second electrode 102, the charge-generation layer 106 injects electrons into the organic compound layer 103a and injects holes into the organic compound layer 103b.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

FIG. 3C illustrates a stacked-layer structure of the organic compound layer 103 in the light-emitting device of one embodiment of the present invention. In this case, the first electrode 101 is regarded as functioning as an anode, and the second electrode 102 is regarded as functioning as a cathode. The organic compound layer 103 has a structure in which a hole-injection layer 111, a hole-transport layer 112, the light-emitting layer 113, an electron-transport layer 114, and an electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can extend driving lifetime; in other words, the structure can achieve higher reliability than a single-layer structure in some cases. In the case where a plurality of EL layers are provided as in the tandem structure illustrated in FIG. 3B, the layers in each EL layer are sequentially stacked from the anode side as described above. When the first electrode 101 is the cathode and the second electrode 102 is the anode, the stacking order of the layers in the organic compound layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the EL layers (103, 103a, and 103b) contains an appropriate combination of a light-emitting substance and a plurality of substances, so that fluorescent light of a desired color or phosphorescent light of a desired color can be obtained. The plurality of EL layers (103a and 103b) in FIG. 3B may exhibit their respective emission colors. In that case, the light-emitting substances and other substances can be different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 3C. Thus, light from the light-emitting layer 113 in the organic compound layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: λ) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

FIG. 3D illustrates a modification example of the stacked-layer structure illustrated in FIG. 3C. Also in this case, the first electrode 101 is regarded as functioning as an anode, and the second electrode 102 is regarded as functioning as a cathode. In this modification example, the hole-transport layer 112 and the electron-transport layer 114 each have a stacked-layer structure of two layers. In other words, the organic compound layer 103 has a structure in which a hole-injection layer 111, a first hole-transport layer 112-1, a second hole-transport layer 112-2, a light-emitting layer 113, a second electron-transport layer 114-2, a first electron-transport layer 114-1, and an electron-injection layer 115 are stacked in this order over the first electrode 101. Note that the light-emitting layer 113 is positioned between the first electrode 101 and the second electrode 102. The first hole-transport layer 112-1 is positioned between the first electrode 101 and the light-emitting layer 113. The first electron-transport layer 114-1 is positioned between the light-emitting layer 113 and the second electrode 102. The hole-injection layer 111 is positioned between the first electrode 101 and the hole-transport layer 112. The electron-injection layer 115 is positioned between the electron-transport layer 114 and the second electrode 102. The second hole-transport layer 112-2 is positioned between the first hole-transport layer 112-1 and the light-emitting layer 113. In other words, the second electron-transport layer 114-2 is positioned between the light-emitting layer 113 and the first electron-transport layer 114-1. In the case where the organic compound layer 103 has such a stacked-layer structure, one or more of the hole-injection layer 111, the hole-transport layer 112, and the light-emitting layer 113 preferably contains the organic compound represented by any of General Formulae (G1) to (G9).

The second hole-transport layer 112-2 is provided to prevent passing of electrons from the light-emitting layer 113 to the first electrode 101 side, for example. Accordingly, the second hole-transport layer 112-2 can also be referred to as an electron-blocking layer. It is particularly preferable that the second hole-transport layer 112-2 in contact with the light-emitting layer 113 include the organic compound represented by any of General Formulae (G1) to (G9). The second electron-transport layer 114-2 is provided to prevent passing of holes from the light-emitting layer 113 to the second electrode 102 side, for example. Accordingly, the second electron-transport layer 114-2 can also be referred to as a hole-blocking layer.

The light-emitting device illustrated in FIG. 3E is a light-emitting device having a tandem structure. Owing to a microcavity structure of the light-emitting device, light (monochromatic light) with different wavelengths from the EL layers (103a and 103b) can be extracted. It is therefore unnecessary to separately form EL layers for obtaining a plurality of emission colors (e.g., R, G, and B). Thus, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 3F is an example of the light-emitting device having the tandem structure illustrated in FIG. 3B, and includes three EL layers (103a, 103b, and 103c) stacked with charge-generation layers (106a and 106b) positioned therebetween, as illustrated in FIG. 3F. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² fcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² fcm or less.

### <<Specific Structure of Light-Emitting Device>>

Next, a specific structure of the light-emitting device of one embodiment of the present invention will be described. Here, the description is made using FIG. 3E illustrating the tandem structure. Note that the structure of the EL layer applies also to the structure of the light-emitting devices having a single structure in FIGS. 3A and 3C. When the light-emitting device in FIG. 3E has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the organic compound layer 103b, with the use of a material selected as appropriate.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 3E, when the first electrode 101 is the anode, a hole-injection layer 111a and a hole-transport layer 112a of the organic compound layer 103a are sequentially stacked over the first electrode 101 by a vacuum evaporation method. After the organic compound layer 103a and the charge-generation layer 106 are formed, a hole-injection layer 111b and a hole-transport layer 112b of the organic compound layer 103b are sequentially stacked over the charge-generation layer 106 in a similar manner.

The light-emitting device illustrated in FIG. 3E can have a micro optical resonator (microcavity) structure when the first electrode 101 is a reflective electrode and the second electrode 102 is a transflective electrode. Thus, light from the light-emitting layer 113 in the organic compound layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: λ) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; accordingly, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; accordingly, it is assumed that the above effect can be sufficiently obtained wherever the reflective region and the light-emitting region may be set in the first electrode 101 and the light-emitting layer that emits the desired light, respectively.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² fcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² fcm or less.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain an organic acceptor material and a material having a high hole-injection property.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound including an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferable because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 of the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among the above oxides, molybdenum oxide is preferable because it is stable in atmospheric air, has a low hygroscopic property, and is easily handled. Other examples include a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI); (C₆₀-Iₕ) [5,6]fullerene (abbreviation: C₆₀); (C₇₀-D₅ₕ) [5,6]fullerene (abbreviation: C₇₀); an organic compound such as phthalocyanine (abbreviation: H₂Pc); and a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). A phthalocyanine-based metal complex such as CuPc or ZnPc and 2,3,8,9,14,15-hexafluorodiquinoxalino[2,3-a:2′,3′-c]phenazine are especially preferable. Among these materials, CuPc and ZnPc are preferable because they are inexpensive and have favorable characteristics. Using ZnPc, which has a low diffusion coefficient with respect to silicon, reduces the probability that metal diffusion to a semiconductor adversely affects the semiconductor characteristics; accordingly, ZnPc is particularly suitable for a display device manufactured using a silicon semiconductor.

Other examples are aromatic amine compounds, which are low-molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/polystyrenesulfonic acid (abbreviation: PEDOT/PSS) or polyaniline/polystyrenesulfonic acid (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that other substances can also be used as long as the substances have hole-transport properties higher than electron-transport properties.

As the hole-transport material, materials having a high hole-transport property, such as a compound including a π-electron rich heteroaromatic ring (e.g., a carbazole derivative, a furan derivative, and a thiophene derivative) and an aromatic amine (an organic compound including an aromatic amine skeleton), are preferable. The organic compound described in Embodiments 1 and 2 has a hole-transport property and can be used as a hole-transport material.

Examples of the carbazole derivative (an organic compound including a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bi-9H-carbazole (abbreviation: βNCCP).

Specific examples of the aromatic amine having a carbazolyl group include 4-phenyl-4′-(9-phenyl-9H-carbazol-3-yl)triphenylamine (abbreviation: PCBA1BP), N-(biphenyl-4-yl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-(1,1′:3′,1″-terphenyl-4-yl)-9,9-dimethyl-9H-fluoren-2-amine, N-[4-(9-phenyl-9H-carbazol-3-yl)phenyl]-N-(1,1′:4′,1″-terphenyl-4-yl)-9,9-dimethyl-9H-fluoren-2-amine, N-[4-(9-phenyl-9H-carbazol-3-yl)phenyl]-N-(1,1′:3′,1″-terphenyl-4-yl)-9,9-dimethyl-9H-fluoren-4-amine, N-[4-(9-phenyl-9H-carbazol-3-yl)phenyl]-N-(1,1′:4′,1″-terphenyl-4-yl)-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), N-(9,9-spirobi[9H-fluoren]-2-yl)-N,9-diphenylcarbazol-3-amine (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazol-3-yl)phenyl]phenanthrene (abbreviation: PCPPn), 3-[4-(1-naphthyl)phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthryl)phenyl]-9H-carbazole (abbreviation: CzPA).

Specific examples of the furan derivative (an organic compound including a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound including a thiophene ring) include 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N′,N′-triphenyl-1,4-phenyldiamine (abbreviation: DPASF), N,N′-diphenyl-N,N′-bis(4-diphenylaminophenyl)spirobi[9H-fluorene]-2,7-diamine (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAPNB), 4-[4-(2-naphthyl)phenyl]-4′,4″-diphenyltriphenylamine (abbreviation: BBAβNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other examples of the hole-transport material include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/polystyrenesulfonic acid (abbreviation: PEDOT/PSS) or polyaniline/polystyrenesulfonic acid (abbreviation: PAni/PSS), for example.

Note that the hole-transport material is not limited to the above examples, and any of a variety of known materials may be used alone or in combination as the hole-transport material.

The hole-injection layers (111, 111a, and 111b) can be formed by any of known film formation methods such as a vacuum evaporation method.

### <Hole-Transport Layer>

The hole-transport layers (112, 112a, and 112b) transport the holes, which are injected from the first electrode 101 by the hole-injection layers (111, 111a, and 111b), to the light-emitting layers (113, 113a, and 113b). Note that the hole-transport layers (112, 112a, and 112b) each include a hole-transport material. Thus, the hole-transport layers (112, 112a, and 112b) can be formed using any of the hole-transport materials that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, and 113b). The same organic compound is preferably used for the hole-transport layers (112, 112a, and 112b) and the light-emitting layers (113, 113a, and 113b), in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) include a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables exhibiting different emission colors (e.g., white light emission obtained by a combination of complementary emission colors). When a plurality of light-emitting layers are provided, the light-emitting layers can exhibit the same color. The structure in which a plurality of light-emitting layers that emit light of the same color are stacked can sometimes achieve higher reliability than a single-layer structure. Furthermore, a stacked-layer structure in which one light-emitting layer contains two or more kinds of light-emitting substances may be employed.

The light-emitting layers (113, 113a, and 113b) may each contain one or more kinds of organic compounds (e.g., a host material) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S₁ level) of the second host material is higher than that of the first host material, and the lowest triplet excitation energy level (T₁ level) of the second host material is higher than that of the guest material. Preferably, the lowest triplet excitation energy level (T₁ level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and a long lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the S₁ level and the T₁ level and functions as a thermally activated delayed fluorescent (TADF) material capable of converting triplet excitation energy into singlet excitation energy. In an example of a preferable combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound of the combination for forming an exciplex. The organic compound represented by any of General Formulae (G1) to (G9) described in Embodiments 1 and 2 have a hole-transport property and thus can be used as the host material.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that emit fluorescent light (fluorescent substances) can be given as examples of the light-emitting substance that converts singlet excitation energy into light emission and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine](abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine](abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf(IV)-02). In particular, a pyrenediamine compound such as 1,6FLPAPrn, 1,6mMemFLPAPrn, or 1,6BnfAPrn-03 can be used, for example.

<<Light-Emitting Substance that Converts Triplet Excitation Energy into Light Emission>>

Examples of the light-emitting substance that converts triplet excitation energy into light and that can be used in the light-emitting layer 113 include substances that emit phosphorescent light (phosphorescent substances) and TADF materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably includes a metal element with large spin-orbit interaction, and can be an organometallic complex, a metal complex (platinum complex), or a rare earth metal complex, for example. Specifically, the phosphorescent substance preferably includes a transition metal element. It is preferable that the phosphorescent substance include a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (from 450 nm to 570 nm: Blue or Green)>>

As examples of a phosphorescent substance which emits blue or green light and whose emission spectrum has a peak wavelength higher than or equal to 450 nm and lower than or equal to 570 nm, the following substances can be given.

Examples of the phosphorescent substance include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[i-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III)tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm: Green or Yellow)>>

As examples of a phosphorescent substance which emits green or yellow light and whose emission spectrum has a peak wavelength higher than or equal to 495 nm and lower than or equal to 590 nm, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), {2-(methyl-d₃)-8-[4-(1-methylethyl-1-d]-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

<<Phosphorescent Substance (from 570 nm to 750 nm: Yellow or Red)>>

As examples of a phosphorescent substance which emits yellow or red light and whose emission spectrum has a peak wavelength higher than or equal to 570 nm and lower than or equal to 750 nm, the following substances can be given.

Examples of the phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-N]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N,C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C²)iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials described below can be used as the TADF material. The TADF material is a material that has a small difference between its S₁ and T₁ levels (preferably less than or equal to 0.20 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0.00 eV and less than or equal to 0.20 eV, preferably greater than or equal to 0.00 eV and less than or equal to 0.10 eV. Delayed fluorescent light by the TADF material refers to light emission having a spectrum similar to that of normal fluorescent light and an extremely long lifetime. The lifetime is longer than or equal to 1×10⁻⁶ seconds, or longer than or equal to 1×10⁻³ seconds.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

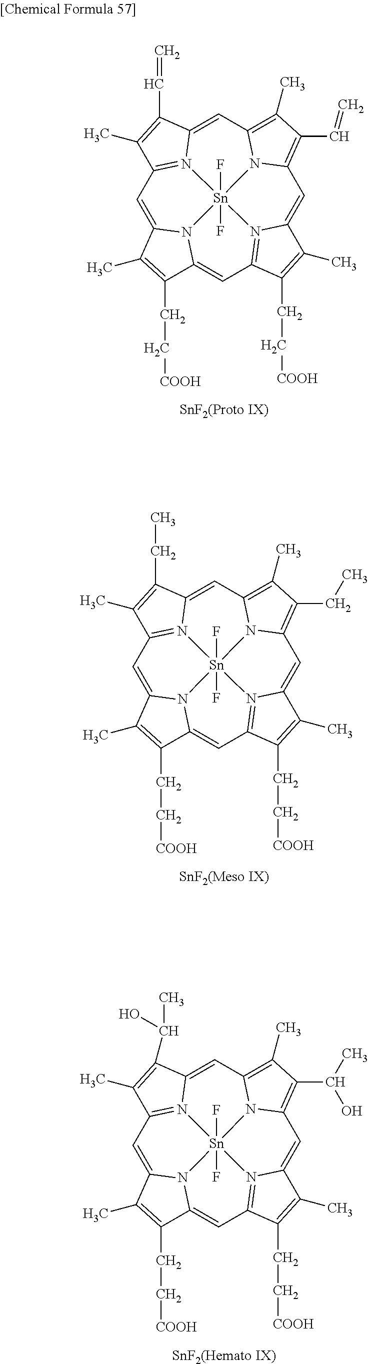

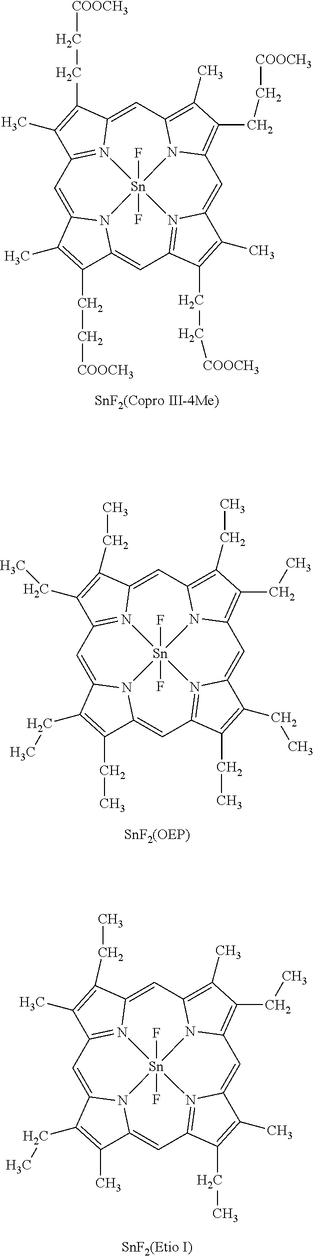

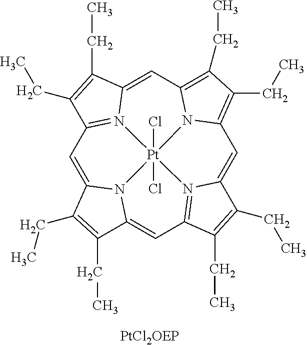

Additionally, a heteroaromatic compound including a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-[3,3′-bi-9H-carbazol]-9-yl)benzofuro[3,2-d]pyrimidine (abbreviation: 4PCCzBfpm), 4-[4-(9′-phenyl-[3,3′-bi-9H-carbazol]-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are enhanced and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

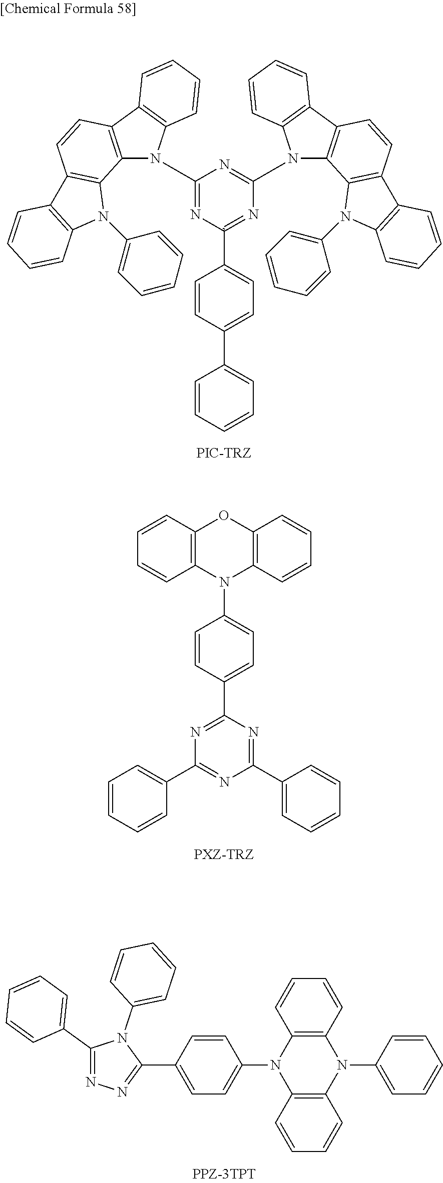 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

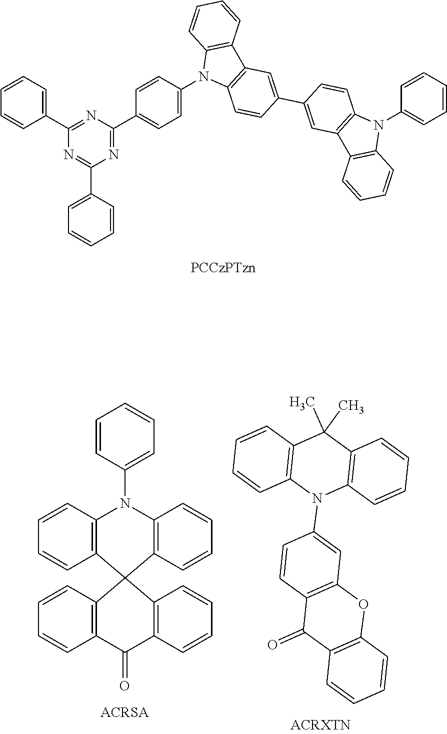 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21]

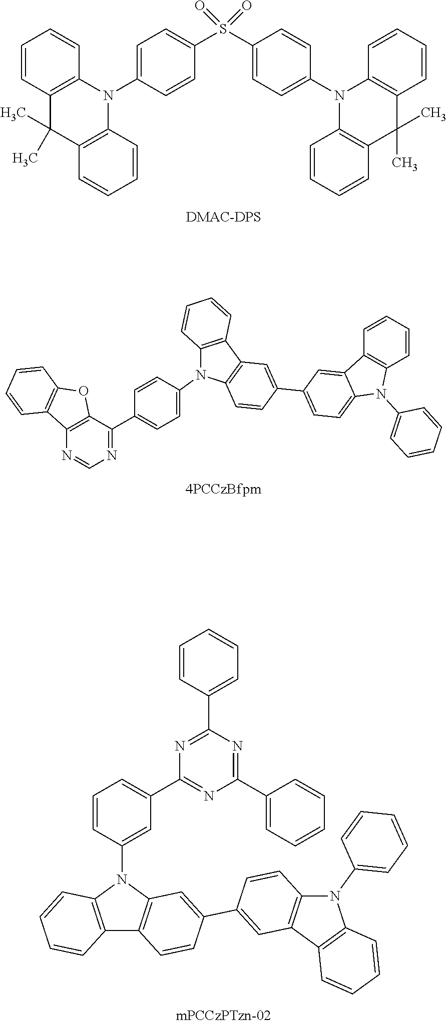 [SMILES: CC1(C)c2ccccc2N(c2ccc(S(=O)(=O)c3ccc(N4c5ccccc5C(C)(C)c5ccccc54)cc3)cc2)c2ccccc21 ; c1ccc(-n2c3ccccc3c3cc(-c4ccc5c(c4)c4ccccc4n5-c4ccc(-c5ncnc6c5oc5ccccc56)cc4)ccc32)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

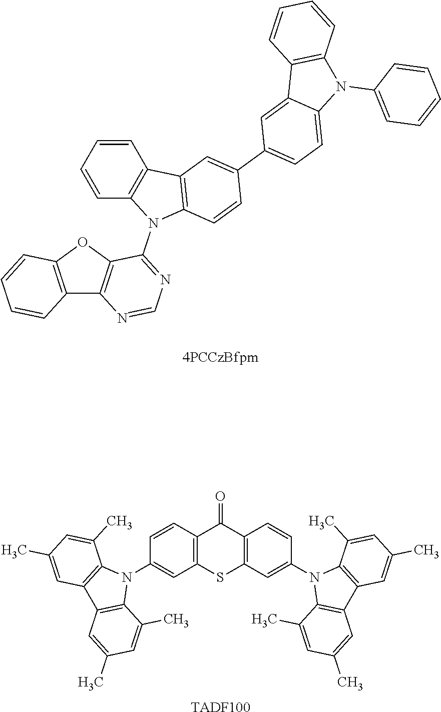

In addition to the above, another example of a material having a function of converting triplet excitation energy into light emission is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, and 113b), one or more kinds selected from substances having a larger energy gap than the light-emitting substance (guest material) can be used.

### <<Host Material for Fluorescence>>

In the case where the light-emitting substance used in the light-emitting layers (113, 113a, and 113b) is a fluorescent substance, an organic compound (a host material) used in combination with the fluorescent substance is preferably an organic compound that has a high energy level in a singlet excited state and has a low energy level in a triplet excited state or an organic compound having a high fluorescence quantum yield. Therefore, the hole-transport material (described above) and the electron-transport material (described below) shown in this embodiment, for example, can be used as long as they are organic compounds that satisfy such a condition. In addition, the organic compounds described in Embodiments 1 and 2 can be used.

In terms of a preferable combination with the light-emitting substance (fluorescent substance), examples of the organic compound (host material), some of which overlap the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), 9-[4-(10-phenyl-9-anthryl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthryl)-benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-{4-[10-(biphenyl-4-yl)-9-anthryl]phenyl}-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescence>>

In the case where the light-emitting substance used in the light-emitting layers (113, 113a, and 113b) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance. In addition, the organic compounds described in Embodiments 1 and 2 can be used.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferable, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compounds (the host material and the assist material), some of which are mentioned in the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Specific examples of the aromatic amine and the carbazole derivative, which are organic compounds having a high hole-transport property among the above-described organic compounds, are the same as the specific examples of the hole-transport materials described above, and those materials are preferable as the host material.

Specific examples of the dibenzothiophene derivative and the dibenzofuran derivative, which are organic compounds having a high hole-transport property among the above-described organic compounds, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Other examples of preferable host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include an organic compound including a heteroaromatic ring having a polyazole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound including a heteroaromatic ring having a phenanthroline ring such as bathophenanthroline (abbreviation: BPhen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P); and an organic compound including a heteroaromatic ring having a dibenzoquinoxaline ring such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f;h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), or 2-[4′-(9-phenyl-9H-carbazol-3-yl)-3,1′-biphenyl-1-yl]dibenzo[f;h]quinoxaline (abbreviation: 2mpPCBPDBq). These organic compounds are preferable as the host material.

Among the above organic compounds, specific examples of the pyridine derivative, the diazine derivative (e.g., the pyrimidine derivative, the pyrazine derivative, and the pyridazine derivative), the triazine derivative, and the furodiazine derivative, which are organic compounds having a high electron-transport property, include organic compounds including a heteroaromatic ring having a diazine ring such as 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-benzofuro[3,2-d]pyrimidine (abbreviation: 8BP-4mDBtPBfpm), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 11-[3′-(dibenzothiophen-4-yl)biphenyl-3-yl]phenanthro[9′,10′:4,5]furo[2,3-b]pyrazine (abbreviation: 11mDBtBPPnfpr), 11-[3′-(dibenzothiophen-4-yl)biphenyl-4-yl]phenanthro[9′,10′:4,5]furo[2,3-b]pyrazine, 11-[(3′-9H-carbazol-9-yl)biphenyl-3-yl]phenanthro[9′,10′:4,5]furo[2,3-b]pyrazine, 12-(9′-phenyl-[3,3′-bi-9H-carbazol]-9-yl)phenanthro[9′,10′:4,5]furo[2,3-b]pyrazine (abbreviation: 12PCCzPnfpr), 9-[3′-(9-phenyl-9H-carbazol-3-yl)biphenyl-4-yl]naphtho[1′,2′:4,5]furo[2,3-b]pyrazine (abbreviation: 9pmPCBPNfpr), 9-(9′-phenyl-[3,3′-bi-9H-carbazol]-9-yl)naphtho[1′,2′:4,5]furo[2,3-b]pyrazine (abbreviation: 9PCCzNfpr), 10-(9′-phenyl-[3,3′-bi-9H-carbazol]-9-yl)naphtho[1′,2′:4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), 9-[3′-(6-phenyldibenzothiophen-4-yl)biphenyl-3-yl]naphtho[1′,2′:4,5]furo[2,3-b]pyrazine (abbreviation: 9mDBtBPNfpr-02), 9-[3-(9′-phenyl-[3,3′-bi-9H-carbazol]-9-yl)phenyl]naphtho[1′,2′:4,5]furo[2,3-b]pyrazine (abbreviation: 9mPCCzPNfpr), 9-[3′-(2,8-diphenyldibenzothiophen-4-yl)biphenyl-3-yl]naphtho[1′,2′:4,5]furo[2,3-b]pyrazine, 11-[3′-(2,8-diphenyldibenzothiophen-4-yl)biphenyl-3-yl]phenanthro[9′,10′:4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-[8-([1,1′:4′,1″-terphenyl]-4-yl)-1-dibenzofuranyl]-1,3,5-triazine (abbreviation: mBP-TPDBfTzn), 6-(biphenyl-3-yl)-4-[3,5-bis(9H-carbazol-9-yl)phenyl)-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm), and those materials are preferable as the host material.

Among the above organic compounds, specific examples of metal complexes that are organic compounds having a high electron-transport property include zinc- or aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), and bis(8-quinolinolato)zinc(II) (abbreviation: Znq), and metal complexes having a quinoline ring or a benzoquinoline ring. These metal complexes are preferable as the host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) are preferable as the host material.

Furthermore, the following organic compounds with a diazine ring, which have a bipolar property, a high hole-transport property, and a high electron-transport property, can be used as the host material: 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <Electron-Transport Layer>

The electron-transport layers (114, 114a, and 114b) transport electrons, which are injected from the second electrode 102 and the charge-generation layers (106, 106a, and 106b) by electron-injection layers (115, 115a, and 115b) described later, to the light-emitting layers (113, 113a, and 113b). The heat resistance of the light-emitting device of one embodiment of the present invention can be improved by including the stacked electron-transport layers. The electron-transport material used in the electron-transport layers (114, 114a, and 114b) is preferably a substance having an electron mobility higher than or equal to 1×10⁻⁶ cm²/Vs in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. When a photolithography process is performed over the electron-transport layer including the above-described mixed material, which has heat resistance, an adverse effect of the thermal process on the device characteristics can be reduced.

### <<Electron-Transport Material>>

As the electron-transport material that can be used for the electron-transport layers (114, 114a, and 114b), an organic compound having a high electron-transport property can be used, and for example, a heteroaromatic compound can be used. The term heteroaromatic compound refers to a cyclic compound including at least two different kinds of elements in a ring. Examples of cyclic structures include a three-membered ring, a four-membered ring, a five-membered ring, a six-membered ring, and the like, among which a five-membered ring and a six-membered ring are particularly preferable. The elements included in the heteroaromatic compound are preferably one or more of nitrogen, oxygen, sulfur, and the like in addition to carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used.

Note that the electron-transport material can be different from the materials used in the light-emitting layer. Not all excitons formed by recombination of carriers in the light-emitting layer can contribute to light emission and some excitons are diffused into a layer in contact with the light-emitting layer or a layer in the vicinity of the light-emitting layer. In order to avoid this phenomenon, the energy level (the lowest singlet excitation level or the lowest triplet excitation level) of a material used for the layer in contact with the light-emitting layer or the layer in the vicinity of the light-emitting layer is preferably higher than that of a material used for the light-emitting layer. Thus, when a material different from the material of the light-emitting layer is used as the electron-transport material, a device having high efficiency can be obtained.

The heteroaromatic compound is an organic compound including at least one heteroaromatic ring.

The heteroaromatic ring includes any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like include a heteroaromatic compound having a heteroaromatic ring, such as a pyridine ring, a diazine ring (a pyrimidine ring, a pyrazine ring, a pyridazine ring, or the like), a triazine ring, or a polyazole ring. Other examples include a heteroaromatic compound having a bipyridine structure, a heteroaromatic compound having a terpyridine structure, and the like, which are included in examples of a heteroaromatic compound in which pyridine rings are connected.

Examples of the heteroaromatic compound having a fused ring structure partly including the above six-membered ring structure include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the above-described heteroaromatic compound having a five-membered ring structure (a polyazole ring (including an imidazole ring, a triazole ring, or an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include: a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB); a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′:4,5]furo[3,2-d]pyrimidine (abbreviation: 8mDBtBPNfpm), or 8-([2,2′-binaphthalen]-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a fused heteroaromatic ring.

Other examples include heteroaromatic compounds including a heteroaromatic ring having a diazine (pyrimidine) ring, such as 2,2′-(pyridine-2,6-diyl)bis(4-phenylbenzo[h]quinazoline) (abbreviation: 2,6(P-Bqn)₂Py), 2,2′-([2,2′-bipyridine]-6,6′-diyl)bis(4-phenylbenzo[h]quinazoline) (abbreviation: 6,6′(P-Bqn)₂BPy), 2,2′-(pyridine-2,6-diyl)bis{4-[4-(2-naphthyl)phenyl]-6-phenylpyrimidine}(abbreviation: 2,6(NP-PPm)₂Py), or 6-(biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tzn), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure partly including a six-membered ring structure (the heteroaromatic compound having a fused ring structure) include a heteroaromatic compound having a quinoxaline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBphen), 2,2′-(1,3-phenylene)bis(9-phenyl-1,10-phenanthroline) (abbreviation: mPPhen2P), 2,2′-(pyridin-2,6-diyl)bis(4-phenylbenzo[h]quinazoline) (abbreviation: 2,6(P-Bqn)2Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), and 2mpPCBPDBq.

For the electron-transport layers (114, 114a, and 114b), any of the metal complexes given below can be used as well as the heteroaromatic compounds described above. Examples of the metal complexes include a metal complex having a quinoline ring or a benzoquinoline ring, such as tris(8-quinolinolato)aluminum(III) (abbreviation: Alq₃), Almq₃, 8-quinolinolato-lithium (abbreviation: Liq), BeBq₂, bis(2-methyl-8-quinolinolato) (4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

High-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) can be used as the electron-transport material.

Each of the electron-transport layers (114, 114a, and 114b) is not limited to a single layer and may be a stack of two or more layers each including any of the above substances.

### <Electron-Injection Layer>

The electron-injection layers (115, 115a, and 115b) include a substance having a high electron-injection property. The electron-injection layers (115, 115a, and 115b) are layers for increasing the efficiency of electron injection from the second electrode 102 and are preferably formed using a material whose value of the LUMO level has a small difference (less than or equal to 0.50 eV) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal or a compound of a rare earth metal, such as erbium fluoride (ErF₃) or ytterbium (Yb), can also be used. It is also possible to use a compound including a 1,3,4,6,7,8-tetrahydro-2H-pyrimido[1,2-a]pyrimidine skeleton, such as 1-(9,9′-spirobi[9H-fluoren]-2-yl)-1,3,4,6,7,8-hexahydro-2H-pyrimido[1,2-a]pyrimidine (abbreviation: 2hppSF), 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF), or 1,1′-pyridine-2,6-diyl-bis(1,3,4,6,7,8-hexahydro-2H-pyrimido[1,2-a]pyrimidine) (abbreviation: hpp2Py). To form the electron-injection layers (115, 115a, and 115b), two or more of the above materials may be mixed or stacked. Electride may also be used for the electron-injection layers (115, 115a, and 115b). Examples of an electride include substances in which electrons are added at high concentration to a calcium oxide-aluminum oxide. Any of the substances for forming the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound is preferably used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a LUMO level higher than or equal to −3.60 eV and lower than or equal to −2.30 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure partly including a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples thereof include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

When the two EL layers (103a and 103b) are provided and the charge-generation layer 106 is provided between the plurality of EL layers as in the light-emitting device in FIG. 3E, a structure in which a plurality of EL layers are stacked between the pair of electrodes (the structure is also referred to as a tandem structure) can be obtained.

### <Charge-Generation Layer>

The charge-generation layer 106 has a function of injecting electrons into the organic compound layer 103a and injecting holes into the organic compound layer 103b when a voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these structures may be stacked. Furthermore, an electron-relay layer may be provided between the p-type layer and the electron-injection buffer layer. Note that forming the charge-generation layer 106 with the use of any of the above materials can inhibit an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films including the respective materials may be used.

In the case where the charge-generation layer 106 is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An alkali metal compound such as Liq may be used. An organic compound such as tetrathianaphthacene may be used as the electron donor. An organic compound including a 1,3,4,6,7,8-tetrahydro-2H-pyrimido[1,2-a]pyrimidine skeleton, such as 2hppSF, 2,7hpp2SF, or hpp2Py may be used as the electron donor. When any of these organic compounds is used as the electron donor, the electron-transport material to be combined with the electron donor is preferably an organic compound including a heteroaromatic ring having a phenanthroline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P), in which case driving voltage of the light-emitting device can be reduced.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer can be higher than or equal to −5.00 eV, further preferably higher than or equal to −5.00 eV and lower than or equal to −3.00 eV. Note that as the substance having an electron-transport property in the electron-relay layer, a phthalocyanine-based material or a metal complex having a metal-oxygen bond and an aromatic ligand is preferably used.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

Although FIG. 3E illustrates the structure in which two of the organic compound layers 103 are stacked, three or more EL layers may be stacked with charge-generation layers each provided between two adjacent EL layers.

### <Cap Layer>

Although not illustrated in FIGS. 3A to 3F, a cap layer may be provided over the second electrode 102 of the light-emitting device. For example, a material with a high refractive index can be used for the cap layer. When the cap layer is provided over the second electrode 102, extraction efficiency of light emitted through the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II).

### <Substrate>

The light-emitting device described in this embodiment can be formed over a variety of substrates. Note that the type of the substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, and paper or a base material film including a fibrous material.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as an acrylic resin, polypropylene, polyester, polyvinyl fluoride, polyvinyl chloride, polyamide, polyimide, aramid, an epoxy resin, an inorganic vapor deposition film, and paper.

For fabrication of the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers with various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high-molecular compound (e.g., an oligomer, a dendrimer, or a polymer), a middle-molecular compound (a compound between a low-molecular compound and a high-molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the organic compound layer 103 of the light-emitting device described in this embodiment are not limited to the materials described in this embodiment, and other materials can be used in combination as long as the functions of the layers are fulfilled.

The structures described in this embodiment can be used in appropriate combination with any of the structures described in the other embodiments.

### Embodiment 4

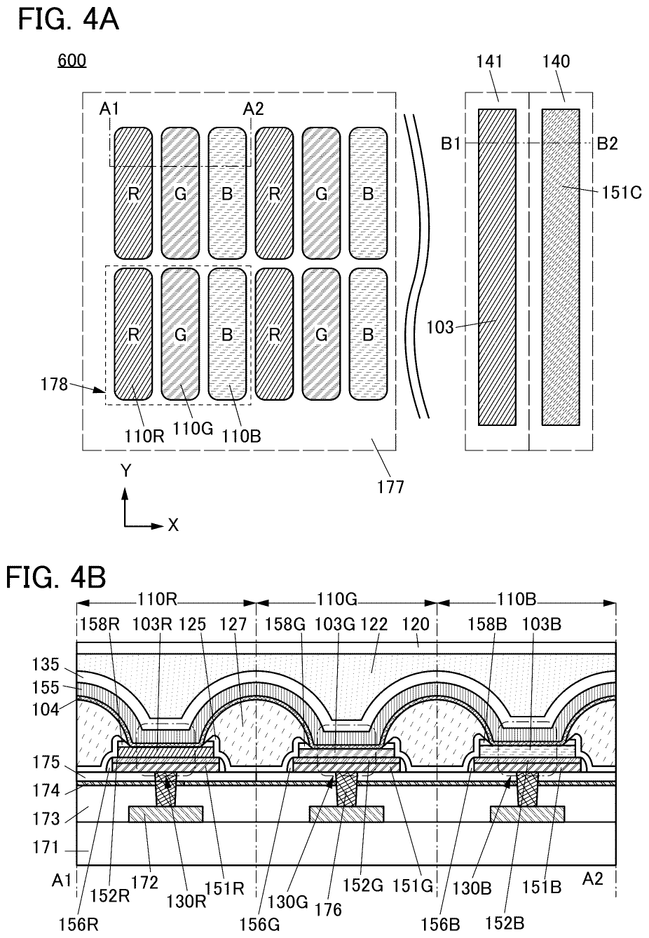

In this embodiment, a display device of one embodiment of the present invention is described in detail using FIGS. 4A and 4B.

A display device 600 includes a pixel portion 177 in which a plurality of pixels 178 are arranged in a matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and Y, and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 4A illustrates an example in which subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 4A illustrates an example in which the region 141 and the connection portion 140 are positioned on the right side of the pixel portion 177, the positions of the region 141 and the connection portion 140 are not particularly limited. The number of the regions 141 and the number of the connection portions 140 can each be one or more.

FIG. 4B is an example of a cross-sectional view along the dashed-dotted line A1-A2 in FIG. 4A. As shown in FIG. 4A, the display device 600 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not shown). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 135 is provided to cover the light-emitting device 130. A substrate 120 is bonded onto the protective layer 135 with a resin layer 122. An inorganic insulating layer 125 and an insulating layer 127 over the inorganic insulating layer 125 are preferably provided between the adjacent light-emitting devices 130.

Although each of the inorganic insulating layer 125 and the insulating layer 127 looks like a plurality of layers in the cross-sectional view in FIG. 4B, each of the inorganic insulating layer 125 and the insulating layer 127 is preferably one continuous layer when the display device 600 is seen from above. That is, the inorganic insulating layer 125 and the insulating layer 127 preferably include opening portions over first electrodes.

In FIG. 4B, a light-emitting device 130R, a light-emitting device 130G, and a light-emitting device 130B are illustrated as the light-emitting devices 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light.

The display device of one embodiment of the present invention can be, for example, a top-emission display device where light is emitted in the direction opposite to a substrate over which light-emitting devices are formed. Note that the display device of one embodiment of the present invention may be of a bottom emission type.

Examples of a light-emitting substance included in the light-emitting device 130 include organic compounds or organometallic complexes such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure described in Embodiment 3. The light-emitting device 130R includes the first electrode (pixel electrode) including a conductive layer 151R and a conductive layer 152R, an organic compound layer 103R over the first electrode, a common layer 104 over the organic compound layer 103R, and a common electrode 155 over the common layer 104. The common electrode 155 corresponds to the second electrode 102 in Embodiments 2 and 3. Although the common layer 104 is not necessarily provided, it is preferable to provide the common layer 104 to reduce damage to the organic compound layer 103R during processing. In the case where the common layer 104 is provided, the common layer 104 is preferably an electron-injection layer.

The light-emitting device 130G has a structure described in Embodiment 3. The light-emitting device 130G includes the first electrode (pixel electrode) including a conductive layer 151G and a conductive layer 152G, an organic compound layer 103G over the first electrode, the common layer 104 over the organic compound layer 103G, and the common electrode 155 over the common layer 104. The common electrode 155 corresponds to the second electrode 102 in Embodiments 2 and 3. Although the common layer 104 is not necessarily provided, it is preferable to provide the common layer 104 to reduce damage to the organic compound layer 103G during processing.

The light-emitting device 130B has a structure described in Embodiment 3. The light-emitting device 130B includes the first electrode (pixel electrode) including a conductive layer 151B and a conductive layer 152B, an organic compound layer 103B over the first electrode, the common layer 104 over the organic compound layer 103B, and the common electrode 155 over the common layer 104. The common electrode 155 corresponds to the second electrode 102 in Embodiments 2 and 3. Although the common layer 104 is not necessarily provided, it is preferable to provide the common layer 104 to reduce damage to the organic compound layer 103B during processing.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layer 103R, the organic compound layer 103G, and the organic compound layer 103B are island-shaped layers and are isolated on a light-emitting device basis or on an emission color basis. Providing the island-shaped organic compound layer 103 in each of the light-emitting devices 130 can inhibit leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped organic compound layer 103 is formed by forming an EL film and processing the EL film by a lithography method. Note that the organic compound layer 103 is referred to as an EL layer in some cases.

In the display device of one embodiment of the present invention, the first electrode (pixel electrode) of the light-emitting device preferably has a stacked-layer structure. For example, in the example shown in FIG. 4B, the first electrode of the light-emitting device 130 is a stack of the conductive layer 151 (conductive layers 151R, 151G, and 151B) and the conductive layer 152 (conductive layers 152R, 152G, and 152B). In the case where the display device 600 is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display device 600 is of a top-emission type, the higher the visible light reflectance of the pixel electrode is, the higher the efficiency of extraction of the light emitted by the organic compound layer 103 is. In the case where the pixel electrode functions as the anode, the higher the work function of the pixel electrode is, the easier hole injection into the organic compound layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage. In this specification and the like, description common to the conductive layers 151R, 151G, and 151B is sometimes made using the collective term “conductive layer 151”.

In the case where the conductive layer 151 has high reflectance for visible light, the visible light reflectance of the conductive layer 151 is preferably higher than or equal to 40% and lower than or equal to 100%, or higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

Thus, in the display device 600 of this embodiment, an insulating layer 156 (insulating layers 156R, 156G, and 156B) is formed on the side surfaces of the conductive layers 151 and 152. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display device 600 to be manufactured by a high-yield method and to be accordingly inexpensive. In addition, generation of a defect in the display device 100 can be inhibited, which makes the display device 600 highly reliable. In this specification and the like, description common to the conductive layers 156R, 156G, and 156B is sometimes made using the collective term “conductive layer 156”.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy including an appropriate combination of any of these metals, for example.

For the conductive layer 152, an oxide including one or more selected from indium, tin, zinc, gallium, titanium, aluminum, and silicon can be used. For example, it is preferable to use a conductive oxide including one or more of indium oxide, indium tin oxide, indium zinc oxide, zinc oxide, zinc oxide including gallium, titanium oxide, indium zinc oxide including gallium, indium zinc oxide including aluminum, indium tin oxide including silicon, indium zinc oxide including silicon, and the like. In particular, an indium tin oxide including silicon can be suitably used for the conductive layer 152 because of having a work function of higher than or equal to 4.0 eV, for example.

The conductive layer 151 and the conductive layer 152 may each be a stack of a plurality of layers including different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The structure described in this embodiment can be used in combination with any of the structures described in other embodiments as appropriate.

### Embodiment 5

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and laptop personal computers, a monitor of a computer and the like, digital signage, and a large game machine such as a pachinko machine.

### [Display Module]

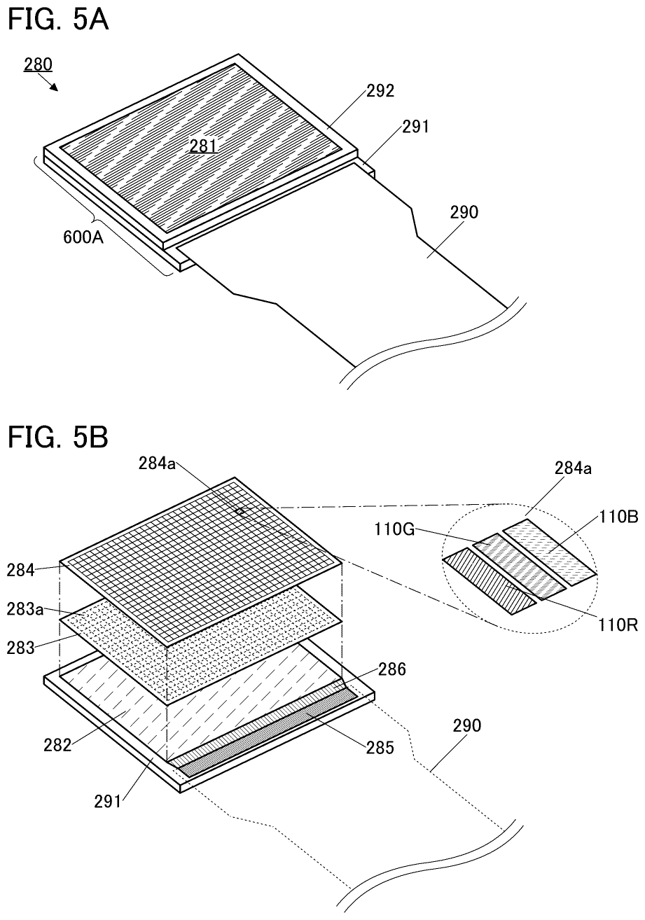

FIG. 5A is a perspective view of a display module 280. The display module 280 includes a display device 600A and an FPC 290. Note that the display device included in the display module 280 is not limited to the display device 600A and may be any of display devices 600B to 600F described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 5B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 5B. The pixels 284a can employ any of the structures described in the above embodiments. FIG. 5B illustrates an example where the pixel 284a has a structure similar to that of the pixel 178 illustrated in FIG. 4A.

The pixel circuit portion 283 includes a plurality of pixel circuits 283a arranged periodically.

One pixel circuit 283a is a circuit that controls driving of a plurality of elements included in one pixel 284a. For example, the pixel circuit 283a can include at least one selection transistor, one current control transistor (driving transistor), and a capacitor per light-emitting device. A gate signal is input to a gate of the selection transistor, and a video signal is input to a source or a drain of the selection transistor. Thus, an active-matrix display device is achieved.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high definition. For example, the pixels 284a are preferably arranged in the display portion 281 to give a definition higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

Such a display module 280 has extremely high resolution, and thus can be suitably used for a VR device such as an HID or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be favorably used in a display portion of a wearable electronic appliance, such as a wrist watch.

### [Display Device 600A]

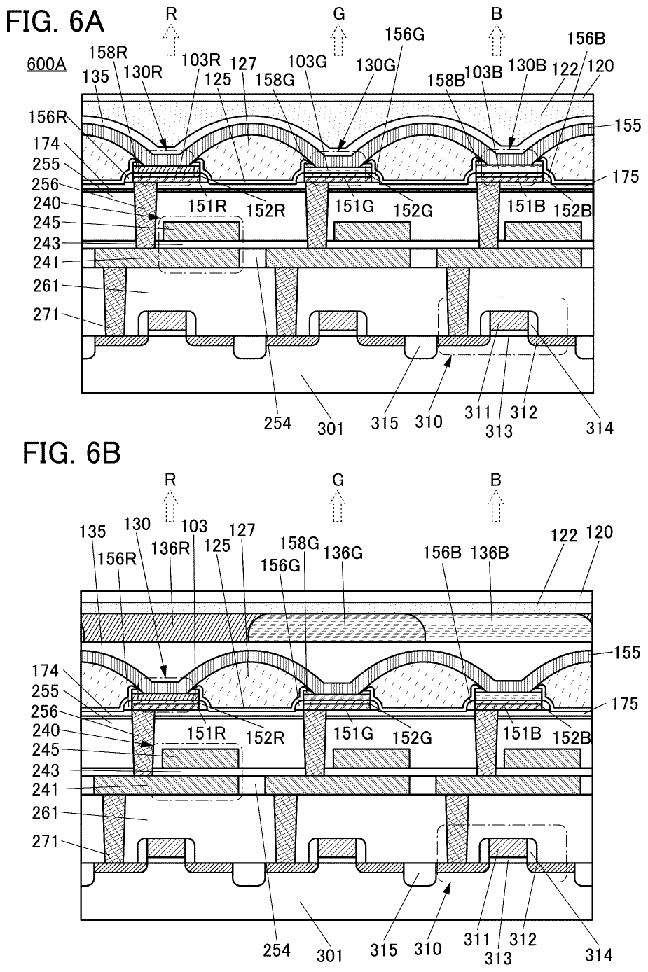

The display device 600A illustrated in FIG. 6A includes a substrate 301, the light-emitting devices 130R, 130G, and 130B, a capacitor 240, and a transistor 310.

The substrate 301 corresponds to the substrate 291 in FIGS. 5A and 5B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 6A illustrates an example in which the light-emitting devices 130R, 130G, and 130B each have the stacked-layer structure illustrated in FIG. 1A. An insulator is provided in regions between adjacent light-emitting devices. For example, in FIG. 6A, the inorganic insulating layer 125 and the insulating layer 127 over the inorganic insulating layer 125 are provided in those regions.

The insulating layer 156R is provided to include a region overlapping with the side surface of the conductive layer 151R of the light-emitting device 130R. The insulating layer 156G is provided to include a region overlapping with the side surface of the conductive layer 151G of the light-emitting device 130G. The insulating layer 156B is provided to include a region overlapping with the side surface of the conductive layer 151B of the light-emitting device 130B. The conductive layer 152R is provided to cover the conductive layer 151R and the insulating layer 156R. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R. The sacrificial layer 158G is positioned over the organic compound layer 103G. A sacrificial layer 158R is positioned over the organic compound layer 103R of the light-emitting device 130R. A sacrificial layer 158G is positioned over the organic compound layer 103G of the light-emitting device 130G. A sacrificial layer 158B is positioned over the organic compound layer 103B of the light-emitting device 130B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 135 is provided over the light-emitting devices 130R, 130G, and 130B. A substrate 120 is bonded onto the protective layer 135 with a resin layer 122. Embodiment 4 can be referred to for the details of the light-emitting device 130 and the components thereover up to the substrate 120. The substrate 120 corresponds to the substrate 292 in FIG. 5A.

FIG. 6B illustrates a variation example of the display device 600A illustrated in FIG. 6A. The light-emitting device illustrated in FIG. 6B includes the coloring layers 136R, 136G, and 136B, and each of the light-emitting devices 130 includes a region overlapping with one of the coloring layers 136R, 136G, and 136B. In the display device illustrated in FIG. 6B, the light-emitting device 130 can emit white light, for example. For example, the coloring layer 136R, the coloring layer 136G, and the coloring layer 136B can transmit red light, green light, and blue light, respectively.

### [Display Device 600B]

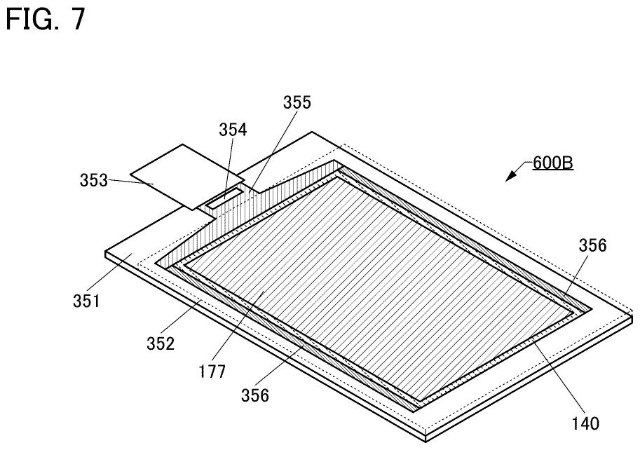

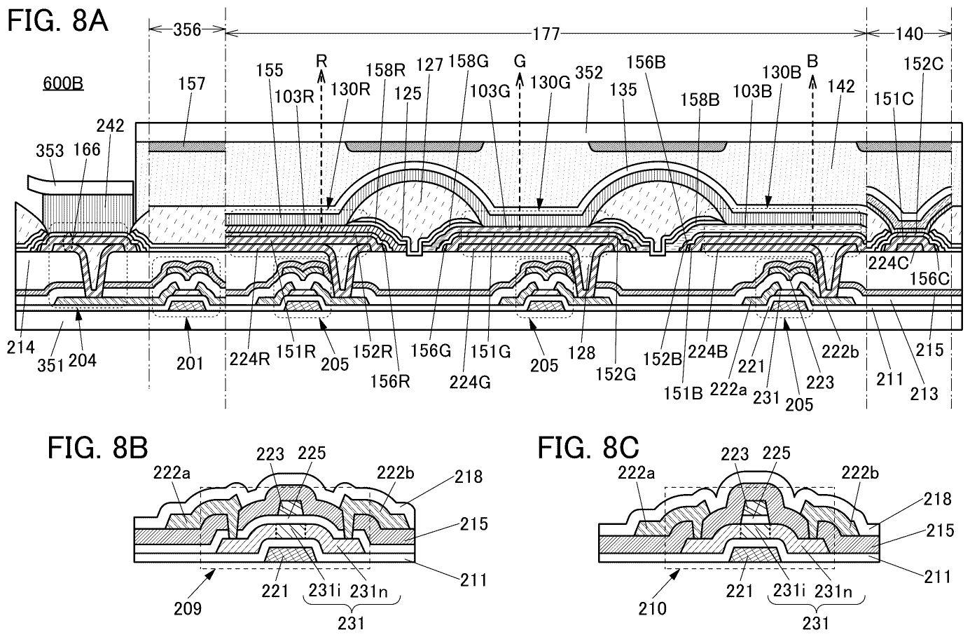

FIG. 7 is a perspective view of the display device 600B, and FIG. 8A is a cross-sectional view of the display device 600B.

In the display device 600B, a substrate 352 and a substrate 351 are bonded to each other. In FIG. 7, the substrate 352 is denoted by a dashed line.

The display device 600B includes the pixel portion 177, the connection portion 140, a circuit 356, a wiring 355, and the like. FIG. 7 shows an example in which an integrated circuit (IC) 354 and an FPC 353 are mounted on the display device 600B. Thus, the structure illustrated in FIG. 7 can be regarded as a display module including the display device 600B, the IC, and the FPC. Here, a display device in which a substrate is equipped with a connector such as an FPC or mounted with an IC is referred to as a display module.

The connection portion 140 is provided outside the pixel portion 177. The connection portion 140 can be provided along one side or a plurality of sides of the pixel portion 177. The number of connection portions 140 may be one or more. FIG. 7 illustrates an example in which the connection portion 140 is provided to surround the four sides of the pixel portion 177. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 7 illustrates an example in which the IC 354 is provided for the substrate 351 by a chip on glass (COG) method, a chip on film (COF) method, or the like. An IC including a scan line driver circuit, a signal line driver circuit, or the like can be used as the IC 354, for example. Note that the display device 600B and the display module are not necessarily provided with an IC. Alternatively, the IC may be mounted on the FPC by a COF method, for example.

FIG. 8A shows an example of cross sections of part of a region including the FPC 353, part of the circuit 356, part of the pixel portion 177, part of the connection portion 140, and part of a region including an end portion of the display device 600B.

The display device 600B shown in FIG. 8A includes a transistor 201, a transistor 205, the light-emitting device 130R that emits red light, the light-emitting device 130G that emits green light, the light-emitting device 130B that emits blue light, and the like between the substrate 351 and the substrate 352.

The stacked-layer structure of each of the light-emitting devices 130R, 130G, and 130B is the same as that shown in FIG. 1A except for the structure of the pixel electrode. The above embodiments can be referred to for the details of the light-emitting devices.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B. Here, the conductive layers 224R, 151R, and 152R can be collectively referred to as the pixel electrode of the light-emitting device 130R; the conductive layers 151R and 152R excluding the conductive layer 224R can also be referred to as the pixel electrode of the light-emitting device 130R. Similarly, the conductive layers 224G, 151G, and 152G can be collectively referred to as the pixel electrode of the light-emitting device 130G; the conductive layers 151G and 152G excluding the conductive layer 224G can also be referred to as the pixel electrode of the light-emitting device 130G. The conductive layers 224B, 151i, and 152B can be collectively referred to as the pixel electrode of the light-emitting device 130B; the conductive layers 151B and 152B excluding the conductive layer 224B can also be referred to as the pixel electrode of the light-emitting device 130B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G, and the insulating layer 156G in the light-emitting device 130G are not described in detail because they are respectively similar to the conductive layers 224R, 151R, and 152R, and the insulating layer 156R in the light-emitting device 130R; the same applies to the conductive layers 224B, 151B, and 152B, and the insulating layer 156B in the light-emitting device 130B.

The conductive layers 224R, 224G, and 224B each have a depressed portion covering the opening provided in the insulating layer 214. A layer 128 is embedded in the depressed portion.

The layer 128 has a function of filling the depressed portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depressed portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 135 is provided over the light-emitting devices 130R, 130G, and 130B. The protective layer 135 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 8A, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided in a frame shape not to overlap with the light-emitting device. Furthermore, the space may be filled with a resin other than the frame-like adhesive layer 142.

FIG. 8A shows an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and a conductive layer 152C obtained by processing the same conductive film as the conductive layers 152R, 152G, and 152B. In the example illustrated in FIG. 8A, the insulating layer 156C is provided to include a region overlapping with the side surface of the conductive layer 151C.

The display device 600B has a top-emission structure. Light from the light-emitting device is emitted toward the substrate 352. For the substrate 352, a material with a high visible-light-transmitting property is preferably used. The pixel electrode includes a material that reflects visible light, and a counter electrode (the common electrode 155) includes a material that transmits visible light.

The transistor 201 and the transistor 205 are formed over the substrate 351. These transistors can be fabricated using the same materials in the same steps.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

A material that does not easily allow diffusion of impurities such as water and hydrogen is preferably used for at least one of the insulating layers covering the transistors. This is because such an insulating layer can function as a barrier layer. Such a structure can effectively inhibit diffusion of impurities into the transistors from the outside and improve the reliability of a display device.

An inorganic insulating film is preferably used as each of the insulating layers 211, 213, and 215. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may be used. Two or more of the above insulating films may also be stacked.

An organic insulating layer is suitable as the insulating layer 214 functioning as a planarization layer. Examples of materials that can be used for the organic insulating layer include an acrylic resin, a polyimide resin, an epoxy resin, a polyamide resin, a polyimide-amide resin, a siloxane resin, a benzocyclobutene-based resin, a phenol resin, and precursors of these resins. The insulating layer 214 may have a stacked-layer structure of an organic insulating layer and an inorganic insulating layer. The outermost layer of the insulating layer 214 preferably functions as an etching protective layer. This can inhibit formation of a depressed portion in the insulating layer 214 at the time of processing of the conductive layer 224R, 151R, or 152R or the like. Alternatively, a depressed portion may be provided in the insulating layer 214 at the time of processing of the conductive layer 224R, 151R, or 152R or the like.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate. Here, a plurality of layers obtained by processing the same conductive film are shown with the same hatching pattern. The insulating layer 211 is positioned between the conductive layer 221 and the semiconductor layer 231. The insulating layer 213 is positioned between the conductive layer 223 and the semiconductor layer 231.

There is no particular limitation on the structure of the transistors included in the display device of this embodiment. For example, a planar transistor, a staggered transistor, or an inverted staggered transistor can be used. A top-gate transistor or a bottom-gate transistor can be used. Alternatively, gates may be provided above and below a semiconductor layer where a channel is formed.

The structure in which the semiconductor layer where a channel is formed is provided between two gates is employed for each of the transistors 201 and 205. The two gates may be connected to each other and supplied with the same signal to drive the transistor. Alternatively, the threshold voltage of the transistor may be controlled by applying a potential for controlling the threshold voltage to one of the two gates and a potential for driving to the other of the two gates.

There is no particular limitation on the crystallinity of a semiconductor material used for the transistors, and either an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case degradation of transistor characteristics can be inhibited.

The semiconductor layer of the transistor preferably includes a metal oxide. That is, a transistor including a metal oxide in its channel formation region (hereinafter referred to as an OS transistor) is preferably used in the display device of this embodiment.

Examples of an oxide semiconductor having crystallinity include a c-axis-aligned crystalline oxide semiconductor (CAAC-OS) and a nanocrystalline oxide semiconductor (nc-OS).

Alternatively, a transistor including silicon in its channel formation region (a Si transistor) may be used. Examples of silicon include single crystal silicon, polycrystalline silicon, and amorphous silicon. In particular, a transistor including low-temperature polysilicon (LTPS) in its semiconductor layer (hereinafter also referred to as an LTPS transistor) can be used. The LTPS transistor has high field-effect mobility and excellent frequency characteristics.

With the use of Si transistors such as LTPS transistors, a circuit required to be driven at a high frequency (e.g., a source driver circuit) can be formed on the same substrate as the display portion. This allows simplification of an external circuit mounted on the display device and a reduction in costs of parts and mounting costs.

An OS transistor has much higher field-effect mobility than a transistor including amorphous silicon. In addition, the OS transistor has an extremely low leakage current between a source and a drain in an off state, and charge accumulated in a capacitor that is connected in series to the transistor can be held for a long period. Furthermore, the use of an OS transistor can reduce the power consumption of the display device.

To increase the luminance of the light-emitting device included in the pixel circuit, the amount of current fed through the light-emitting device needs to be increased. To increase the current amount, the source-drain voltage of a driving transistor included in the pixel circuit needs to be increased. An OS transistor has a higher withstand voltage between a source and a drain than a Si transistor; hence, a high voltage can be applied between the source and the drain of the OS transistor. Therefore, when an OS transistor is used as the driving transistor in the pixel circuit, the amount of current flowing through the light-emitting device can be increased, so that the luminance of the light-emitting device can be increased.

Regarding saturation characteristics of a current flowing when transistors operate in a saturation region, even in the case where the source-drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source-drain current hardly changes with an increase in the source-drain voltage; hence, the luminance of the light-emitting device can be stable.

As described above, by using OS transistors as the driving transistors included in the pixel circuits, it is possible to suppress black-level degradation, increase the luminance, increase the number of gray levels, and suppress variations in light-emitting devices, for example.

The semiconductor layer preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

It is particularly preferable that an oxide containing indium (In), gallium (Ga), and zinc (Zn) (also referred to as IGZO) be used for the semiconductor layer. It is preferable to use an oxide containing indium, tin, and zinc. It is preferable to use an oxide containing indium, gallium, tin, and zinc. It is preferable to use an oxide containing indium (In), aluminum (Al), and zinc (Zn) (also referred to as IAZO). It is preferable to use an oxide containing indium (In), aluminum (Al), gallium (Ga), and zinc (Zn) (also referred to as IAGZO). Alternatively, it is preferable to use an oxide containing indium (also referred to as IO).

When the semiconductor layer is an In-M-Zn oxide, the atomic proportion of In is preferably higher than or equal to the atomic proportion of M in the In-M-Zn oxide. Examples of the atomic ratio of the metal elements in such an In-M-Zn oxide are In:M:Zn=1:1:1, 1:1:1.2, 2:1:3, 3:1:2, 4:2:3, 4:2:4.1, 5:1:3, 5:1:6, 5:1:7, 5:1:8, 6:1:6, and 5:2:5 and a composition in the neighborhood of any of the above atomic ratios. Note that the neighborhood of the atomic ratio includes ±30% of an intended atomic ratio.

When the atomic ratio is described as In:Ga:Zn=4:2:3 or a composition in the neighborhood thereof, the case is included where the atomic proportion of Ga is greater than or equal to 1 and less than or equal to 3 and the atomic proportion of Zn is greater than or equal to 2 and less than or equal to 4 with the atomic proportion of In being 4. In addition, when the atomic ratio is described as In:Ga:Zn=5:1:6 or a composition in the neighborhood thereof, the case is included where the atomic proportion of Ga is greater than 0.1 and less than or equal to 2 and the atomic proportion of Zn is greater than or equal to 5 and less than or equal to 7 with the atomic proportion of In being 5. Furthermore, when the atomic ratio is described as In:Ga:Zn=1:1:1 or a composition in the neighborhood thereof, the case is included where the atomic proportion of Ga is greater than 0.1 and less than or equal to 2 and the atomic proportion of Zn is greater than 0.1 and less than or equal to 2 with the atomic proportion of In being 1.

The transistors included in the circuit 356 and the transistors included in the pixel portion 177 may have the same structure or different structures. One structure or two or more kinds of structures may be employed for a plurality of transistors included in the circuit 356. Similarly, one structure or two or more kinds of structures may be employed for a plurality of transistors included in the pixel portion 177.

All transistors included in the pixel portion 177 may be OS transistors, or all transistors included in the pixel portion 177 may be Si transistors. Alternatively, some of the transistors included in the pixel portion 177 may be OS transistors and the others may be Si transistors.

For example, when both an LTPS transistor and an OS transistor are used in the pixel portion 177, the display device can have low power consumption and high driving capability. Note that a structure in which an LTPS transistor and an OS transistor are used in combination is referred to as LTPO in some cases. For example, it is preferable that an OS transistor be used as a transistor functioning as a switch for controlling electrical continuity between wirings and an LTPS transistor be used as a transistor for controlling a current.

For example, one transistor included in the pixel portion 177 functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. In that case, the amount of current flowing through the light-emitting device can be increased in the pixel circuit.

Another transistor included in the pixel portion 177 functions as a switch for controlling selection or non-selection of a pixel and can also be referred to as a selection transistor. A gate of the selection transistor is electrically connected to a gate line, and one of a source and a drain thereof is electrically connected to a source line (signal line). An OS transistor is preferably used as the selection transistor. In that case, the gray level of the pixel can be maintained even with an extremely low frame frequency (e.g., lower than or equal to 1 fps); thus, power consumption can be reduced by stopping the driver in displaying a still image.

As described above, the display device of one embodiment of the present invention can have all of a high aperture ratio, high resolution, high display quality, and low power consumption.

Note that the display device of one embodiment of the present invention has a structure including the OS transistor and the light-emitting device having a metal maskless (MML) structure. This structure can significantly reduce a leakage current that would flow through a transistor and a leakage current that would flow between adjacent light-emitting devices (sometimes referred to as a horizontal leakage current or a lateral leakage current). Displaying images on the display device having this structure can bring one or more of image crispness, image sharpness, high color saturation, and a high contrast ratio to the viewer. When a leakage current that would flow through the transistor and a lateral leakage current that would flow between the light-emitting devices are extremely low, leakage of light at the time of black display (black-level degradation) or the like can be minimized.

In particular, in the case where a light-emitting device having an MML structure employs a side-by-side (SBS) structure, which is the above-described structure for separately forming or coloring light-emitting layers, a layer provided between light-emitting devices (for example, also referred to as an organic layer or a common layer which is shared by the light-emitting devices) is disconnected; accordingly, side leakage can be prevented or be made extremely low.

FIGS. 8B and 8C illustrate other structure examples of transistors.

Transistors 209 and 210 each include the conductive layer 221 functioning as a gate, the insulating layer 211 functioning as a gate insulating layer, the semiconductor layer 231 including a channel formation region 231i and a pair of low-resistance regions 231n, the conductive layer 222a connected to one of the pair of low-resistance regions 231n, the conductive layer 222b connected to the other of the pair of low-resistance regions 231n, an insulating layer 225 functioning as a gate insulating layer, the conductive layer 223 functioning as a gate, and the insulating layer 215 covering the conductive layer 223. The insulating layer 211 is positioned between the conductive layer 221 and the channel formation region 231i. The insulating layer 225 is positioned at least between the conductive layer 223 and the channel formation region 231i. Furthermore, an insulating layer 218 covering the transistor may be provided.

FIG. 8B illustrates an example of the transistor 209 in which the insulating layer 225 covers the top and side surfaces of the semiconductor layer 231. The conductive layer 222a and the conductive layer 222b are connected to the corresponding low-resistance regions 231n through openings provided in the insulating layer 225 and the insulating layer 215. One of the conductive layers 222a and 222b functions as a source, and the other functions as a drain.

In the transistor 210 illustrated in FIG. 8C, the insulating layer 225 overlaps with the channel formation region 231i of the semiconductor layer 231 and does not overlap with the low-resistance regions 231n. The structure illustrated in FIG. 8C can be obtained by processing the insulating layer 225 with the conductive layer 223 used as a mask, for example. In FIG. 8C, the insulating layer 215 is provided to cover the insulating layer 225 and the conductive layer 223, and the conductive layer 222a and the conductive layer 222b are connected to the corresponding low-resistance regions 231n through openings in the insulating layer 215.

A connection portion 204 is provided in a region of the substrate 351 not overlapping with the substrate 352. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 600C]

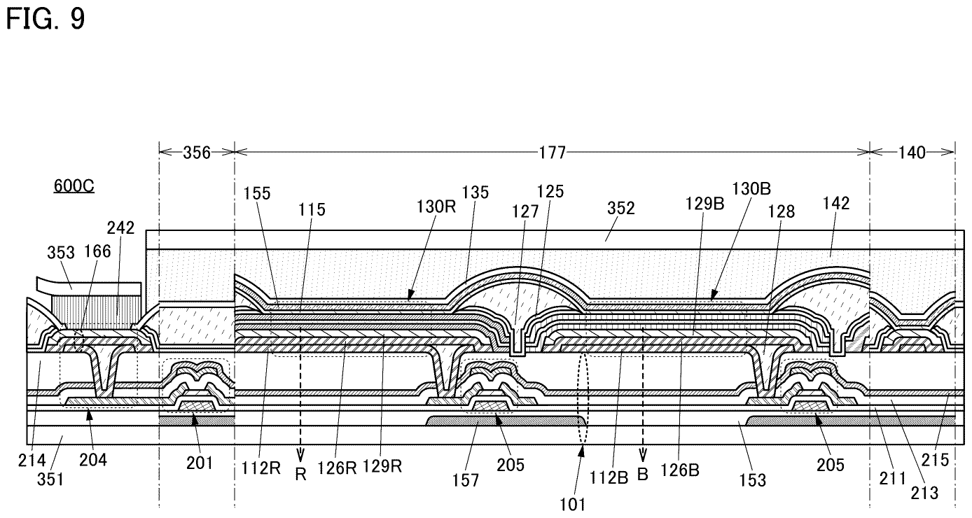

The display device 600C illustrated in FIG. 9 differs from the display device 600B illustrated in FIG. 8A mainly in having a bottom-emission structure.

Light from the light-emitting device is emitted toward the substrate 351. For the substrate 351, a material with a high visible-light-transmitting property is preferably used. By contrast, there is no limitation on the light-transmitting property of a material used for the substrate 352.

The light-blocking layer 157 is preferably formed between the substrate 351 and the transistor 201 and between the substrate 351 and the transistor 205. FIG. 9 illustrates an example in which the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material with a high visible-light-transmitting property is used for each of the conductive layers 112R, 112B, 126R, 126B, 129R, and 129B. A material that reflects visible light is preferably used for the common electrode 155.

Although not illustrated in FIG. 9, the light-emitting device 130G is also provided.

Although FIG. 9 and the like illustrate an example where the top surface of the layer 128 includes a flat portion, the shape of the layer 128 is not particularly limited.

### [Display Device 600D]

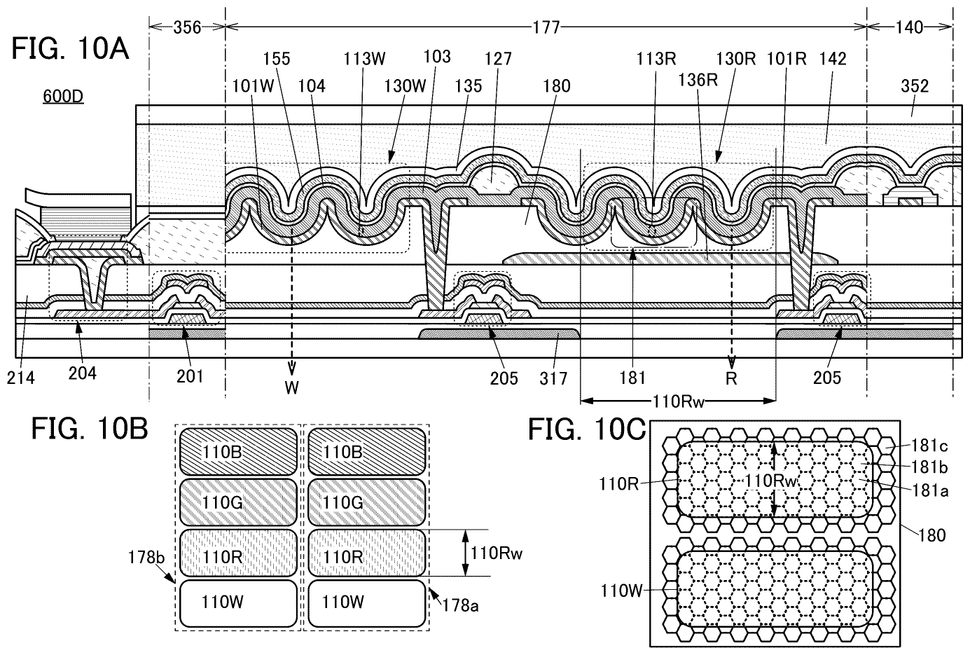

The display device 600D with a bottom-emission structure illustrated in FIG. 10A is an example of a bottom-emission display device different from the display device 600C illustrated in FIG. 9. The display device 600D is different from the display device 600C in including an organic resin layer 180. Note that in the drawings, reference numerals of some of the components that are shown in FIG. 9 are omitted; for the details of the components, the description made with reference to FIG. 9 is to be referred to.

FIG. 10B shows a top-view layout of the pixels 178 (a pixel 178a and a pixel 178b) each including the subpixels 110 (the subpixels 110R, 110G, 110B, and 110W), and FIG. 10C shows a top view of the organic resin layer 180 in a region where the subpixels 110R and 110W of the pixel 178 are formed. A region of the subpixel 110R between the light-blocking layers 317 can be represented as a width 110Rw in a light-emitting region.

As shown in FIG. 10A, the organic resin layer 180 is provided over the insulating layer 214. As shown in FIG. 10C and the region surrounded by the dashed-dotted line in FIG. 10A, the organic resin layer 180 includes a depressed portion 181 (depressed portions 181a and 181b) having a curved surface at least in a region where the subpixel is formed. Note that the depressed portion 181 may be provided outside the light-emitting region, like a depressed portion 181c. With the depressed portion 181c, light emission caused in a region overlapping with the light-blocking layer 317 or light travelled into the region overlapping with the light-blocking layer 317 can be refracted and extracted from the light-emitting region, whereby emission efficiency can be improved.

A plurality of depressed portions 181 may be formed in a matrix. The depressed portions 181a and 181b may be provided in contact with each other or may be provided to have a flat surface therebetween.

In FIGS. 10A and 10C, although the top surface shape and the cross-sectional shape of the depressed portion are hexagonal (FIG. 10C) and semicircular (FIG. 10A), respectively, other shapes may be employed as needed. Examples of the top-view shape of the depressed portion include polygons such as a triangle, a tetragon (including a rectangle and a square), and a pentagon; these polygons with rounded corners; an ellipse; and a circle.

An insulating layer including an organic material can be used as the organic resin layer 180. Examples of materials used for the organic resin layer 180 include an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. The organic resin layer 180 may be formed using an organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin.

A photosensitive resin can also be used for the organic resin layer 180. A photoresist may be used for the photosensitive resin. As the photosensitive resin, a positive photosensitive material or a negative photosensitive material can be used.

The organic resin layer 180 may include a material absorbing visible light. For example, the organic resin layer 180 itself may be made of a material absorbing visible light, or the organic resin layer 180 may include a pigment absorbing visible light. For example, the organic resin layer 180 can be formed using a resin that can be used as a color filter transmitting red, blue, or green light and absorbing light of the other colors; or a resin that includes carbon black as a pigment and functions as a black matrix.

The first electrode 101 (the first electrode 101R and a first electrode 101W) is over the organic resin layer 180 and the organic compound layer 103 is over the first electrode 101. End portions of the first electrode 101 and the organic compound layer 103 may be covered with the insulating layer 127.

The first electrode 101 formed over the organic resin layer 180 also has a depressed portion along the depressed portion of the organic resin layer 180. The organic compound layer 103 formed over the first electrode 101 also has a depressed portion along the depressed portion of the first electrode 101. The common layer 104 formed over the organic compound layer 103 also has a depressed portion along the depressed portion of the organic compound layer 103. The common electrode 155 formed over the common layer 104 also has a depressed portion along the depressed portion of the common layer 104. That is, the depressed portions of the organic resin layer 180, the first electrode 101, the organic compound layer 103, the common layer 104, and the common electrode 155 overlap with each other.

The common layer 104 is over the organic compound layer 103 and the insulating layer 127, and the common electrode 155 is over the common layer 104. The protective layer 135 is provided over the common electrode 155, and the substrate 352 is bonded with the use of the adhesive layer 142.

Although not shown in FIG. 10A, the light-emitting devices 130G and 130B are also provided.

### [Display Device 600E]

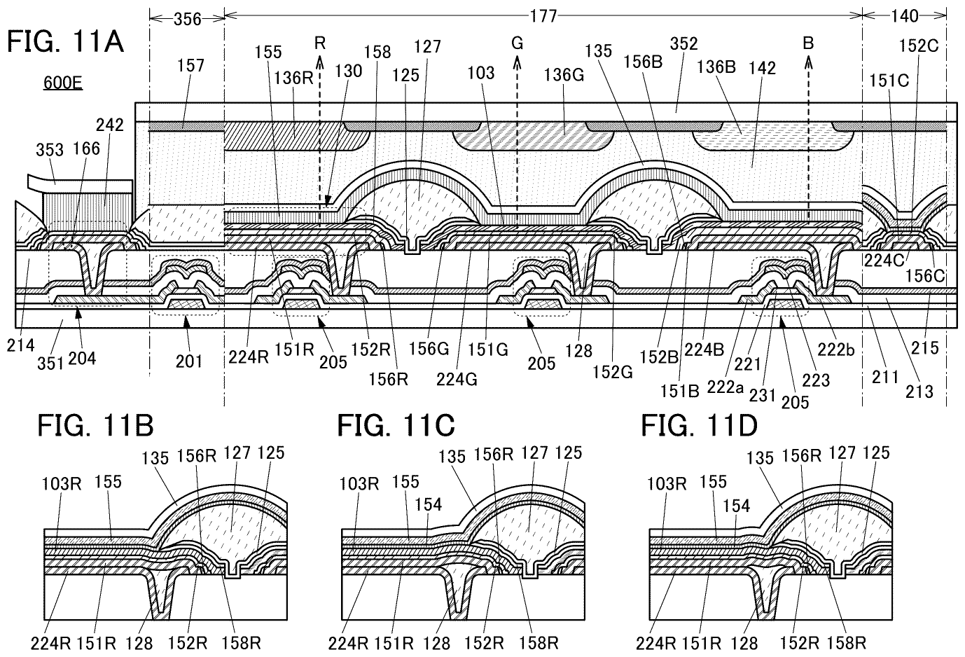

The display device 600E shown in FIG. 11A is a modification example of the top-emission display device 600B shown in FIG. 8A and differs from the display device 600B mainly in including the coloring layers 136R, 136G, and 136B.

In the display device 600E, the light-emitting device 130 includes a region overlapping with one of the coloring layers 136R, 136G, and 136B. The coloring layers 136R, 136G, and 136B can be provided on the surface of the substrate 352 on the substrate 351 side. End portions of the coloring layers 136R, 136G, and 136B can overlap with the light-blocking layer 157.

In the display device 600E, the light-emitting device 130 can emit white light, for example. For example, the coloring layer 136R, the coloring layer 136G, and the coloring layer 136B can transmit red light, green light, and blue light, respectively. Note that in the display device 600E, the coloring layers 136R, 136G, and 136B may be provided between the protective layer 135 and the adhesive layer 142.

Although FIG. 8A, FIG. 11A, and the like each illustrate an example in which the top surface of the layer 128 includes a flat portion, the shape of the layer 128 is not particularly limited. FIGS. 11B to 11D show modification examples of the layer 128.

As shown in FIGS. 11B and 11D, the top surface of the layer 128 can have a shape such that its middle and the vicinity thereof are depressed (i.e., a shape including a concave surface) in a cross-sectional view. A common layer 154 may be provided so as to be in contact with the common electrode 155.

As shown in FIG. 11C, the top surface of the layer 128 can have a shape in which its center and the vicinity thereof bulge, i.e., a shape including a convex surface, in a cross-sectional view.

The top surface of the layer 128 may include one or both of a convex surface and a concave surface. The number of convex surfaces and the number of concave surfaces included in the top surface of the layer 128 are not limited and can each be one or two or more.

The level of the top surface of the layer 128 and the level of the top surface of the conductive layer 224R may be the same or substantially the same, or may be different from each other. For example, the level of the top surface of the layer 128 may be lower or higher than the level of the top surface of the conductive layer 224R.

In the example shown in FIG. 11B, it can be said that the layer 128 fits inside the depressed portion of the conductive layer 224R. By contrast, as shown in FIG. 11D, the layer 128 is also present outside the depressed portion of the conductive layer 224R, i.e., the top surface of the layer 128 may extend beyond the depressed portion.

### [Display Device 600F]

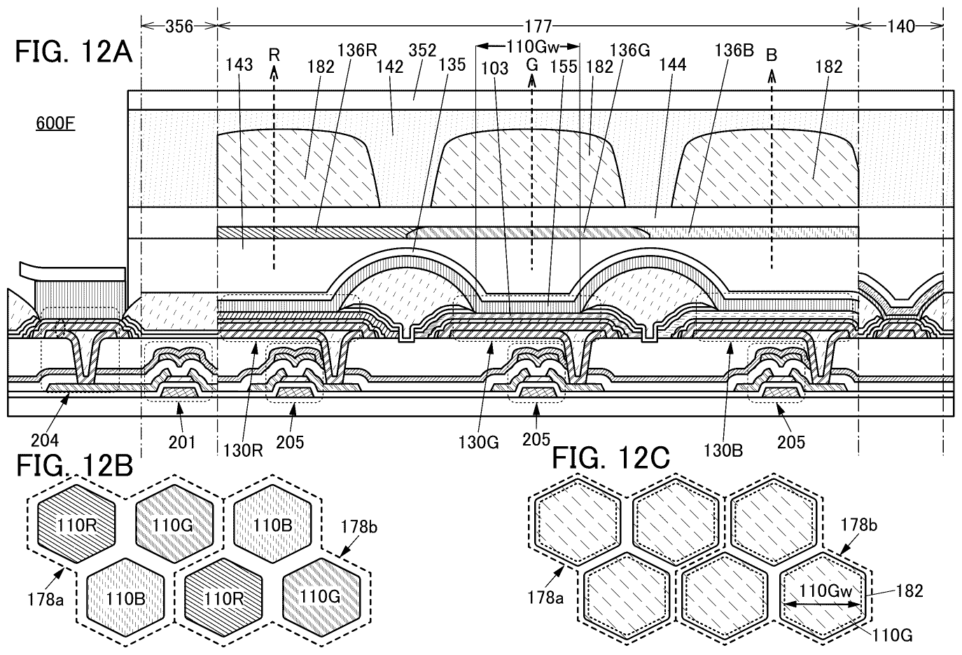

The display device 600F shown in FIG. 12A is a modification example of the top-emission display device 600B shown in FIGS. 8A to 8C and includes microlenses 182 over the coloring layers 136R, 136G, and 136B. Note that the reference numerals of the components that are the same as those in FIGS. 8A to 8C are sometimes omitted and the description for FIGS. 8A to 8C is referred to for the details of such components.

FIG. 12B shows a top-view layout of the pixels 178 (the pixels 178a and 178b) each including the subpixels 110 (the subpixels 110R, 110G, and 110B), and FIG. 12C shows a top view of the microlenses 182 in a region where the subpixels 110R, 110G, and 110B of the pixels 178 are formed. Note that the width of the region where the common electrode 155 and the organic compound layer 103 are in contact with each other corresponds to a width 110Gw in the light-emitting region of the subpixel 110G.

In the display device 600F shown in FIGS. 12A to 12C, a planarization film 143 is provided over the protective layer 135, and the coloring layers 136R, 136G, and 136B are provided over a planarization film 144. The planarization film 144 is provided to cover the coloring layers 136R, 136G, and 136B. The microlenses 182 are provided over the planarization film 144.

Note that as shown in FIG. 12C, the microlens 182 is preferably provided for each of the subpixels in a region where the subpixel is formed.

Although the top surface shape of the microlens 182 is shown as a hexagon in FIG. 12C, other shapes may be employed as needed. Examples of the top-view shape of the microlens 182 include polygons such as a triangle, a tetragon (including a rectangle and a square), and a pentagon; these polygons with rounded corners; an ellipse; and a circle.

The microlens 182 can be formed using a material similar to that for the organic resin layer 180.

This embodiment can be combined as appropriate with the other embodiments or the examples. In this specification, in the case where a plurality of structure examples are shown in one embodiment, the structure examples can be combined as appropriate.

### Embodiment 6

In this embodiment, electronic appliances of embodiments of the present invention will be described.

Electronic appliances of this embodiment include the display device of one embodiment of the present invention in their display portions. The display device of one embodiment of the present invention is highly reliable and can be easily increased in resolution and definition. Thus, the display device of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the display device of one embodiment of the present invention can have high resolution, and thus can be favorably used for an electronic appliance having a relatively small display portion. Examples of such an electronic appliance include watch-type and bracelet-type information terminals (wearable devices) and wearable devices capable of being worn on a head, such as a VR device like a head-mounted display, a glasses-type AR device, and an MR device.

The definition of the display device of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the display device of one embodiment of the present invention is preferably higher than or equal to 100 ppi, further preferably higher than or equal to 300 ppi, still further preferably higher than or equal to 500 ppi, yet still further preferably higher than or equal to 1000 ppi, yet still further preferably higher than or equal to 2000 ppi, yet still further preferably higher than or equal to 3000 ppi, yet still further preferably higher than or equal to 5000 ppi, yet still further preferably higher than or equal to 7000 ppi. With such a display device having high definition and/or high resolution, the electronic appliance can provide higher realistic sensation, sense of depth, and the like. There is no particular limitation on the screen ratio (aspect ratio) of the display device of one embodiment of the present invention. For example, the display device is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

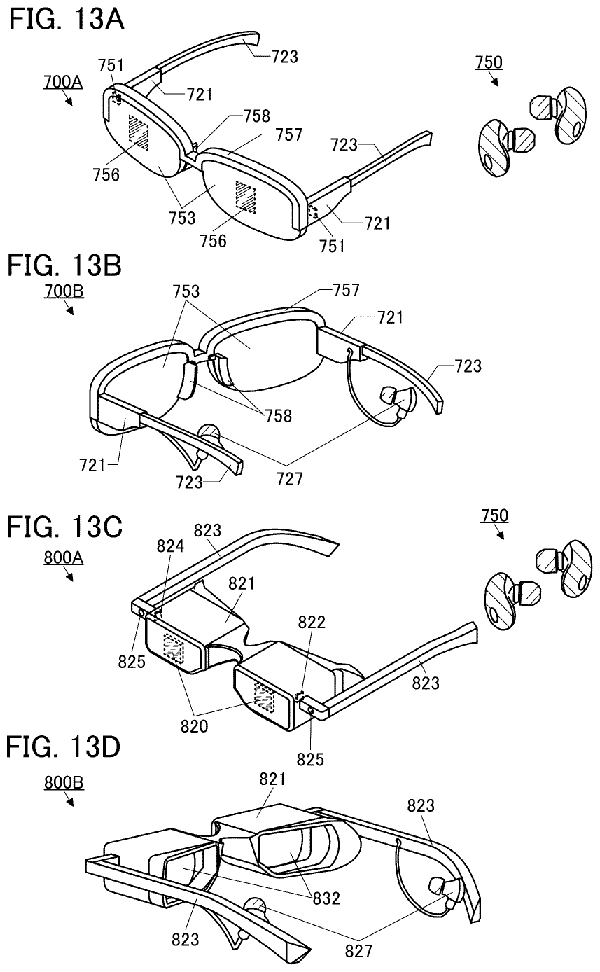

Examples of a wearable device capable of being worn on a head will be described with reference to FIGS. 13A to 13D. These wearable devices have at least one of a function of displaying AR contents, a function of displaying VR contents, a function of displaying SR contents, and a function of displaying MR contents. The electronic appliance having a function of displaying contents of at least one of AR, VR, SR, MR, and the like enables the user to feel a higher level of immersion.

An electronic device 700A illustrated in FIG. 13A and an electronic device 700B illustrated in FIG. 13B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, a highly reliable electronic appliance is obtained.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliances 700A and 700B are electronic appliances capable of performing AR display.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic device 800A illustrated in FIG. 13C and an electronic device 800B illustrated in FIG. 13D each include a pair of display portions 820, a housing 821, a communication portion 822, a pair of wearing portions 823, a control portion 824, a pair of image capturing portions 825, and a pair of lenses 832.

The display device of one embodiment of the present invention can be used in the display portions 820. Thus, a highly reliable electronic appliance is obtained.

The display portions 820 are positioned inside the housing 821 so as to be seen through the lenses 832. When the pair of display portions 820 display different images, three-dimensional display using parallax can be performed.

The electronic appliances 800A and 800B can be regarded as electronic appliances for VR. The user who wears the electronic appliance 800A or 800B can see images displayed on the display portions 820 through the lenses 832.

The electronic appliances 800A and 800B preferably include a mechanism for adjusting the lateral positions of the lenses 832 and the display portions 820 so that the lenses 832 and the display portions 820 are positioned optimally in accordance with the positions of the user's eyes. Moreover, the electronic appliances 800A and 800B preferably include a mechanism for adjusting focus by changing the distance between the lenses 832 and the display portions 820.

The electronic appliance 800A or the electronic appliance 800B can be mounted on the user's head with the wearing portions 823. FIG. 13C, for instance, shows an example where the wearing portion 823 has a shape like a temple (also referred to as a joint or the like) of glasses; however, one embodiment of the present invention is not limited thereto. The wearing portion 823 can have any shape with which the user can wear the electronic appliance, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to support a plurality of fields of view, such as a telescope field of view and a wide field of view.

Although an example where the image capturing portions 825 are provided is described here, a range sensor (hereinafter also referred to as a sensing portion) capable of measuring the distance between the user and an object just needs to be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic appliance 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic appliance 800A.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic device 700A in FIG. 13A has a function of transmitting information to the earphones 750 with the wireless communication function. For another example, the electronic device 800A in FIG. 13C has a function of transmitting information to the earphones 750 with the wireless communication function.

The electronic appliance may include an earphone portion. The electronic device 700B in FIG. 13B includes earphone portions 727. For example, the earphone portion 727 can be connected to the control portion by wire. Part of a wiring that connects the earphone portion 727 and the control portion may be positioned inside the housing 721 or the wearing portion 723.

Similarly, the electronic device 800B in FIG. 13D includes earphone portions 827. For example, the earphone portion 827 can be connected to the control portion 824 by wire. Part of a wiring that connects the earphone portion 827 and the control portion 824 may be positioned inside the housing 821 or the wearing portion 823. Alternatively, the earphone portions 827 and the wearing portions 823 may include magnets. This is preferable because the earphone portions 827 can be fixed to the wearing portions 823 with magnetic force and thus can be easily housed.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

As described above, both the glasses-type device (e.g., the electronic appliances 700A and 700B) and the goggles-type device (e.g., the electronic appliances 800A and 800B) are preferable as the electronic appliance of one embodiment of the present invention.

The electronic appliance of one embodiment of the present invention can transmit information to earphones by wire or wirelessly.

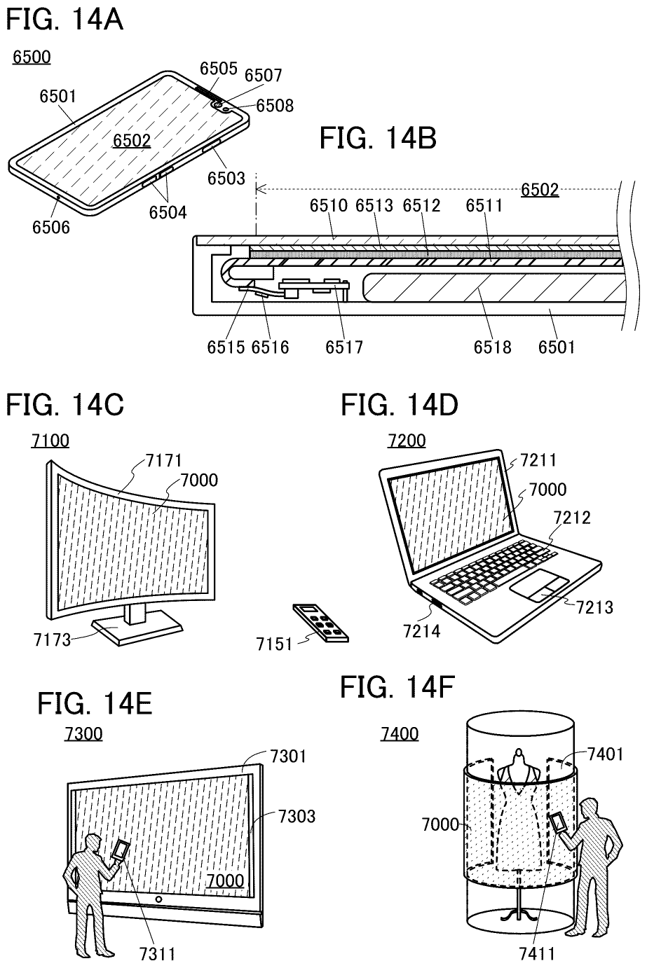

An electronic device 6500 illustrated in FIG. 14A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used in the display portion 6502. Thus, a highly reliable electronic appliance is obtained.

FIG. 14B is a schematic cross-sectional view including an end portion of the housing 6501 closer to the microphone 6506.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with a bonding layer (not shown).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, an extremely lightweight electronic appliance can be achieved. Since the display panel 6511 is extremely thin, the battery 6518 with high capacity can be mounted without an increase in the thickness of the electronic appliance. Moreover, part of the display panel 6511 is folded back so that a connection portion with the FPC 6515 is provided on the back side of the pixel portion, whereby an electronic appliance with a narrow bezel can be achieved.

FIG. 14C illustrates an example of a television device. In a television device 7100, a display portion 7000 is incorporated in a housing 7171. Here, the housing 7171 is supported by a stand 7173.

The display device of one embodiment of the present invention can be used in the display portion 7000. Thus, a highly reliable electronic appliance is obtained.

Operation of the television device 7100 illustrated in FIG. 14C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel of the remote control 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 14D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display device of one embodiment of the present invention can be used in the display portion 7000. Thus, a highly reliable electronic appliance is obtained.

FIGS. 14E and 14F illustrate examples of digital signage that can be used for store windows, showcases, and the like.

Digital signage 7300 illustrated in FIG. 14E includes a housing 7301, the display portion 7000, a speaker 7303, and the like. The digital signage 7300 can also include an LED lamp, an operation key (including a power switch or an operation switch), a connection terminal, a variety of sensors, a microphone, and the like.

FIG. 14F illustrates digital signage 7400 attached to a cylindrical pillar 7401. The digital signage 7400 includes the display portion 7000 provided along a curved surface of the pillar 7401.

In FIGS. 14E and 14F, the display device of one embodiment of the present invention can be used in the display portion 7000. Thus, a highly reliable electronic appliance is obtained.

A larger area of the display portion 7000 can increase the amount of information that can be provided at a time. The larger display portion 7000 attracts more attention, so that the effectiveness of the advertisement can be increased, for example.

Specifically, in the case where the display device of one embodiment of the present invention is used for the digital signage 7300 and the digital signage 7400 shown in FIGS. 14E and 14F that display advertisements and the like, the display device being a light-transmitting panel can increase the flexibility of representation. A light-transmitting display device can be manufactured, for example, by using a wiring and a support member each of which is formed of a conductive film that transmits visible light and adjusting the distance between pixel electrodes.

The use of the tandem light-emitting device of one embodiment of the present invention in addition to the wiring and the support member each of which is formed of the conductive film that transmits visible light can increase the luminance per pixel. That is, favorable display can be performed even when the aperture ratio of the display device is decreased; thus, the light-transmitting property of the display portion of the display device can be increased. Accordingly, such a structure is suitably used in the light-transmitting display device of one embodiment of the present invention.

As illustrated in FIGS. 14E and 14F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

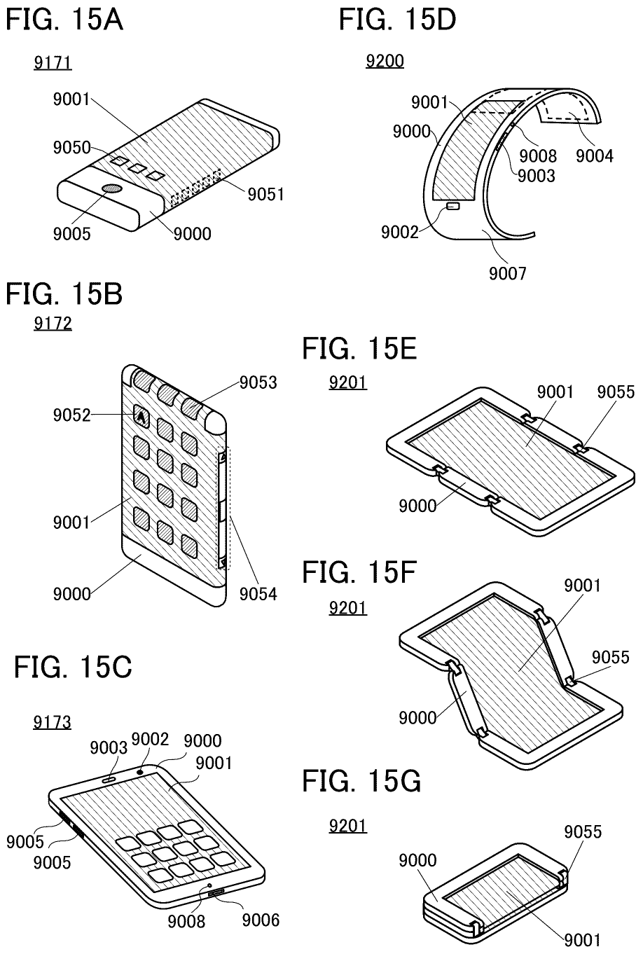

Electronic appliances illustrated in FIGS. 15A to 15G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic devices illustrated in FIGS. 15A to 15G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with use of a variety of software (programs), a wireless communication function, and a function of reading out and processing a program or data stored in a recording medium. Note that the functions of the electronic appliances are not limited thereto, and the electronic appliances can have a variety of functions. The electronic appliances may include a plurality of display portions. The electronic appliances may be provided with a camera or the like and have a function of taking a still image or a moving image, a function of storing the taken image in a storage medium (an external storage medium or a storage medium incorporated in the camera), a function of displaying the taken image on the display portion, and the like.

The electronic devices in FIGS. 15A to 15G will be described in detail below.

FIG. 15A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 15A illustrates an example where three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 15B is a perspective view of a portable information terminal 9172. The portable information terminal 9172 has a function of displaying information on three or more surfaces of the display portion 9001. In the example shown here, information 9052, information 9053, and information 9054 are displayed on different surfaces. For example, the user of the portable information terminal 9172 can check the information 9053 displayed such that it can be seen from above the portable information terminal 9172, with the portable information terminal 9172 put in a breast pocket of his/her clothes. Thus, the user can see the display without taking out the portable information terminal 9172 from the pocket and decide whether to answer the call, for example.

FIG. 15C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, a camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 15D is a perspective view of a watch-type portable information terminal 9200. The portable information terminal 9200 can be used as a Smartwatch (registered trademark), for example. The portable information terminal 9200 may include the operation key 9005 as a button for operation on the left side surface of the housing 9000 and the sensor 9007 on the bottom surface of the housing 9000. Although the housing 9000 having a curved bangle shape is shown as an example, a belt or the like may be used in combination with the housing 9000 to make the portable information terminal 9200 wearable. The display surface of the display portion 9001 is curved, and an image can be displayed on the curved display surface. A power storage device 9004 may have a curved shape along the housing 9000. The power storage device 9004 has flexibility and can be bent in accordance with a change in shape when the user puts on or takes off the portable information terminal 9200. Note that a charge control IC connected to the power storage device 9004 may be provided. Furthermore, for example, mutual communication between the portable information terminal 9200 and a headset capable of wireless communication can be performed, and thus hands-free calling is possible. The portable information terminal 9200 can perform mutual data transmission wirelessly with another information terminal and can be charged with wireless power feeding. Note that the connection terminal 9006 may be provided in the housing 9000 so that data transmission and charging operation may be performed by wire.

FIGS. 15E to 15G are perspective views of a foldable portable information terminal 9201. FIG. 15E is a perspective view illustrating the portable information terminal 9201 that is opened. FIG. 15G is a perspective view illustrating the portable information terminal 9201 that is folded. FIG. 15F is a perspective view illustrating the portable information terminal 9201 that is shifted from one of the states in FIGS. 15E and 15G to the other. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature greater than or equal to 0.1 mm and less than or equal to 150 mm, for example.

This embodiment can be combined as appropriate with the other embodiments or the examples. In this specification, in the case where a plurality of structure examples are shown in one embodiment, the structure examples can be combined as appropriate.

### Example 1

### Synthesis Example 1

Described in this synthesis example is a method for synthesizing the organic compound of the present invention represented by Structural Formula (100) in Embodiment 1, N-[4-(1-naphthyl)phenyl]-N-(9,9′-spirobi[9H-fluoren]-2-yl)benzo[b]naphtho[2,1-d]furan-10-amine (abbreviation: SFNBaBnf(10)). The structure of SFNBaBnf(10) is shown below.

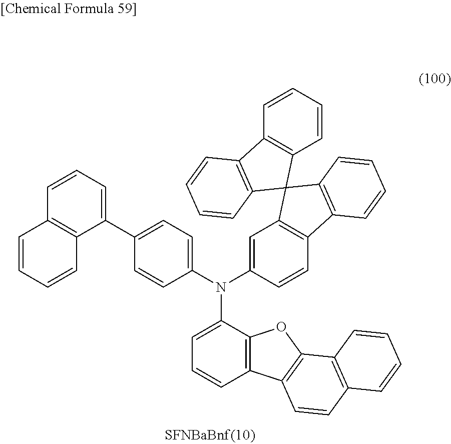 [SMILES: c1ccc2c(c1)-c1ccccc1C21c2ccccc2-c2ccc(N(c3ccc(-c4cccc5ccccc45)cc3)c3cccc4c3oc3c5ccccc5ccc43)cc21]

### <Synthesis of SFNBaBnf(10)>

Into a 100 mL three-neck flask were put 3.5 g (6.6 mmol) of N-[4-(1-naphthyl)phenyl]-N-(9,9′-spirobi[9H-fluoren]-2-amine and 2.0 g (6.7 mmol) of 10-bromobenzo[b]naphtho[2,1-d]furan. After the air in the flask was replaced with nitrogen, 2.1 g (22 mmol) of sodium tert-butoxide (abbreviation: ^{t}BuONa) and 34 mL of toluene were added thereto. This mixture was degassed by being stirred under reduced pressure. After that, the mixture was heated at 60° C. To this reaction solution were added 0.40 mL (0.15 mmol) of tri-tert-butylphosphine (abbreviation: P(Bu)₃) (10 wt % hexane solution) and 41 mg (71 μmol) of bis(dibenzylideneacetone)palladium(0), and stirring was performed at 120° C. for 4 hours. Toluene was added to this mixture and the obtained mixture was subjected to suction filtration through alumina, Celite (FUJIFILM Wako Pure Chemical Corporation, Catalog No: 537-02305), and Florisil (FUJIFILM Wako Pure Chemical Corporation, Catalog No: 066-05265). The resulting filtrate was concentrated to give 5.8 g of a white solid containing the target substance. This solid was purified by high performance liquid chromatography (HPLC) (mobile phase: chloroform) to give 5.2 g of a white solid containing the target substance. By a train sublimation method, 3.1 g of the obtained white solid was purified. In the purification by sublimation, the solid was heated at 310° C. under a pressure of 1.60 Pa for 24 hours. After the purification by sublimation, 2.1 g of a target white solid was obtained (yield: 43%, collection rate: 68%). Synthesis Scheme (s-1) of SFNBaBnf(10) is shown below.

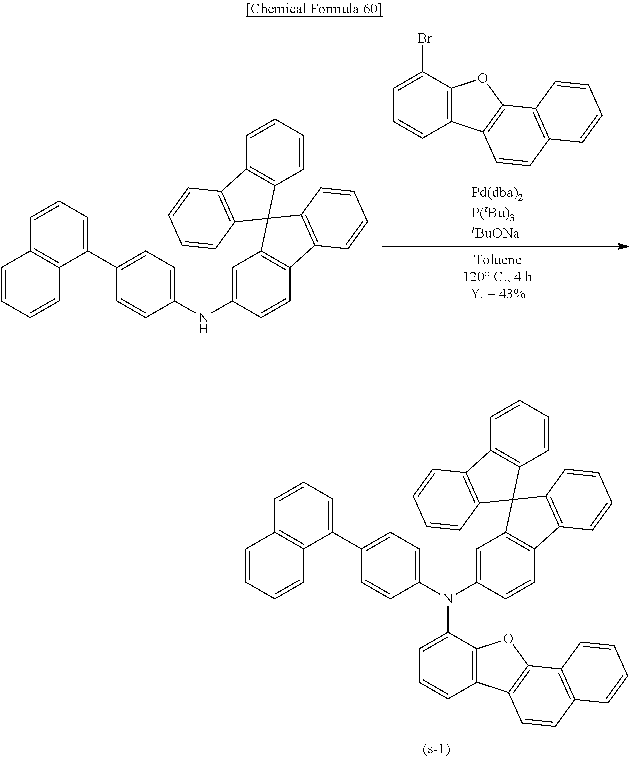 [SMILES: c1ccc2c(c1)-c1ccccc1C21c2ccccc2-c2ccc(Nc3ccc(-c4cccc5ccccc45)cc3)cc21 ; Brc1cccc2c1oc1c3ccccc3ccc21 ; c1ccc2c(c1)-c1ccccc1C21c2ccccc2-c2ccc(N(c3ccc(-c4cccc5ccccc45)cc3)c3cccc4c3oc3c5ccccc5ccc43)cc21]

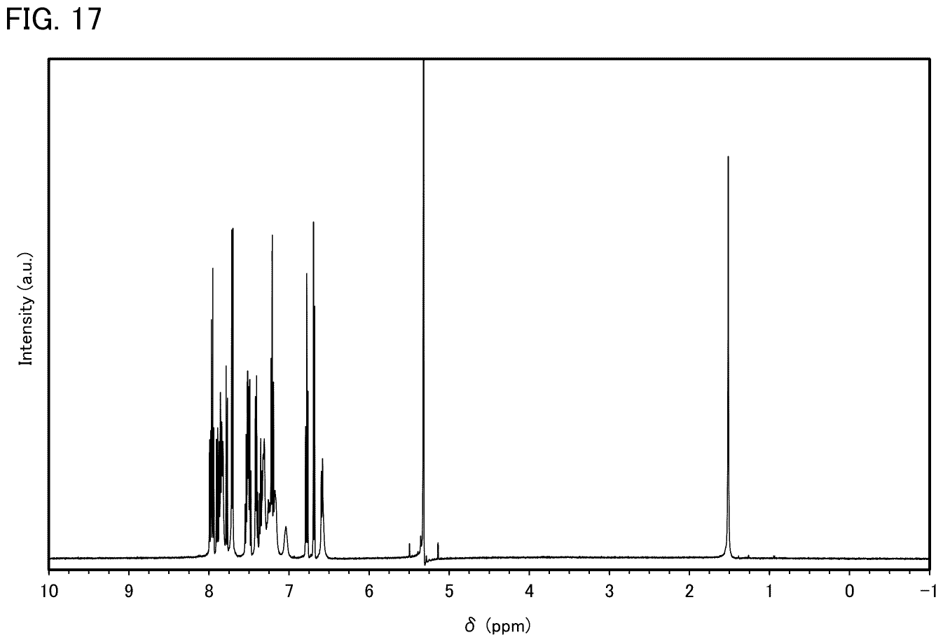

The results of ¹H NMR measurement of the obtained white solid are given below. FIG. 17 shows a ¹H NMR spectrum. This shows that SFNBaBnf(10) was obtained in this synthesis example.

¹H NMR (dichloromethane-d₂, 500 MHz): δ=7.99-7.94 (m, 3H), 7.91-7.83 (m, 5H), 7.78 (d, J=9.0 Hz, 1H), 7.71 (d, J=7.5 Hz, 3H), 7.55-7.47 (m, 4H), 7.42-7.14 (br-m, 12H), 7.04 (br, 1H), 6.78 (t, J=7.0 Hz, 2H), 6.69 (d, J=7.5 Hz, 2H), 6.59 (br-d, J=8.0 Hz, 2H).

The molecular weight of the white solid obtained was measured by LC/MS analysis. Note that in the LC/MS analysis, liquid chromatography (LC) separation was performed with UltiMate 3000 manufactured by Thermo Fisher Scientific K. K., and mass spectrometry (MS) was performed with Q Exactive manufactured by Thermo Fisher Scientific K. K.

As a result, a signal was observed at a m/z of 749 while the mass of the target substance was calculated to be 749, revealing that SFNBaBnf(10) was obtained.

### <Measurement of Physical Properties>

Next, the ultraviolet-visible absorption spectra (hereinafter, simply referred to as absorption spectra) and photoluminescence (PL) spectra (hereinafter, simply referred to as “emission spectra”) of a toluene solution and a thin film of SFNBaBnf(10) were measured. The absorption spectrum was measured with an ultraviolet-visible spectrophotometer (V-770DS, produced by JASCO Corporation). The emission spectrum was measured with a fluorescence spectrophotometer (FP-8600DS, JASCO Corporation).

To calculate the absorption spectrum of the toluene solution of SFNBaBnf(10), the absorption spectrum of toluene put in a quartz cell was measured and then subtracted from the absorption spectrum of the toluene solution of SFNBaBnf(10) put in a quartz cell.

To obtain the absorption spectrum and the emission spectrum of the thin film, a measurement sample was measured. The measurement sample was fabricated in the following manner: SFNBaBnf(10) was formed over a quartz substrate by a vacuum evaporation method and sealed using another quartz substrate as a counter substrate. Note that the emission spectrum was obtained by measuring the sealed sample, and the absorption spectrum was obtained by measuring the sample from which the sealing was removed and the counter substrate was detached. The absorption spectrum was obtained by subtraction of the absorption spectrum of the quartz substrate from the absorption spectrum of SFNBaBnf(10) formed over the quartz substrate.

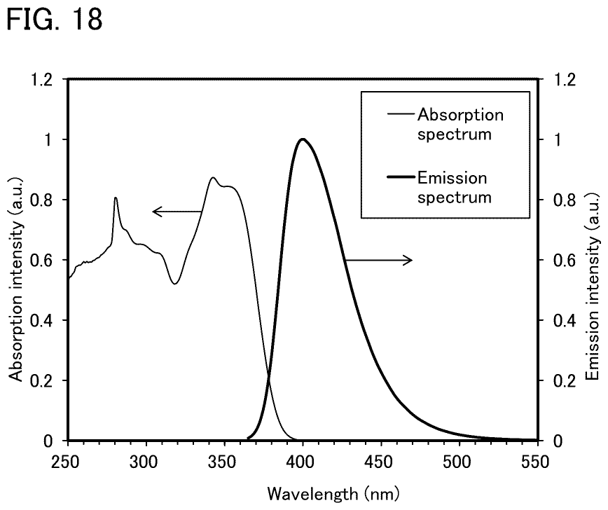

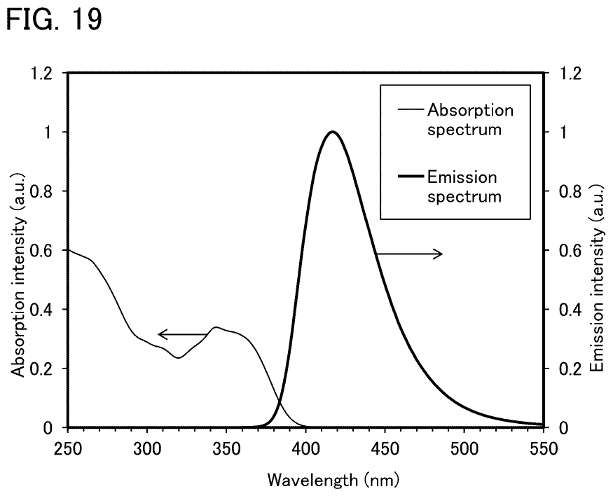

FIG. 18 and FIG. 19 show the measurement results of the toluene solution and the thin film, respectively. According to the measurement results, the toluene solution of SFNBaBnf(10) has an absorption peak at around 357 nm, the thin film of SFNBaBnf(10) has an absorption peak at around 362 nm, and neither the toluene solution nor the thin film exhibits an absorption band at wavelengths longer than 430 nm. The results suggests that the absorption does not reduce the efficiency of emission at the wavelengths employed for a display, showing the suitability of SFNBaBnf(10) for a light-emitting device. The measurement results also show that the toluene solution of SFNBaBnf(10) exhibits an emission wavelength peak at around 400 nm (excitation wavelength: 352 nm), and the thin film of SFNBaBnf(10) exhibits an emission wavelength peak at around 417 nm (excitation wavelength: 343 nm).

The HOMO level and the LUMO level of SFNBaBnf(10) were obtained through a cyclic voltammetry (CV) measurement. The calculation method is described below.

An electrochemical analyzer (ALS model 600A or 600C, BAS Inc.) was used as a measurement apparatus. A solution used for the CV measurement was prepared as follows: with use of dehydrated dimethylformamide (DMF, product of FUJIFILM Wako Pure Chemical Corporation, 99.5+%, catalog No. 043-32361) as a solvent, tetra-n-butylammonium perchlorate (n-Bu₄NClO₄, product of Tokyo Chemical Industry Co., Ltd., catalog No. T0836), which was a supporting electrolyte, was dissolved in the solvent to give a concentration of 100 mmol/L, and the object to be measured was further dissolved therein to give a concentration of 2 mmol/L.

A platinum electrode (PTE platinum electrode, manufactured by BAS Inc.) was used as the working electrode, another platinum electrode (Pt counter electrode for VC-3 (5 cm), manufactured by BAS Inc.) was used as an auxiliary electrode, and an Ag/Ag⁺ electrode (RE7 reference electrode for nonaqueous solvent, manufactured by BAS Inc.) was used as the reference electrode. Note that the measurement was performed at room temperature (higher than or equal to 20° C. and lower than or equal to 25° C.). The scan speed in the CV measurement was fixed to 0.1 V/sec, and an oxidation potential Eₐ [V] and a reduction potential E_{c} [V] with respect to the reference electrode were measured. The potential Eₐ is an intermediate potential of an oxidation-reduction wave, and the potential E_{c} is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Eₐ and LUMO level [eV]=−4.94−E_{c}.

The CV measurements waves repeated 100 times, and the oxidation-reduction wave in the 100th cycle was compared with the oxidation-reduction wave in the first cycle to examine the electrical stability of the compound.

As a result, the HOMO level of SFNBaBnf(10) was found to be −5.52 eV in the oxidation potential Eₐ [V] measurement. The LUMO level was found to be −2.18 eV in the reduction potential E_{c} [V] measurement. Comparison of the waveforms in the first cycle and the 100th cycle in repeated measurements of the oxidation-reduction wave shows that the peak intensity in the 100th cycle of the Eₐ measurement was maintained at 89% of that in the first cycle and the peak intensity in the 100th cycle of the E_{c} measurement was maintained at 97% of that in the first cycle. These results revealed that SFNBaBnf(10) is highly resistant to repeated oxidation and repeated reduction.

Next, the lowest triplet excitation energy level (T₁ level) of SFNBaBnf(10) was calculated through the measurement of an emission spectrum (a phosphorescence spectrum). The calculation method is described below.

For calculation of the lowest triplet excitation energy level (T₁ level), an emission spectrum (a phosphorescence spectrum) was measured at a measurement temperature of 10 K using a 50-nm-thick thin film of a sample formed over a quartz substrate. The measurement was performed with a PL microscope (LabRAM HR-PL, HORIBA, Ltd.) and a He—Cd laser (325 nm) as excitation light. Note that the emission edge was determined as the intersection of a tangent and the horizontal axis (representing wavelength) or the baseline. The tangent is drawn to have the maximum slope at a point on a shorter wavelength side of the shortest-wavelength peak (or the shortest-wavelength shoulder peak) of the emission spectrum (phosphorescence spectrum).

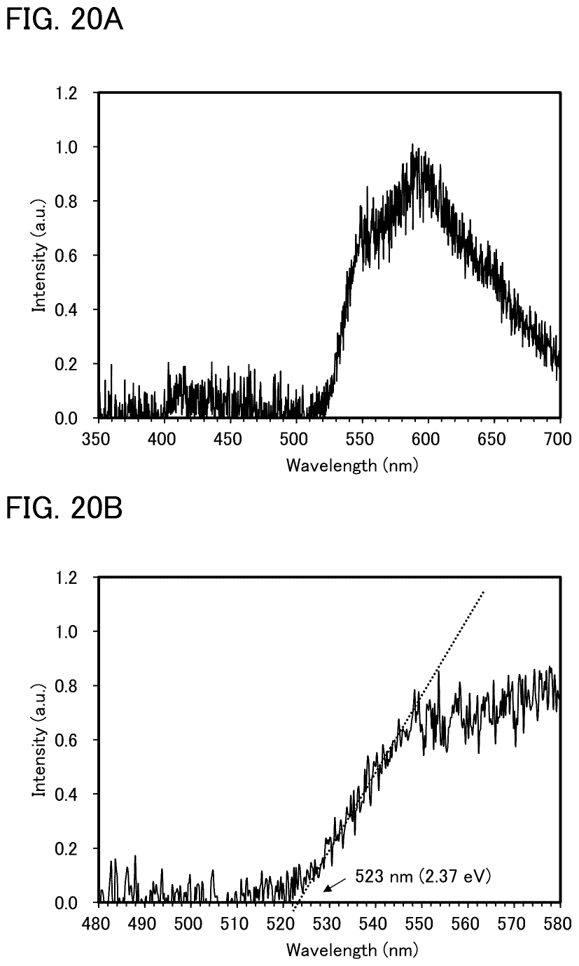

FIG. 20A shows the measurement results of the phosphorescence spectrum (10 K) of SFNBaBnf(10). FIG. 20B shows the phosphorescence spectrum (10 K) of SFNBaBnf(10) in the wavelength range of 480 nm to 580 nm. According to FIG. 20B, the wavelength of the emission edge on the short wavelength side of the phosphorescence spectrum (10 K) of SFNBaBnf(10) is 523 nm, which indicates that the lowest triplet excitation energy level (T₁ level) of SFNBaBnf(10) is 2.37 eV.

Differential scanning calorimetry (DSC) measurement of SFNBaBnf(10) was performed with DSC8500 manufactured by PerkinElmer, Inc. The DSC measurement was performed in the following manner: the temperature was raised from −10° C. to 330° C. at a temperature rising rate of 40° C./min and held for 3 minutes, and then the temperature was decreased to −10° C. at a temperature decreasing rate of 40° C./min and held for 3 minutes. This operation was performed twice in succession. The DSC measurement result of a second cycle showed that the glass transition point of SFNBaBnf(10) was 151° C. The crystallization temperature and the melting point were not observed. This indicates that SFNBaBnf(10) is a substance having high heat resistance and the film of SFNBaBnf(10) can maintain a thermally stable quality.

For comparison, differential scanning calorimetry measurement (DSC measurement) of N-phenyl-N-(9,9′-spirobi[9H-fluoren]-2-yl)benzo[b]naphtho[2,1-d]furan-10-amine (abbreviation: SFAaBnf(10)), which has a structure in which the 1-naphthyl group is eliminated from SFNBaBnf(10), was performed under the same conditions. The results reveal that SFAaBnf(10) has a glass transition point of 129° C., which is lower than that of SFNBaBnf(10). Therefore, inclusion of the 1-naphthyl group enables SFNBaBnf(10) to have a higher glass transition point.

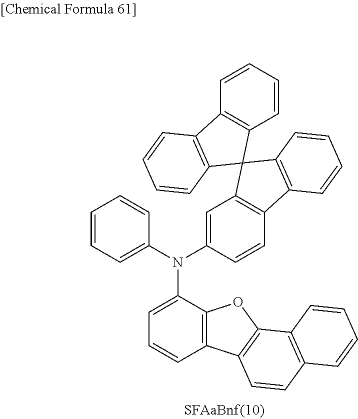 [SMILES: c1ccc(N(c2ccc3c(c2)C2(c4ccccc4-c4ccccc42)c2ccccc2-3)c2cccc3c2oc2c4ccccc4ccc32)cc1]

The thermogravimetry-differential thermal analysis (TG-DTA) of SFNBaBnf(10) was performed. The measurement was conducted using a high vacuum differential type differential thermal balance (TG-DTA 2410SA, manufactured by Bruker AXS K.K.). The measurement was performed under an atmospheric pressure at a temperature rising rate of 10° C./min under a nitrogen stream (flow rate: 200 mL/min). In the thermogravimetry-differential thermal analysis, the temperature (decomposition temperature) at which the weight obtained by thermogravimetry was reduced by 5% of the weight at the beginning of the measurement was found to be 388° C., which shows that SFNBaBnf(10) is a substance having high heat resistance.

The above results reveal that SFNBaBnf(10), which is the organic compound of one embodiment of the present invention, is an organic compound with high electrical stability and high heat resistance and can be suitably used for an organic semiconductor device such as a light-emitting device.

### Example 2

### Synthesis Example 2

Described in this synthesis example is a method for synthesizing the organic compound of the present invention represented by Structural Formula (101) in Embodiment 1, N-[4-(1-naphthyl)phenyl]-N-(9,9′-spirobi[9H-fluoren]-2-yl)benzo[b]naphtho[2,3-d]furan-4-amine (abbreviation: SFNBBnf(II)(4)). The structure of SFNBBnf(II)(4) is shown below.

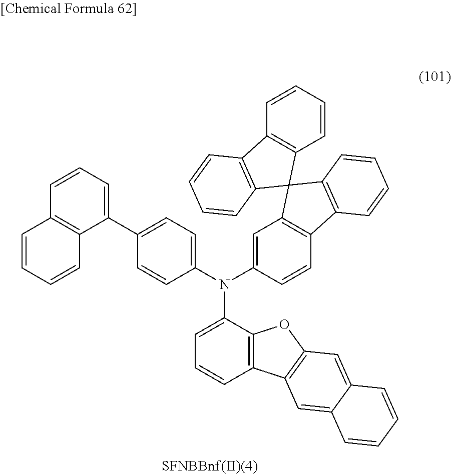 [SMILES: c1ccc2c(c1)-c1ccccc1C21c2ccccc2-c2ccc(N(c3ccc(-c4cccc5ccccc45)cc3)c3cccc4c3oc3cc5ccccc5cc34)cc21]

### <Synthesis of SFNBBnf(II)(4)>

Into a 100 mL three-neck flask were put 3.5 g (6.6 mmol) of N-[4-(1-naphthyl)phenyl]-N-(9,9′-spirobi[9H-fluoren]-2-amine, 1.7 g (6.6 mmol) of 4-chlorobenzo[b]naphtho[2,3-d]furan, and 70 mg (0.17 mmol) of 2-dicyclohexylphosphino-2′,6′-dimethoxybiphenyl (commonly known name: SPhos). After the air in the flask was replaced with nitrogen, 2.1 g (22 mmol) of sodium tert-butoxide (abbreviation: ^{t}BuONa) and 33 mL of toluene were added thereto. This mixture was degassed by being stirred under reduced pressure. After that, the mixture was heated at 60° C. To this reaction solution was added 41 mg (71 μmol) of bis(dibenzylideneacetone)palladium(0), and stirring was performed at 120° C. for 7 hours. Toluene was added to this mixture and the obtained mixture was subjected to suction filtration through alumina, Celite, and Florisil. The resulting filtrate was concentrated to give 6.3 g of a light yellow solid containing the target substance. By a train sublimation method, 3.2 g of the obtained light yellow solid was purified. In the purification by sublimation, the solid was heated at 325° C. under a pressure of 1.75 Pa for 18 hours. After the purification by sublimation, 2.6 g of a target light yellow solid was obtained (yield: 51%, collection rate: 79%). Synthesis Scheme (s-2) of SFNBBnf(II)(4) is shown below.

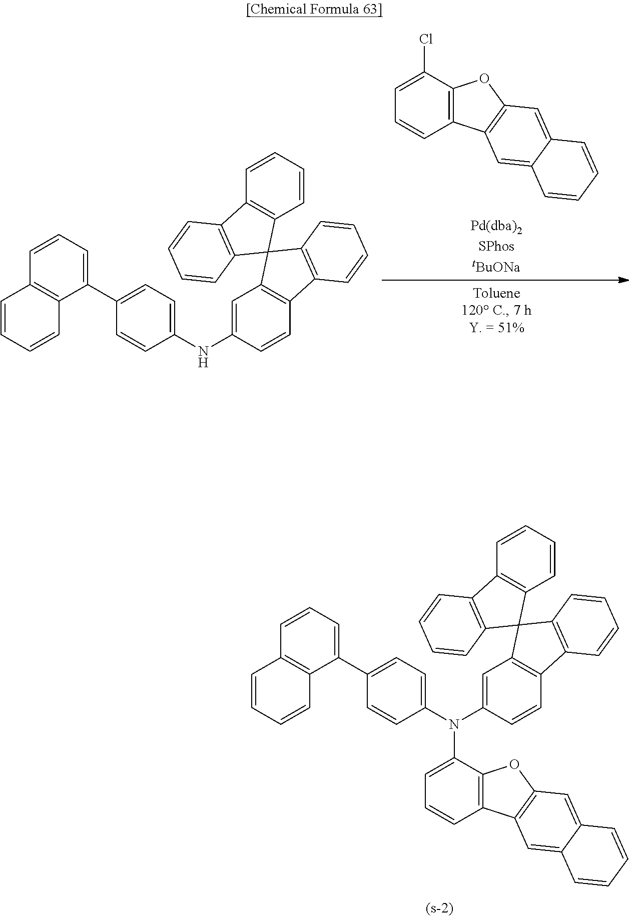 [SMILES: c1ccc2c(c1)-c1ccccc1C21c2ccccc2-c2ccc(Nc3ccc(-c4cccc5ccccc45)cc3)cc21 ; Clc1cccc2c1oc1cc3ccccc3cc12 ; c1ccc2c(c1)-c1ccccc1C21c2ccccc2-c2ccc(N(c3ccc(-c4cccc5ccccc45)cc3)c3cccc4c3oc3cc5ccccc5cc34)cc21]

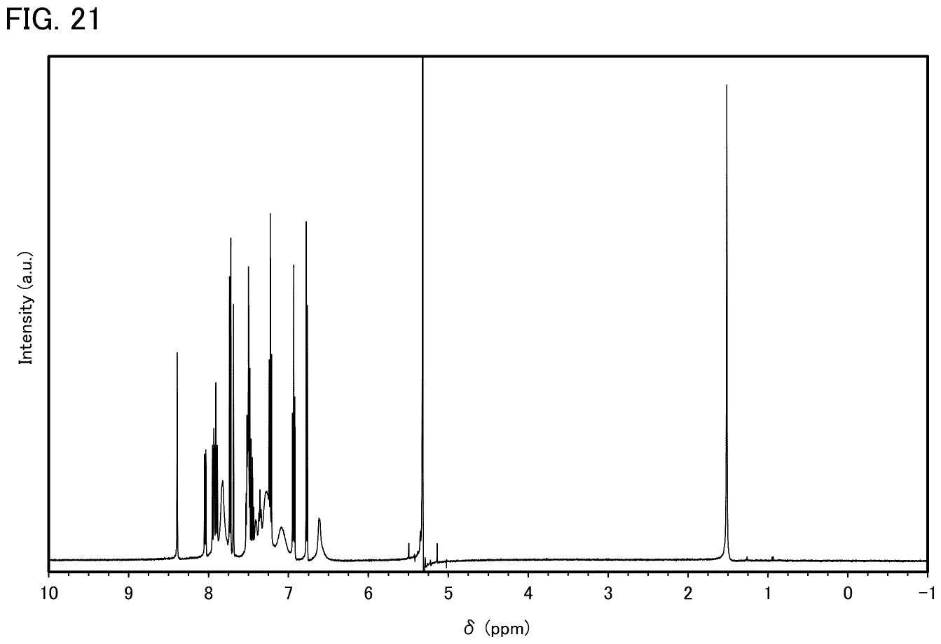

The results of ¹H NMR measurement of the obtained light yellow solid are given below. FIG. 21 shows a ¹H NMR spectrum. This shows that SFNBBnf(II)(4) was obtained in this synthesis example.

¹H NMR (dichloromethane-d₂, 500 MHz): δ=8.39 (s, 1H), 8.04 (d, J=8.0 Hz, 1H), 7.95-7.89 (m, 4H), 7.82 (br, 3H), 7.73 (d, J=8.0 Hz, 2H), 7.69 (s, 1H), 7.5-7.21 (br, 14H), 7.09 (br, 3H), 6.94 (t, J=7.5 Hz, 2H), 6.76 (d, J=7.5 Hz, 2H), 6.62 (br, 2H).

The molecular weight of the light yellow solid obtained was measured by LC/MS analysis. Note that the LC/MS analysis was performed by a method similar to that described above.

As a result, a signal was observed at a m/z of 749 while the mass of the target substance was calculated to be 749, revealing that SFNBBnf(II)(4) was obtained.

### <Measurement of Physical Properties>

Next, the ultraviolet-visible absorption spectra (hereinafter, simply referred to as absorption spectra) and photoluminescence (PL) spectra (hereinafter, simply referred to as “emission spectra”) of a toluene solution and a thin film of SFNBBnf(II)(4) were measured. The absorption and emission spectra of the toluene solution and the thin film of SFNBBnf(II)(4) were measured by the same method as above.

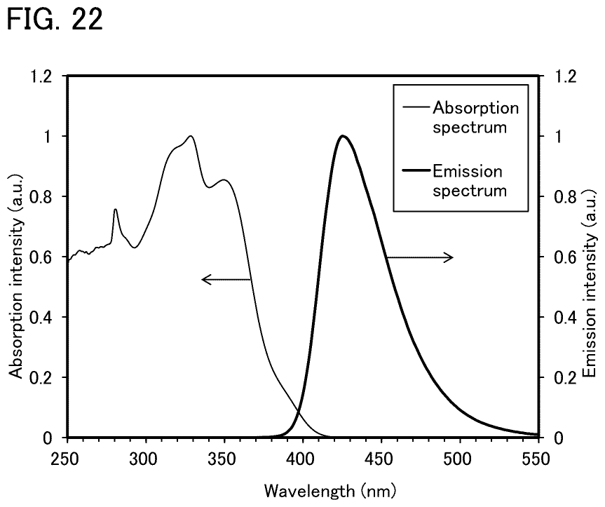

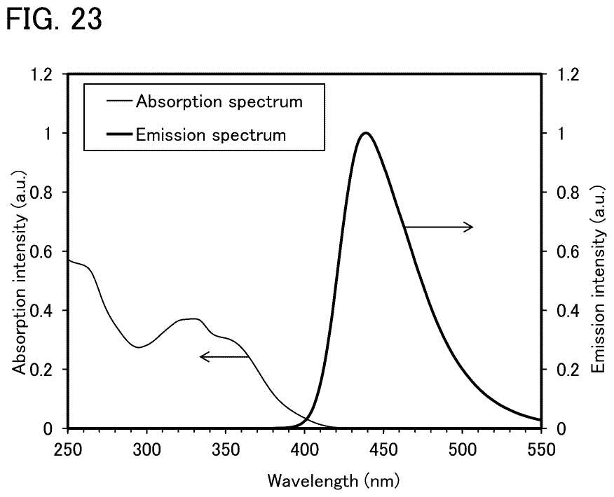

FIG. 22 and FIG. 23 show the measurement results of the toluene solution and the thin film, respectively. According to the measurement results, the toluene solution of SFNBBnf(II)(4) has a shoulder absorption peak at around 393 nm and an absorption peak at around 354 nm, the thin film of SFNBBnf(II)(4) has a shoulder absorption peak at around 386 nm and an absorption peak at around 357 nm, and neither the toluene solution nor the thin film exhibits an absorption band at wavelengths longer than 430 nm. The results suggests that the absorption does not reduce emission efficiency at the wavelength employed for display, showing the suitability of SFNBBnf(II)(4) for a light-emitting device. The measurement results also show that the toluene solution of SFNBBnf(II)(4) exhibits an emission wavelength peak at around 425 nm (excitation wavelength: 350 nm), and the thin film of SFNBBnf(II)(4) exhibits an emission wavelength peak at around 439 nm (excitation wavelength: 358 nm).

The HOMO level and the LUMO level of SFNBBnf(II)(4) were obtained through a cyclic voltammetry (CV) measurement. The calculation method is described below. The CV measurement of SFNBBnf(II)(4) was performed by a method similar to that described in Example 1.

The HOMO level of SFNBBnf(II)(4) was found to be −5.53 eV in the oxidation potential Eₐ [V] measurement. The LUMO level was found to be −2.47 eV in the reduction potential E_{c} [V] measurement. Comparison of the waveforms in the first cycle and the 100th cycle in repeated measurements of the oxidation-reduction wave shows that the peak intensity in the 100th cycle of the Eₐ measurement was maintained at 92% of that in the first cycle and the peak intensity in the 100th cycle of the E_{c} measurement was maintained at 98% of that in the first cycle. These results revealed that SFNBaBnf(10) is highly resistant to repeated oxidation and repeated reduction.

Next, the lowest triplet excitation energy level (T₁ level) of SFNBBnf(II)(4) was calculated through the measurement of an emission spectrum (a phosphorescence spectrum). The calculation method is similar to that described in Example 1.

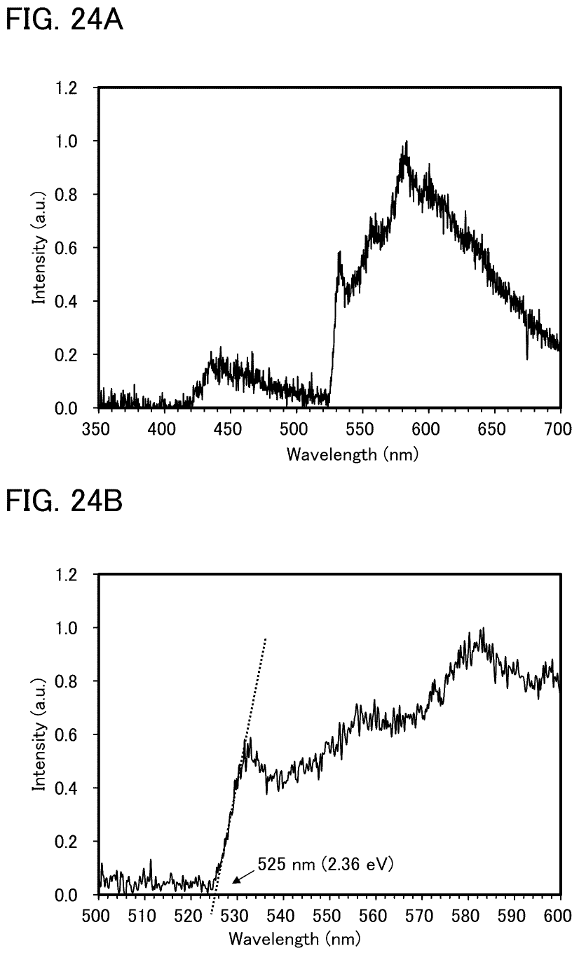

FIG. 24A shows the measurement results of the phosphorescence spectrum (10 K) of SFNBBnf(II)(4). FIG. 24B shows the phosphorescence spectrum (10 K) of SFNBBnf(II)(4) in the wavelength range of 500 nm to 600 nm. According to FIG. 24B, the wavelength of the emission edge on the short wavelength side of the phosphorescence spectrum (10 K) of SFNBBnf(II)(4) is 525 nm, which indicates that the lowest triplet excitation energy level (T₁ level) of SFNBBnf(II)(4) is 2.36 eV.

Differential scanning calorimetry (DSC) measurement of SFNBBnf(II)(4) was performed with DSC8500 manufactured by PerkinElmer, Inc. The DSC measurement was performed in the following manner: the temperature was raised from −10° C. to 350° C. at a temperature rising rate of 40° C./min and held for 3 minutes, and then the temperature was decreased to −10° C. at a temperature decreasing rate of 40° C./min and held for 3 minutes. This operation was performed twice in succession. The DSC measurement result of a second cycle showed that the glass transition point of SFNBBnf(II)(4) was 160° C. The crystallization temperature and the melting point were not observed. This indicates that SFNBaBnf(10) is a substance having high heat resistance and the film of SFNBBnf(II)(4) can maintain a thermally stable quality.

For comparison, differential scanning calorimetry measurement (DSC measurement) of N-phenyl-N-(9,9′-spirobi[9H-fluoren]-2-yl)benzo[b]naphtho[2,3-d]furan-4-amine (abbreviation: SFABnf(II)(4)), which has a structure in which the 1-naphthyl group is eliminated from SFNBBnf(II)(4), was performed under the same conditions. The results reveal that SFABnf(II)(4) has a glass transition point of 137° C., which is lower than that of SFNBBnf(II)(4). Therefore, inclusion of the 1-naphthyl group enables SFNBBnf(II)(4) to have a higher glass transition point.

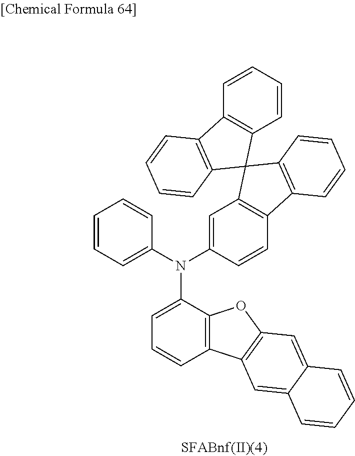 [SMILES: c1ccc(N(c2ccc3c(c2)C2(c4ccccc4-c4ccccc42)c2ccccc2-3)c2cccc3c2oc2cc4ccccc4cc23)cc1]

The thermogravimetry-differential thermal analysis of SFNBBnf(II)(4) was performed in a manner similar to that described in Example 1. In the thermogravimetry-differential thermal analysis, the temperature (decomposition temperature) at which the weight obtained by thermogravimetry was reduced by 5% of the weight at the beginning of the measurement was found to be 397° C., which shows that SFNBBnf(II)(4) is a substance having high heat resistance.

The above results reveal that SFNBBnf(II)(4), which is the organic compound of one embodiment of the present invention, is an organic compound with high electrical stability and high heat resistance and can be suitably used for an organic semiconductor device such as a light-emitting device.

### Example 3

This example describes the fabrication of phosphorescent devices (a light-emitting device 1, a light-emitting device 2, a light-emitting device 5, and a light-emitting device 6) each including an organic compound of one embodiment of the present invention with a high LUMO level and high electron resistance in the second hole-transport layer in contact with the light-emitting layer and phosphorescent devices (a comparative light-emitting device 3, a comparative light-emitting device 4, a comparative light-emitting device 7, and a comparative light-emitting device 8) each including a comparative organic compound, and the measurement results of the device characteristics.

Structural formulae of organic compounds used for the light-emitting devices are shown below.

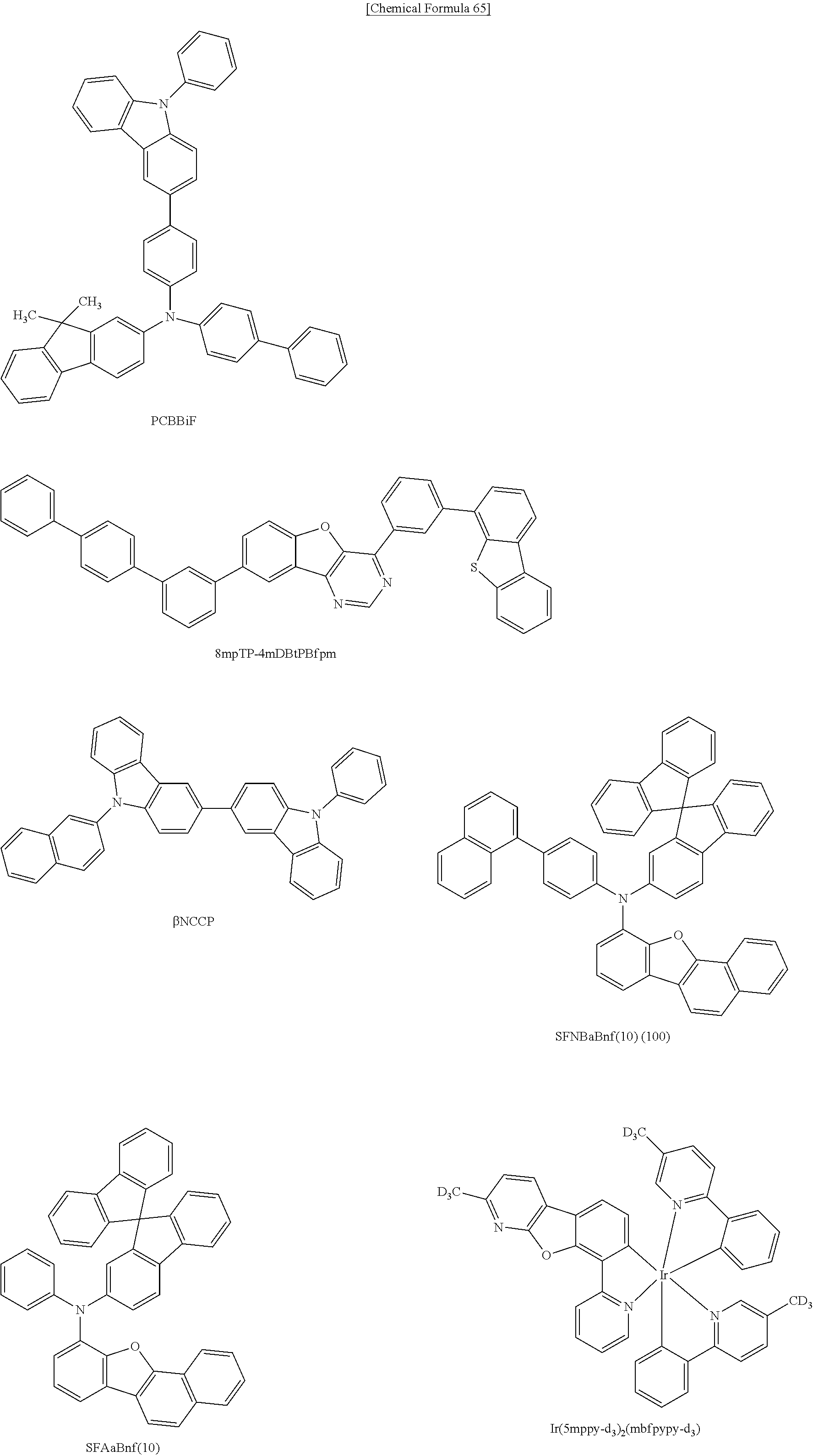

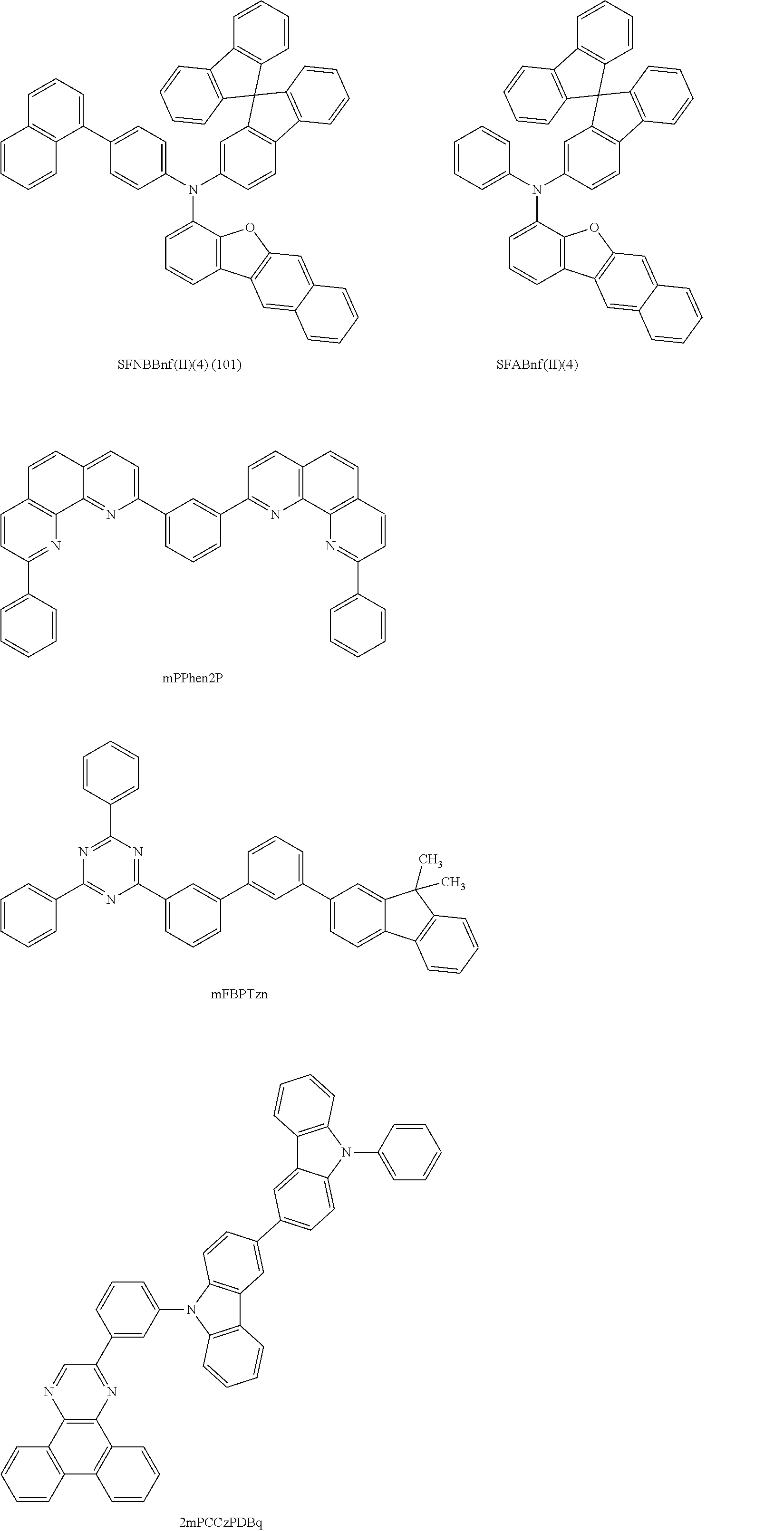 [SMILES: c1ccc2c(c1)-c1ccccc1C21c2ccccc2-c2ccc(N(c3ccc(-c4cccc5ccccc45)cc3)c3cccc4c3oc3cc5ccccc5cc34)cc21 ; c1ccc(N(c2ccc3c(c2)C2(c4ccccc4-c4ccccc42)c2ccccc2-3)c2cccc3c2oc2cc4ccccc4cc23)cc1 ; c1ccc(-c2ccc3ccc4ccc(-c5cccc(-c6ccc7ccc8ccc(-c9ccccc9)nc8c7n6)c5)nc4c3n2)cc1 ; CC1(C)c2ccccc2-c2ccc(-c3cccc(-c4cccc(-c5nc(-c6ccccc6)nc(-c6ccccc6)n5)c4)c3)cc21 ; c1ccc(-n2c3ccccc3c3cc(-c4ccc5c(c4)c4ccccc4n5-c4cccc(-c5cnc6c7ccccc7c7ccccc7c6n5)c4)ccc32)cc1]

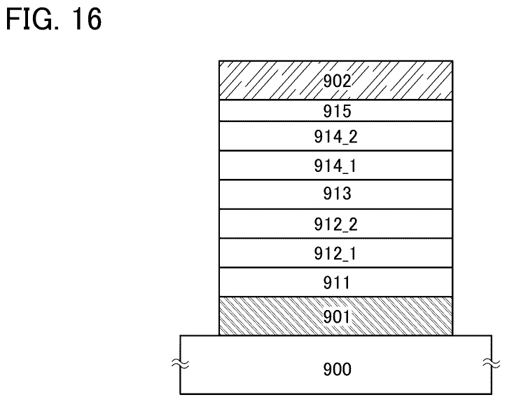

As illustrated in FIG. 16, the light-emitting devices each have a structure in which a hole-injection layer 911, hole-transport layers (a first hole-transport layer 912_1 and a second hole-transport layer 912_2), a light-emitting layer 913, electron-transport layers (a first electron-transport layer 914_1 and a second electron-transport layer 914_2), and an electron-injection layer 915 are stacked in this order over a first electrode 901 formed over a glass substrate 900, and a second electrode 902 is formed over the second electron-injection layer 915.

### <Fabrication Method of Light-Emitting Device 1>

Indium tin oxide containing silicon oxide (ITSO) was deposited by a sputtering method over the glass substrate 900 to a thickness of 70 nm, so that the first electrode 901 as a transparent electrode was formed. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed.

Then, the substrate provided with the first electrode 901 was fixed to a substrate holder provided in the vacuum evaporation apparatus such that the surface on which the first electrode 901 was formed faced downward. Over the first electrode 901, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were co-deposited to a thickness of 10 nm by evaporation at the weight ratio of 1:0.03 (PCBBiF: OCHD-003), whereby the hole-injection layer 911 was formed.

Next, over the hole-injection layer 911, PCBBiF was deposited by evaporation to a thickness of 40 nm to form the first hole-transport layer 912_1, and then SFNBaBnf(10), which is the organic compound of one embodiment of the present invention and whose synthesis method is described in Example 1, was deposited by evaporation to a thickness of 10 nm to form the second hole-transport layer 912_2.

Then, over the second hole-transport layer 912_2, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) as the first host material, 9-(2-naphthyl)-9′-phenyl-3,3′-bi-9H-carbazole (abbreviation: βNCCP) as the second host material, and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) as a phosphorescent substance were deposited by co-evaporation to a thickness of 40 nm at the weight ratio of 0.6:0.4:0.1 (8mpTP-4mDBtPBfpm: βNCCP: Ir(5mppy-d₃)₂(mbfpypy-d₃)), whereby the light-emitting layer 913 was formed.

Next, over the light-emitting layer 913, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm to form the first electron-transport layer 914_1, and then 2,2′-(1,3-phenylene)bis(9-phenyl-1,10-phenanthroline) (abbreviation: mPPhen2P) was deposited by evaporation to a thickness of 20 nm to form the second electron-transport layer 914_2.

Then, over the second electron-transport layer 914_2, lithium fluoride (LiF) was deposited by evaporation to a thickness of 1 nm, whereby the electron-injection layer 915 was formed.

Then, aluminum (Al) was deposited to a thickness of 150 nm over the electron-injection layer 915 by evaporation as the second electrode 902. The above is the fabrication method of the light-emitting device 1.

### <Fabrication Method of Light-Emitting Device 2>

The light-emitting device 2 is different from the light-emitting device 1 in that 2mPCCzPDBq, which was used in the first electron-transport layer 914_1 of the light-emitting device 1, was replaced with 2-[3′-(9,9-dimethyl-9H-fluoren-2-yl)biphenyl-3-yl]-4,6-diphenyl-1,3,5-triazine (abbreviation: mFBPTzn). Other components were fabricated in a manner similar to that for the light-emitting device 1.

### <Fabrication Method of Comparative Light-Emitting Device 3>

The comparative light-emitting device 3 is different from the light-emitting device 1 in that SFNBaBnf(10), the organic compound of one embodiment of the present invention used in the second hole-transport layer 912_2 of the light-emitting device 1, was replaced with N-phenyl-N-(9,9′-spirobi[9H-fluoren]-2-yl)benzo[b]naphtho[2,1-d]furan-10-amine (abbreviation: SFAaBnf(10)), which is a comparative organic compound. Other components were fabricated in a manner similar to that for the light-emitting device 1.

### <Fabrication Method of Comparative Light-Emitting Device 4>

The comparative light-emitting device 4 is different from the comparative light-emitting device 3 in that 2mPCCzPDBq, which was used in the first electron-transport layer 914_1 of the comparative light-emitting device 3, was replaced with mFBPTzn. Other components were fabricated in a manner similar to that for the comparative light-emitting device 3.

### <Fabrication Method of Light-Emitting Device 5>

The light-emitting device 5 is different from the light-emitting device 1 in that SFNBaBnf(10), the organic compound of one embodiment of the present invention used in the second hole-transport layer 912_2 of the light-emitting device 1, was replaced with SFNBBnf(II)(4), which is the organic compound of one embodiment of the present invention and whose synthesis method is described in Example 2. Other components were fabricated in a manner similar to that for the light-emitting device 1.

### <Fabrication Method of Light-Emitting Device 6>

The light-emitting device 6 is different from the light-emitting device 5 in that 2mPCCzPDBq, which was used in the first electron-transport layer 914_1 of the light-emitting device 5, was replaced with mFBPTzn. Other components were fabricated in a manner similar to that for the light-emitting device 5.

### <Fabrication Method of Comparative Light-Emitting Device 7>

The comparative light-emitting device 7 is different from the light-emitting device 5 in that SFNBBnf(II)(4), the organic compound of one embodiment of the present invention used in the second hole-transport layer 912_2 of the light-emitting device 5, was replaced with N-phenyl-N-(9,9′-spirobi[9H-fluoren]-2-yl)benzo[b]naphtho[2,3-d]furan-4-amine (abbreviation: SFABnf(II)(4)), which is a comparative organic compound. Other components were fabricated in a manner similar to that for the light-emitting device 5.

### <Fabrication Method of Comparative Light-Emitting Device 8>

The comparative light-emitting device 8 is different from the comparative light-emitting device 7 in that 2mPCCzPDBq, which was used in the first electron-transport layer 914_1 of the comparative light-emitting device 7, was replaced with mFBPTzn. Other components were fabricated in a manner similar to that for the comparative light-emitting device 7.

Table 1 lists the device structures of the light-emitting devices 1 and 2 and the comparative light-emitting devices 3 and 4. Table 2 lists the device structures of the light-emitting devices 5 and 6 and the comparative light-emitting devices 7 and 8.

TABLE 1

| | | | | | Comparative | Comparative |
|---|---|---|---|---|---|---|
| | | | Light-emitting | Light-emitting | light-emitting | light-emitting |
| | | Thickness | device 1 | device 2 | device 3 | device 4 |
| Second electrode | — | 150 nm | Al | | | |
| Electron-injection | — | 1 nm | LiF | | | |
| layer | | | | | | |
| Electron-transport | 2 | 20 nm | mPPhen2P | | | |
| layer | 1 | 10 nm | 2mPCCzPDBq | mFBPTzn | 2mPCCzPDBq | mFBPTzn |

| Light-emitting | — | 40 nm | 8mpTP-4mDBtPBfpm:βNCCP:Ir(5mppy-d₃)₂(mbfpypy-d₃) | |
|---|---|---|---|---|
| layer | | | (0.6:0.4:0.1) | |
| Hole-transport | 2 | 10 nm | SFNBaBnf(10) | SFAaBnf(10) |

| | | | |
|---|---|---|---|
| layer | 1 | 40 nm | PCBBiF |
| Hole-injection | — | 10 nm | PCBBiF:OCHD-003 (1:0.03) |
| layer | | | |
| First electrode | — | 70 nm | ITSO |

TABLE 2

| | | | | | Comparative | Comparative |
|---|---|---|---|---|---|---|
| | | | Light-emitting | Light-emitting | light-emitting | light-emitting |
| | | Thickness | device 5 | device 6 | device 7 | device 8 |
| Second electrode | — | 150 nm | Al | | | |
| Electron-injection | — | 1 nm | LiF | | | |
| layer | | | | | | |
| Electron-transport | 2 | 20 nm | mPPhen2P | | | |
| layer | 1 | 10 nm | 2mPCCzPDBq | mFBPTzn | 2mPCCzPDBq | mFBPTzn |

| Light-emitting | — | 40 nm | 8mpTP-4mDBtPBfpm:βNCCP:Ir(5mppy-d₃)₂(mbfpypy-d₃) | |
|---|---|---|---|---|
| layer | | | (0.6:0.4:0.1) | |
| Hole-transport | 2 | 10 nm | SFNBBnf(II)(4) | SFABnf(II)(4) |

| | | | |
|---|---|---|---|
| layer | 1 | 40 nm | PCBBiF |
| Hole-injection | — | 10 nm | PCBBiF:OCHD-003 (1:0.03) |
| layer | | | |
| First electrode | — | 70 nm | ITSO |

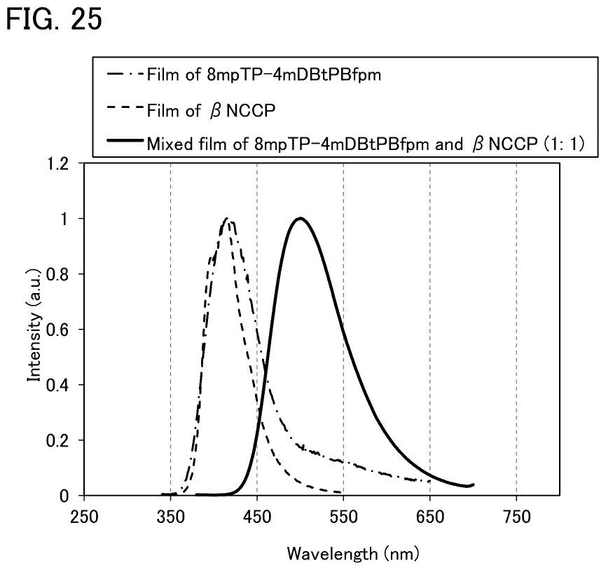

FIG. 25 shows the measurement results of emission spectra (PL spectra) of a thin film of 8mpTP-4mDBtPBfpm, a thin film of βNCCP, and a mixed film that was formed by co-evaporation of 8mpTP-4mDBtPBfpm and βNCCP at a weight ratio of 1:1; the spectra were measured at room temperature. Note that the above films whose emission spectra were measured were each in the form of a 50-nm-thick thin film deposited by evaporation over a quartz substrate. An FP-8600DS fluorescence spectrophotometer (produced by JASCO Corporation) was used for the emission spectra measurements.

As shown in FIG. 25, the peak wavelengths of the emission spectra of the film of 8mpTP-4mDBtPBfpm, the film of βNCCP, and the mixed film of 8mpTP-4mDBtPBfpm and βNCCP are 416 nm, 415 nm, and 500 nm, respectively, revealing that the peak wavelength of the emission spectrum of the mixed film of 8mpTP-4mDBtPBfpm and βNCCP is longer than that of the emission spectrum of each of the film of 8mpTP-4mDBtPBfpm and the film of βNCCP. Thus, it was found that the emission spectrum of the mixed film of 8mpTP-4mDBtPBfpm and βNCCP is different the superimposed spectra of the films of 8mpTP-4mDBtPBfpm and βNCCP, and shifted to the longer wavelength side than each of the emission spectra of the films of 8mpTP-4mDBtPBfpm and βNCCP. The above indicates that 8mpTP-4mDBtPBfpm and βNCCP form, in combination, an exciplex when excited at room temperature, and the observed emission spectrum of the mixed film of 8mpTP-4mDBtPBfpm and βNCCP originates from the exciplex.

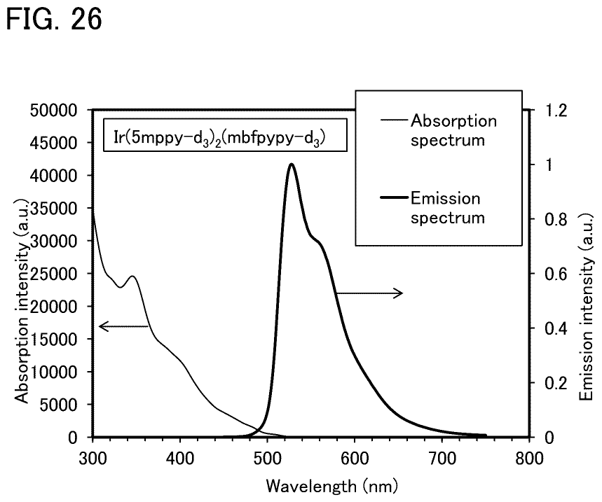

FIG. 26 shows the measurement results of the absorption spectrum and the emission spectrum (PL spectrum) of Ir(5mppy-d₃)₂(mbfpypy-d₃) at room temperature. The absorption spectrum of Ir(5mppy-d₃)₂(mbfpypy-d₃) was measured with an ultraviolet-visible spectrophotometer (V-770DS, produced by JASCO Corporation). The emission spectrum (PL spectrum) was measured with a fluorescence spectrophotometer (FP-8600 produced by JASCO Corporation). The absorption and emission spectra of Ir(5mppy-d₃)₂(mbfpypy-d₃) were measured using a solution with chloroform as a solvent. An emission edge on a shorter wavelength side of each of the emission spectra was determined as the intersection between a tangent and the horizontal axis or the baseline. The tangent was drawn at a point at which the slope on a shorter wavelength side of the shortest-wavelength peak (or the shortest-wavelength shoulder peak) of the emission spectrum has the maximum absolute value. An absorption edge of each of the absorption spectra was determined as the intersection between a tangent and the horizontal axis or the baseline. The tangent was drawn at a point at which the slope on a longer wavelength side of the longest-wavelength peak (or the longest-wavelength shoulder peak) of the absorption spectrum has the maximum absolute value.

As shown in FIG. 26, the maximum peak wavelength of the emission spectrum (PL spectrum) of Ir(5mppy-d₃)₂(mbfpypy-d₃) is 528 nm. As can be seen from FIG. 25, the maximum peak wavelength of the emission spectrum of the mixed film of 8mpTP-4mDBtPBfpm and βNCCP (the PL spectrum of the exciplex) is 500 nm, which indicates that the difference between the peak wavelengths of the emission spectra of the exciplex and the light-emitting substance is less than or equal to 30 nm. This reveals that the light-emitting devices 1, 2, 5, and 6 and the comparative light-emitting devices 3, 4, 7, and 8 each has a structure utilizing ExTET.

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for one hour were performed at the time of sealing). Then, the characteristics of the light-emitting devices were measured.

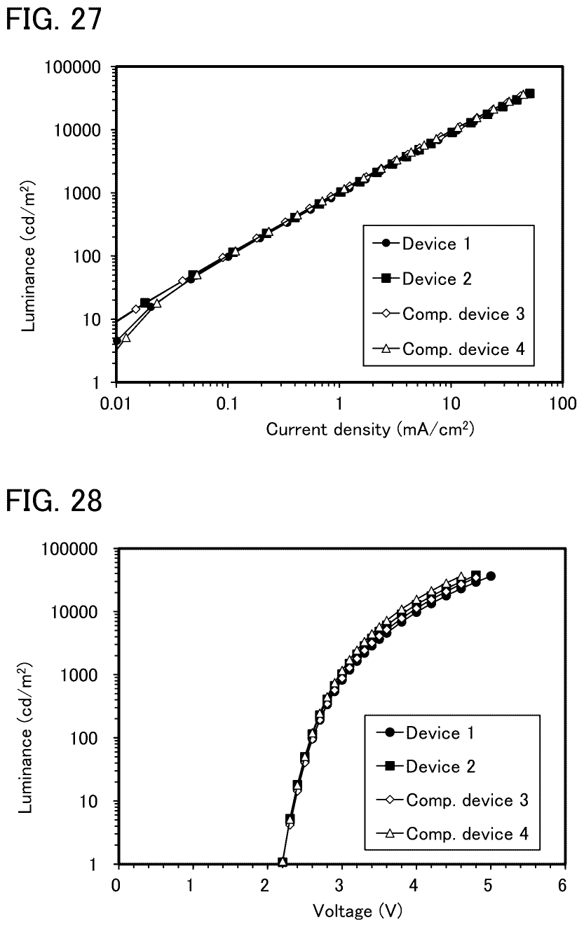

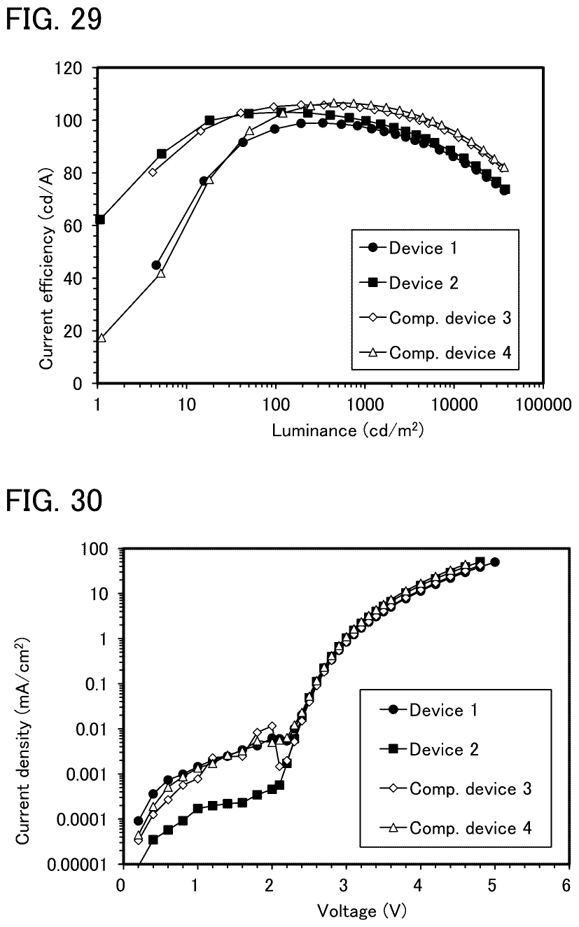

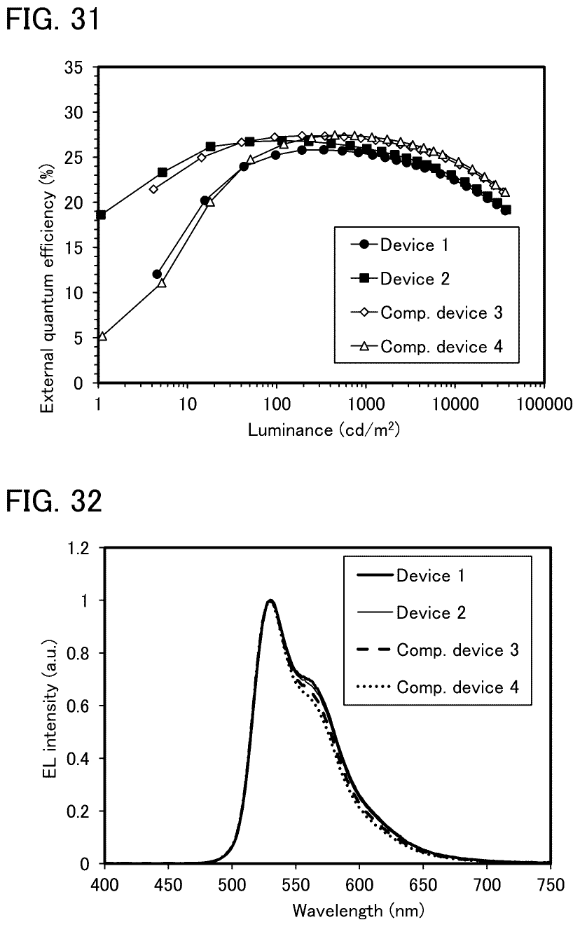

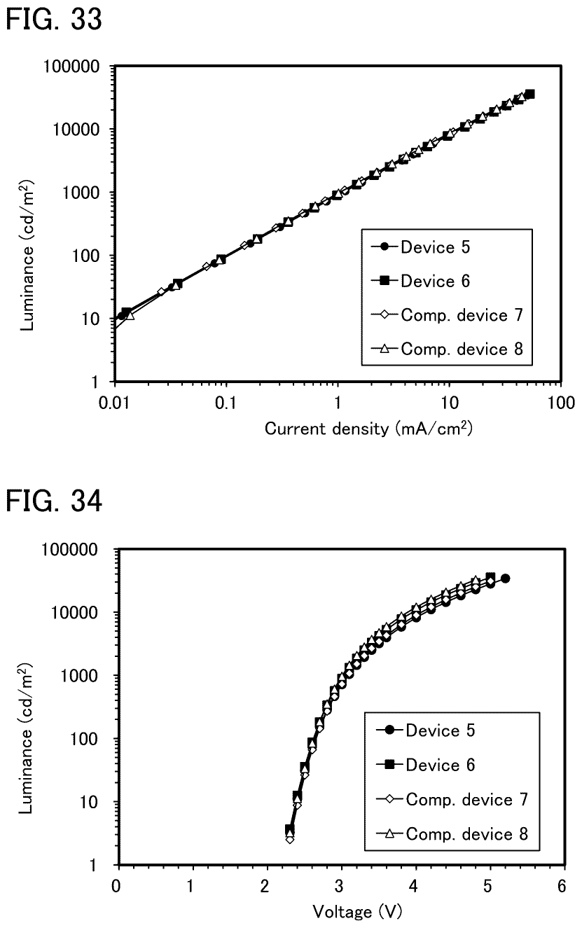

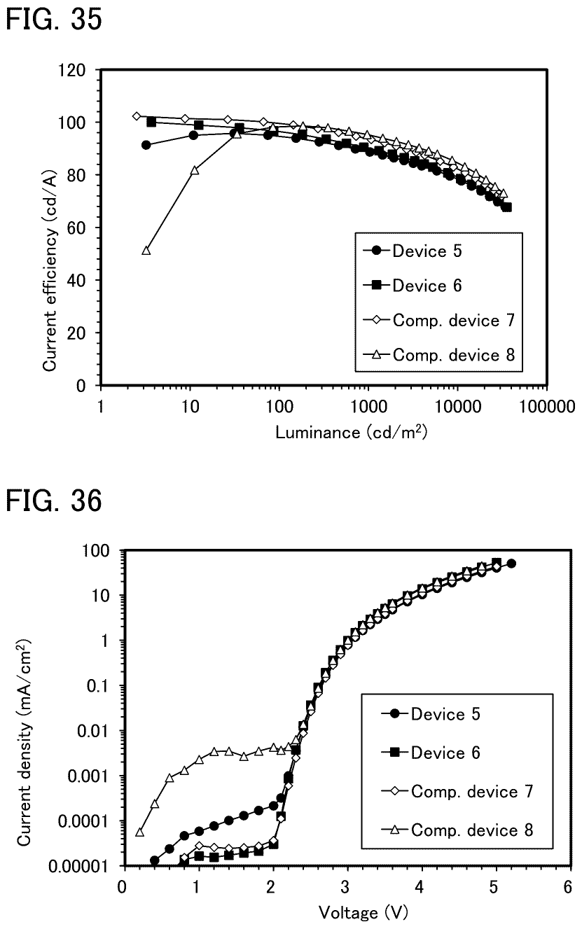

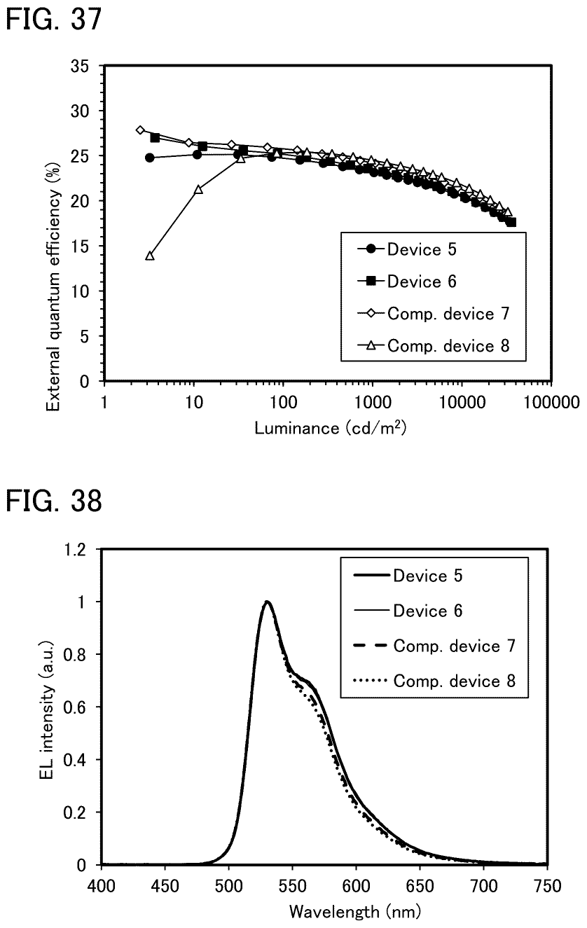

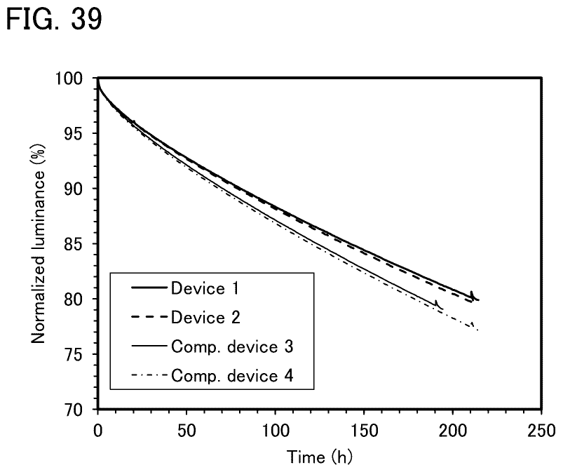

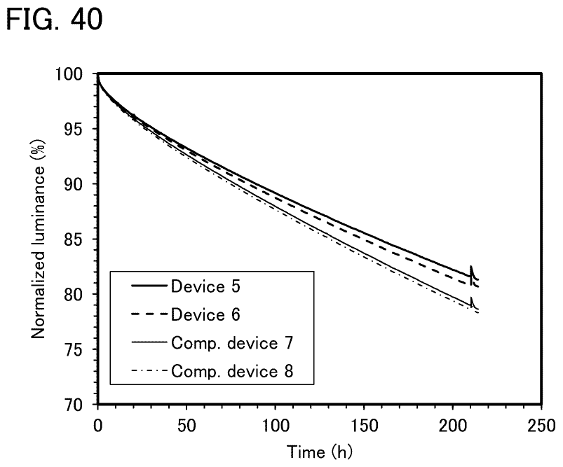

FIG. 27 shows luminance-current density characteristics of the light-emitting devices 1 and 2 and the comparative light-emitting devices 3 and 4. FIG. 28 shows luminance-voltage characteristics thereof. FIG. 29 shows current efficiency-luminance characteristics thereof. FIG. 30 shows current density-voltage characteristics thereof. FIG. 31 shows external quantum efficiency-luminance characteristics thereof. FIG. 32 shows electroluminescence spectra thereof. FIG. 33 shows luminance-current density characteristics of the light-emitting devices 5 and 6 and the comparative light-emitting devices 7 and 8. FIG. 34 shows luminance-voltage characteristics thereof. FIG. 35 shows current efficiency-luminance characteristics thereof. FIG. 36 shows current density-voltage characteristics thereof. FIG. 37 shows external quantum efficiency-luminance characteristics thereof. FIG. 38 shows electroluminescence spectra thereof. FIG. 39 and FIG. 40 each show a luminance change over driving time when each light-emitting device was driven at a constant current of 2 mA (50 mA/cm²). In the legends in FIG. 27 to FIG. 40, the light-emitting device 1, the light-emitting device 2, the light-emitting device 5, and the light-emitting device 6 are denoted by Device 1, Device 2, Device 5, and Device 6, respectively; the comparative light-emitting device 3, the comparative light-emitting device 4, the comparative light-emitting device 7, and the comparative light-emitting device 8 are denoted by Comp. device 3, Comp. device 4, Comp. device 7, and Comp. device 8, respectively.

Table 3 shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured from the front of the emission surface of the substrate with the spectroradiometer, on the assumption that the devices had Lambertian light-distribution characteristics.

TABLE 3

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Light-emitting | 3.00 | 0.0337 | 0.843 | 0.363 | 0.614 | 826 | 97.9 | 25.5 |
| device 1 | | | | | | | | |
| Light-emitting | 3.00 | 0.0412 | 1.03 | 0.360 | 0.617 | 1028 | 99.8 | 25.9 |
| device 2 | | | | | | | | |
| Comparative | 3.00 | 0.0335 | 0.838 | 0.354 | 0.621 | 879 | 105 | 27.1 |
| light-emitting | | | | | | | | |
| device 3 | | | | | | | | |
| Comparative | 3.00 | 0.0441 | 1.10 | 0.348 | 0.626 | 1167 | 106 | 27.2 |
| light-emitting | | | | | | | | |
| device 4 | | | | | | | | |
| Light-emitting | 3.10 | 0.0468 | 1.17 | 0.364 | 0.613 | 1037 | 88.7 | 23.1 |
| device 5 | | | | | | | | |
| Light-emitting | 3.00 | 0.0392 | 0.980 | 0.362 | 0.614 | 887 | 90.5 | 23.6 |
| device 6 | | | | | | | | |
| Comparative | 3.10 | 0.0460 | 1.15 | 0.355 | 0.620 | 1074 | 93.4 | 24.1 |
| light-emitting | | | | | | | | |
| device 7 | | | | | | | | |
| Comparative | 3.00 | 0.0405 | 1.01 | 0.350 | 0.624 | 965 | 95.3 | 24.5 |
| light-emitting | | | | | | | | |
| device 8 | | | | | | | | |

From FIG. 27 to FIG. 38 and Table 3, the light-emitting devices 1, 2, 5, and 6 were found to be light-emitting devices with favorable characteristics that emit green light derived from Ir(5mppy-d₃)₂(mbfpypy-d₃).

FIG. 39 shows that the light-emitting device 1 has a smaller change in luminance over driving time than the comparative light-emitting device 3, and the light-emitting device 2 has a smaller change in luminance over driving time than the comparative light-emitting device 4. This indicates that the light-emitting device using SFNBaBnf(10), which is the organic compound of one embodiment of the present invention, for the second hole-transport layer 912_2 has a smaller change in luminance over driving time, that is, a longer lifetime, than the light-emitting device using SFAaBnf(10) as the comparative organic compound.

FIG. 40 shows that the light-emitting device 5 has a smaller change in luminance over driving time than the comparative light-emitting device 7, and the light-emitting device 6 has a smaller change in luminance over driving time than the comparative light-emitting device 8. This indicates that the light-emitting device using SFNBBnf(II)(4), which is the organic compound of one embodiment of the present invention, for the second hole-transport layer 912_2 has a smaller change in luminance over driving time, that is, a longer lifetime, than the light-emitting device using SFABnf(II)(4) as the comparative organic compound.

Here, SFNBaBnf(10), which is the organic compound of one embodiment of the present invention, has a structure in which a 1-naphthyl group is introduced into the phenyl group in SFAaBnf(10), which is a comparative organic compound. In addition, SFNBBnf(II)(4), which is the organic compound of one embodiment of the present invention, has a structure in which a 1-naphthyl group is introduced into the phenyl group in SFABnf(II)(4), which is a comparative organic compound. These indicate that, in each of the organic compounds of embodiments of the present invention, the introduction of a 1-naphthyl group into the phenyl group stabilizes the excited state of the compound to inhibit deterioration. Accordingly, each of SFNBaBnf(10) and SFNBBnf(II)(4) was found to be an organic compound that can increase the lifetime and reliability of a light-emitting device having a structure utilizing ExTET when used in a layer in contact with a light-emitting layer of the light-emitting device.

### Example 4

This example describes the fabrication of fluorescent devices (a light-emitting device 9 and a light-emitting device 10) each including an organic compound of one embodiment of the present invention with a high LUMO level and high electron resistance in the second hole-transport layer in contact with the light-emitting layer and fluorescent devices (a comparative light-emitting device 11 and a comparative light-emitting device 12) each including a comparative organic compound, and the measurement results of the device characteristics.

Structural formulae of organic compounds used for the light-emitting devices are shown below.

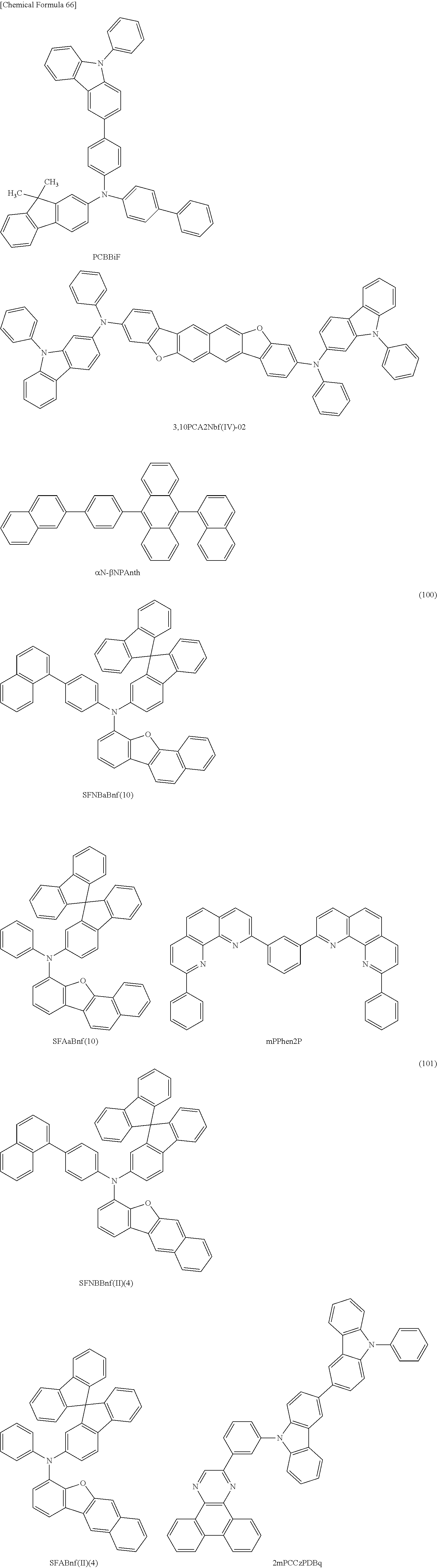

As illustrated in FIG. 16, the light-emitting devices each have a structure in which a hole-injection layer 911, hole-transport layers (a first hole-transport layer 912_1 and a second hole-transport layer 912_2), a light-emitting layer 913, electron-transport layers (a first electron-transport layer 914_1 and a second electron-transport layer 914_2), and an electron-injection layer 915 are stacked in this order over a first electrode 901 formed over a glass substrate 900, and a second electrode 902 is formed over the second electron-injection layer 915.

### <Fabrication Method of Light-Emitting Device 9>

Indium tin oxide containing silicon oxide (ITSO) was deposited by a sputtering method over the glass substrate 900 to a thickness of 110 nm, so that the first electrode 901 as a transparent electrode was formed. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed.

Then, the substrate provided with the first electrode 901 was fixed to a substrate holder provided in the vacuum evaporation apparatus so that the surface on which the first electrode 901 was formed faced downward. Over the first electrode 901, PCBBiF and OCHD-003 were deposited by co-evaporation to a thickness of 10 nm at the weight ratio of 1:0.03 (PCBBiF: OCHD-003), whereby the hole-injection layer 911 was formed.

Next, over the hole-injection layer 911, PCBBiF was deposited by evaporation to a thickness of 90 nm to form the first hole-transport layer 912_1, and then SFNBaBnf(10), which is the organic compound of one embodiment of the present invention and whose synthesis method is described in Example 1, was deposited by evaporation to a thickness of 10 nm to form the second hole-transport layer 912_2.

Next, over the second hole-transport layer 912_2, 9-(1-naphthyl)-10-[4-(2-naphthyl)phenyl]anthracene (abbreviation: αN-βNPAnth) as a host material and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) as a fluorescent substance were deposited by co-evaporation to a thickness of 25 nm at the weight ratio of 1:0.015 (αN-βNPAnth: 3,10PCA2Nbf(IV)-02), whereby the light-emitting layer 913 was formed.

Next, over the light-emitting layer 913, 2mPCCzPDBq was deposited by evaporation to a thickness of 10 nm to form the first electron-transport layer 914_1, and then mPPhen2P was deposited by evaporation to a thickness of 15 nm to form the second electron-transport layer 914_2.

Then, over the second electron-transport layer 914_2, lithium fluoride (LiF) was deposited by evaporation to a thickness of 1 nm, whereby the electron-injection layer 915 was formed.

Then, aluminum (Al) was deposited to a thickness of 150 nm over the electron-injection layer 915 by evaporation as the second electrode 902. The above is the fabrication method of the light-emitting device 9.

### <Fabrication Method of Light-Emitting Device 10>

The light-emitting device 10 is different from the light-emitting device 9 in that SFNBaBnf(10), the organic compound of one embodiment of the present invention used in the second hole-transport layer 912_2 of the light-emitting device 9, was replaced with SFNBBnf(II)(4), which is the organic compound of one embodiment of the present invention and whose synthesis method is described in Example 2. Other components were fabricated in a manner similar to that for the light-emitting device 9.

### <Fabrication Method of Comparative Light-Emitting Device 11>

The light-emitting device 11 is different from the light-emitting device 9 in that SFNBaBnf(10), the organic compound of one embodiment of the present invention used in the second hole-transport layer 912_2 of the light-emitting device 9, was replaced with SFAaBnf(10), which is a comparative organic compound. Other components were fabricated in a manner similar to that for the light-emitting device 9.

### <Fabrication Method of Comparative Light-Emitting Device 12>

The light-emitting device 12 is different from the light-emitting device 10 in that SFNBBnf(II)(4), the organic compound of one embodiment of the present invention used in the second hole-transport layer 912_2 of the light-emitting device 10, was replaced with SFABnf(II)(4), which is a comparative organic compound. Other components were fabricated in a manner similar to that for the light-emitting device 10.

Table 4 lists the device structures of the light-emitting devices 9 and 10 and the comparative light-emitting devices 11 and 12.

TABLE 4

| | | | | | Comparative | Comparative |
|---|---|---|---|---|---|---|
| | | | Light-emitting | Light-emitting | light-emitting | light-emitting |
| | | Thickness | device 9 | device 10 | device 11 | device 12 |
| Second electrode | — | 150 nm | Al | | | |
| Electron-injection | — | 1 nm | LiF | | | |
| layer | | | | | | |
| Electron-transport | 2 | 15 nm | mPPhen2P | | | |
| layer | 1 | 10 nm | 2mPCCzPDBq | | | |
| Light-emitting | — | 25 nm | αN-βNPAnth:3,10PCA2Nbf(IV)-02 (1:0.015) | | | |
| layer | | | | | | |
| Hole-transport | 2 | 10 nm | SFNBaBnf(10) | SFNBBnf(II)(4) | SFAaBnf(10) | SFABnf(II)(4) |

| | | | |
|---|---|---|---|
| layer | 1 | 90 nm | PCBBiF |
| Hole-injection | — | 10 nm | PCBBiF:OCHD-003 (1:0.03) |
| layer | | | |
| First electrode | — | 110 nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for one hour were performed at the time of sealing). Then, the characteristics of the light-emitting devices were measured.

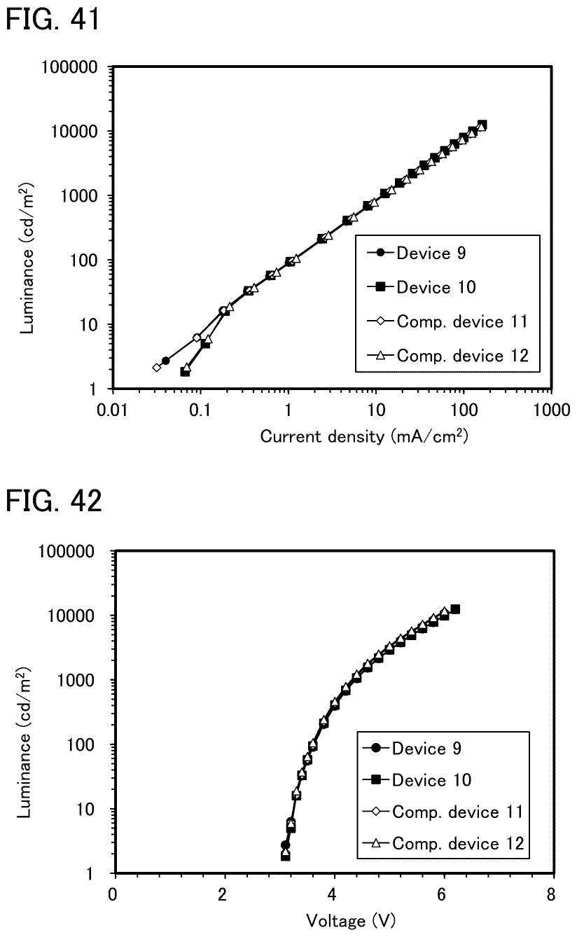

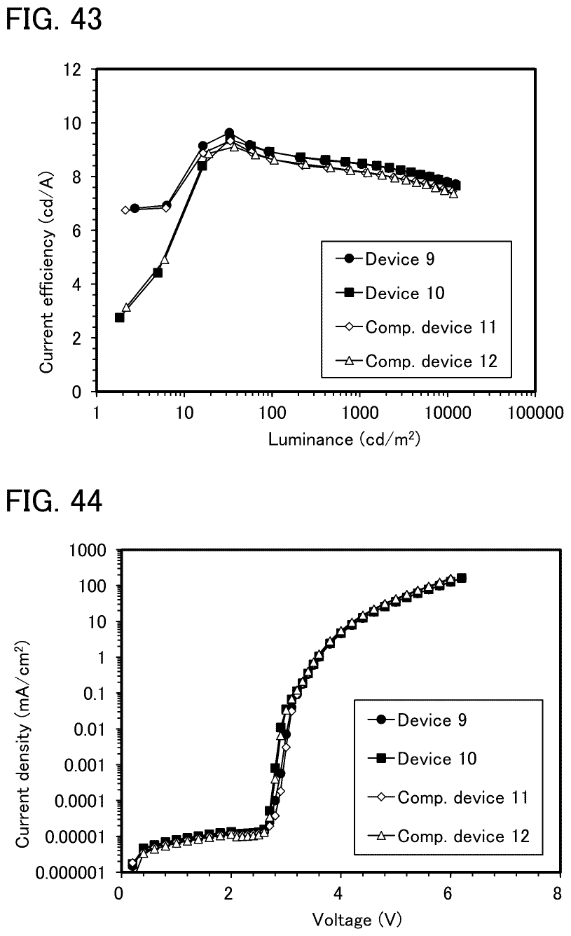

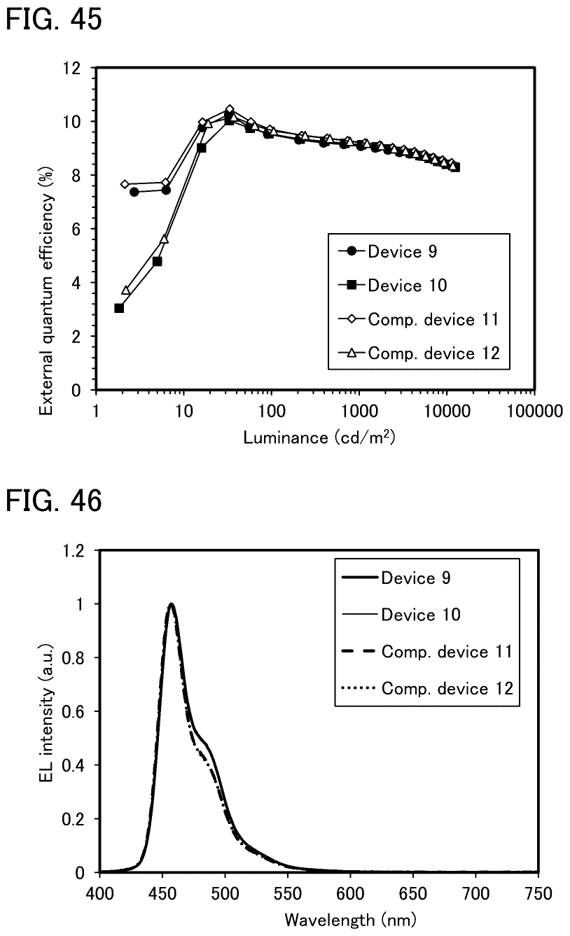

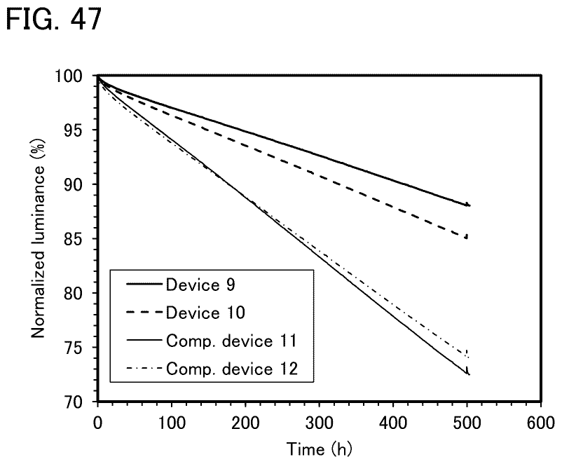

FIG. 41 shows luminance-current density characteristics of the light-emitting devices 9 and 10 and the comparative light-emitting devices 11 and 12. FIG. 42 shows luminance-voltage characteristics thereof. FIG. 43 shows current efficiency-luminance characteristics thereof. FIG. 44 shows current density-voltage characteristics thereof. FIG. 45 shows external quantum efficiency-luminance characteristics thereof. FIG. 46 shows electroluminescence spectra thereof. FIG. 47 shows a luminance change over driving time when each light-emitting device was driven at a constant current of 2 mA (50 mA/cm²). Note that in the legends in FIG. 41 to FIG. 47, the light-emitting device 9 and the light-emitting device 10 are denoted by Device 9 and Device 10, respectively; the comparative light-emitting device 11 and the comparative light-emitting device 12 are denoted by Comp. device 11 and Comp. device 12, respectively.

Table 5 shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured from the front of the emission surface of the substrate with the spectroradiometer, on the assumption that the devices had Lambertian light-distribution characteristics.

TABLE 5

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Light-emitting | 4.40 | 0.494 | 12.4 | 0.136 | 0.108 | 1045 | 8.46 | 9.07 |
| device 9 | | | | | | | | |
| Light-emitting | 4.40 | 0.504 | 12.6 | 0.136 | 0.107 | 1070 | 8.49 | 9.13 |
| device 10 | | | | | | | | |
| Comparative | 4.40 | 0.571 | 14.3 | 0.138 | 0.099 | 1162 | 8.14 | 9.20 |
| light-emitting | | | | | | | | |
| device 11 | | | | | | | | |
| Comparative | 4.20 | 0.382 | 9.54 | 0.138 | 0.100 | 786 | 8.23 | 9.25 |
| light-emitting | | | | | | | | |
| device 12 | | | | | | | | |

From FIG. 41 to FIG. 46 and Table 5, the light-emitting devices 9 and 10 were found to be light-emitting devices with favorable characteristics that emit blue light derived from 3,10PCA2Nbf(IV)-02.

FIG. 47 shows that the light-emitting device 9 has a smaller change in luminance over driving time than the comparative light-emitting device 11, and the light-emitting device 10 has a smaller change in luminance over driving time than the comparative light-emitting device 12. This indicates that the light-emitting device using SFNBaBnf(10) or SFNBBnf(II)(4), which is the organic compound of one embodiment of the present invention, for the second hole-transport layer 912_2 has a smaller change in luminance over driving time, that is, a longer lifetime, than the light-emitting device using SFAaBnf(10) or SFABnf(II)(4) as the comparative organic compound.

Here, SFNBaBnf(10), which is the organic compound of one embodiment of the present invention, has a structure in which a 1-naphthyl group is introduced into the phenyl group in SFAaBnf(10), which is a comparative organic compound. In addition, SFNBBnf(II)(4), which is the organic compound of one embodiment of the present invention, has a structure in which a 1-naphthyl group is introduced into the phenyl group in SFABnf(II)(4), which is a comparative organic compound. These indicate that, in each of the organic compounds of embodiments of the present invention, the introduction of a 1-naphthyl group into the phenyl group stabilizes the excited state of the compound to inhibit deterioration. These show that, when each of SFNBaBnf(10) and SFNBBnf(II)(4) is used in a layer in contact with a light-emitting layer including a fluorescent substance, these organic compounds inhibit deterioration of the other layers due to electrons passing from the light-emitting layer to the anode side, thereby increasing the lifetime and reliability of the light-emitting device.

### Example 5

### Synthesis Example 3

Described in this synthesis example is a method for synthesizing the organic compound of the present invention represented by Structural Formula (144) in Embodiment 1, N-[4-(1-naphthyl)phenyl]-N-(9,9′-diphenyl-9H-fluoren-2-yl)benzo[b]naphtho[2,3-d]furan-4-amine (abbreviation: FLP(2)NBBnf(II)(4)). The structure of FLP(2)NBBnf(II)(4) is shown below.

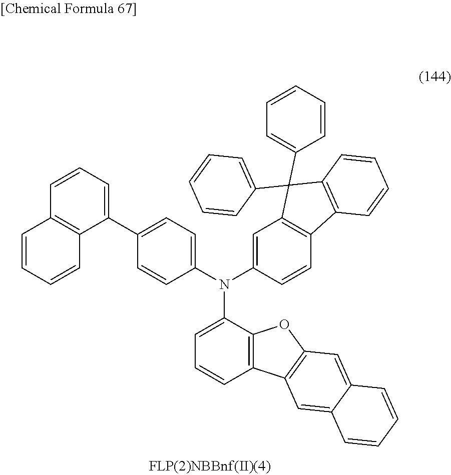 [SMILES: c1ccc(C2(c3ccccc3)c3ccccc3-c3ccc(N(c4ccc(-c5cccc6ccccc56)cc4)c4cccc5c4oc4cc6ccccc6cc45)cc32)cc1]

Into a 200 mL three-neck flask were put 5.1 g (9.4 mmol) of N-(4-(1-naphthyl)phenyl)-N-(9,9′-diphenyl-9H-fluoren-2-yl)amine, 2.4 g (9.4 mmol) of 4-chlorobenzo[b]naphtho[2,3-d]furan, and 86 mg (0.21 mmol) of 2-dicyclohexylphosphino-2′,6′-dimethoxybiphenyl (commonly known name: SPhos). After the air in the flask was replaced with nitrogen, 2.7 g (28 mmol) of sodium tert-butoxide (abbreviation: ^{t}BuONa) and 47 mL of toluene were added thereto. After that, the mixture was heated at 60° C. To this reaction solution was added 58 mg (0.10 mmol) of bis(dibenzylideneacetone)palladium(0) (commonly known name: Pd(dba)₂) was added, and stirring was performed at 120° C. for 7 hours. Toluene was added to this mixture and the obtained mixture was subjected to suction filtration through alumina, Celite, and Florisil. The resulting filtrate was concentrated to give 7.3 g of a yellow solid containing the target substance. This solid was purified by high performance liquid chromatography (mobile phase: chloroform) and then dissolved in toluene, ethanol was added to this solution, and the precipitated solid was collected by suction filtration to give 2.9 g of a target pale yellow solid in a yield of 41%. Synthesis Scheme (s-3) is shown below.

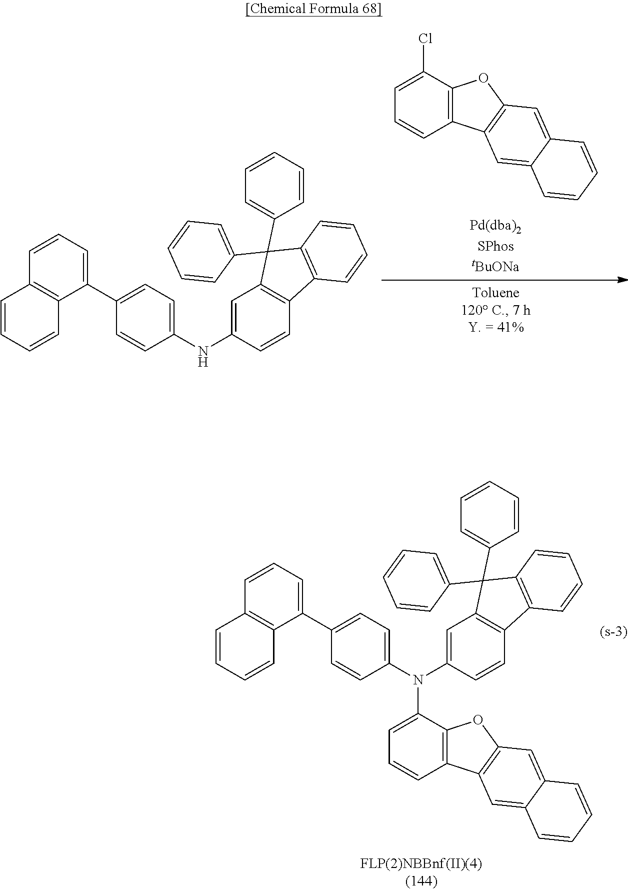 [SMILES: c1ccc(C2(c3ccccc3)c3ccccc3-c3ccc(Nc4ccc(-c5cccc6ccccc56)cc4)cc32)cc1 ; Clc1cccc2c1oc1cc3ccccc3cc12 ; c1ccc(C2(c3ccccc3)c3ccccc3-c3ccc(N(c4ccc(-c5cccc6ccccc56)cc4)c4cccc5c4oc4cc6ccccc6cc45)cc32)cc1]

By a train sublimation method, 1.9 g of the obtained pale yellow solid was purified. In the purification by sublimation, the solid was heated at 300° C. under a pressure of 2.91 Pa for 23 hours. After the sublimation purification, 1.1 g of a target pale yellow solid was obtained at a collection rate of 75%.

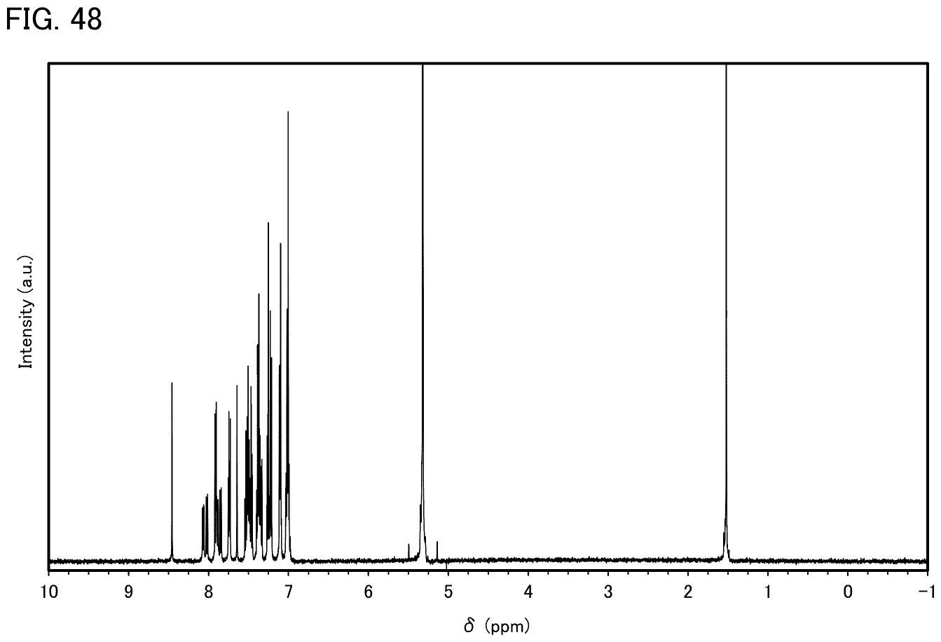

The results of ¹H NMR measurement of the obtained pale yellow solid are given below. FIG. 48 shows a ¹H NMR spectrum. This shows that FLP(2)NBBnf(II)(4) was obtained in this synthesis example.

¹H NMR (dichloromethane-d₂, 500 MHz): δ=8.46 (s, 1H), 8.07 (dd, J=7.5 Hz, 2.0 Hz, 1H), 8.02 (d, J=9.0 Hz, 1H), 7.92-7.88 (m, 3H), 7.85 (d, J=8.5 Hz, 1H), 7.75-7.73 (m, 2H), 7.65 (s, 1H), 7.55-7.45 (m, 6H), 7.40-7.33 (m, 6H), 7.27-7.21 (m, 5H), 7.12-7.09 (m, 4H), 7.04-6.98 (m, 6H).

The molecular weight of the obtained pale yellow solid was measured by LC/MS analysis. As a result, a signal was observed at a mass-to-charge ratio (m/z) of 752 (corresponding to the m/z of a proton adduct of FLP(2)NBBnf(II)(4)) while the mass of the target substance was calculated to be 751, revealing that FLP(2)NBBnf(II)(4) was obtained.

### <Measurement of Physical Properties>

Next, the ultraviolet-visible absorption spectra (hereinafter, simply referred to as absorption spectra) and photoluminescence (PL) spectra (hereinafter, simply referred to as “emission spectra”) of a toluene solution and a thin film of FLP(2)NBBnf(II)(4), which was obtained, were measured.

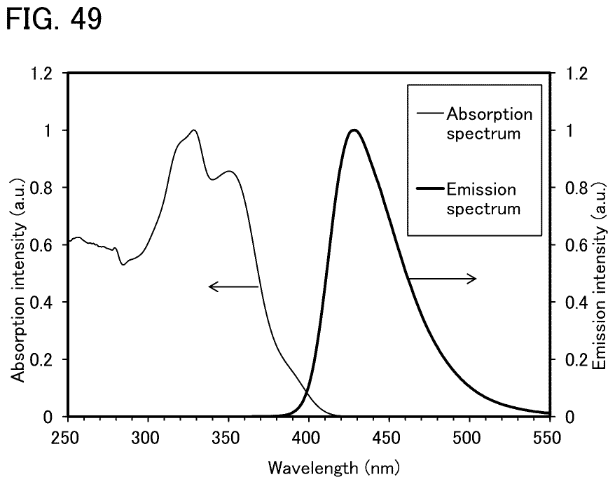

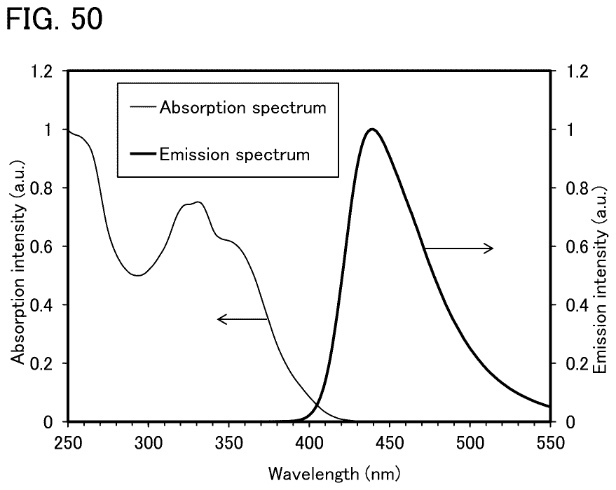

The absorption spectrum of the solution was measured with an ultraviolet-visible spectrophotometer (V-770DS, JASCO Corporation), and the absorption spectrum of the thin film was measured with an ultraviolet-visible spectrophotometer (U-4100, manufactured by Hitachi, Ltd.). To calculate the absorption spectrum of the toluene solution of FLP(2)NBBnf(II)(4), the absorption spectrum of toluene put in a quartz cell was measured and then subtracted from the absorption spectrum of the toluene solution of FLP(2)NBBnf(II)(4) put in a quartz cell. The emission spectrum was measured with a fluorescence spectrophotometer (FP-8600DS, JASCO Corporation). FIG. 49 shows the measurement results of the absorption spectrum and the emission spectrum of the toluene solution of FLP(2)NBBnf(II)(4), and FIG. 50 shows the measurement results of the absorption spectrum and the emission spectrum of the thin film of FLP(2)NBBnf(II)(4).

As shown in FIG. 49, the absorption spectrum of the toluene solution of FLP(2)NBBnf(II)(4) exhibited an absorption peak at around 350 nm. The results reveal that the solution of FLP(2)NBBnf(II)(4) shows no absorption at wavelengths greater than or equal to 440 nm and that the material of the present invention can be suitably used for a light-emitting device. As shown in FIG. 49, the emission spectrum of the toluene solution of FLP(2)NBBnf(II)(4) exhibited an emission peak at around 428 nm (excitation wavelength: 350 nm).

As shown in FIG. 50, the absorption spectrum of the thin film of FLP(2)NBBnf(II)(4) exhibited the maximum absorption peak at around 331 nm and a shoulder peak at around 360 nm. The results reveal that the thin film of FLP(2)NBBnf(II)(4) also shows no absorption at wavelengths greater than or equal to 440 nm and that the material of the present invention can be suitably used for a light-emitting device. As shown in FIG. 50, the emission spectrum of the thin film of FLP(2)NBBnf(II)(4) exhibited an emission peak at around 439 nm (excitation wavelength: 360 nm).

The thermogravimetry-differential thermal analysis (TG-DTA) of FLP(2)NBBnf(II)(4) was performed. For the measurement, a high-sensitivity differential type differential thermogravimeter (STA 2500 Regulus, NETZSCH Japan K. K.) was used. The measurement was performed under first conditions and second conditions. Under the first conditions, the measurement was performed at a temperature rising rate of 10° C./min under atmospheric pressure and a nitrogen stream (flow rate: 200 mL/min). Under the second conditions, the measurement was performed at a temperature rising rate of 10° C./min under 10 Pa.

The thermogravimetry-differential thermal analysis performed under the first measurement conditions reveals that the temperature at which the weight of FLP(2)NBBnf(II)(4) obtained by thermogravimetry decreases by 5% of the weight at the start of the measurement (i.e., the sublimation or decomposition temperature of FLP(2)NBBnf(II)(4)) is 455° C. under atmospheric pressure. The results show that the sublimation or decomposition temperature of FLP(2)NBBnf(II)(4) under atmospheric pressure is 455° C., which indicates high heat resistance.

The thermogravimetry-differential thermal analysis performed under the second measurement conditions reveals that the temperature at which the weight of FLP(2)NBBnf(II)(4) obtained by thermogravimetry decreases by 5% of the weight at the start of the measurement (i.e., the sublimation or decomposition temperature of FLP(2)NBBnf(II)(4)) is 247° C. under 10 Pa. The results show that the sublimation temperature of FLP(2)NBBnf(II)(4) at 10 Pa is 247° C.

The above results show that the sublimation temperature (247° C.) of FLP(2)NBBnf(II)(4) under 10 Pa is lower than the sublimation or decomposition temperature (455° C.) thereof under atmospheric pressure by 208° C. This indicates that FLP(2)NBBnf(II)(4) can be deposited by evaporation at a temperature sufficiently lower than the decomposition temperature under atmospheric pressure. It is thus suggested that the organic compound of one embodiment of the present invention is a material that is less likely to be decomposed during deposition by evaporation and can be formed into a high-purity film by being deposited by evaporation.

Differential scanning calorimetry (DSC) measurement of FLP(2)NBBnf(II)(4) was performed with DSC8500 manufactured by PerkinElmer, Inc. The DSC measurement was performed in the following manner. The temperature was raised from −10° C. to 400° C. at a temperature rising rate of 40° C./min and held for three minutes; then, the temperature was lowered to −10° C. at a temperature falling rate of 100° C./min and held for three minutes. This operation was performed twice in succession. Subsequently, the temperature was raised from −10° C. to 400° C. at a temperature rising rate of 50° C./min and held for three minutes; then, the temperature was lowered to −10° C. at a temperature falling rate of 100° C./min. This operation was performed once.

According to the results of the DSC measurement in the second temperature raising process, the glass transition point of FLP(2)NBBnf(II)(4) is 151° C., and the melting point and the crystallization temperature are not observed. It was found that an organic semiconductor device such as a light-emitting device can have increased heat resistance by including the organic compound of one embodiment of the present invention. Since the melting point and the crystallization temperature were not observed, it was suggested that FLP(2)NBBnf(II)(4) of the present invention can be formed into a thin film having high heat resistance and stable quality and can be suitably used for an organic semiconductor device.

The HOMO level and the LUMO level of FLP(2)NBBnf(II)(4) were calculated through CV measurement. The CV measurement was performed in a manner similar to that described in Example 1 except that dehydrated dimethylformamide (DMF) (manufactured by Sigma-Aldrich Co., Ltd., 99.8%, catalog No. 22705-6) was used as a solvent.

In the measurement of the oxidation potential Eₐ [V] of FLP(2)NBBnf(II)(4), the HOMO level was found to be −5.53 eV. By contrast, the LUMO level was found to be −2.47 eV in the measurement of the reduction potential E_{c} [V]. When the oxidation-reduction wave was repeatedly measured, in the Eₐ measurement, the peak intensity of the oxidation-reduction wave after the 100th cycle was maintained to be 93% of that of the oxidation-reduction wave at the first cycle, and in the E_{c} measurement, the peak intensity of the oxidation-reduction wave after the 100th cycle was maintained to be 99% of that of the oxidation-reduction wave at the first cycle; thus, the resistance of FLP(2)NBBnf(II)(4) to repetitive oxidation and repetitive reduction was found to be extremely high.

Next, the lowest triplet excitation energy level (T₁ level) of FLP(2)NBBnf(II)(4) was calculated through the measurement of an emission spectrum (a phosphorescence spectrum). The calculation method is similar to that described in Example 1.

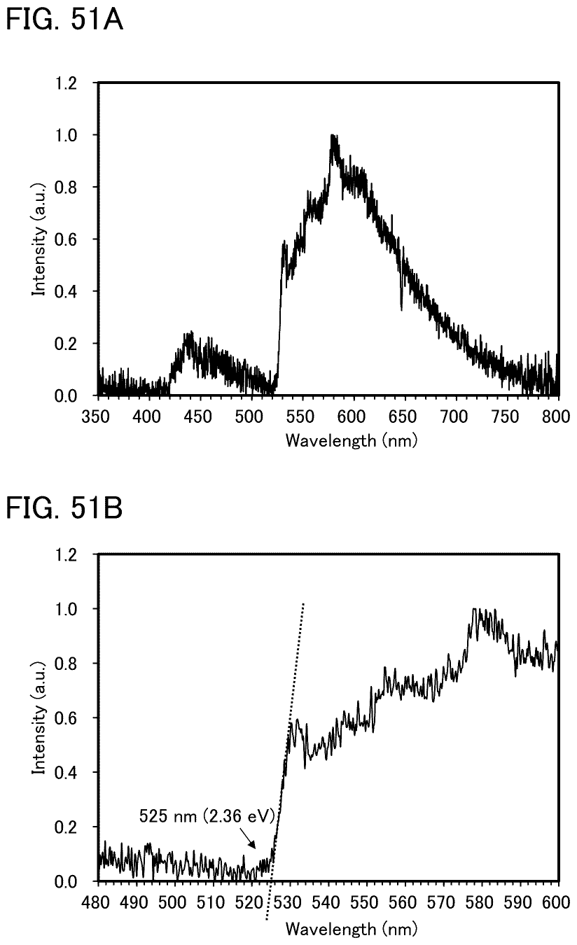

FIG. 51A shows the measurement results of the phosphorescence spectrum (10 K) of FLP(2)NBBnf(II)(4). FIG. 51B shows the phosphorescence spectrum (10 K) of FLP(2)NBBnf(II)(4) in the wavelength range of 480 nm to 600 nm. According to FIG. 51B, the wavelength of the emission edge on the short wavelength side of the phosphorescence spectrum (10 K) of FLP(2)NBBnf(II)(4) is 525 nm, which indicates that the lowest triplet excitation energy level (T₁ level) of FLP(2)NBBnf(II)(4) is 2.36 eV.

### Example 6

This example describes the fabrication of a light-emitting device 13 that is a fluorescent device including an organic compound of one embodiment of the present invention with a high LUMO level and high electron resistance in the second hole-transport layer in contact with the light-emitting layer and a comparative device 14 that is a fluorescent device including a comparative organic compound, and the measurement results of the device characteristics.

Structural formulae of organic compounds used for the light-emitting devices are shown below.

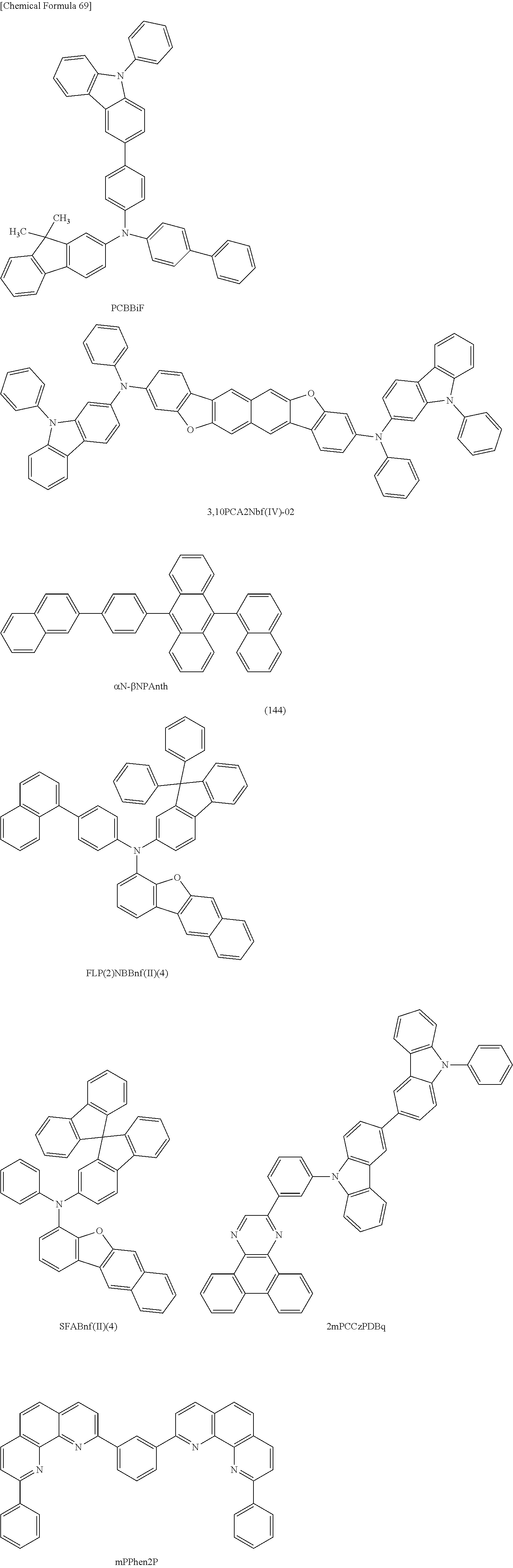

As illustrated in FIG. 16, the light-emitting devices each have a structure in which a hole-injection layer 911, hole-transport layers (a first hole-transport layer 912_1 and a second hole-transport layer 912_2), a light-emitting layer 913, electron-transport layers (a first electron-transport layer 914_1 and a second electron-transport layer 914_2), and an electron-injection layer 915 are stacked in this order over a first electrode 901 formed over a glass substrate 900, and a second electrode 902 is formed over the second electron-injection layer 915.

### <Fabrication Method of Light-Emitting Device 13>

The light-emitting device 13 is different from the light-emitting device 9 in that SFNBaBnf(10), the organic compound of one embodiment of the present invention used in the second hole-transport layer 912_2 of the light-emitting device 9 described in Example 4, was replaced with FLP(2)NBBnf(II)(4), which is the organic compound of one embodiment of the present invention and whose synthesis method is described in Example 5. Other components were fabricated in a manner similar to that for the light-emitting device 9.

### <Fabrication Method of Comparative Light-Emitting Device 14>

The fabrication method of the comparative light-emitting device 14 is similar to that of the comparative light-emitting device 12. That is, SFABnf(II)(4) as the comparative organic compound was used in the second hole-transport layer 912_2.

Table 6 lists the device structures of the light-emitting device 13 and the comparative light-emitting device 14.

TABLE 6

| | | | | Comparative |
|---|---|---|---|---|
| | | | Light- | light- |
| | | | emitting | emitting |
| | | Thickness | device 13 | device 14 |
| Second electrode | — | 150 nm | Al | |
| Electron- | — | 1 nm | LiF | |
| injection layer | | | | |
| Electron- | 2 | 15 nm | mPPhen2P | |
| transport layer | | | | |
| | 1 | 10 nm | 2mPCCzPDBq | |
| Light- | — | 25 nm | αN-βNP Anth:3,10PCA2Nbf(IV)-02 | |
| emitting layer | | | (1:0.015) | |
| Hole- | 2 | 10 nm | FLP(2)NBBnf(II)(4) | SFABnf(II)(4) |
| transport layer | | | | |

| | | | |
|---|---|---|---|
| | 1 | 90 nm | PCBBiF |
| Hole- | — | 10 nm | PCBBiF:OCHD-003 |
| injection layer | | | (1:0.03) |
| First electrode | — | 110 nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for one hour were performed at the time of sealing). Then, the characteristics of the light-emitting devices were measured.

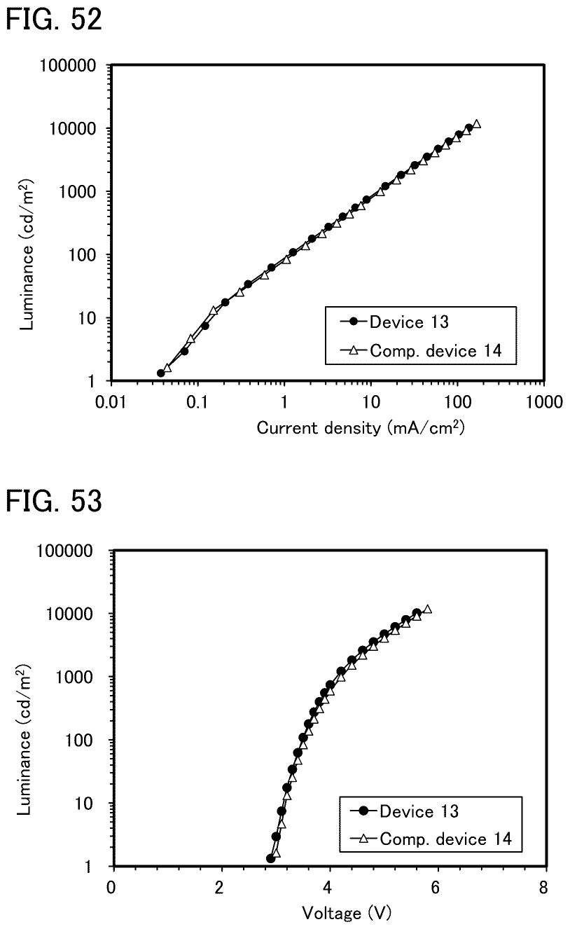

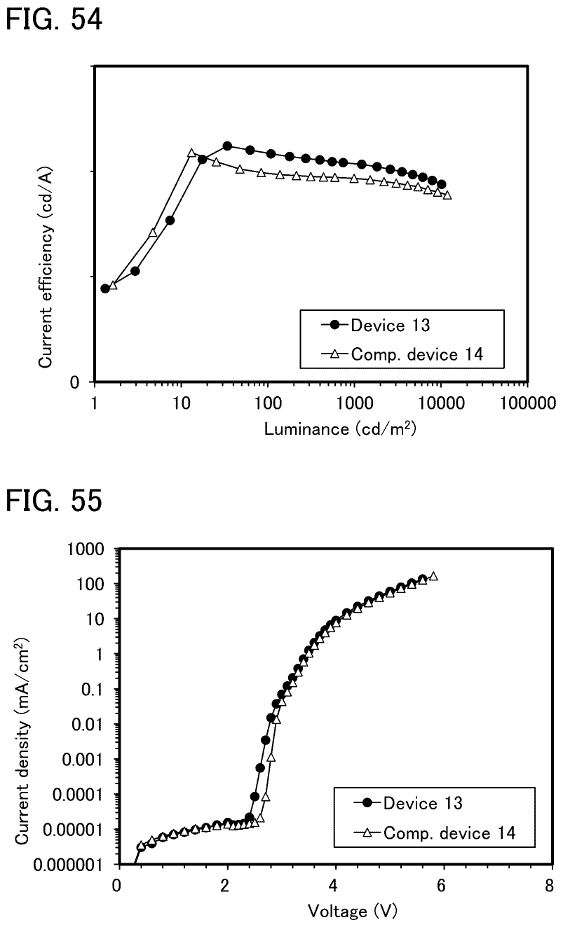

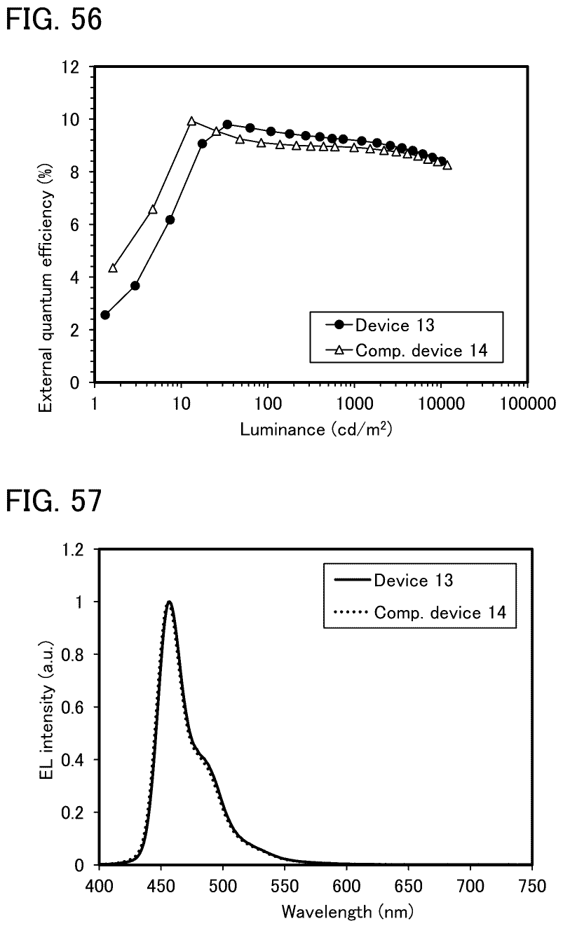

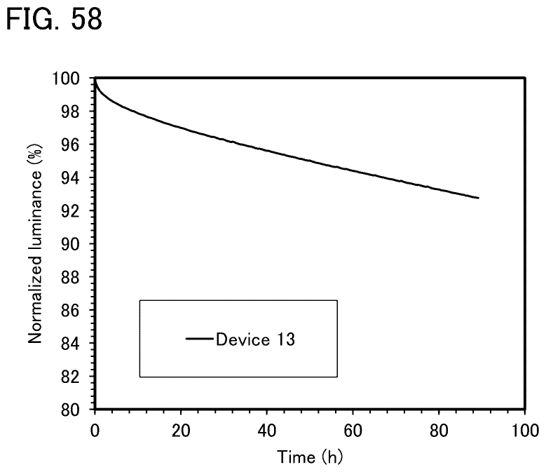

FIG. 52 shows luminance-current density characteristics of the light-emitting device 13 and the comparative light-emitting device 14. FIG. 53 shows luminance-voltage characteristics thereof. FIG. 54 shows current efficiency-luminance characteristics thereof. FIG. 55 shows current density-voltage characteristics thereof. FIG. 56 shows external quantum efficiency-luminance characteristics thereof. FIG. 57 shows electroluminescence spectra thereof. FIG. 58 shows a luminance change over driving time when the light-emitting device 13 was driven at a constant current of 2 mA (50 mA/cm²). Note that in the legends in FIG. 52 to FIG. 58, the light-emitting device 13 is denoted by Device 13 and the comparative light-emitting device 14 is denoted by Comp. device 14.

Table 7 shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured from the front of the emission surface of the substrate with the spectroradiometer, on the assumption that the devices had Lambertian light-distribution characteristics.

TABLE 7

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Light-emitting | 4.20 | 0.587 | 14.7 | 0.138 | 0.101 | 1213 | 8.26 | 9.17 |
| device 13 | | | | | | | | |
| Comparativelight- | 4.20 | 0.513 | 12.8 | 0.140 | 0.096 | 991 | 7.73 | 8.93 |
| emitting device 14 | | | | | | | | |

From FIG. 52 to FIG. 57 and Table 7, the light-emitting device 13 was found to be a light-emitting device with favorable characteristics that emits blue light derived from 3,10PCA2Nbf(IV)-02. From FIG. 58, the light-emitting device 13 was found to have a long lifetime.

FIG. 54, FIG. 56, and Table 7 show that the light-emitting device 13 has higher current efficiency and higher external quantum efficiency than the comparative light-emitting device 14. This indicates that the light-emitting device using FLP(2)NBBnf(II)(4), which is the organic compound of one embodiment of the present invention, in the second hole-transport layer 912_2 has higher emission efficiency than that using SFABnf(II)(4) as the comparative organic compound.

The above results show that the use of the organic compound of one embodiment of the present invention enables fabrication of a light-emitting device with favorable characteristics and a long lifetime.

This application is based on Japanese Patent Application Serial No. 2024-193065 filed with Japan Patent Office on Nov. 1, 2024, the entire contents of which are hereby incorporated by reference.

What is claimed is:

1. An organic compound represented by General Formula (G1), 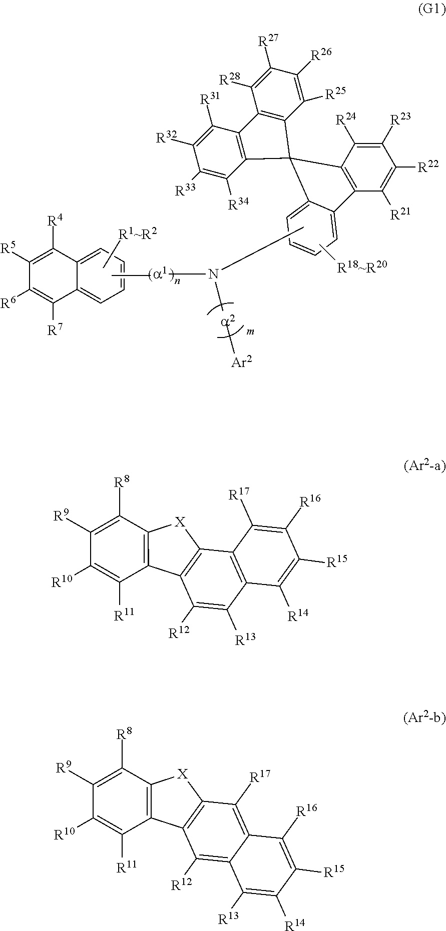
wherein:
α¹ represents a substituted or unsubstituted phenylene group;
n is 1 or 2;
α² represents a substituted or unsubstituted phenylene group or a substituted or unsubstituted naphthalene-diyl group;
m is 0, 1, or 2;
each of R¹ to R⁷ independently represents any one of hydrogen, a straight-chain or branched-chain alkyl group having 1 to 6 carbon atoms, a substituted or unsubstituted cycloalkyl group having 3 to 10 carbon atoms, and a substituted or unsubstituted phenyl group;
Ar² represents a group represented by General Formula (Ar²-a) or General Formula (Ar²-b);
any one of R⁸ to R¹⁷ represents a bond;
each of R⁸ to R¹⁷ other than the bond and each of R¹⁸ to R²⁸ and R³¹ to R³⁴ independently represents any one of hydrogen, a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, and a substituted or unsubstituted heteroaryl group having 2 to 30 carbon atoms; and
X represents oxygen or sulfur.

2. The organic compound according to claim 1,
wherein the organic compound is represented by General Formula (G2), and 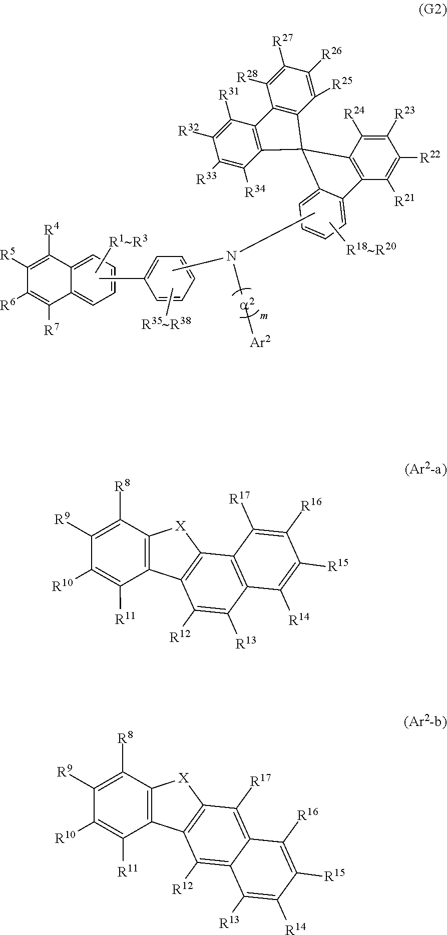
wherein each of R¹ to R⁷ and R³⁵ to R³⁸ independently represents any one of hydrogen, a straight-chain or branched-chain alkyl group having 1 to 6 carbon atoms, and a substituted or unsubstituted cycloalkyl group having 3 to 10 carbon atoms.

3. The organic compound according to claim 2, wherein m in General Formula (G2) is 0.

4. The organic compound according to claim 1,
wherein the organic compound is represented by General Formula (G4), and 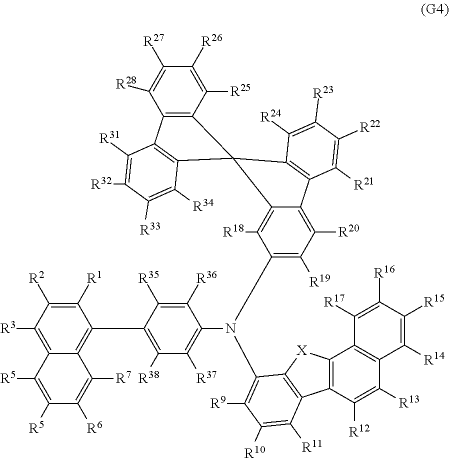
wherein:
each of R³⁵ to R³⁸ independently represents any one of hydrogen, a straight-chain or branched-chain alkyl group having 1 to 6 carbon atoms, and a substituted or unsubstituted cycloalkyl group having 3 to 10 carbon atoms; and
each of R⁹ to R¹⁷, R¹⁸ to R²⁸, and R³¹ to R³⁴ independently represents any one of hydrogen, a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, and a substituted or unsubstituted heteroaryl group having 2 to 30 carbon atoms.

5. The organic compound according to claim 1,
wherein the organic compound is represented by General Formula (G5), and 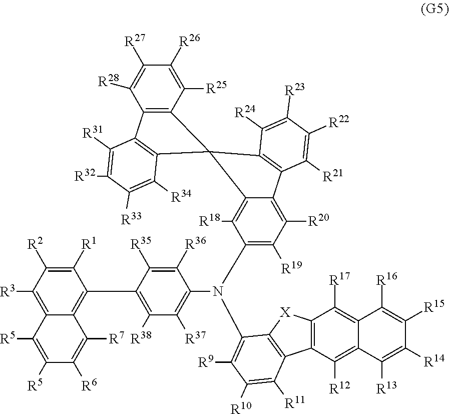
wherein:
each of R³⁵ to R³⁸ independently represents any one of hydrogen, a straight-chain or branched-chain alkyl group having 1 to 6 carbon atoms, and a substituted or unsubstituted cycloalkyl group having 3 to 10 carbon atoms; and
each of R⁹ to R¹⁷, R¹⁸ to R²⁸, and R³¹ to R³⁴ independently represents hydrogen, a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, or a substituted or unsubstituted heteroaryl group having 2 to 30 carbon atoms.

6. The organic compound according to claim 1,
wherein the organic compound is represented by Structural Formula (100) or Structural Formula (101),

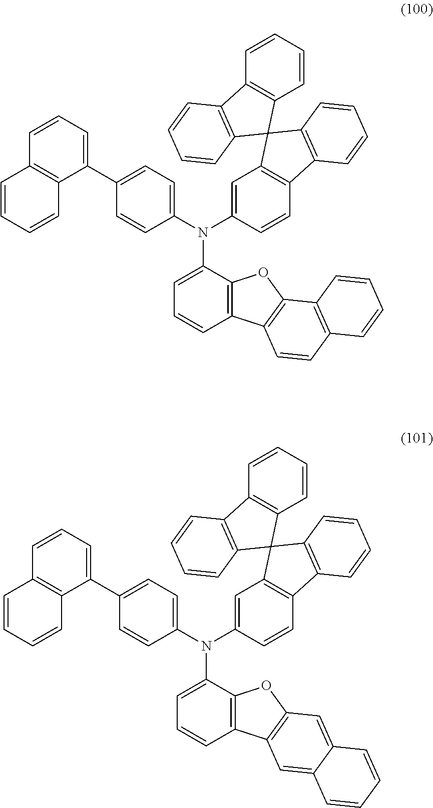 [SMILES: c1ccc2c(c1)-c1ccccc1C21c2ccccc2-c2ccc(N(c3ccc(-c4cccc5ccccc45)cc3)c3cccc4c3oc3c5ccccc5ccc43)cc21 ; c1ccc2c(c1)-c1ccccc1C21c2ccccc2-c2ccc(N(c3ccc(-c4cccc5ccccc45)cc3)c3cccc4c3oc3cc5ccccc5cc34)cc21]

7. An organic semiconductor device comprising:
the organic compound according to claim 1.

8. A light-emitting device comprising:
the organic compound according to claim 1.

9. An organic compound represented by General Formula (G7), 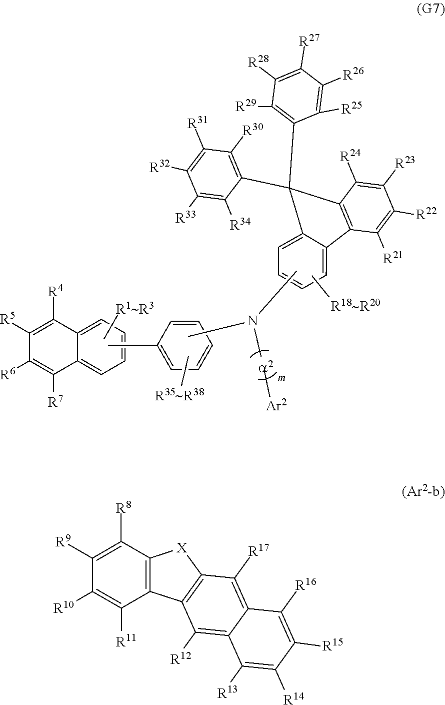
wherein:
α² represents a substituted or unsubstituted phenylene group or a substituted or unsubstituted naphthalene-diyl group;
m is 0, 1, or 2;
each of R¹ to R⁷ and R³⁵ to R³⁸ independently represents any one of hydrogen, a straight-chain or branched-chain alkyl group having 1 to 6 carbon atoms, and a substituted or unsubstituted cycloalkyl group having 3 to 10 carbon atoms;
Ar² represents a group represented by General Formula (Ar²-b);
any one of R⁸ to R¹⁷ represents a bond;
each of R⁸ to R¹⁷ other than the bond and each of R¹⁸ to R³⁴ independently represents any one of hydrogen, a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, and a substituted or unsubstituted heteroaryl group having 2 to 30 carbon atoms; and
X represents oxygen or sulfur.

10. The organic compound according to claim 9, wherein m is 0.

11. An organic semiconductor device comprising:
the organic compound according to claim 9.

12. A light-emitting device comprising:
the organic compound according to claim 9.

13. A light-emitting device comprising:
a first electrode;
a second electrode;
a light-emitting layer; and
a first layer,
wherein the light-emitting layer is between the first electrode and the second electrode,
wherein the first layer is between the first electrode and the light-emitting layer,
wherein the first layer comprises an organic compound represented by General Formula (G8), and 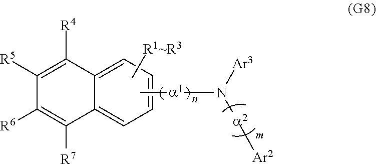
wherein:
each of α¹ and α² independently represents a substituted or unsubstituted phenylene group or a substituted or unsubstituted naphthalene-diyl group;
n is 1 or 2;
m is 0, 1, or 2;
each of R¹ to R⁷ independently represents any one of hydrogen, a straight-chain or branched-chain alkyl group having 1 to 6 carbon atoms, a substituted or unsubstituted cycloalkyl group having 3 to 10 carbon atoms, and a substituted or unsubstituted phenyl group;
Ar² represents any one of a substituted or unsubstituted benzo[b]naphtho[2,1-d]furanyl group, a substituted or unsubstituted benzo[b]naphtho[2,3-d]furanyl group, a substituted or unsubstituted benzo[b]naphtho[2,1-d]thiophenyl group, and a substituted or unsubstituted benzo[b]naphtho[2,3-d]thiophenyl group; and
Ar³ represents a substituted or unsubstituted fluorenyl group or a substituted or unsubstituted spirobifluorenyl group.

14. The light-emitting device according to claim 13,
wherein the organic compound in the first layer is represented by General Formula (G9), and 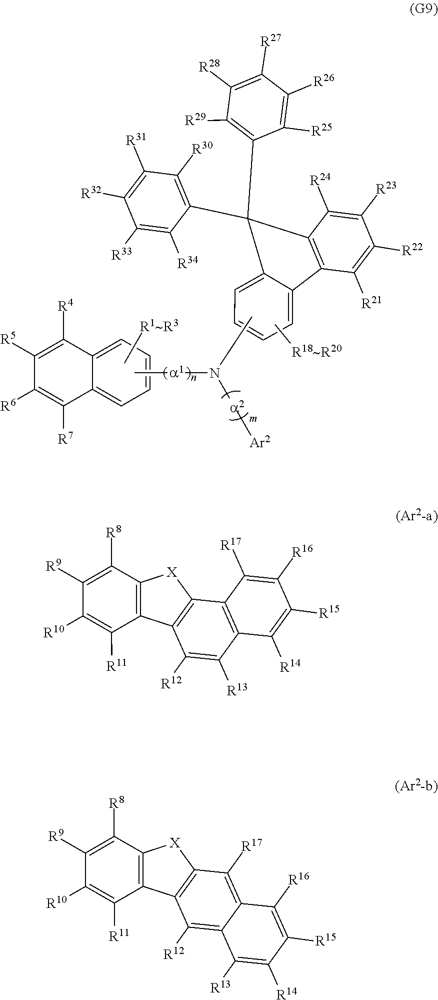
wherein:
Ar² represents a group represented by General Formula (Ar²-a) or General Formula (Ar²-b);
any one of R⁸ to R¹⁷ represents a bond;
each of R⁸ to R¹⁷ other than the bond and each of R¹⁸ to R²⁸ and R³¹ to R³⁴ independently represents any one of hydrogen, a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, and a substituted or unsubstituted heteroaryl group having 2 to 30 carbon atoms;
when each of R²⁹ and R³⁰ represents a bond, R²⁹ and R³⁰ are bonded to each other to form a ring;
when neither R²⁹ nor R³⁰ represents a bond, each of R²⁹ and R³⁰ independently represents any one of hydrogen, a halogen, a cyano group, a straight-chain or branched-chain alkyl group having 1 to 6 carbon atoms, an alkenyl group having 2 to 6 carbon atoms, an alkynyl group having 2 to 6 carbon atoms, a substituted or unsubstituted cycloalkyl group having 3 to 10 carbon atoms, an alkoxy group having 1 to 6 carbon atoms, a silyl group having 3 to 18 carbon atoms, a substituted or unsubstituted aryl group having 6 to 30 carbon atoms, and a substituted or unsubstituted heteroaryl group having 2 to 30 carbon atoms; and
X represents oxygen or sulfur.

15. The light-emitting device according to claim 13,
wherein the first layer is in contact with the light-emitting layer.

16. The light-emitting device according to claim 13,
wherein the first layer is in contact with the light-emitting layer,
wherein the light-emitting layer comprises a first host material, a second host material, and a light-emitting substance,
wherein the first host material and the second host material form an exciplex in combination, and
wherein a difference between a peak wavelength of an emission spectrum of the exciplex and a peak wavelength of an emission spectrum of the light-emitting substance is less than or equal to 30 nm.

17. The light-emitting device according to claim 13,
wherein the first layer is in contact with the light-emitting layer, and
wherein the light-emitting layer comprises a host material and a fluorescent substance.
